(19) United States
(12) Patent Application Publication
(10) Pub. No.: US 2025/0195685 A1
(43) Pub. Date: Jun. 19, 2025

(54) HUMANIZED ANTI-CD8 ANTIBODIES AND USES THEREOF

(71) Applicant: Capstan Therapeutics, Inc., San Diego, CA (US)

(72) Inventors: Duy Nguyen, San Diego, CA (US); Leonard G. Presta, San Francisco, CA (US); Alexander Martinko, San Francisco, CA (US); Qian-Chen Yong, San Diego, CA (US); Stuart Aaron Sievers, Van Nuys, CA (US)

(21) Appl. No.: 18/983,294

(22) Filed: Dec. 16, 2024

Related U.S. Application Data
(60) Provisional application No. 63/610,917, filed on Dec. 15, 2023.
(60) Provisional application No. 63/654,930, filed on May 31, 2024.
(60) Provisional application No. 63/708,461, filed on Oct. 17, 2024.

(51) Int. Cl. A61K 47/69; A61K 31/7105; A61K 47/68; C07K 16/28; C12N 15/88

(52) U.S. Cl. CPC A61K 47/6913; A61K 31/7105; A61K 47/6849; C07K 16/2815; C12N 15/88; C07K 2317/24; C07K 2317/52; C07K 2317/565; C07K 2317/567

(57) ABSTRACT

The present disclosure provides humanized antibodies and antigen binding domains thereof that bind CD8α and other antibody formats comprising these antigen binding domains, their use as a targeting moiety on lipid nanoparticles (tLNP) to deliver a therapeutic payload (such as a nucleic acid molecule) or other types of payloads. The present disclosure further relates to pharmaceutical compositions comprising the humanized anti-CD8α antibodies and CD8-targeted tLNP encapsulating a payload.

### CROSS-REFERENCE TO RELATED APPLICATIONS

This application claims priority U.S. provisional application No. 63/610,917, filed Dec. 15, 2023; U.S. provisional application No. 63/654,930, filed May 31, 2024; and U.S. provisional application No. 63/708,461, filed Oct. 17, 2024; the disclosures of which are expressly incorporated by reference herein.

### REFERENCE TO AN ELECTRONIC SEQUENCE LISTING

This application contains a Sequence Listing that has been submitted electronically and is hereby incorporated by reference in its entirety. The Sequence Listing was created on Dec. 15, 2024, is named “23-1742-US_SequenceListing.xml”, and is 250,755 bytes in size.

### BACKGROUND OF THE DISCLOSURE

Cluster of Differentiation 8 (CD8) antigen is a cell surface glycoprotein found on most cytotoxic T lymphocytes that helps to mediate efficient cell-cell interactions. The CD8 antigen binds class I major histocompatibility complex (MHC) molecules and acts as a co-receptor with the T cell receptor (TCR) on the T lymphocyte to recognize antigens displayed by an antigen presenting cell (APC) in the context of class I MHC molecules. The co-receptor functions as either a homodimer composed of two alpha chains (CD8 subunit alpha—CD8α) or as a heterodimer composed of one CD8α and one CD8 beta chain (CD8β).

CD8-positive T cells are mediators of adaptive immunity. They include cytotoxic T cells, which are important for killing cancerous, virally infected, or other pathogenic cells, and CD8-positive suppressor T cells, which restrain certain types of immune response.

A number of anti-CD8 antibodies have been developed. However, many have been produced in mice wherein these antibodies have the capacity to provoke an immune response if introduced into a human. Accordingly, there remains a need for a humanized (or human) anti-CD8 antibody that has reduced immunogenic potential while retaining bioactivity, inter alia, binding to CD8-expressing cells. In addition, such humanized anti-CD8 antibodies must also have biophysical properties that are suitable for clinical development and manufacturing. Thus, there is a need to develop improved anti-CD8 antibodies for therapeutic uses.

### BRIEF DESCRIPTION OF THE DRAWINGS

The disclosure will be better understood and features, aspects, and advantages other than those set forth above will become apparent when consideration was given to the following detailed description thereof. Such detailed description refers to the following drawings.

FIG. 1A shows humanized sequences of CBD1017vh (mouse anti-CD8 antibody clone CT8) variable heavy chain (VH) in IGHV1-46*01 (VH1-46) germline. Bolded texts show CDR regions based on the AbM definition (see bioinf.org.uk/abs/). Underlined text indicates framework residues back mutated to the amino acid found in the parental mouse antibody. Other potential changes are shown below the sequences. Seq: simple sequential amino acid numbering. AbM: Chothia amino acid numbering.

FIG. 1B shows humanized sequences of CBD1017vl (mouse anti-CD8 antibody clone CT8) variable light chain (VL) in IGKV1-39*01 (VK1-39) germline. Bolded texts show CDR regions based on the AbM definition. Underlined text indicates framework residues back mutated to the amino acid found in the parental mouse antibody. Other potential changes are shown below the sequences. Seq: simple sequential amino acid numbering. AbM: Chothia amino acid numbering.

FIG. 1C shows humanized sequences of CBD1017vh (mouse anti-CD8 antibody clone CT8) variable heavy chain (VH) in a modified IGHV1-18*01 (VH1-18) germline. Dark bars show CDR regions. Amino acid residues in CBD1017vh that differ from the VH1-18 sequence are shaded as are the residues in the humanized sequences that utilize the mouse residue. Simple sequential amino acid numbering was used.

FIG. 1D shows humanized sequences of CBD1017vl (mouse anti-CD8 antibody clone CT8) variable light chain (VL) in a modified IGVK3D-11*01 (VK3D-11) germline. Dark bars show CDR regions. Amino acid residues in CBD1017vh that differ from the VH1-18 sequence are shaded as are the residues in the humanized sequences that utilize the mouse residue. Simple sequential amino acid numbering is used.

FIG. 2A is a summary of the binding constants of different humanized anti-CD8 antibody binding fragments (Fab) based on VH1-46 and VK1-39 germlines as measured by biolayer interferometry (BLI) kinetic assays at 30° C. and 37° C.

FIG. 2B shows a binding kinetics sensorgram of CBD1033 Fab to CD8αα antigen at indicated concentrations in biolayer interferometry kinetic assays at 30° C. and 37° C. The global fitting was performed with a 1:1 fitting model using GatorOne software as described below using GatorOne software as described below.

FIG. 2C is a summary of the steady state binding constants of different humanized anti-CD8 antibodies with an hIgG1-LALAPA (human IgG1 (hIgG1) isotype with Fc-silencing mutations L234A, L235A, and P329A (LALAPA) (SEQ ID NO: 43) humanized with VH1-46 and VK1-39 germline framework regions as measured by biolayer interferometry kinetic assays.

FIG. 2D shows a binding kinetics sensorgram of the interaction of CBD1033 IgG1 with human CD8αα antigen at indicated concentrations in the biolayer interferometry kinetic assays. The global fitting was performed with a 1:1 fitting model using GatorOne software as described below.

FIG. 2E shows a steady state analysis of the humanized antibody, CBD1033, with a human IgG1 constant region with Fc-silencing mutations L234A, L235A, and P329A (LALAPA) (CBD1033-hIgG1-LALAPA) (SEQ IDNO: 43) interacting with human CD8αα based on the data in FIG. 2D.

FIG. 2F is a summary of the steady state binding constants of different humanized anti-CD8 antibodies with hIgG1-LALAPA (human IgG1 (hIgG1) isotype with Fc-silencing mutations L234A, L235A, and P329A (LALAPA) (SEQ ID NO: 43)) based on VH1-18 and VK3D-11 germlines interacting with human CD8αα as measured by biolayer interferometry kinetic assays.

FIG. 2G shows a biolayer interferometry binding kinetics sensorgram plots for the humanized CBD1043 anti-CD8 antibody at the indicated concentrations of human CD8αα used in the determination of K_{D} reported in FIG. 2F. Global fitting was performed with a 1:1 fitting model using GatorOne software as described below.

FIG. 2H shows a steady state analysis of the humanized CBD1043 anti-CD8 antibody-based on the data in FIG. 2G and used in the determination of K_{D} reported in FIG. 2F.

FIG. 2I is a summary of the binding constants of CBD1033 and CBD1017ch Fabs against human and cynomolgus CD8αα homodimer and CD8αβ heterodimer as measured by biolayer interferometry kinetic assays at 37° C. or 30° C.

FIG. 2J shows a binding kinetics sensorgram of humanized CBD1033 and CBD1017ch Fabs interacting with cynomolgus macaque CD8αα homodimer antigen at indicated concentrations in biolayer interferometry kinetic assays at 30° C. The global fitting was performed with a 1:1 fitting model using GatorOne software as described below.

FIG. 2K is a summary of the binding constants of CBD1033 whole antibody against human or cynomolgus macaque CD8αα homodimer or CD8αβ heterodimer as measured by surface plasmon resonance (SPR).

FIG. 2L shows binding kinetics sensorgrams of humanized CBD1033 whole antibody to human or cynomolgus CD8αα homodimer or CD8αβ heterodimer in SPR assays. Except for cynomolgus CD8αβ heterodimer, binding was analyzed with a two-fold dilution series of recombinant CD8αα homodimer or CD8αβ heterodimer protein from 200 nM to 6.25 nM. For cynomolgus CD8αβ heterodimer, binding was analyzed with a two-dilution series from 400 nM to 12.5 nM.. The global fitting was performed with a 1:1 fitting model using GatorOne software as described below.

FIG. 3A shows binding of anti-CD8 whole antibodies with a human IgG1 (hIgG1) isotype with Fc-silencing mutations L234A, L235A, and P329A (LALAPA) (SEQ ID NO: 43)) at different concentrations to CD8-overexpressing HEK293T cells using normalized geometric median fluorescence intensity (gMFI) assays. Binding was measured by fluorescence from anti-human Fc secondary antibody conjugated to a BV421 fluorophore that recognized the binders. The gMFI was normalized to the gMFI of the secondary antibody in the absence of the anti-CD8α binders.

FIG. 3B is a summary of the EC₅₀ of anti-CD8α antibodies determined by the normalized gMFI in experiments shown in FIG. 3A.

FIG. 4A through FIG. 4D show transfection rates (FIGS. 4A-4B) and expression levels (FIGS. 4C-4D) of primary human T cells derived from different donors using a set of targeted lipid nanoparticles (tLNPs) conjugated with indicated anti-CD8 antibodies and encapsulating mCherry-encoding mRNA. The tLNPs added to the cells provided 0.6, 0.3, 0.15, 0.075, and 0 μg of mRNA. The transfection rate was measured by the percentage of CD4− CD8+ T cells expressing mCherry.

FIG. 5A through FIG. 5C show comparisons of mCherry expression following transfection with mCherry-encoding mRNA encapsulated in CD8-targeted tLNPs using CBD1017ch; humanized anti-CD8 binders derived from anti-CD8 antibody clones CT8 and OKT8; and cetuximab (negative control) as the targeting moieties.

FIG. 6 shows the fold-increase of normalized gMFI of fluorescence measured by flow cytometry upon binding of the anti-CD8α binder CBD1033 at different concentrations to CD8-expressing lymphoma T cells SupT1 and HPB-ALL. Binding was measured by fluorescence from the anti-human Fc secondary antibody conjugated to a BV421 fluorophore that recognize the binders. The gMFI was normalized to the gMFI of the secondary antibody in the absence of the anti-CD8α binder.

FIG. 7A and FIG. 7B show transfection rates (% mCherry positive) and % mCherry positivity vs molecules of equivalent soluble fluorochrome (MESF) as measured by flow cytometry of cynomolgus macaque CD8+ T cells transfected with mCherry-encoding mRNA encapsulated in anti-CD8α binder-targeted lipid nanoparticles (tLNPs).

FIG. 8A and FIG. 8B are normalized to show the fold increase over background of gMFI measured by flow cytometry demonstrating binding of the chimeric anti-CD8α binder and humanized anti-CD8α binder respectively at different concentrations to CD4− CD8+ rhesus, cynomolgus macaque, or human T cells. Binding was measured by fluorescence from the BV421 fluorophore-conjugated anti-human Fc secondary antibody that recognize the binders. The gMFI was normalized to the gMFI of the secondary antibody in the absence of the anti-CD8α binders.

FIG. 9A and FIG. 9B list EC₅₀ values in μg/mL and nM respectively showing comparable CD8-specificity across T cells from different species (cross-species) of the anti-CD8 binders, and their lack of binding to CD4+ T cells in comparison with humanized 5D7 antibody (anti-CD5 antibody).

FIG. 10 shows efficient and cell specific in vivo delivery of mCherry mRNA to CD8+ T cells using anti-CD8 binder CBD1033 Fc silenced IgG1 whole antibody as a targeting moiety on a tLNP. The antibody to mRNA (w/w) ratios of the tLNPs were 0.37, 0.72, and 1.09 as indicated.

FIG. 11A shows affinity-capture self-interaction nanoparticle spectroscopy (AC-SINS) assay scores of 6 anti-CD8α hIgG1-LALAPA antibodies. All 6 binders showed low propensity to self-associate. Bococizumab was used as a positive control while Alirocumab and Bococizumab NEI were used as negative controls.

FIG. 11B is a graphical summary of the results shown in FIG. 11A. Results for the positive and negative controls are plotted as horizontal lines and for the 6 anti-CD8α antibodies as circles.

FIG. 12A shows a dot plot distribution of melting temperatures (Tₘ) of anti-CD8α hIgG1-LALAPA antibodies. The shaded area, above 65° C., indicates a range of good developability. All results were within ranges sufficient for chemistry, manufacturing, and controls (CMC) developability.

FIG. 12B is a summary of Tₘ as measured by differential scanning fluorimetry (DSF) and aggregation temperature (T_{agg}) as measured by static light scattering (SLS) of anti-CD8 antibodies.

FIG. 13A shows a dot plot distribution of polyreactive ELISA scores of 6 anti-CD8 binders to double strand DNA (dsDNA) and insulin.

FIG. 13B is a table of the experimental values plotted in FIG. 13A.

FIG. 14 depicts data from a baculovirus particle (BVP) polyreactivity ELISA. PC: positive control. NC: negative control. The assay measured non-specific binding to an array of membrane proteins on the baculovirus particles which carried a plurality of proteins from the host cells from which the virus was produced.

FIG. 15 shows binding interactions of anti-CD8α binders to human cell membrane proteins in a membrane proteome array assay. No additional significant specific interactions, aside from the gene product of CD8A, was observed.

FIG. 16A shows percentage of deamidation at N55 position in VH-CDR1 region at high pH (8.5) and high temperature (40° C.) for CBD1017ch after 7 days under these conditions.

FIG. 16B shows percentage of deamidation at N55 position in VH-CDR2 region at high pH (8.5) and high temperature (40° C.) for anti-CD8α binders CBD1033, CBD1035, and CBD1039 after 7 days under these conditions.

FIG. 16C shows percentage of deamidation at N55 position in VH-CDR2 region at high pH (8.5) and high temperature (40° C.) over time for CBD1033.

FIG. 16D shows minimal loss of binding affinity of anti-CD8α binders due to high pH stress.

FIG. 16E shows the effect of mutations in the N55 position of VH-CDR2 region on binding affinity.

FIG. 16F shows the effect of mutations in the N55 position of VH-CDR2 region or D30 position of VL-CDR1 or both on binding affinity of Fab fragments.

FIG. 17A and FIG. 17B show reduction in mCherry transfection by anti-CD8α tLNP in two separate donors by the mutation of N55 to aspartate (D) mimicking deamidation. Mutations in the N55 position of VH-CDR2 region to encode glutamine (Q), serine (S), or alanine (A) do not lead to this loss of function.

FIG. 17C is a summary of the mutations made and tested in FIG. 17A and FIG. 17B.

FIG. 18A and FIG. 18B show the transfection efficiency and expression levels of mCherry, respectively, in T cells by anti-CD8α-targeted tLNPs. The anti-CD8α antigen binding domain CBD1033 in various antibody formats (diabody synthetic hinge [Groups (GRP) 1-3], diabody IgG3 hinge [Groups 4-7], recombinant IgG1 F(ab′)₂ [Groups 8-10], recombinant IgG4 F(ab′)₂ [Groups 11-13], or IgG1 F(ab′)₂ enzymatically digested [Groups 14-16]) was conjugated to tLNPs. Antibody density and reducing condition were also varied (see Example 8 and Table 15 therein for details). tLNPs with CBD1033 Fc silenced IgG1 whole antibody was used as a positive control (Group 17) and non-transfected cells (NTD) as a negative control.

FIG. 19A summarizes the design strategy for disulfide engineered F(ab′) constructs.

FIG. 19B shows absence of F(ab′) dimer due to purification under reducing conditions without disruption of the engineered interchain disulfide bond of the F(ab′).

FIG. 20A shows sodium dodecyl sulfate-polyacrylamide gel electrophoresis (SDS-PAGE) analysis of anti-CD8 F(ab′) analogs CBD1033.37 or CBD1033.24 under fully reducing (R) or non-reducing (NR) conditions.

FIG. 20B shows 280 nm absorbance peaks corresponding to anti-CD8 Fab′ binders CBD1033.37 and CBD1033.24 in size exclusion high performance liquid chromatography (SEC-HPLC) chromatograms.

FIG. 20C shows results obtained from non-reducing liquid chromatography mass spectrometry (LC-MS) analysis of anti-CD8 F(ab′) fragments of CBD1033.37 or CBD1033.24 with high abundance peaks corresponding to F(ab′) fractions.

FIG. 21A is a summary of the binding constants of various engineered, anti-CD8 binding F(ab′) fragments as measured by biolayer interferometry kinetic assays.

FIG. 21B shows a binding kinetics sensorgram of CBD1033.37 F(ab′) interacting with the CD8αα homodimer at indicated concentrations in a biolayer interferometry kinetic assay. The global fitting was performed with a 1:1 fitting model using GatorOne software as described below.

FIG. 22A demonstrates the conjugation reaction of humanized anti-CD8 F(ab′) with maleimide-PEG-biotin.

FIG. 22B shows immunoblotting of biotin conjugateed F(ab′) analogs detected by streptavidin-horseradish peroxide (HRP).

FIG. 22C shows 280 nm absorbance peaks corresponding to biotin-conjugated F(ab′) fractions in SEC-HPLC chromatograms.

FIG. 22D shows results obtained from LC-MS analysis of maleimide-PEG-biotin conjugated CBD1033.24 and CBD1033.37 with high abundance peaks corresponding respective to F(ab′) fractions.

FIG. 22E shows the abundance of detected modifications on conjugated CBD1033.24 and CBD1033.37 from peptide mapping analysis. The results demonstrate the site-specific and quantitative conjugation of maleimide-PEG-biotin to CBD1033.24 and CBD1033.37. Amino acid position follows sequential numbering. C227 and C230 correspond to C239 in Kabat numbering of IgG4 and IgG1, respectively.

FIG. 23A is a summary of the binding constants of maleimide-PEG-biotin conjugated CBD1033.24 and CBD1033.37 F(ab′) against human CD8α mouse Fc fusion protein as measured by biolayer interferometry kinetic assays at 37° C.

FIG. 23B shows a binding kinetics sensorgram of maleimide-PEG-biotin conjugated CBD1033.37 to human CD8α mouse Fc fusion protein at indicated concentrations in biolayer interferometry kinetic assays at 37° C. The global fitting was performed with a 1:1 fitting model using GatorOne software as described below.

FIG. 24A shows mCherry expression levels as gMFI of primary human activated T cells transfected with anti-CD8α F(ab′)-conjugated lipid nanoparticles (tLNPs) encapsulating mCherry-encoding mRNA. The tLNPs added to the cells were formulated with binder to mRNA ratios (w/w) of 0.1, 0.3, 0.5, 0.75, or 0.35. The transfection rate was measured by the percentage of CD4− CD8+ T cells expressing mCherry. tLNP conjugated to CBD1033.29, a whole IgG1 comprising the LALAPA Fc silencing mutations (CBD1033.3) and thiolated by the AJICAP process, was used a positive control.

FIG. 24B shows efficient and cell-specific in vivo delivery of mCherry mRNA to human CD8+ T cells in blood and spleen of NCG mice engrafted with human PBMCs using native and engineered disulfide anti-CD8 F(ab′) as the targeting moiety on a tLNP. The antibody to mRNA (w/w) ratio on the tLNPs was 0.35 for the whole antibody (CBD1033.29) and 0.3 for the F(ab′)s. CBD1033.29 is the CBD1033 antigen binding domain joined to SEQ ID NO: 43, an IgG1 constant region bearing the Fc silencing LALAPA mutations (CBD1033.3) and thiolated by the AJICAP process.

FIG. 25A and FIG. 25B show expression levels in expanded human CD8+ T cells from two donors transfected in vitro with a modified tLNP-98219 in which various engineered anti-CD8 F(ab′) analogs have been substituted for whole antibody as the targeting moiety. Transfections were carried out with a dose of 0.6 pg of mRNA in duplicates. CAR expression from transfection of CD8+ T cells from two donors with each tLNP in duplicates is shown as transfection efficiency (percentage CAR+) (FIG. 25A) and CAR expression level (median fluorescence intensity) (FIG. 25B) at 24 hours post transfection. tLNP-98219 is anti-CD8 targeting composition F9 tLNP encapsulating RM_61461 mRNA (SEQ ID NO: 195) encoding anti-CD19 CAR2. The CBD1033.29 positive control was CBD1033.3 Fc-silenced whole antibody IgG1 (LALAPA) thiolated by the AJICAP process and conjugated to the LNP.

FIG. 26A and FIG. 26B show CAR transfection efficiency of CD4+ and CD8+ T cells, respectively, from expanded human T cells of two donors transfected with anti-CD8-targeted tLNP encapsulating anti-CD19 CAR-encoding mRNA. Two mRNA constructs encoding anti-CD19 were used: improved RM_61461 construct (SEQ ID NO: 195) or base RM_61512 construct (SEQ ID NO: 196). The improved construct was known to have higher expression than the base construct and both were used as assay controls. Group 1-16 were tLNPs conjugated to various anti-CD8 F(ab′) with different antibody format designs and liability-engineered mutations in the variable domains. Group 1-16 expressed the improved mRNA, RM_61461 (SEQ ID NO: 195). The improved (RM_61461) and base (RM_61512) control mRNAs were encapsulated in tLNP in which the targeting moiety comprised CBD1033.29 conjugated to the tLNP (control improved and control base respectively).

FIG. 26C and FIG. 26D show CAR expression levels, as measured by phycoerythrin fluorescence, of CD4+ and CD8+ T cells, respectively in similar experiments to FIG. 26A and FIG. 26B.

FIG. 26E shows the CBD number and the summary of the design and the liability-engineered mutations of each group in FIG. 26A-26D.

FIG. 27A is the workflow of the cross-linking mass spectrometry study to identify interaction sites between an antibody and its antigen. This was applied to identification of CBD1033's epitope.

FIG. 27B demonstrates identification and mapping of crosslinked amino acid positions on an existing structural model of human CD8αα homodimer.

FIG. 27C shows structure of human CD8αα homodimer with identified epitope.

FIG. 28A shows a binding kinetics sensorgram of a competition binding experiment between CBD1033.3 and either OKT8 or TRX2. After capturing CD8αα, CBD1033.3 was loaded to form a complex with CD8αα. While subsequent addition of OKT8 resulted in a response shift indicating binding with the CD8αα-CBD1033.3 complex, TRX2 did not cause a response shift, indicating that TRX2 could not bind to the CD8αα-CBD1033.3 complex.

FIG. 28B shows a summary of competition binding to human CD8αα homodimer, with spectral shift values under 0.7 indicating competitive binding.

FIG. 28C represents two epitope bins with each group containing the same or overlapping epitopes bound by the indicated antibodies.

FIG. 29A shows mCherry expression levels in vitro in primary human T cells using CBD1033, TRX2, SK1, OKT8, humanized OKT8 variant 1 (VL and VH as shown Table 16 as SEQ ID NO: 229 and 230 respectively, taken from U.S. Ser. No. 11/254,744B2), or humanized OKT8 variant 2 (LC and HC as shown Table 16 as SEQ ID NO: 231 and 232 respectively, taken from U.S. Pat. No. 11,739,150B2) antibody as binding moiety of tLNP encapsulating mRNA-encoded mCherry.

FIG. 29B show comparable in vivo mCherry delivery to CD8+ T cells in blood or spleen tissues using CBD1033 or TRX2 antibody as binding moiety of tLNP encapsulating mRNA-encoded mCherry.

### DESCRIPTION OF THE DISCLOSURE

Provided herein are humanized antibody antigen binding domains that specifically bind CD8α (also known as CD8a, and CD8 alpha, and the encoding gene as CD8A), whole antibody and other antibody formats comprising these antigen binding domains, their use as a targeting moiety in lipid nanoparticles (tLNP) to deliver a payload (e.g., a nucleic acid molecule), and compositions of the anti-CD8α tLNPs. Also provided herein are compositions comprising humanized anti-CD8α antibodies, anti-CD8 tLNPs encapsulating a payload, and methods of using the same. In particular embodiments, the payload is an mRNA. In further embodiments, the mRNA encodes a protein the reprograms the antigen specificity of CD8+ cells. In some embodiments, the reprograming agent encoded by the mRNA is a chimeric antigen receptor (CAR), a T cell receptor (TCR), or a T cell engager.

Specifically provided herein is a CD8α binding moiety comprising an immunoglobulin antigen binding domain that specifically binds to human CD8α in the CD8αα homodimer and the CD8αβ heterodimer comprising framework regions derived from human germline heavy and light chain variable domain genes.

In some embodiments, a humanized anti-CD8α antibody and antigen binding fragments thereof of this disclosure specifically bind to both human and non-human primate (NHP) CD8. In certain embodiments, an isolated humanized anti-CD8α monoclonal antibody or antigen binding fragment thereof of this disclosure has a temperature of aggregation (T_{agg})≥60° C. and a melting temperature of ≥65° C., a low propensity for self-interaction (i.e., tendency for aggregation), preserves an absence of cross-reactivity of CT8, and lacks polyreactivity to (a) double-stranded DNA and insulin; (b) baculovirus particles; or (c) human cell surface and secreted proteins.

A further aspect is a tLNP comprising an anti-CD8 targeting moiety wherein the targeting moiety binds a membrane-proximal epitope close to the CD8 dimerization interface. The antibodies CT8, TRX2, and YTC182.20 compete for binding to the epitope. The epitope is a structural (that is, non-linear) epitope comprising or adjacent to amino acids 40-47, 86-95, and 103-106 of CD8α. This epitope, whether defined by cross-competition of antibody binding, antibody-antigen cross-linking, or location within the secondary or tertiary structure of CD8, will be referred to herein as the CT8 epitope. Accordingly, the antigen binding domains of CT8, TRX2, and YTC182.20 constitute means for binding this CT8 epitope or means for targeting a tLNP to CD8+ cells or CD8+ T cells. tLNPs in which the targeting moiety comprises an antigen binding domain that binds the CT8 epitope transfect CD8+ cells more efficiently than tLNP in which the targeting moiety comprises an antigen binding domain recognizing some other CD8α epitopes. Accordingly, tLNPs in which the targeting moiety comprises an antigen binding domain that binds the CT8 epitope constitute means for efficiently transfecting CD8+ cells or CD8+ T cells.

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

### Definitions

Prior to setting forth this disclosure in more detail, it may be helpful to an understanding thereof to provide definitions of certain terms to be used throughout the disclosure. Additional definitions are set forth throughout the disclosure.

Throughout this specification, unless the context specifically indicates otherwise, the terms “comprise” and “include” and variations thereof (e.g., “comprises,” “comprising,” “includes,” and “including”) are understood to indicate the inclusion of a stated component, feature, element, or step or group of components, features, elements, or steps but not the exclusion of any other component, feature, element, or step or group of components, features, elements, or steps. Any of the terms “comprising”, “consisting essentially of”, and “consisting of” may be replaced with either of the other two terms, while retaining their ordinary meanings.

As used herein, the singular forms “a,” “an,” and “the” include plural referents unless the context clearly indicates otherwise. It should be understood that the terms “a” and “an” as used herein refer to “one or more” of the enumerated components.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein and in the drawings, ranges and amounts can be expressed as “about” a particular value or range. About also includes the exact amount. For example, “about 5%” means “about 5%” and also “5%.” The term “about” can also refer to +10% of a given value or range of values. For example, about 5% means 4.5%-5.5%.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

Throughout this disclosure, any concentration range, percentage range, ratio range, or integer range is to be understood to include the value of any integer within the recited range and, when appropriate, fractions thereof (such as one tenth and one hundredth of an integer), unless otherwise indicated. Also, any number range of this disclosure relating to any physical feature, such as polymer subunits, size, or thickness, are to be understood to include any integer within the recited range, unless otherwise indicated. Throughout this disclosure, numerical ranges are inclusive of their recited endpoints, unless specifically stated otherwise.

The phrase “at least one of” when followed by a list of items or elements refers to an open-ended set of one or more of the elements in the list, which may, but does not necessarily, include more than one of the elements.

As used herein, the term “subject” refers to a warm-blooded animal such as a mammal, preferably a human, or a human child, which is afflicted with, or has the potential to be afflicted with one or more diseases and disorders.

“Derivative,” as used herein, refers to a chemically or biologically modified version of a compound that is structurally similar to a parent compound and (actually or theoretically) derivable from that parent compound. Generally, a “derivative” differs from an “analogue” in that a parent compound can be the starting material to generate a “derivative,” whereas the parent compound is not necessarily used as the starting material to generate an “analogue.” For example, a derivative may be more hydrophilic or hydrophobic, or it may have altered reactivity as compared to the parent compound. Although a derivative can be obtained by physical (for example, biological or chemical) modification of the parent compound, a derivative can also be conceptually derived, for example, as when a protein sequence is designed based on one or more known sequences, an encoding nucleic acid is constructed, and the derived protein obtained by expression of the encoding nucleic acid.

As used herein, “lipid nanoparticle” (LNP) means a solid particle, as distinct from a liposome having an aqueous lumen. The core of an LNP, like the lumen of a liposome, is surrounded by a layer of lipid that can be, but is not necessarily, a continuous lipid monolayer, a bilayer, or multi-layer having three or more lipid layers.

“Artificial sequence,” or “synthetic sequence” as used herein, refers to an amino acid or nucleotide sequence that is devised to serve a specific purpose and that is not derived from a particular sequence existing in nature. The purpose of such sequences can include linkers, spacers, restrictions sites, and untranslated regions, among others.

As used herein “transfection” or “transfecting” refers to the introduction of nucleic acids into cells by non-viral methods. Transfection can be mediated by calcium phosphate, cationic polymers, magnetic beads, electroporation, and lipid-based reagents. In preferred embodiments disclosed herein transfection is mediated by solid lipid nanoparticles (LNP) including targeted LNP (tLNP). The term transfection is used in distinction to transduction—transfer of genetic material from cell to cell or virus to cell—and transformation—the uptake of extracellular genetic material by the natural processes of a cell. As used herein, phrases such as “delivering a nucleic acid into a cell” are synonymous with transfection.

“Reprogramming,” as used herein with respect to immune cells, refers to changing the functionality of an immune cell with respect to antigenic specificity by causing expression of an exogenous T cell receptor (TCR), a chimeric antigen receptor (CAR), or an immune cell engager (collectively termed “reprogramming agents”). Generally, T lymphocytes and natural killer (NK) cells can be reprogrammed with a TCR, a CAR, or an immune cell engager while only a CAR or an immune cell engager is used in reprogramming monocytes. In the case of an immune cell engager, the immune cells engaged and redirected against the pursued antigen of the immune cell engager are reprogrammed cells whether or not they express the reprogramming agent. Reprogramming can be transient or durable depending on the nature of the engineering agent.

“Engineering agent,” as used herein, refers to agents that confer the expression of a reprogramming agent by an immune cell, particularly a non-B lymphocyte or monocyte. Engineering agents can include nucleic acids, including mRNA that encode the reprogramming agent. Engineering agents can also include nucleic acids that are or encode components of gene editing systems such as RNA-guided nucleases, guide RNA, and nucleic acid templates for knocking-in a reprogramming agent or knocking-out an endogenous antigen receptor. Gene editing systems comprise base-editors, prime-editors or gene-writers. RNA-guided nucleases include CRISPR nucleases such as Cas9, Cas12, Cas13, Cas3, CasMINI, Cas7-11, and CasX. For transient expression of a reprogramming agent, such as a CAR, an mRNA encoding the reprogramming agent can be used as the engineering agent. For durable expression of the reprogramming agent, such as an exogenous, modified, or corrected gene (and its gene product), the engineering agent can comprise mRNA-encoded RNA-directed nucleases, guide RNAs, nucleic acid templates and other components of gene/genome editing systems.

Examples of gene editing components that are encoded by a nucleic acid molecule include an mRNA encoding an RNA-guided nuclease, a gene or base editing protein, a prime editing protein, a Gene Writer protein (e.g., a modified or modularized non-long terminal repeat (LTR) retrotransposon), a retrotransposase, an RNA writer, a zinc finger nuclease (ZFN), a transcription activator-like effector nuclease (TALEN), a meganuclease, a transposase, a retrotransposon, a reverse transcriptase (e.g., M-HLV reverse transcriptase), a nickase or inactive nuclease (e.g., Cas9, nCas9, dCas9), a DNA recombinase, a CRISPR nuclease (e.g., Cas9, Cas12, Cas13, Cas3, CasMINI, Cas7-11, CasX), a DNA nickase, a Cas9 nickase (e.g., D10A or H840A), or any fusion or combination thereof. Other components include a guide RNA (gRNA), a single guide RNA (sgRNA), a prime editing guide RNA (pegRNA), a clustered regularly interspaced short palindromic repeat (CRISPR) RNA (crRNA), a trans-activating clustered regularly interspaced short palindromic repeat (CRISPR) RNA (tracrRNA), or a DNA molecule to be inserted or serve as a template for double-strand break (DSB) repair at a specific genomic locus. Genome-, gene-, and base-editing technology are reviewed in Anzalone et al., Nature Biotechnology 38:824-844, 2020, Sakuma, Gene and Genome Editing 3-4:100017, 2022, and Zhou et al., MedComm 3(3):e155, 2022, each of which is incorporated by reference for all that they teach about the components and uses of this technology to the extent that it does not conflict with the present disclosure.

“Target antigen” or “targeted antigen”, as used herein refers to a surface antigen of an immune cell that can be specifically bound by the targeting moiety of a tLNP.

“Pursued antigen,” as used herein, refers to the antigen recognized by the reprogramming agent (such as a TCR, CAR or immune cell engager). It is common in the art to use the term target (or targeted) antigen with reference to any antigen that is bound by an antigen (or other) receptor. This has potential to be confusing where two distinct functional classes of antigen are concerned. In an effort to avoid this confusion, target (or targeted) antigen has been used herein to refer to the antigen bound by the targeting moiety of a nanoparticle and pursued antigen (or cell or tissue or indication, etc.) has been used to refer to an antigen bound by a reprogramming agent. (The substitution is not used in the terms “effector to target ratio,” “target cell, “off-target,” and “on-target” as that would tend to increase potential confusion rather than reduce it.) In the treatment of diseases, the pursued antigen will be expressed by a pathogenic cell but may also be expressed by normal cells.

“Conditioning agent,” as used herein, refers to a biological response modifier (BRM) that enhances the efficiency of engineering an immune cell, expands the number of immune cells available to be engineered or the number of engineered cells in a target tissue (for example, a tumor, fibrotic tissue, or tissue undergoing autoimmune attack), promotes activity of the engineered cell in a target tissue, or broadens the range of operative mechanisms contributing to a therapeutic immune reaction. A conditioning agent may be provided by delivering an encoding nucleic acid in a tLNP. Exemplary BRMs include cytokines, such as IL-7, IL-15, or IL-18.

The term “immune cell,” as used herein, can refer to any cell of the immune system. However, particular aspects can exclude polymorphonuclear leukocytes and/or B cells, or be limited to non-B lymphocytes such as T cell and/or NK cells, or to monocytes such as dendritic cells and/or macrophages in their various forms.

The term “nucleic acid” or “nucleic acid molecule,” as used herein, refers to either an RNA or DNA molecule, especially those encoding an expressible polypeptide, where context does not dictate otherwise. Description of disclosed (t)LNP payloads focuses on mRNA molecules having the structure of a canonical mRNA. However, polypeptides can also be encoded in and expressed from circular and self-amplifying (also known as self-replicating) RNA molecules. Accordingly, the sequence of any of the herein disclosed linear mRNA molecules can be incorporated into a circular or self-amplifying/self-replicating RNA molecule. Similarly, each of these RNA molecules can be encoded as a DNA molecule. Each of the disclosed nucleic acid sequences, RNA or DNA, should be understood to disclose the corresponding DNA or RNA sequence, respectively.

As used herein, “antibody” refers to a protein comprising an immunoglobulin domain having hypervariable regions determining the specificity with which the antibody binds antigen, termed complementarity determining regions (CDRs). The term antibody can thus refer to whole antibodies (also referred to as intact or full-length antibodies) as well as antibody fragments and constructs comprising an antigen binding portion of a whole antibody. While the canonical natural antibody has a pair of heavy and light chains, camelids (from camels, alpacas, llamas, and the like) produce antibodies with both the canonical structure and antibodies comprising only heavy chains. The variable region of the camelid heavy chain-only antibody has a distinct structure with a lengthened CDR3 referred to as VHH or, when produced as a fragment, a nanobody. Antigen binding fragments and constructs of antibodies include F(ab′)₂, F(ab′), F(ab), (sometimes equivalently denoted Fab′₂, Fab′, and Fab) minibodies, Fv, single-chain Fv (scFv), diabodies, and VH. Such elements may be combined to produce bi- and multi-specific reagents, such as BiTEs (bi-specific T-cell engagers). The term “monoclonal antibody” arose out of hybridoma technology but is now used to refer to any singular molecular species of antibody regardless of how it was originated or produced. Similarly, the terms F(ab) F(ab′), and Fc originated from the proteolytic analysis of antibodies but now refer to such fragments however obtained and whether or not they have the precise termini produced by the historic proteolysis. Antibodies can be obtained through immunization, selection from a naïve or immunized library (for example, by phage display), alteration of an isolated antibody-encoding sequence, or any combination thereof. Numerous antibodies that can be used as binding moieties are known in the art. An excellent source of information about antibodies for an International Non-proprietary Name (INN) has been proposed or recommended, including sequence information, is Wilkinson & Hale, 2022, MAbs 14(1):2123299, including its Supplementary Tables, which is incorporated by reference herein for all that it teaches about individual antibodies and the various antibody formats that can be constructed. U.S. Pat. No. 11,326,182 and especially its Table 9 entitled “Cancer, Inflammation and Immune System Antibodies,” is a source of sequence and other information for a wide range of antibodies including many that do not have an INN and is incorporated herein by reference for all that it teaches about individual antibodies.

An antibody or a binding fragment thereof or other binding moiety (or a fusion protein thereof) “specifically binds” to a target if it binds the target with an affinity or Kₐ (i.e., an equilibrium association constant of a particular binding interaction with units of 1/M) equal to or greater than 10⁵ M⁻¹, while not significantly binding other components present in a test sample. Binding domains (or fusion proteins thereof) can be classified as “high affinity” binding domains (or fusion proteins thereof) and “low affinity” binding domains (or fusion proteins thereof). “High affinity” binding domains refer to those binding domains with a Kₐ of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Kₐ of up to 10⁸ M⁻¹, up to 10⁷ M⁻¹, up to 10⁶ M⁻¹, up to 10⁵ M⁻¹. Alternatively, affinity may be defined as an equilibrium dissociation constant (K_{D}) of a particular binding interaction with units of Molarity (M) (e.g., 10⁻⁵ M to 10⁻¹³ M). Affinities of binding domain polypeptides and fusion proteins according to this disclosure can be readily determined using conventional techniques (see, e.g., Scatchard et al., 1949, Ann. N.Y. Acad. Sci. 51:660; and U.S. Pat. Nos. 5,283,173; 5,468,614, or the equivalent).

As used herein, a “binder”, “binding moiety”, or “targeting moiety” refers to a protein, polypeptide, oligopeptide or peptide, a carbohydrate, a nucleic acid, or combinations thereof capable of specifically binding to a target or multiple targets. A binder includes any naturally occurring, synthetic, semi-synthetic, or recombinantly produced binding partner for a biological molecule or another target of interest. Exemplary binding moieties of this disclosure include an antibody or an antigen binding domain thereof, a Fab′, F(ab′)₂, Fab, Fv, rIgG, scFv, hcAb (heavy chain antibody), a single domain antibody (sdAb), VHH, Variable New Antigen Receptor (VNAR), nanobody, receptor ectodomain or ligand-binding portions thereof, or ligand (e.g., cytokines, chemokines). A “Fab” (antigen binding fragment) is the part of an antibody that binds to antigens and includes the variable region and first heavy chain constant (CH1) domain linked to the light chain via an inter-chain disulfide bond. In other embodiments, a binding moiety comprises a ligand-binding domain of a receptor or a receptor ligand. In some embodiments, a binding moiety can have more than one specificity including, for example, bispecific or multispecific binders. A variety of assays are known for identifying binding moieties of this disclosure that specifically bind a particular target, including Western blot, ELISA, biolayer interferometry, and surface plasmon resonance. A binding moiety, such as a binding moiety comprising immunoglobulin light and heavy chain variable domains (e.g., scFv), can be incorporated into a variety of protein scaffolds or structures as described herein, such as an antibody or an antigen binding fragment thereof, a scFv-Fc fusion protein, or a fusion protein comprising two or more of such immunoglobulin binding domains.

“Framework” or “FW” refers to variable domain residues other than CDR residues. The FW of a variable domain generally consists of four FW regions: FW1, FW2, FW3, and FW4. Accordingly, the CDR and FR sequences generally appear in the following sequence in either a VH or VL: FW1-CDR1-FW2-CDR2-FW3-CDR3-FW4.

Various schemes exist for identification of the regions of hypervariability, simple sequential numbering of the antibody sequence is used throughout the application. In some instances, Chothia numbering is used and specifically indicated. There are several CDR numbering systems in common use. Chothia numbering, and its differences from Kabat numbering (as well as Kabat, Chothia, AbM, and Contact CDR definitions) is described on the Antibody Information page at bioinf.org.uk—Prof Andrew C. R. Martin's group at UCL.

A “humanized antibody” is a chimera, a genetically engineered antibody in which the CDRs from an antibody, e.g. a mouse antibody (donor antibody), are grafted into a human antibody (acceptor antibody) in the CDR positions of the acceptor sequence. Thus, a humanized antibody is an antibody having CDRs from a donor, non-human antibody and variable region framework and constant regions, when present, from a human antibody. In certain embodiments, the human framework sequences in a humanized antibody may be modified at certain positions to contain the residue present at that position in the donor antibody in an attempt to better maintain (or improve upon) the affinity, specificity, stability, and/or other property of the donor antibody.

Although a humanized antibody is a chimera, the term “chimeric antibody” is commonly reserved to refer to an antibody comprising the variable regions of a donor antibody and the constant regions of an acceptor antibody (for example, the constant regions of a human antibody) as distinct from a CDR-grafted antibody in which the variable regions are themselves chimeras. Such convention is observed herein. Although a chimeric antibody will be less immunogenic upon administration to the species of the acceptor antibody, most often repeated or prolonged exposure induces an immune response that limits or eliminates clinical usefulness whereas humanized antibodies avoid or reduce the occurrence of such deleterious immune responses.

As used herein, the terms “monovalent” or “bivalent” refer to one or two antigen binding sites on the whole antibody or antibody fragments.

As used herein, the mouse anti-CD8α antibody clone RPA-T8 is referred to as “CT8” antibody and is used as a donor for humanization. As expressed on human cells, CD8 is a dimer, commonly of two a chains or one each of an α and β chain. Most human CD8+ T cells express the αβ heterodimer. CT8 recognizes an epitope on the α chain. CT8 and its humanized derivatives can bind to both the α₂ and αβ dimers.

The humanized anti-CD8α antigen binding domains of this disclosure can be incorporated into different antibody formats such as antigen-binding fragment (F(ab), F(ab′), or F(ab′)₂), single-chain fragment variable (scFv), diabody, minibody, and other antibody formats described elsewhere (Wilkinson & Hale, 2022, Mabs 14(1): e2123299). The term “F(ab)” denotes an antigen-binding monovalent fragment having a molecular weight of about 50,000 Daltons and antigen binding activity, and consisting of VH and VL, the light chain constant domain (CL) and the first heavy chain constant domain (CH1) domains. The term “F(ab′)₂” refers to an antibody bivalent fragment having a molecular weight of about 100,000 Daltons and antigen binding activity, which comprises two antigen-binding fragments (F(ab)) linked by a disulfide bridge at the hinge region. F(ab′) refers to monovalent antigen binding fragments comprising some hinge region and can be produced by partial reduction of F(ab′)₂ or through recombinant DNA methods involving truncation or substitution of the relevant hinge cysteine residue. While the various Fab fragments were classically produced by proteolytic digestion it has become standard to produce them through recombinant DNA methods, especially for monoclonal reagents. This allows for variation and modification of their amino acid sequences and termini but the Fab terms are nonetheless applied to such analogous molecules. The term “scFv” refers to the N-terminal part of the Fab fragment and consists of the variable portions of a light chain and a heavy chain (VH and VL) connected by a short linker peptide of 10-25 amino acids in either order. The term “diabody” refers to bivalent fragment composed of two chains, each comprising a VH and VL domain, either from the same or from different antibodies. In the diabody format, the two variable domains (VH and VL) are connected by a short linker that is usually 5 residues. In contrast to a scFv, the linker in a diabody is generally too short for the two domains in the same polypeptide chain to associate with each other. The term “minibody” refers to scFv-derived bivalent fragment with two scFvs, each fused to a constant heavy domain 3 (CH3), and in some embodiments, bispecific.

The term “monoclonal antibody” or “mAb” as used herein refers to an antibody molecule of a single amino acid composition, that is directed against a specific antigen and which may be produced by a single clone of B cells or hybridoma, or by recombinant methods. The use of antibody components derived from humanized monoclonal antibodies obviates potential problems associated with the immunogenicity of murine constant and/or framework regions. Rodent monoclonal antibodies to specific antigens may be obtained by methods known to those skilled in the art (see, e.g., Kohler and Milstein, Nature 256: 495 (1975), and Coligan et al. (eds.), Current Protocols In Immunology, VOL. 1, pages 2.5.1-2.6.7 (John Wiley & Sons 1991)).

The various anti-human CD8α antigen binding domains described herein are frequently referred to by the initials CBD followed by a 4-digit number. In various experiments these anti-CD8α antigen binding domains are constructed into whole antibodies (e.g., as a human IgG1 with the LALAPA Fc silencing mutations; see SEQ ID NO: 43 or 44), F(ab), and other antigen binding formats. The initials CBD may also be followed by a number in the form xxxx.y or xxxx.yy in which the four digits again indicate the antigen binding domain and the one or two digits following the decimal indicate the F(ab′) or other antibody format (see Table 17).

As used herein, term “polypeptide” refers to a molecule composed of monomers (amino acids) linearly linked by amide bonds (also known as peptide bonds). The term “polypeptide” refers to any chain of two or more amino acids and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms.

As used throughout this disclosure, “identical” or “identity” refer to the similarity between a DNA, RNA, nucleotide, amino acid, or protein sequence to another DNA, RNA, nucleotide, amino acid, or protein sequence, respectively. Identity can be expressed in terms of a percentage of sequence identity of a first sequence to a second sequence. Percent (%) sequence identity with respect to a reference DNA sequence can be the percentage of DNA nucleotides in a candidate sequence that are identical with the DNA nucleotides in the reference DNA sequence after aligning the sequences. Percent (%) sequence identity with respect to a reference amino acid sequence can be the percentage of amino acid residues in a candidate sequence that are identical with the amino acid residues in the reference amino acid sequence after aligning the sequences and introducing gaps, if necessary, to achieve the maximum percent sequence identity, and not considering any conservative substitutions as part of the sequence identity. As used throughout this disclosure, the percent sequence identity values is generated using the NCBI BLAST 2.0 software as defined by Altschul et al., “Gapped BLAST and PSI-BLAST: a new generation of protein database search programs,” Nucleic Acids Res. 2007, 25, 3389-3402, with the parameters set to default values.

### Humanized Anti-CD8 Binding Moiety

This disclosure provides anti-CD8α antibodies (e.g., isolated monoclonal antibodies), also referred to as anti-CD8α antibodies or antigen-binding fragments thereof. In some embodiments of this disclosure, an anti-CD8α antibody or antigen binding fragment thereof comprises two light chain polypeptides (light chains) and two heavy chain polypeptides (heavy chains), held together covalently by disulfide linkages.

In particular embodiments, VH and VL of this disclosure may be expressed as separate polypeptides that associate with each other to form an antigen binding fragment specific for CD8α, as they do in natural antibodies or in various F(ab) fragments known in the art. In other embodiments, VH and VL of this disclosure can be contained in a single polypeptide chain connected by a linker peptide. If the linker is of sufficient length, VH and VL of the same polypeptide chain can associate, forming a single chain Fv (scFv) that specifically binds to CD8α. A shorter linker can be used so the VH and VL in one polypeptide chain associate with the VL and VH, respectively, of a second polypeptide chain to form a diabody. In general, an antigen binding domain can be used in modular fashion and be combined with other protein domains.

In some embodiments, a heavy chain comprises a heavy chain variable region (VH) and a heavy chain constant region. In some embodiments, the heavy chain constant region comprises three domains, CH1, CH2, and CH3. In certain embodiments, humanized anti-CD8α variants are grafted on all or a portion of a heavy chain constant region. Non-limiting exemplary heavy chain constant regions include human IgG1, IgG2, IgG3, IgG4, IgA1, IgA2, IgE, IgM or IgD constant regions. In some embodiments, an antibody of this disclosure comprises an IgG1 constant region. Exemplary heavy chain constant regions include human IgG1 heavy chain constant region (SEQ ID NO:42) and human IgG1null heavy chain constant regions (SEQ ID NO:43 or 44).

In some embodiments, the light chain comprises a light chain variable region (VL) and a light chain constant region. The humanized anti-CD8α variants of this disclosure are grafted on all or a portion of a kappa light chain constant region or a lambda light chain constant region, or a portion thereof. Non-limiting exemplary light chain constant regions include kappa and lambda constant regions. A non-limiting exemplary human kappa constant region is shown in SEQ ID NO: 41.

The constant domains provide the general framework of the antibody and may not be involved directly in binding the antibody to an antigen, but can be involved in various effector functions, such as participation of the antibody in antibody-dependent cellular cytotoxicity (ADCC), ADCP (antibody-dependent cellular phagocytosis), CDC (complement-dependent cytotoxicity), and complement fixation, binding to Fc receptors (e.g., CD16, CD32, FcRn). As used herein, “Fc” or “Fc region” refers to the heavy chain constant region segment of the Fc fragment (the “fragment crystallizable” region or Fc region) from an antibody, which can in include one or more constant domains, such as CH2, CH3, CH4, or any combination thereof. In some embodiments, an Fc region includes the CH2 and CH3 domains of an IgG, IgA, or IgD antibody and any combination thereof, or the CH3 and CH4 domains of an IgM or IgE antibody and any combination thereof.

An Fc region may interact with different types of Fc receptors (FcRs). The different types of FcRs may include, for example, FcγRI, FcγRIIA, FcγRIIB, FcγRIIIA, FcγRIIIB, FcαRI, FcμR, FcεRI, FcεRII, and FcRn. FcRs may be located on the membrane of certain immune cells including, for example, B lymphocytes, natural killer cells, macrophages, neutrophils, follicular dendritic cells, eosinophils, basophils, platelets, and mast cells. Once the FcR is engaged by the Fc region, the FcR may initiate various effector functions noted above. FcRs may deliver signals when FcRs are aggregated by antibodies at the cell surface. The aggregation of FcRs with immunoreceptor tyrosine-based activation motifs (ITAMs) may sequentially activate SRC family tyrosine kinases and SYK family tyrosine kinases. The SRC and SYK kinases may connect the transduced signals with common activation pathways. Such signals may be undesirable in the treatment of certain indications, such as autoimmune diseases.

In some embodiments, an Fc region can exhibit reduced binding affinity to one or more Fc receptors. In some embodiments, an Fc region can exhibit reduced binding affinity to one or more Fcγ receptors, FcRn receptors, or both. In some embodiments, an Fc domain is an Fc null or Fc silenced region. As used herein, an “Fc null” or “Fc silenced” region refers to a domain that exhibits weak to no binding to any of the Fcγ receptors.

The Fc region or domain may have one or more, two or more, three or more, or four or more, or up to five amino acid substitutions that decrease binding of the Fc region to an FcR. In some embodiments, an Fc region exhibits decreased binding to FcγRI (CD64), FcγRIIA (CD32), FcγRIIIA (CD16a), FcγRIIIB (CD16b), or any combination thereof. In order to decrease binding affinity of an Fc region to an FcR, an Fc region may comprise one or more amino acid substitutions that has the effect of reducing the affinity of the Fc region to an FcR. In some embodiments, the Fc region is an IgG1 and the one or more substitutions in the Fc region comprise any one or more of IgG1 heavy chain mutations corresponding to E233P, L234V, L234A, L235A, L235E, ΔG236, G237A, E318A, K320A, K322A, A327G, P329A, A330S, or P331S according to the EU index of Kabat numbering.

In some embodiments, the Fc region can comprise a sequence of the IgG1 isoform that has been modified from the wild-type IgG1 sequence. A modification can comprise a substitution at more than one amino acid residues, such as at two different amino acid residues including S239D/I332E (IgG1 SDIE) according to the EU index of Kabat numbering. A modification can comprise a substitution at more than one amino acid residue, such as at three different amino acid residues including L234A/L235A/P329A (IgG1 LALAPA) or S298A/E333A/K334A (IgG1 SAEAKA) according to the EU index of Kabat numbering. A modification can comprise a substitution at more than one amino acid residue, such as at 5 different amino acid residues including L235V/F243L/R292P/Y300L/P396L (IgG1 LVFLRPYLPL) according to the EU index of Kabat numbering. Non-limiting exemplary human IgG1 heavy chain constant regions having Fc silencing mutations are shown in SEQ ID NOS: 43 and 44.

The Fc portion of an antibody can also mediate functional interaction with other agents in addition to Fc receptors, including the mannose receptor, complement component C1q, and TRIM21. To prevent these interactions and their functional effects, antibody formats without an Fc region can be used including scFv, F(ab), F(ab′), F(ab′)₂, and variations thereof. Table 17 presents exemplary sequences for wild type and engineered Ckappa and F(ab′) heavy constant domains (in some cases truncated to remove some or all of the hinge region found in classical F(ab′).

Binding affinity, generally reported as dissociation constant K_{D}, can be determined by kinetic or steady state (equilibrium) analysis, that is, from the ratio of binding off and on rates or from a binding-concentration curve, respectively. In some embodiments, anti-CD8α antigen binding fragments having framework regions from human germline heavy and light chain variable domains of this disclosure in the form of a F(ab), F(ab′), or a full-length antibody (e.g., combined with an Fc silenced IgG1 antibody constant domain of SEQ ID NO: 43). To determine the K_{D} of these different anti-CD8α binders, kinetic analysis was performed on the F(ab) and F(ab′); and steady state analysis was performed on the Fc silenced IgG1 antibody. Humanized anti-CD8 antigen binding fragments CBD1033 to CBD1050 were tested in both biolayer interferometry (BLI) kinetics and surface plasmon resonance (SPR) binding assays at two temperatures (30° C. and physiological 37° C.). The assays measured binding affinities against CD8αα homodimer. In these embodiments, a humanized anti-CD8α F(ab), F(ab′), or Fc null full-length antibody has a K_{D} of about 10 nM or less. In some embodiments, CBD1032, CBD1033, CBD1035, CBD1036, CBD1037, CBD1038, CBD1039, or CBD1040 F(ab) has a K_{D} of less than 8 nM. In some embodiments, CBD1032, CBD1033, CBD1037, or CBD1039 F(ab) has a K_{D} of less than 5 nM. In some embodiments, CBD1032, CBD1033, CBD1034, CBD1035, CBD1036, CBD1037, CBD1038, CBD1039, CBD1040, CBD1042, CBD1043, CBD1045, CBD1047, CBD1048, CBD1049, or CBD1050 Fc null full-length antibody has a K_{D} of less than 7 nM. In some embodiments, CBD1032, CBD1033, CBD1034, CBD1035, CBD1037, CBD1038, CBD1039, CBD1040, CBD1042, CBD1043, CBD1045, CBD1047, or CBD1049 Fc null full-length antibody has a K_{D} of less than 5 nM. In some embodiment, CBD1033.24 or CBD1033.37 F(ab′) has a K_{D} of less than 7 nM. A widely used surrogate for affinity is EC₅₀, the concentration producing half-maximal effect. In some embodiments, an anti-CD8α Fc null antibody of this disclosure has an EC₅₀ of about 6 nM, about 3 nM, about 2 nM, about 1 nM, or about 0.5 nM. It was found that the humanized anti-CD8 antigen binding fragments disclosed and tested in this application showed binding affinities to both CD8αα homodimer and CDαβ heterodimer and that the strength of the binding to the CT8 epitope was sufficient for tLNP delivery function to CD8+ cells. Particularly, CBD1033 F(ab) bound to human and cynomolgus macaque CD8αα homodimer and CD8αβ heterodimer. However, the binding affinities of CBD1033 F(ab) were 5-fold weaker than the parent CBD1017ch F(ab). Additionally, CBD1033 F(ab) or Fc null full-length antibody has 6 to 10-fold weaker binding affinity towards CD8αβ heterodimer compared to CD8αα homodimer. Nevertheless, CBD1033-targeted tLNP showed comparable and in some instances better transfection efficiency than CBD1017ch in vitro; and up to 80% transfection efficiency in vivo in CD8+ T cells which are known to express primarily CD8αβ heterodimer. Further studies on binding site of CD8 revealed that CBD1033 binds to a specific epitope on CD8 (called CT8 epitope) that conveyed superior transfection efficiency compared to other epitopes bound by other antibodies such as SK1 and OKT8. Thus, the binding affinity of humanized anti-CD8 binding fragments disclosed herein to CT8 epitope of either CD8αα homodimer or CD8αβ dimer is sufficient to retain tLNP transfection function in vivo.

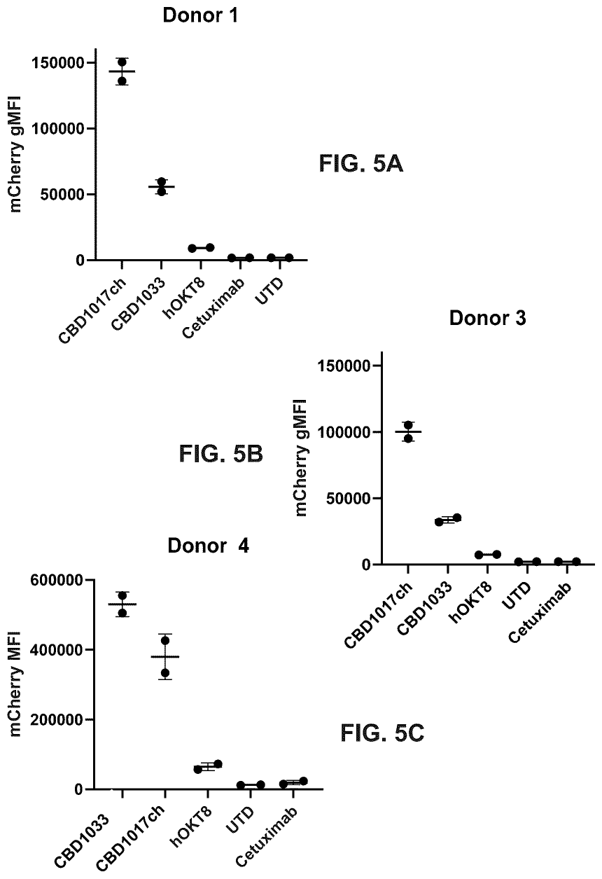

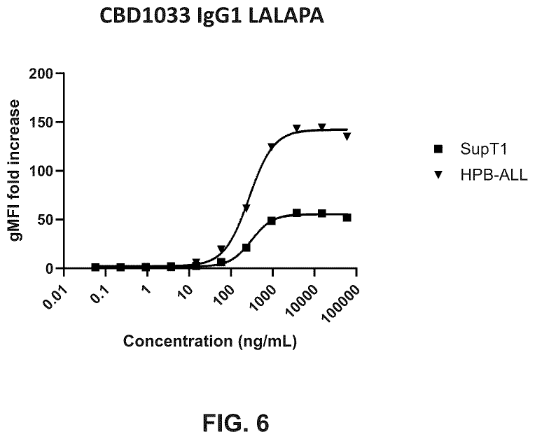

In some embodiments, an anti-CD8α antibody or antigen binding fragment thereof of this disclosure specifically binds to a non-human primate and human CD8αα homodimer and CDαβ heterodimer. In some instances, a humanized anti-CD8α antibody or antigen binding fragment thereof of this disclosure specifically binds cynomolgus macaque or rhesus macaque CD8. In some embodiments, a humanized anti-CD8α antibody or antigen binding fragment thereof of this disclosure competes for binding to the same epitope on CD8α that is bound by TRX2 and vice versa, the humanized anti-CD8 antibody described in FIGS. 5 and 6 of US20060002921 which is incorporated by references for all that it teaches about TRX2. Sequences of TRX2 antibody are shown in Table 19. In some embodiments, a humanized anti-CD8 antibody or antigen binding fragment thereof of this disclosure competes for binding to the same epitope on CD8α that is bound by YTC182.20 (described in Jonker, M. et al. (1989) Reactivity of mAb specific for human CD markers with Rhesus monkey leucocytes. Leucocyte Typing IV. Oxford University Press p 1058-1063 which is incorporated by references for all that it teaches about YTC182.20) and vice versa. The CT8 epitope bound by these antibodies is located in a membrane proximal portion of CD8α ectodomain above the stalk (or hinge) emerging from the cell membrane and near the dimer interface. Crosslinking analysis showed that CBD1033 bound at or near amino acid residues 40, 45, 47, 86, 91, 95, 103, 105, and 106 of CD8α indicating that CBD1033 interacted with the CC′ loop, C′ strand, turn before F strand, F strand, and G strand of CD8α (as predicted by AlphaFold2) and thereby identifying the location of the CT8 epitope. (See Srinivasan et al., 2024 Front. Immunol. 15:1412513, which is incorporated by reference for its teachings about the structure of CD8 and its interactions with mAbs.) Residues 40 and 45 are int the CC′ loop, residue 47 is in the C′ strand, residue 86 is in the turn before the F strand, residues 91 and 95 are in the F strand, and residues 103, 105, and 106 are in the G strand. Competition binding analysis shows that the anti-CD8 antibody OKT8 does not compete for binding to the same epitope. The anti-CD8 antibody SK1, also does not compete for binding to the CT8 epitope but blocks T cell activation and competes for binding to the same epitope as the anti-CD8 antibody HIT8α. In various embodiments, antigen binding domains that bind the CT8 epitope are designated means for binding the CT8 epitope or means for competing for binding to the same epitope as bound by CT8, TRX2, and/or YTC182.20.

The variable region of an antibody contains the antigen-binding site of the molecule. The variable heavy chain (VH) and the variable light chain (VL) are domains of the larger and smaller polypeptide subunits, respectively, of an antibody and form the antigen-binding site. The VH and VL domains of an antibody generally have similar structures, with each domain including four conserved framework regions (FWs) and three hypervariable regions. The majority of sequence variability of an antibody occurs in the six hypervariable regions, each termed a “complementarity determining region” (CDR), three each per VH and VL chain (VH-CDR1, VH-CDR2, VH-CDR3, VL-CDR1, VL-CDR2, VL-CDR3). In certain embodiments, antigen-recognition regions of an anti-CD8 antibody variable domain of this disclosure comprises six CDRs, or hypervariable regions, that lie within the framework of the heavy chain variable region and light chain variable region at the N-terminal ends of the two heavy and two light chains. For example, a CD8 binding domain comprises a heavy chain complementary determining region 1 (VH-CDR1), a heavy chain complementary determining region 2 (VH-CDR2), a heavy chain complementary determining region 3 (VH-CDR3), a light chain complementary determining region 1 (VL-CDR1), a light chain complementary determining region 2 (VL-CDR2), and a light chain complementary determining region 3 (VL-CDR3).

In certain aspects, an anti-CD8α antibody or antigen binding fragment thereof having framework regions from human germline heavy and light chain variable domains comprises: (a) a VH comprising a VH-CDR1 having the amino acid sequence RYTFTDYX₁LH (SEQ ID NO: 45) wherein X₁ is N, S, Q, or A, an VH-CDR2 having the amino acid sequence FIYPYX₁GGTG (SEQ ID NO: 46) or FIYPYX₂GGTG (SEQ ID NO: 47) wherein X₂ is N, Q, D, S, or A, an VH-CDR3 having the amino acid sequence DHRYX₁EGVSFDY (SEQ ID NO: 48), and a VL comprising a VL-CDR1 having the amino acid sequence RASESVX₃GFGX₂SFMN (SEQ ID NO: 49) wherein X₃ is an amino acid identified by the symbol D, E, S, or A; a VL-CDR2 having the amino acid sequence LASX₂LES (SEQ ID NO: 50), and a VL-CDR3 having the amino acid sequence QQX₂X₂EX₃PYT (SEQ ID NO: 51). In some embodiments, VH-CDR1 has the amino acid sequence RYTFTDYNLH (SEQ ID NO: 2). In some embodiments, VH-CDR2 has the amino acid sequence FIYPYNGGTG (SEQ ID NO: 3), FIYPYSGGTG (SEQ ID NO: 58), FIYPYQGGTG (SEQ ID NO: 59), or FIYPYAGGTG (SEQ ID NO: 60). In some embodiments, VH-CDR3 has the amino acid sequence DHRYNEGVSFDY (SEQ ID NO: 4). In some embodiments, VL-CDR1 has the amino acid sequence RASESVDGFGNSFMN (SEQ ID NO: 6), SEQ ID NO: 227, or SEQ ID NO: 228. In some embodiments, VL-CDR2 has the amino acid sequence LASNLES (SEQ ID NO: 7). In some embodiments, VL-CDR3 has the amino acid sequence QQNNEDPYT (SEQ ID NO: 8).

In further embodiments, a humanized anti-CD8α antibody or antigen binding fragment thereof comprises: (a)(i) a VH-CDR1 comprising the amino acid sequence of SEQ ID NO: 2, a VH-CDR2 comprising the amino acid sequence of SEQ ID NO: 3, and a VH-CDR3 comprising the amino acid sequence SEQ ID NO: 4; (ii) a VH-CDR1 comprising the amino acid sequence of SEQ ID NO: 2, a VH-CDR2 comprising the amino acid sequence of SEQ ID NO: 58, and a VH-CDR3 comprising the amino acid sequence SEQ ID NO: 4; (iii) a VH-CDR1 comprising the amino acid sequence of SEQ ID NO: 2, a VH-CDR2 comprising the amino acid sequence of SEQ ID NO: 59, and a VH-CDR3 comprising the amino acid sequence SEQ ID NO: 4; or (iv) a VH-CDR1 comprising the amino acid sequence of SEQ ID NO: 2, a VH-CDR2 comprising the amino acid sequence of SEQ ID NO: 60, and a VH-CDR3 comprising the amino acid sequence SEQ ID NO: 4; and (b) a VL-CDR1 comprising the amino acid sequence SEQ ID NO: 6, a VL-CDR2 comprising the amino acid sequence SEQ ID NO: 7; and a VL-CDR3 comprising the amino acid sequence SEQ ID NO: 8.

In any of the aforementioned embodiments, an acceptor sequence from which: (a) heavy chain framework regions are derived are from IGHV1-46*01/IGHJ6*01, as shown in SEQ ID NO: 9; (b) light chain framework regions are derived are from IGKV1-39*01/IGKJ2*01, as shown in SEQ ID NO: 15; (c) heavy chain framework regions are derived are from a modified IGHV1-18*01, as shown in SEQ ID NO: 31; (d) light chain framework regions are derived are from a modified version of IGKV3D-11*01, as shown in SEQ ID NO: 37; (e) heavy chain framework regions are derived are from IGHV1-46*01/IGHJ6*01 and the light chain framework regions are derived are from IGKV1-39*01/IGKJ2*01; (f) heavy chain framework regions are derived are from a modified IGHV1-18*01 and light chain framework regions are derived are from a modified version of IGKV3D-11*01; (g) heavy chain framework regions are derived are from IGHV1-46*01/IGHJ6*01 and light chain framework regions are derived are from a modified version of IGKV3D-11*01; (h) heavy chain framework regions are derived are from a modified IGHV1-18*01 and light chain framework regions are derived are from IGKV1-39*01/IGKJ2*01.

In certain embodiments, an anti-CD8α antibody or antigen binding fragment thereof comprises a heavy chain variable region (VH) comprising an amino acid sequence that is at least 90%, 91%, 92%, 93%, 94%, 95%, 96%, 97%, 98%, 99%, or 100% identical to the amino acid sequence of SEQ ID NO: 10, SEQ ID NO: 11, SEQ ID NO: 12, SEQ ID NO: 13, SEQ ID NO: 14, SEQ ID NO: 27, SEQ ID NO: 28, SEQ ID NO: 29, SEQ ID NO: 33, SEQ ID NO 34, SEQ ID NO: 35, SEQ ID NO: 36, SEQ ID NO: 66, SEQ ID NO: 67, or SEQ ID NO: 68; and a light chain variable region (VL) comprising an amino acid sequence that is at least 90%, 91%, 92%, 93%, 94%, 95%, 96%, 97%, 98%, 99%, or 100% identical to the amino acid sequence of SEQ ID NO: 16, SEQ ID NO: 17, SEQ ID NO: 18, SEQ ID NO: 38, SEQ ID NO: 39, SEQ ID NO: 40, SEQ ID NO: 64, or SEQ ID NO: 65.

In certain embodiments, an anti-CD8α antibody or antigen binding fragment thereof comprises a heavy chain variable region (VH) comprising an amino acid sequence that is at least 90%, 91%, 92%, 93%, 94%, 95%, 96%, 97%, 98%, 99%, or 100% identical to the amino acid sequence of SEQ ID NO: 10, SEQ ID NO: 11, SEQ ID NO: 12, SEQ ID NO: 13, SEQ ID NO: 14, SEQ ID NO: 35, or SEQ ID NO: 36, and wherein the VH-CDR1 comprises the amino acid sequence of SEQ ID NO: 2, the VH-CDR2 comprises the amino acid sequence of SEQ ID NO: 3, 58, 59, or 60, and the VH-CDR3 comprises the amino acid sequence of SEQ ID NO: 4; and a light chain variable region (VL) comprising an amino acid sequence that is at least 90%, 91%, 92%, 93%, 94%, 95%, 96%, 97%, 98%, 99%, or 100% identical to the amino acid sequence of SEQ ID NO: 16, SEQ ID NO: 17, SEQ ID NO: 18, or SEQ ID NO: 39, and wherein the VL-CDR1 comprises the amino acid sequence of SEQ ID NO: 6, 227, or 228, the VL-CDR2 comprises the amino acid sequence of SEQ ID NO: 7, and the VL-CDR3 comprises the amino acid sequence of SEQ ID NO: 8. In some embodiments, an anti-CD8α antibody or antigen binding fragment thereof comprises a VH comprising an amino acid sequence that is at least 90%, 91%, 92%, 93%, 94%, 95%, 96%, 97%, 98%, 99%, or 100% identical to the amino acid sequence of SEQ ID NO:10, 11, 12, 13, or 14; and a VL comprising an amino acid sequence that is at least 90%, 91%, 92%, 93%, 94%, 95%, 96%, 97%, 98%, 99%, or 100% identical to the amino acid sequence of SEQ ID NO:16, provided that the amino acid sequences of the VH-CDRs (i.e., SEQ ID NOS: 2, 3, and 4) and VL-CDRs (i.e., SEQ ID NOS: 6, 7, and 8) are unchanged. In further embodiments, an anti-CD8α antibody or antigen binding fragment thereof comprises a VH comprising an amino acid sequence that is at least 90%, 91%, 92%, 93%, 94%, 95%, 96%, 97%, 98%, 99%, or 100% identical to the amino acid sequence of SEQ ID NO: 10, 11, 12, 13, or 14,; and a VL comprising an amino acid sequence that is at least 90%, 91%, 92%, 93%, 94%, 95%, 96%, 97%, 98%, 99%, or 100% identical to the amino acid sequence of SEQ ID NO:17, provided that the amino acid sequences of the VH-CDRs and VL-CDRs are unchanged. In still further embodiments, an anti-CD8α antibody or antigen binding fragment thereof comprises a VH comprising an amino acid sequence that is at least 90%, 91%, 92%, 93%, 94%, 95%, 96%, 97%, 98%, 99%, or 100% identical to the amino acid sequence of SEQ ID NO: 10, 11, 12, 13, or 14; and a VL comprising an amino acid sequence that is at least 90%, 91%, 92%, 93%, 94%, 95%, 96%, 97%, 98%, 99%, or 100% identical to the amino acid sequence of SEQ ID NO:18, provided that the amino acid sequences of the VH-CDRs and VL-CDRs are unchanged. In yet further embodiments, an anti-CD8α antibody or antigen binding fragment thereof comprises a VH comprising an amino acid sequence that is at least 90%, 91%, 92%, 93%, 94%, 95%, 96%, 97%, 98%, 99%, or 100% identical to the amino acid sequence of SEQ ID NO:35 or 36; and a VL comprising an amino acid sequence that is at least 90%, 91%, 92%, 93%, 94%, 95%, 96%, 97%, 98%, 99%, or 100% identical to the amino acid sequence of SEQ ID NO:39, provided that the amino acid sequences of the VH-CDRs and VL-CDRs are unchanged.

In some embodiments, an anti-CD8α antibody or antigen binding fragment thereof comprises a heavy chain variable region (VH) comprising an amino acid sequence that is at least 90%, 91%, 92%, 93%, 94%, 95%, 96%, 97%, 98%, 99%, or 100% identical to the amino acid sequence of SEQ ID NO: 33, or SEQ ID NO: 34, and wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4; and a light chain variable region (VL) comprising an amino acid sequence that is at least 90%, 91%, 92%, 93%, 94%, 95%, 96%, 97%, 98%, 99%, or 100% identical to the amino acid sequence of SEQ ID NO: 38 or SEQ ID NO: 40, and wherein the VL-CDR1 comprises the amino acid sequence of SEQ ID NO: 6, the VL-CDR2 comprises the amino acid sequence of SEQ ID NO: 7, and the VL-CDR3 comprises the amino acid sequence of SEQ ID NO: 8.

In certain embodiments, an anti-CD8α antibody or antigen binding fragment thereof comprises a VL region having the amino acid sequence of one of SEQ ID NOs: 16-18, 38-40, 64, or 65. In some embodiments, an anti-CD8α antibody or antigen binding fragment thereof comprises a VH region having the amino acid sequence of one of SEQ ID NOs: 10-14, 27-30, 32-36, or 66-68. In further embodiments, an anti-CD8α antibody or antigen binding fragment thereof comprises a VL region having the amino acid sequence of SEQ ID NO: 17 and a VH region having the amino acid sequence of one of SEQ ID NOs: 11 or 27-30. In still further embodiments, an anti-CD8α antibody or antigen binding fragment thereof comprises (a) a VH comprising the amino acid sequence of SEQ ID NO: 10 and a VL comprising the amino acid sequence of SEQ ID NO: 16; (b) a VH comprising the amino acid sequence of one of SEQ ID NO: 11-14, and a VL comprising the amino acid sequence of SEQ ID NO: 17; or (c) a VH comprising the amino acid sequence of one of SEQ ID NO: 11-14, and a VL comprising the amino acid sequence of SEQ ID NO: 18. In further embodiments, an anti-CD8α antibody or antigen binding fragment thereof comprises a VL region having the amino acid sequence of SEQ ID NO: 64 and a VH region having the amino acid sequence of one of SEQ ID NOs: 11, 13, 28, 29, 67, or 68. In further embodiments, an anti-CD8α antibody or antigen binding fragment thereof comprises a VL region having the amino acid sequence of SEQ ID NO: 65 and a VH region having the amino acid sequence of one of SEQ ID NOs: 11 and 13.

In further embodiments, an anti-CD8α antibody or antigen binding fragment thereof comprises: (a) a VH comprising the amino acid sequence of SEQ ID NO: 10, 11, 12, 13, 14, 35, or 36, wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4, and the VL comprising the amino acid sequence of SEQ ID NO: 16; (b) a VH comprising the amino acid sequence of SEQ ID NO: 27 or 66, wherein the VH-CDR1 comprises the amino acid sequence of SEQ ID NO: 2, the VH-CDR2 comprises the amino acid sequence of SEQ ID NO: 58, and the VH-CDR3 comprises the amino acid sequence of SEQ ID NO: 4, and the VL comprising the amino acid sequence of SEQ ID NO: 16; (c) a VH comprising the amino acid sequence of SEQ ID NO: 28 or 67, wherein the VH-CDR1 comprises the amino acid sequence of SEQ ID NO: 2, the VH-CDR2 comprises the amino acid sequence of SEQ ID NO: 59, and the VH-CDR3 comprises the amino acid sequence of SEQ ID NO: 4, and the VL comprising the amino acid sequence of SEQ ID NO: 16; (d) a VH comprising the amino acid sequence of SEQ ID NO: 29 or 68, wherein the VH-CDR1 comprises the amino acid sequence of SEQ ID NO: 2, the VH-CDR2 comprises the amino acid sequence of SEQ ID NO: 60, and the VH-CDR3 comprises the amino acid sequence of SEQ ID NO: 4, and the VL comprising the amino acid sequence of SEQ ID NO: 16; (e) a VH comprising the amino acid sequence of SEQ ID NO: 10, 11, 12, 13, 14, 35, or 36, wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4, and the VL comprising the amino acid sequence of SEQ ID NO: 17; (f) a VH comprising the amino acid sequence of SEQ ID NO: 27 or 66, wherein the VH-CDR1 comprises the amino acid sequence of SEQ ID NO: 2, the VH-CDR2 comprises the amino acid sequence of SEQ ID NO: 58, and the VH-CDR3 comprises the amino acid sequence of SEQ ID NO: 4, and the VL comprising the amino acid sequence of SEQ ID NO: 17; (g) a VH comprising the amino acid sequence of SEQ ID NO: 10, 11, 12, 13, 14, 35, or 36, wherein the VH-CDR1 comprises the amino acid sequence of SEQ ID NO: 2, the VH-CDR2 comprises the amino acid sequence of SEQ ID NO: 59, and the VH-CDR3 comprises the amino acid sequence of SEQ ID NO: 4, and the VL comprising the amino acid sequence of SEQ ID NO: 17; (h) a VH comprising the amino acid sequence of SEQ ID NO: 29 or 68, wherein the VH-CDR1 comprises the amino acid sequence of SEQ ID NO: 2, the VH-CDR2 comprises the amino acid sequence of SEQ ID NO: 60, and the VH-CDR3 comprises the amino acid sequence of SEQ ID NO: 4, and the VL comprising the amino acid sequence of SEQ ID NO: 17; (i) a VH comprising the amino acid sequence of SEQ ID NO: 10, 11, 12, 13, 14, 35, or 36, wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4, and the VL comprising the amino acid sequence of SEQ ID NO: 18; (j) a VH comprising the amino acid sequence of SEQ ID NO: 27 or 66, wherein the VH-CDR1 comprises the amino acid sequence of SEQ ID NO: 2, the VH-CDR2 comprises the amino acid sequence of SEQ ID NO: 58, and the VH-CDR3 comprises the amino acid sequence of SEQ ID NO: 4, and the VL comprising the amino acid sequence of SEQ ID NO: 18; (k) a VH comprising the amino acid sequence of SEQ ID NO: 28 or 67, wherein the VH-CDR1 comprises the amino acid sequence of SEQ ID NO: 2, the VH-CDR2 comprises the amino acid sequence of SEQ ID NO: 59, and the VH-CDR3 comprises the amino acid sequence of SEQ ID NO: 4, and the VL comprising the amino acid sequence of SEQ ID NO: 18; (1) a VH comprising the amino acid sequence of SEQ ID NO: 29 or 68, wherein the VH-CDR1 comprises the amino acid sequence of SEQ ID NO: 2, the VH-CDR2 comprises the amino acid sequence of SEQ ID NO: 60, and the VH-CDR3 comprises the amino acid sequence of SEQ ID NO: 4, and the VL comprising the amino acid sequence of SEQ ID NO: 18; (m) a VH comprising the amino acid sequence of SEQ ID NO: 10, 11, 12, 13, 14, 35, or 36, wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4, and the VL comprising the amino acid sequence of SEQ ID NO: 39; (n) a VH comprising the amino acid sequence of SEQ ID NO: 27 or 66, wherein the VH-CDR1 comprises the amino acid sequence of SEQ ID NO: 2, the VH-CDR2 comprises the amino acid sequence of SEQ ID NO: 58, and the VH-CDR3 comprises the amino acid sequence of SEQ ID NO: 4, and the VL comprising the amino acid sequence of SEQ ID NO: 39; (o) a VH comprising the amino acid sequence of SEQ ID NO: 28 or 67, wherein the VH-CDR1 comprises the amino acid sequence of SEQ ID NO: 2, the VH-CDR2 comprises the amino acid sequence of SEQ ID NO: 59, and the VH-CDR3 comprises the amino acid sequence of SEQ ID NO: 4, and the VL comprising the amino acid sequence of SEQ ID NO: 39; (p) a VH comprising the amino acid sequence of SEQ ID NO: 29 or 68, wherein the VH-CDR1 comprises the amino acid sequence of SEQ ID NO: 2, the VH-CDR2 comprises the amino acid sequence of SEQ ID NO: 60, and the VH-CDR3 comprises the amino acid sequence of SEQ ID NO: 4, and the VL comprising the amino acid sequence of SEQ ID NO: 39; (q) a VH comprising the amino acid sequence of SEQ ID NO: 10, 11, 12, 13, 14, 33, 34, 35, or 36, wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4, and the VL comprising the amino acid sequence of SEQ ID NO: 64; (r) a VH comprising the amino acid sequence of SEQ ID NO: 27 or 66, wherein the VH-CDR1 comprises the amino acid sequence of SEQ ID NO: 2, the VH-CDR2 comprises the amino acid sequence of SEQ ID NO: 58, and the VH-CDR3 comprises the amino acid sequence of SEQ ID NO: 4, and the VL comprising the amino acid sequence of SEQ ID NO: 64; (s) a VH comprising the amino acid sequence of SEQ ID NO: 28 or 67, wherein the VH-CDR1 comprises the amino acid sequence of SEQ ID NO: 2, the VH-CDR2 comprises the amino acid sequence of SEQ ID NO: 59, and the VH-CDR3 comprises the amino acid sequence of SEQ ID NO: 4, and the VL comprising the amino acid sequence of SEQ ID NO: 64; (t) a VH comprising the amino acid sequence of SEQ ID NO: 29 or 68, wherein the VH-CDR1 comprises the amino acid sequence of SEQ ID NO: 2, the VH-CDR2 comprises the amino acid sequence of SEQ ID NO: 60, and the VH-CDR3 comprises the amino acid sequence of SEQ ID NO: 4, and the VL comprising the amino acid sequence of SEQ ID NO: 64; (u) a VH comprising the amino acid sequence of SEQ ID NO: 10, 11, 12, 13, 14, 33, 34, 35, or 36, wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4, and the VL comprising the amino acid sequence of SEQ ID NO: 65; (v) a VH comprising the amino acid sequence of SEQ ID NO: 27 or 66, wherein the VH-CDR1 comprises the amino acid sequence of SEQ ID NO: 2, the VH-CDR2 comprises the amino acid sequence of SEQ ID NO: 58, and the VH-CDR3 comprises the amino acid sequence of SEQ ID NO: 4, and the VL comprising the amino acid sequence of SEQ ID NO: 65; (w) a VH comprising the amino acid sequence of SEQ ID NO: 28 or 67, wherein the VH-CDR1 comprises the amino acid sequence of SEQ ID NO: 2, the VH-CDR2 comprises the amino acid sequence of SEQ ID NO: 59, and the VH-CDR3 comprises the amino acid sequence of SEQ ID NO: 4, and the VL comprising the amino acid sequence of SEQ ID NO: 65; (x) a VH comprising the amino acid sequence of SEQ ID NO: 29 or 68, wherein the VH-CDR1 comprises the amino acid sequence of SEQ ID NO: 2, the VH-CDR2 comprises the amino acid sequence of SEQ ID NO: 60, and the VH-CDR3 comprises the amino acid sequence of SEQ ID NO: 4, and the VL comprising the amino acid sequence of SEQ ID NO: 65.

Examples of humanized anti-CD8α variants comprising variable domains described in this disclosure are made and shown in Tables 3-5. The VH or VL domains described herein can be grafted on classic or engineered heavy chain or light chain constant regions, respectively. The constant regions can be of full-length, F(ab), F(ab′), F(ab′)₂, single-chain fragment variable (scFv), diabody, minibody, or other antibody formats. Non-limiting exemplary heavy chain constant regions include human IgG1, IgG2, IgG3, IgG4, IgA1, IgA2, IgE, IgM or IgD constant regions. Non-limiting exemplary light chain constant regions include kappa and lambda constant regions.

The term “engineered” in the context of F(ab′) refers to modification or mutation of the amino acid residues of the constant regions. In some embodiments, the modification is truncation of the constant regions; for example, truncation after proline in position 245 (P245), P240, or P241 of IgG1 F(ab′) or IgG4 F(ab′) hinge regions; or truncation after T238 of IgG1 F(ab′) hinge region (Table 17). In some embodiments, the mutation is a mutation of cysteine residue into serine or other non-cysteine amino acid to remove disulfide bond; for examples C214S on kappa chain constant region, C233S of IgG1 CH1 domain, or C127S of IgG4 F(ab′) CH1 domain (Table 17). In some embodiments, the mutation is a mutation of non-cysteine amino acid into cysteine to support formation of a new disulfide bond; for examples, F174C of IgG1 F(ab′) or IgG4 F(ab′) CH1 domains and S162C of kappa constant region to form CH1174-Cκ162 disulfide bond (Table 17).

The VH domains described herein can be grafted on heavy chain constant regions comprising the amino acid sequence SEQ ID NO: 42, SEQ ID NO: 43, SEQ ID NO: 44, SEQ ID NO: 76, SEQ ID NO: 79, SEQ ID NO: 81, SEQ ID NO: 83, SEQ ID NO: 85, SEQ ID NO: 87, SEQ ID NO: 90, SEQ ID NO: 93, SEQ ID NO: 95, SEQ ID NO: 97, SEQ ID NO: 99, or SEQ ID NO: 103. The VL domains described herein can be grafted on light chain constant regions comprising the amino acid sequence SEQ ID NO: 41, SEQ ID NO: 89, or SEQ ID NO: 100. The combinations of VH and VL domain disclosed herein with the heavy and light constant regions respectively are shown in Table 17.

### Biophysical Properties of Humanized Anti-CD8 Binding Moiety

In addition to binding affinity and binding specificity to a desired target molecule, a therapeutic antibody beneficially meets a set of criteria regarding the feasibility of their manufacture, stability in storage, and absence of off-target binding (“stickiness”). This suite of characteristics is often termed “developability.” The biophysical properties of the antibody greatly influence developability. For example, the “melting temperature” (Tₘ) of a protein is the temperature at which half of the protein population is in a folded state, and thus is an indicator of thermostability, which is helpful in determining the antibody's stability in storage and manufacturing. Similarly, the “aggregation temperature” (T_{agg}) detects the onset of aggregation, the temperature at which molecules have a tendency to aggregate together and is associated with protein unfolding. In addition, several studies have suggested that monoclonal antibodies can interact non-specifically with themselves (self-aggregation) and other serum proteins; thus, a low self-aggregation property can avoid antibody aggregation, off-target effects, and fast antibody clearance in vivo.

“Off-target” assessments, including polyreactivity and cross-reactivity assessments (e.g., DNA and insulin polyreactive ELISA assay, baculovirus particle (BVP) polyreactive assay, and analysis of human cell membrane proteome array comprising cell surface and secreted proteins), of the humanized anti-CD8α antibodies and antigen binding fragments thereof disclosed herein were used to measure binding to targets other than CD8 antigen. As used herein, the term “polyreactivity” refers to the ability of the antibody to bind a variety of self and foreign antigens that lack structural similarity. As used herein, the term “cross-reactivity” refers to the ability of the antibody to bind antigens in addition to the target antigen due to structural similarity. Particularly for humanized anti-CD8α antibodies, cross-reactivity and polyreactivity assays are used to assess the ability of these antibodies to bind to other antigens beside CD8 that have similar and different structure to CD8, respectively. Polyreactivity and cross-reactivity (or off-target) effects influence various factors including pharmacokinetics, bioavailability, clearance, and toxicity in vivo which all contribute to successful drug/antibody developments.

In some embodiments, anti-CD8α binders of this disclosure have similar binding activity and affinity to CD8 in non-human primates as in humans. The term “cross-species binding” refers to the ability of an antibody to bind to the same or related antigens (“target specific”) across different species while retaining its antigenic specificity to CD8 molecules in such different species. This property is particularly useful in research and clinical development because experimental data in non-human species can be obtained and be reliably translatable to humans.

The skilled artisan will appreciate that properties of anti-CD8α antibodies and antigen binding fragments thereof disclosed herein, including any of the aforementioned embodiments, are often unrelated to an antibody's binding affinity and specificity. In addition, a skilled person would recognize that changes in the amino acid sequence can influence the antibody's biophysical properties. Thus, preservation of affinity alone will not ensure the developability of a humanized antibody, nor can these biophysical properties be reliably predicted from sequence alone. However, testing of at least some variants, in addition to revealing their properties, can provide some indication of the robustness of the particular combination of framework and CDR sequences and what variations in sequence may be problematic, or not.

Thermostability of an antibody is used as an indicator of antibody developability, reflecting stability of the antibody upon storage and during various purification steps that potentially require utilize harsh or stressful conditions. A sensitive measure of the thermostability of an antibody is temperature of aggregation onset, Tagg, indicating the beginning of denaturation of a protein. Generally, a T_{agg} >60° C. is preferable. By this criterion whole antibodies incorporating anti-CD8α antigen binding fragments CBD1033, CBD1034, CBD1035, CBD1039 and CBD1040 all had acceptable T_{agg}, while T_{agg} for CBD1032 was less desirable. Melting temperature, Tm, another measure of thermostability, indicates the midpoint of denaturation of the protein. For potential good developability, an antibody having a Tm of >65° C. is preferable. By this criterion whole antibodies incorporating anti-CD8α antigen binding fragments CBD1033, CBD1035, CBD1039 and CBD1040 all had acceptable Tm. Accordingly, in some embodiments, humanized CT8 antibodies and antigen binding fragments thereof have desirable thermostability and CBD1033, CBD1035, CBD1039 and CBD1040 constitute humanized means for binding CD8α and having desirable thermostability.

One problem that can be encountered in CDR grafting is that the engineered antibody will become polyreactive or have an increased propensity for self-interaction or self-association. These properties can contribute to aggregation, off-target effects, and fast clearance of the antibody (reducing its effectiveness or potency).

Self-interaction can be assessed with affinity-capture self-interaction nanoparticle spectroscopy (AC-SINS) (Phan et al., 2022, MAbs 14(1): 2094750). Full-length antibodies having anti-CD8α antigen binding fragments of CBD1032, CBD1033, CBD1034, CBD1035, CBD1039, or CBD1040 whole antibodies were assessed by AC-SINS and found to all have a low propensity for self-interaction. Accordingly, CBD1032, CBD1033, CBD1034, CBD1035, CBD1039, and CBD1040 constitute a humanized means for binding CD8α with low propensity for self-interaction.

Several tests are available that can be used to assess polyreactivity. One test assesses reactivity with double-stranded DNA (dsDNA) and insulin. By this criterion whole antibodies incorporating an anti-CD8α antigen binding fragment of CBD1032, CBD1033, CBD1034, CBD1035, CBD1039, or CBD1040 were not polyreactive and constitute humanized means for binding CD8α lacking polyreactivity for dsDNA and insulin.

Another test of polyreactivity assesses the ability to bind baculovirus particles (BVP). By this criterion whole antibody incorporating an anti-CD8α antigen binding fragment of CBD1033 was not polyreactive, while CBD1032 was weakly polyreactive. Thus, CBD1033 constitutes a humanized means for binding CD8α lacking polyreactivity for BVP.

A more comprehensive test of polyreactivity and cross-reactivity assesses binding to a panel of >6000 human cell surface and secreted proteins in the form of integral membrane proteins, soluble proteins, and the soluble proteins tethered to a cell surface (Retrogenix Platform, Charles River, High Peak, UK). As these are the proteins most likely to be encountered by a product for in vivo use in humans (or similar), it has relevance for the developability and success of such products. By this assessment CBD1017ch whole antibody comprising the parental antigen binding domain exhibited no cross-reactivity with any of the antigens in the panel. CBD1033, CBD1035 and CBD1039 whole antibodies preserved this lack of cross-reactivity and did not introduce any polyreactivity. Thus, CBD1033, CBD1035 and CBD1039 constitute humanized means for binding CD8α lacking polyreactivity and cross-reactivity for human cell-surface and soluble proteins.

In some aspects of the disclosure, certain amino acid modifications in the VH-CDRs (including amino acid sequence changes N33Q, N33S, N33A, N55Q, N55S, N55A, N103Q, N103S, N103A, or a combination thereof) and VL-CDRs (including but not limited to amino acid sequence changes D30E, D30S, D30A, N34Q, N34S, N34A, N57Q, N57S, N57A, N95Q, N95S, N95A, N96Q, N96S, N96A, D98E, D98S, D98A, or a combination thereof) can be performed to remove labile amides and prevent potential deamidation. In some embodiments, these substitutions do not or only minimally affect binding affinity, but may be advantageous for purification, storage, and other processing of the antibody, whether or not such binding moieties are under high stress conditions (such as high pH and high temperature). As used herein, the term “liability-engineered mutations” refers to mutations to remove labile amino acid residues such as asparagine and aspartic acid which are at risk for post-translational modification, including during product manufacture, by deamidation and isoasparate formation, respectively. In some embodiments, the labile-engineered mutations include one or more of the mutations in the VH-CDRs and VL-CDRs as mentioned above.

The term “high stress condition” encompasses extreme environmental conditions that can affect stability of a molecule, such as high pH (pH≥8), low pH (pH≤6), high temperature (≥40° C.), or a combination thereof.

### Humanized Anti-CD8α as Targeting Moiety on LNPs

Since CD8-positive T cells play an important role in adaptive immunity, the capacity for a humanized anti-CD8α antibody to target CD8-positive T cells can provide therapeutic effects or diagnostic benefits in the treatment of cancer, infections, immune disorders, inflammatory diseases or conditions, and autoimmune diseases. CD8 is also expressed on natural killer (NK) cells which are potent and therapeutically attractive mediators of cytotoxic activity.

In some aspects, any of the aforementioned humanized anti-CD8α antibodies or antigen binding fragments thereof can be used as a targeting moiety on a nanoparticle. A variety of nanoparticles suitable for delivering a payload molecule to or into a cell are known in the art, including polymer and/or lipid containing nanoparticles to which the disclosed anti-CD8 antibodies or other polypeptides comprising the antigen binding domain thereof may be attached as a targeting moiety. In some embodiments, the nanoparticles are lipid nanoparticles (LNPs). In particular embodiments disclosed herein, the term “tLNP” refers to an LNP comprising anti-CD8 antibody or antibody binding fragment thereof as targeting moiety. The term “targeting moiety” refers to a component of a molecule that is capable of binding to another target molecule, in particular, the targeting moiety of the LNP is the anti-CD8 antibody capable of binding to CD8 antigen on CD8 expressing cells.

CD8-targeted tLNPs can be used to deliver payloads, particularly negatively charged payloads such as nucleic acids for tLNP incorporating cationic lipids, into CD8+ cells. This can be done ex vivo or extracorporeally (for example as described in PCT/US2024/035902 which is incorporated by reference for all that is teaches about the use of tLNP to transfect cells ex vivo or extracorporeally) or in vivo. Properties such as the immunogenicity of the binding moiety or its cross-reactivity with antigens other than CD8 are of lesser importance in the context of ex vivo or extracorporeal use than in the context of in vivo use. Thus, humanized antigen binding domains of CT8 with a greater number of mouse residues or that have not been characterized in term of polyreactivity or other measures of potential cross-reactivity with non-CD8 antigens, or even antigen binding domains that have not been humanized, can be useful for providing the binding specificity of a targeting moiety of a tLNP that would be used ex vivo or extracorporeally.

Within the range of affinities exhibited by the various antigen binding domains disclosed herein, affinity has not been a result effective variable for transfection efficiency by tLNP incorporating those antigen binding domains in their targeting moiety, nor for expression level of the transfected mRNA. However, emerging data support the concept that the site where the antibody binds may determine whether particle-internalization is initiated. This is consistent with the observed transfection activity of tLNP incorporating the antigen binding domains of various anti-CD8 antibodies disclosed herein. When the anti-CD8 monoclonal antibodies SK1 and OKT8 provided the antigen binding domain for the targeting moiety of the tLNPs transfection efficiency and payload expression level observed in vitro were substantially less than when the antigen binding domain was provided by CBD1033 (a humanized version of the anti-CD8 monoclonal antibody CT8) or TRX2 (another humanized monoclonal anti-CD8 antibody). Payload expression levels were also similar in vivo for tLNP with targeting moieties incorporating the antigen binding domains of CBD1033 or TRX2. Whereas CBD1033 and TRX2 compete with each other for epitope binding, as does the anti-CD8 antibody YTC182.20, OKT8 and SK1 do not compete with these antibodies. Without being bound to any particular theory, these data indicate that binding to the CT8 epitope (as defined above) enables much higher engineering compared to binders that bind elsewhere and suggest that certain molecular conformational changes are brought about by binding onto specific locations may determine cell signaling resulting in active particle uptake. Accordingly, tLNPs incorporating the antigen binding domain of CT8, TRX2, or YTC182.20 into their targeting moiety constitute means for effective particle internalization or means for efficient transfection of a payload nucleic acid such as DNA, RNA, or mRNA. In various embodiments, such means specifically include or exclude any of the antibodies, antibody formats, or antigen binding domains disclosed herein as a component of their targeting moiety.

Humanized anti-CD8-targeted tLNPs of this disclosure incorporating cationic lipids (such as ionizable cationic lipids) can deliver negatively charged cargos/payloads (such as nucleic acids, polypeptides, and small molecules) into cells expressing CD8. Nucleic acids introduced thereby can encode expression of proteins that are beneficial, inter alia, for the treatment of the subject with the disease. In some aspects, disclosed herein are methods of delivering a nucleic acid (or other negatively charged payload) into a cell comprising contacting the cell with a tLNP encapsulating the nucleic acid or other payload. In some embodiments the contacting takes place ex vivo. In some embodiments, the contacting takes place in vivo. In some instances, the in vivo contacting comprises intravenous, intramuscular, subcutaneous, intranodal or intralymphatic administration. Accordingly, each of the herein disclosed genera, subgenera, and or species of LNP or tLNP disclosed herein including those based on the inclusion or exclusion of particular lipids, particular lipid compositions, particular payloads, and/or particular CD8-targeted moieties can be used in defining the scope of the methods of delivering a payload to a CD8+ cell.

The nucleic acid can include messenger (mRNA), small interfering RNA (siRNA), microRNA (miRNA), antisense oligonucleotide (ASO), self-replicating RNA or circular RNA. In some embodiments, the payload is an mRNA encoding a detectable marker, for example, mCherry fluorescent protein. In some embodiments, the method of delivering is a method of transfecting.

In some embodiments, the encapsulated nucleic acid is an mRNA encoding a chimeric antigen receptor (CAR), a T cell receptor (TCR), or an immune cell engager, such as a BiTE (a bispecific T cell engager), a cytokine, chemokine, chemokine receptor, dominant negative cytokine receptor, a cell-identifying protein tag, a fluorescent protein, or a molecular switch.

The encapsulated nucleic acid can also be an mRNA encoding a gene/genome editing enzyme and/or a guide RNA or other component of a gene/genome editing system. The gene/genome editing component can be a guide RNA for an RNA-directed nuclease or other nucleic acid editing enzyme. Examples of gene editing components that are encoded by a nucleic acid molecule include an mRNA encoding an RNA-guided nuclease, a gene or base editing protein, a prime editing protein, a Gene Writer protein (e.g., a modified or modularized non-long terminal repeat (LTR) retrotransoposon), a retrotransposase, an RNA writer, a zinc finger nuclease (ZFN), a transcription activator-like effector nuclease (TALEN), a meganuclease, a transposase, a retrotransposon, a reverse transcriptase (e.g., M-HLV reverse transcriptase), a nickase or inactive nuclease (e.g., Cas9, nCas9, dCas9), a DNA recombinase, a CRISPR nuclease (e.g., Cas9, Cas12, Cas13, Cas3, CasMINI, Cas7-11, CasX), a DNA nickase, a Cas9 nickase (e.g., D10A or H840A), or any fusion or combination thereof. Other components include a guide RNA (gRNA), a single guide RNA (sgRNA), a prime editing guide RNA (pegRNA), a clustered regularly interspaced short palindromic repeat (CRISPR) RNA (crRNA), a trans-activating clustered regularly interspaced short palindromic repeat (CRISPR) RNA (tracrRNA), or a DNA molecule to be inserted or serve as a template for double-strand break (DSB) repair at a specific genomic locus. Genome-, gene-, and base-editing technology are reviewed in Anzalone et al., Nature Biotechnology 38:824-844, 2020, Sakuma, Gene and Genome Editing 3-4:100017, 2022, and Zhou et al., MedComm 3(3):e155, 2022, each of which is incorporated by reference for all that they teach about the components and uses of this technology to the extent that it does not conflict with the present disclosure.

Delivery of an mRNA into a cell provides transient expression, for several days, of the encoded protein (which may be, for example, a CAR, TCR, or immune cell engager). This may be sufficient and even desirable for therapeutic effects and can also be repeated if somewhat longer expression is desired. Delivery of the components of a gene/genome editing system enables more permanent changes while the editing system will be present for only a short time, however the change to the cells' DNA will persist. A wider array of changes is also possible with a gene/genome editing system. In addition to conferring expression of a particular protein, a gene/genome editing system allows regulation of expression of an individual protein to be changed or to knock out expression of a protein.

In some embodiments comprising multiple agents, the nucleic acid can be multicistronic. In other embodiments comprising multiple agents or components, each agent or component is encoded or contained is a separate nucleic acid species. In some embodiments involving multiple payload nucleic acid species, two or more nucleic acid species are packaged together in a single LNP species. In other embodiments, a subset of the payload nucleic acid species to be delivered, (e.g., a single nucleic acid species) is packaged in one LNP species while another subset of the nucleic acid species is packaged in another LNP species. The different tLNP species can differ by only the payload they contain. The different tLNP species may be combined in a single formulation for administration.

### F(ab′) and F(ab′) Analogs

F(ab′) and F(ab′)-like formats offer certain advantages as targeting moieties for a tLNP. Although any antibody fragment with a structure similar to or derived from that of a classical, proteolytically produced F(ab′) is often referred to as an F(ab′), the term “F(ab′) analog” has been adopted herein to refer to engineered sequences comprising amino acid substitutions and/or that have been truncated and to distinguish them from the paradigmatic natural sequence. F(ab′) are smaller than whole antibodies which can be advantageous in manufacturing. When used as a targeting moiety on a tLNP their antigen binding domain is further from the LNP surface than, for example, a scFv, which can facilitate interaction with the target cell surface. F(ab′) molecules have cysteine residues in the partial hinge region that can be readily conjugated to a functionalized PEG-lipid (for example, a maleimide-functionalized PEG-lipid). Moreover, the F(ab′) can be engineered so that there is unique accessible cysteine enabling for site-specific conjugation which is desirable for product consistency. This can be accomplished with recombinant DNA technology by truncating the hinge region of the F(ab′) or by changing cysteine residues to another amino acid, such as serine, or both.

The hinge region cysteines can form a cystine with another F(ab′) molecule forming an F(ab′)₂ which would make the cysteine unavailable for conjugation to an LNP (more specifically, a functionalized lipid thereof). This can be prevented by processing the F(ab′) under mildly reducing conditions, however, this poses a risk of disrupting the interchain disulfide bond between CL and CH1. That risk can be obviated by relocating the interchain bond to a less accessible region in the molecule.

Some aspects combine constant regions of an F(ab′) or F(ab′) analog with a humanized immunoglobulin antigen binding domain derived from the anti-CD8α antibody CT8 as disclosed herein.

Some aspects combine constant regions of an F(ab′) analog with the antigen binding domain of an anti-CD8 antibody. In some embodiments, the anti-CD8 antigen binding domain recognizes the CT8 epitope. In some embodiments, the anti-CD8 antigen binding domain is derived from YTC182.20, TRX2, or CT8. In some embodiments, the anti-CD8 antigen binding domain comprises a humanized immunoglobulin antigen binding domain derived from the anti-CD8α antibody CT8 as disclosed herein.

In some aspects, an F(ab′) analog engineered as disclosed herein is conjugated to an LNP but is generic with respect to the variable domains of the F(ab′) analog and its specificity.

In some aspects, an F(ab′) or F(ab′) analog constant regions are combined with the antigen binding domain of an anti-CD8 antibody which is conjugated to an LNP. In some embodiments, the anti-CD8 antigen binding domain recognizes the CT8 epitope. In some embodiments, the anti-CD8 antigen binding domain is derived from YTC182.20, TRX2, or CT8. In some embodiments, the anti-CD8 antigen binding domain comprises a humanized immunoglobulin antigen binding domain derived from the anti-CD8α antibody CT8 as disclosed herein.

With respect to these forgoing aspects, in some embodiments, the F(ab′) analog, as appropriate, comprises a relocated interchain disulfide bond, for example, a Cκ S162C substitution paired with an IgG1 or IgG4 CH1 F174C substitution. In further embodiments, one, the other, or both cysteines involved in forming the native interchain disulfide bond are mutated, for example, Cκ C214S, IgG1 C233S, or IgG4 CH1 C127S. In some embodiments, a Cκ domain of an F(ab′) has the amino acid sequence of SEQ ID NO: 41. In some embodiments, a Cκ domain retains C214 as the cysteine for conjugating to an LNP, for example, those comprising SEQ ID NO: 100. Such Cκ domains as those comprising SEQ ID NO: 100 are particularly suitable for pairing with a heavy chain that does not retain a readily accessible cysteine for conjugation to an LNP, such as SEQ ID NO: 99 and the 0.45 design exemplified by CBD1033.45 (see Table 17). In some embodiments the Cκ domain does not retain C214, for example, those comprising SEQ ID NO: 89. Such Cκ domains as those comprising SEQ ID NO: 89 are particularly suitable for pairing with a heavy chain that does retain a readily accessible cysteine for conjugation to an LNP, for example, those comprising SEQ ID NOS: 85, 87, 90, 93, 95, 97, or 103. Such Cκ domains as those comprising SEQ ID NO: 89 are particularly suitable for pairing with a heavy chain that does retain a readily accessible cysteine for conjugation to an LNP, for example, those comprising SEQ ID NOS: 85, 87, 90, 93, 95, 97, or 103. In some embodiment, the F(ab′) analog has a CH region truncated at P245, for example, SEQ ID NOS: 81 or 83. In some embodiments, the F(ab′) analog has a CH region truncated at P241 and has substitutions P240A and P241A, for example, SEQ ID NOS: 85, 87, 90, 93. In some embodiments, the F(ab′) analog has an IgG1 CH region truncated at P240 for example, SEQ ID NO: 95. In some embodiments, the F(ab′) analog has an IgG1 CH region truncated at T238, for example, SEQ ID NO: 97. In some embodiments, the F(ab′) analog has an IgG4 CH region truncated at C239, for example, SEQ ID NO: 103. In some embodiments, the F(ab′) analog cysteine for conjugating to an LNP is C239, for example SEQ ID NOS: 85, 87, 90, 93, 95, or 103. In some embodiments, the F(ab′) analog cysteine for conjugating to an LNP is C233, for example SEQ ID NOS: 97 In some embodiments, the F(ab′) comprises a wildtype IgG1 constant region and has the amino acid sequence of SEQ ID NO: 76 or a wildtype IgG4 constant region and has the amino acid sequence of SEQ ID NO: 79. In some embodiments, the F(ab′) analog comprises an IgG1 constant region and comprises the amino acid sequence of SEQ ID NO: 81, SEQ ID NO: 85, SEQ ID NO: 90, SEQ ID NO: 95, SEQ ID NO: 97, or SEQ ID NO: 99. In some embodiments, the F(ab′) analog comprises a heavy chain comprising the amino acid sequence of SEQ ID NO: 78, SEQ ID NO: 82, SEQ ID NO: 86, SEQ ID NO: 92, SEQ ID NO: 96, SEQ ID NO: 98, SEQ ID NO: 102, SEQ ID NO: 105, SEQ ID NO: 106, SEQ ID NO: 108, SEQ ID NO: 109, SEQ ID NO: 110, SEQ ID NO: 111, SEQ ID NO: 113, or SEQ ID NO: 114. In some embodiments, the F(ab′) analog comprises an IgG4 constant region that comprises the amino acid sequence of SEQ ID NO: 83, SEQ ID NO: 87, SEQ ID NO: 93, or SEQ ID NO: 103. In some embodiments, the F(ab′) analog comprises a heavy chain comprising the amino acid sequence of SEQ ID NO: 84, SEQ ID NO: 88, SEQ ID NO: 94, or SEQ ID NO: 104.

CBD1033 variable domains have been joined with the above constant regions in a variety of F(ab′) and F(ab′) analog designs as set out in Table 17 (below). In various embodiments, and of the humanized CT8 variable domains, or the variable domains of other antibodies may be incorporated into these designs. Accordingly, in various embodiments, the targeting moiety of an LNP can be an F(ab′) and F(ab′) analog of any of the designs set out in Table 17. In some embodiments, the targeting domain has a 0.37 design. In some embodiments, the targeting domain has a 0.44 design. In some embodiments, the targeting domain has a 0.45 design.
Lipid Nanoparticles (LNPs) and Targeted LNPs (tLNPs)

A variety of LNP compositions are known in the art that can serve as the basis for a targeted LNP (tLNP). For in vivo use, LNPs made up of a cationic lipid (in particular an ionizable cationic lipid), a neutral lipid (such as a phospholipid), a sterol (such as cholesterol), and a polymer-conjugated lipid (such as a polyethylene glycol (PEG)-lipid) have shown advantageous properties. In particular embodiments of this disclosure, a tLNP comprises an ionizable cation lipid, a phospholipid, a sterol, and a PEG-lipid comprising a non-functionalized PEG-lipid and a functionalized PEG-lipid. Table 14 provides a list of various LNP compositions that have been demonstrated to form LNP encapsulating mRNA and to which a polypeptide comprising an antibody or an antigen binding domain thereof can be conjugated as a targeting moiety. In some embodiments, the targeting moiety is an engineered F(ab′) as disclosed herein. In some embodiments, the targeting moiety comprises an antigen binding domain with specificity for CD8, such as having specificity for human CD8, whether the targeting moiety is a whole antibody, engineered F(ab′), or some other form of antibody. In some instances, the anti-CD8 antigen binding domain is a humanized anti-CD8 antigen binding domain disclosed herein. In some instances, the tLNP has the lipid content of composition F9 in Table 14. In some embodiments, composition F9 in Table 14 is used to generate tLNP comprising an anti-CD8 binding moiety as its targeting moiety.

In any of the aforementioned tLNP embodiments, certain embodiments include tLNPs in which the targeting moiety comprises one of the herein disclosed humanized antigen binding domains of CT8, such as one that comprises a VL region having the amino acid sequence of SEQ ID NO: 17 and a VH region having the amino acid sequence of one of SEQ ID NOs: 11 or 27-29. In some such embodiments, the targeting moiety is a whole humanized anti-CD8 antibody comprising heavy chain with a silenced Fc region such as one having the amino acid sequence of SEQ ID NO: 43 or 44. In certain instances, the whole humanized anti-CD8 antibody comprising a heavy chain with a silenced Fc region comprises the sequence of CBD1033HC (SEQ ID NO: 61) and/or a light chain comprising the sequence of CBD1033LC (SEQ ID NO: 62). In some embodiments, the targeting moiety is an anti-CD8 F(ab′) of a classic F(ab′). In some embodiments, the targeting moiety is an anti-CD8 F(ab′) of an engineered F(ab′). Examples of such anti-CD8 F(ab′) of a classic F(ab′) or an engineered F(ab′) are listed in Table 17. In some embodiments, the anti-CD8 F(ab′) comprises a light chain with a wild-type Kappa constant region, wherein the Kappa constant region has the amino acid sequence SEQ ID NO: 41. In some embodiments, the anti-CD8 F(ab′) comprises a light chain with an engineered Kappa constant region, wherein the Kappa constant region has the amino acid sequence SEQ ID NO: 89, or SEQ ID NO: 100. In some embodiments, the anti-CD8 F(ab′) comprises a heavy chain with a wild-type IgG1 F(ab′), wherein the IgG1 F(ab′) has the amino acid sequence SEQ ID NO: 76. In some embodiments, the anti-CD8 F(ab′) comprises a heavy chain with an engineered IgG1 F(ab′), wherein the IgG1 F(ab′) has the amino acid sequence SEQ ID NO: 81, SEQ ID NO: 85, SEQ ID NO: 90, SEQ ID NO: 95, SEQ ID NO: 97, or SEQ ID NO: 99. In some embodiments, the anti-CD8 F(ab′) comprises a heavy chain with a wild-type IgG4 F(ab′), wherein the IgG4 F(ab′) has the amino acid sequence SEQ ID NO: 79. In some embodiments, the anti-CD8 F(ab′) comprises a heavy chain with an engineered IgG4 F(ab′), wherein the IgG4 F(ab′) has the amino acid sequence SEQ ID NO: 83, SEQ ID NO: 87, SEQ ID NO: 93, or SEQ ID NO: 103. In some embodiments, the anti-CD8 F(ab′) comprises a light chain having the amino acid sequence of SEQ ID NO: 77, SEQ ID NO: 91, SEQ ID NO: 101, SEQ ID NO: 107, or SEQ ID NO: 112. In some embodiments, the anti-CD8 F(ab′) comprises a heavy chain having the amino acid sequence of SEQ ID NO: 78, SEQ ID NO: 82, SEQ ID NO: 86, SEQ ID NO: 92, SEQ ID NO: 96, SEQ ID NO: 98, SEQ ID NO: 102, SEQ ID NO: 105, SEQ ID NO: 106, SEQ ID NO: 108, SEQ ID NO: 109, SEQ ID NO: 110, SEQ ID NO: 111, SEQ ID NO: 113, or SEQ ID NO: 114. In some embodiments, the anti-CD8 F(ab′) comprises a heavy chain having the amino acid sequence of SEQ ID NO: 80, SEQ ID NO: 84, SEQ ID NO: 88, SEQ ID NO: 94, or SEQ ID NO: 104. In any of the aforementioned tLNP embodiments encapsulating a herein disclosed mRNA, in certain embodiments the tLNP comprises as its targeting moiety an antibody or antigen binding portion thereof comprising one of the aforementioned herein disclosed humanized antigen binding domains of CT8.

In some embodiments, the targeting moiety of a tLNP is a classic or engineered F(ab′). In some embodiments, the targeting moiety of a tLNP comprises an engineered F(ab′). Examples of such F(ab′) comprising a wild-type or engineered constant region are listed in Table 17. In some such embodiments, the F(ab′) comprises a light chain with a wild-type Kappa constant region, wherein the Kappa constant region has the amino acid sequence SEQ ID NO: 41. In some such embodiments, the F(ab′) comprises a light chain with an engineered Kappa constant region, wherein the Kappa constant region has the amino acid sequence SEQ ID NO: 89, or SEQ ID NO: 100. In some such embodiments, the F(ab′) comprises a heavy chain with a wild-type IgG1 F(ab′) constant region, wherein the IgG1 F(ab′) constant region has the amino acid sequence SEQ ID NO: 76. In some such embodiments, the F(ab′) comprises a heavy chain with an engineered IgG1 F(ab′) constant region, wherein the IgG1 F(ab′) constant region has the amino acid sequence SEQ ID NO: 81, SEQ ID NO: 85, SEQ ID NO: 90, SEQ ID NO: 95, SEQ ID NO: 97, or SEQ ID NO: 99. In some such embodiments, the F(ab′) comprises a heavy chain with a wild-type IgG4 F(ab′) constant region, wherein the IgG4 F(ab′) constant region has the amino acid sequence SEQ ID NO: 79. In some such embodiments, the F(ab′) comprises a heavy chain with an engineered IgG4 F(ab′) constant region, wherein the IgG4 F(ab′) constant region has the amino acid sequence SEQ ID NO: 83, SEQ ID NO: 87, SEQ ID NO: 93, or SEQ ID NO: 103.

In some embodiments, a tLNP comprises a F(ab′) or anti-CD8 F(ab′) comprising a S162C kappa chain substitution and an IgG1 or IgG4 CH1 F174C substitution. In some embodiments, the F(ab′) or anti-CD8 F(ab′) heavy chain further comprises an IgG1 CH1 C233S or IgG4 CH1 C127S substitution. In some embodiments, the F(ab′) or anti-CD8 F(ab′) light chain further comprises a C214S kappa chain substitution.

In some embodiments of a tLNP comprising a F(ab′) targeting moiety comprising a herein disclosed constant region, the tLNP comprises one or more ionizable cationic lipids disclosed herein. In some embodiments of a tLNP comprising a F(ab′) targeting moiety comprising a herein disclosed constant region, the tLNP comprises a LNP composition as disclosed in Table 14, for example F9.

In certain embodiments, a LNP (or tLNP) comprises a binding moiety specific for an immune cell antigen selected from CD1, CD2*^{†‡}, CD3*^{†‡}, CD4*^{†‡}, CD5^{†‡}, CD7^{†‡}, CD8^{†}, CD11b^{‡}, CD14^{†‡}, CD16, CD25^{†‡}, CD26^{‡}, CD27*^{†‡}, CD28*^{†‡}, CD30*^{†‡}, CD32*, CD38*^{†‡}, CD39^{‡}, CD40*^{†‡}, CD40L (CD154)*^{†‡}, CD44*^{‡}, CD45^{†‡}, CD56^{†‡}, CD64*^{‡}, CD62^{†‡}, CD68, CD69^{‡}, CD73^{†‡}, CD8β*^{‡}, CD83^{‡}, CD86*^{‡}, CD95^{‡}, CD103^{‡}, CD119^{‡}, CD126^{‡}, CD137 (41BB)^{†‡}, CD150^{‡}, CD153^{‡}, CD161^{‡}, CD166^{‡}, CD183 (CXCR3)^{‡}, CD183 (CXCR5)^{‡}, CD223 (LAG-3)*^{†‡}, CD254^{‡}, CD275^{‡}, CD45RA, CTLA-4*^{†}*^{†}, DEC205, OX40^{†}, PD-1*^{†‡}, GITR†, TIM-3*^{†‡}, FasL*^{‡}, IL18R1, ICOS (CD278)^{‡}, leu-12, TCR†, TLR1, TLR2^{†‡}, TLR3*^{‡}, TLR4^{†‡}, TLR6, TREM2^{‡}, NKG2D‡, CCR, CCR1 (CD191)^{‡}, CCR2 (CD192)*^{†‡}, CCR4 (CD194)*^{†‡}, CCR6 (CD196)^{‡}, CCR7^{‡}, low affinity IL-2 receptor^{†‡}, IL-7 receptor^{‡}, IL-12 receptor^{‡}, IL-15 receptor^{‡}, IL-18 receptor^{‡}, and IL-21 receptor^{†‡}. In further embodiments, a tLNP comprises a binding moiety specific for an HSC surface molecule selected from CD117^{†}, CD34*^{‡}, CD44*^{‡}, CD45^{†‡}, CD90 (Thy1)^{‡}, CD105^{‡}, CD133^{‡}, BMPR2^{‡}, and Sca-1; or specific for an MSC surface molecules selected from CD70*^{‡}, CD105^{‡}, CD73^{‡}, Stro-1, SSEA-3^{‡}, SSEA-4^{‡}, CD271^{‡}, CD146^{‡}, GD2*^{†‡}, SUSD2, Stro-4, MSCA-1, CD56^{‡}, CD200*^{†‡}, PODXL^{‡}, CD13^{‡}, CD29*^{‡}, CD44*^{‡}, and CD10‡. In various embodiments, a binding moiety is an antibody or antigen-binding portion thereof. (* indicates that exemplary antibodies with the indicated specificity from which a binding moiety could be derived can be found in U.S. Pat. No. 11,326,182B2 Table 9 or 10. I indicates that exemplary antibodies with the indicated specificity from which a binding moiety could be derived can be found in Wilkinson & Hale, 2022. Both references cited and incorporated by reference above. $ indicates that exemplary antibodies with the indicated specificity from which a binding moiety could be derived can be found in the Therapeutic Antibody Database (TABS) at tabs.craic.com). Other suitable antibodies can be found in Appendix A.

The LNPs of this disclosure are multicomponent compositions comprising a payload and multiple lipid components, including an ionizable cationic lipid, non-functionalized and/or functionalized PEG-lipids, a phospholipid, and a sterol. The tLNPs of this disclosure are multicomponent compositions comprising an LNP and a binding moiety such as a humanized anti-CD8 binder/antibody. As used herein, the term “tLNP composition” refers to the same features as LNP composition with the addition of an anti-CD8 binding moiety that serves as a targeting moiety and the binding moiety's density on the tLNP can be expressed as a ratio to the payload on a w/w basis. Table 14 provides a list of lipid compositions that have been shown to form LNP. In some embodiments, an LNP or tLNP comprises a payload with a net negative charge selected from a peptide, a polypeptide, a protein, a small molecule, or a nucleic acid molecule, and combinations thereof. A payload is generally encompassed by or in the interior of an LNP or tLNP. As disclosed herein, dosages always refer to the amount of payload being provided.

As used herein, the term “LNP composition” refers to the lipid components present in an LNP, their molar ratios (e.g., mol %) relative to each other, and the ratio of payload to total lipid. In certain aspects the payload comprises one or more species of nucleic acid molecule or other negatively charged molecules. That is, in some embodiments, the payload comprises only a single species of nucleic acid or other negatively charged molecule (or consists of such species or molecule) while in other embodiments, the payload comprises multiple species of nucleic acid or other negatively charged molecules, for example, 2, 3, or 4 such species or molecules. In some embodiments in which the payload comprises multiple nucleic acid species or other negatively charged molecules, more than one of the species up to and including all of the species are reactive, or encode a polypeptide that is reactive, with a same target.

LNP and tLNP Compositions

The LNP composition contributes to the formation of stable LNPs and tLNPs, efficient encapsulation of a payload, protection of a payload from degradation until it is delivered into a cell, and promotion of endosomal escape of a payload into the cytoplasm. These functions are primarily independent of the specificity of the binding moiety (or moieties) serving to direct or bias a tLNP to a particular cell type(s). Additional LNP and tLNP compositions are generally disclosed in PCT/US2024/032141, filed 31 May 2024 and entitled Lipid Nanoparticle Formulations and Compositions, which is incorporated by reference for all that it teaches about the design, formation, characterization, properties, and use of LNPs and tLNPs.

The LNPs and/or tLNPs can include the various components in amounts sufficient to provide a nanoparticle with a desired shape, fluidity, and bio-acceptability as described herein. With respect to LNPs or tLNPs of this disclosure, in some embodiments, the LNP (or tLNP) comprises at least one ionizable cationic lipid (e.g., as described herein) in an amount in the range of from about 35 to about 65 mol %, or any integer bound sub-range thereof, e.g., in an amount of from about 40 to about 65 mol %, or about 40 to about 60 mol %, or about 40 molt % to about 62 mol %. In some embodiments, the LNP or tLNP comprises about 58 mol %, about 60 mol %, or 62 mol % ionizable cationic lipid. In some embodiments, the LNP (or tLNP) comprises a phospholipid in an amount in the range of from about 7 to about 30 mol %, or any integer bound sub-range thereof, e.g., in an amount of from about 13 to about 30 mol %. In some embodiments, the LNP or tLNP comprises about 10 mol % phospholipid. In some embodiments, the LNP (or tLNP) comprises a sterol in an amount in the range of from about 20 to about 50 mol % or any integer bound sub-range thereof, e.g., in an amount in the range of from about 20 to about 45 mol %, or about 30 to about 50 mol %, or about 30 to about 45 mol %. In some embodiments, the LNP or tLNP comprises about 30.5, 26.5, or 23.5 mol % sterol. In some embodiments, the LNP (or tLNP) comprises at least one co-lipid in an amount in the range of from about 1 to about 30 mol %. In some embodiments, an LNP or tLNP comprises total PEG-lipid in an amount in the range of from about 1 mol % to about 5 mol % or any integer×101 bound sub-range thereof, e.g., in an amount in the range of from about 1 mol % to about 2 mol % total PEG-lipid. In some embodiments, the LNP (or tLNP) comprises at least one unfunctionalized PEG-lipid in an amount of from 0 to about 5 mol % or any integer×101 bound sub-range thereof, e.g., in the range of amount 0 to about 3 mol %, or about 0.1 to about 5 mol %, or about 0.5 to about 5 mol %, or about 0.5 to about 3 mol %. In some embodiments, the LNP or tLNP comprises about 1.4 mol % unfunctionalized PEG-lipid. In some embodiments, the LNP or tLNP comprises at least one functionalized PEG-lipid in an amount in the range of from about 0.1 to about 5 mol % or any integer×101 bound sub-range thereof, e.g., in the range of from about 0.1 to 0.3 mol %. In certain embodiments, an LNP or tLNP comprises about 0.1 mol %, about 0.2 mol %, or about 0.3 mol % functionalized PEG-lipid. In some embodiments, the LNP or tLNP comprises about 0.1 mol % functionalized PEG-lipid. In some embodiments, the functionalized PEG-lipid is conjugated to a binding moiety. In some embodiments the binding moiety is an engineered F(ab′) as disclosed herein. In some embodiments, the binding moiety comprises an anti-CD8 antigen binding domain such as the humanized anti-CD8α antigen binding domains disclosed herein. In certain instances, a tLNP comprises an anti-CD8α whole antibody as the binding moiety which is present at an antibody:mRNA ratio (w/w) of about 0.3 to about 1.0.

In certain aspects, this disclosure provides an LNP or tLNP, wherein the LNP or tLNP comprises about 35 mol % to about 65 mol % of an ionizable cationic lipid, about 0.5 mol % to about 3 mol % of a PEG-lipid (including non-functionalized PEG-lipid and optionally a functionalized PEG-lipid), about 7 mol % to about 13 mol % of a phospholipid, and about 30 mol % to about 50 mol % of a sterol. In some embodiments, an LNP or tLNP comprises a payload with a net negative charge for example, a peptide, a polypeptide, a protein, a small molecule, or a nucleic acid molecule, and combinations thereof. A payload is generally encompassed by or in the interior of an LNP or tLNP. As disclosed herein dosages always refer to the amount of payload being provided. In some embodiments, a payload comprises one or more species of nucleic acid molecule. For tLNP encapsulating mRNA dosages are typically in the range of 0.05 to 5 mg/kg without regard for recipient species. In some embodiments, the dosage is in the range of 0.1 to 1 mg/kg.

The payload to total lipid ratio can be expressed on a w/w basis or for nucleic acid molecules as an N/P ratio. With respect to LNPs or tLNPs of this disclosure, in some embodiments, the ratio of total lipid to nucleic acid is about 10:1 to about 50:1 on a weight basis. In some embodiments, the ratio of total lipid to nucleic acid is about 10:1, about 20:1, about 30:1, or about 40:1 to about 50:1, or 10:1 to 20:1, 30:1, 40:1 or 50:1, or any range bound by a pair of these ratios. The ratio of lipid to nucleic acid can also be reported as an N/P ratio, the ratio of positively chargeable lipid amine (N=nitrogen) groups to negatively-charged nucleic acid molecule phosphate (P) groups. In some embodiments, the N/P ratio is from about 3 to about 9, about 3 to about 7, about 3 to about 6, about 4 to about 6, about 5 to about 6, or about 6. In some embodiments, the N/P ratio is from 3 to 9, 3 to 7, 3 to 6, 4 to 6, 5 to 6, or 6.

Due to physiologic and manufacturing constraints LNP or tLNP, particles with a hydrodynamic diameter of about 50 to about 150 nm are desirable for in vivo use. Accordingly, in some embodiments, the LNP or tLNP has a hydrodynamic diameter of 50 to 150 nm and in some embodiments the hydrodynamic diameter is ≤120, ≤110, ≤100, or ≤90 nm. Uniformity of particle size is also desirable with a polydispersity index (PDI) of ≤0.2 (on a scale of 0 to 1) being acceptable. Both hydrodynamic diameter and polydispersity index are determined by dynamic light scattering (DLS). Particle diameter as assessed from cryo-transmission electron microscopy (Cryo-TEM) can be smaller than the DLS-determined value.

A binding moiety's density on the tLNP can be defined according to the ratio of antibody (binder) to mRNA (w/w) either based on the amount of antibody input in the conjugation reaction or as measured in the tLNP. For an intact antibody (e.g., whole IgG), in some embodiments, preferred ratios are about 0.3 to about 1.0, about 0.3 to about 0.7, about 0.3 to about 0.5, about 0.5 to about 1.0, and about 0.5 to about 0.7 for either the input or final measured binder ratio. In certain embodiments, a tLNP has an antibody ratio of 0.3 to 1.0, 0.3 to 0.7, 0.3 to 0.5, 0.5 to 1.0, and 0.5 to 0.7 for either the input or final measured binding moiety density ratio. In some embodiments, if the binder is different in size from an intact antibody (for example a scFv, diabody, or minibody, etc.) the w/w ratio is adjusted for the different size of the binding moiety.

### Ionizable Cationic Lipids

In particular embodiments, the ionizable cationic lipids are described in US20230320995A1; International Application PCT/US2024/049627 and PCT/US2024/049649; U.S. provisional applications 63/632,944 and 63/632,940; the disclosures of which are fully incorporated by reference herein. Ionizable cationic lipids are useful components for complexing with negatively charged payloads and for promoting delivery of the payload into the cytoplasm of a cell following endocytosis. Accordingly, each of the genera and species of ionizable cationic lipid disclosed herein can be used in LNP and tLNP formulations and compositions of this disclosure, and methods of using them. In certain embodiments, ionizable cationic lipid(s) of an LNP having a measured pKa of 6 to 7 may remain essentially neutral in the blood stream and interstitial spaces but ionize after uptake into cells as the endosomes acidify. Upon acidification in the endosomal space, the lipid becomes protonated, and associates more strongly with the phosphate backbone of the nucleic acid, which destabilizes the structure of the LNP and promotes nucleic acid release from the LNP into the cell cytoplasm (also referred to as endosomal escape). Thus, the herein disclosed ionizable cationic lipids constitute means for destabilizing LNP structure (when ionized) or means for promoting nucleic acid release or endosomal escape.

For simplicity, chemical moieties are defined and referred to throughout primarily as univalent chemical moieties (e.g., alkyl, aryl, and the like). Nevertheless, such terms may also be used to convey corresponding multivalent moieties under the appropriate structural circumstances clear to those skilled in the art. For example, while an “alkyl” moiety generally refers to a monovalent radical (e.g. CH₃—CH₂—), in certain circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene.) All atoms are understood to have their normal number of valences for bond formation (i.e., 4 for carbon, 3 for nitrogen, 2 for oxygen, and 2, 4, or 6 for sulfur, depending on the oxidation state of the sulfur atom).

The term “alkyl” as employed herein refers to saturated straight and branched chain aliphatic groups having from 1 to 12 carbon atoms. As such, “alkyl” encompasses C₁, C₂, C₃, C₄, C₅, C₆, C₇, C₈, C₉, C₁₀, C₁₁ and C₁₂ groups.

The term “alkenyl” as used herein means an unsaturated straight or branched chain aliphatic group with one or more carbon-carbon double bonds, having from 2 to 12 carbon atoms. As such, “alkenyl” encompasses C₂, C₃, C₄, C₅, C₆, C₇, C₈, C₉, C₁₀, C₁₁ and C₁₂ groups.

In some embodiments, the hydrocarbon chain is unsubstituted. In other embodiments, one or more hydrogens of the alkyl or alkenyl group may be substituted with the same or different substituents.

Aryl refers to an aromatic or heteroaromatic ring lacking one hydrogen leaving a bond that connects to another portion of an organic molecule. Examples of aryl include, without limitation, phenyl, naphthalenyl, pyridine, pyrimidine, pyrazine, pyrrole, furan, thiophene, imidazole, thiazole, oxazole, and the like.

Aryl-alkyl refers to a moiety comprising one or more aryl rings and one or more alkyl moieties. The position of the one or more aryl rings can vary within the alkyl portion of the moiety. For example, the one or more aryl rings may be at an end of the one or more alkyl moieties, be fused into the carbon chain of the one or more alkyl moieties, or substitute one or more hydrogens of one or more alkyl moieties; and the one or more alkyl moieties may substitute one or more hydrogens of the one or more aryl rings. In some embodiments, there is a single ring; while in other embodiments, that are multiple rings.

Branched alkyl is a saturated alkyl moiety wherein the alkyl group is not a straight chain. Alkyl portions such as methyl, ethyl, propyl, butyl, and the like, can be appended to variable positions of the main alkyl chain. In some embodiments, there is a single branch; while in other embodiments, there are multiple branches.

Branched alkenyl refers to an alkenyl group comprising at least one branch off the main chain which may be formed by substituting one or more hydrogens of the main chain with the same or different alkyl groups, e.g., without limitation, methyl, ethyl, propyl, butyl, and the like. In some embodiments, a branched alkenyl is a single branch structure, while in other embodiments, a branched alkenyl may have multiple branches.

Straight chain alkyl is a non-branched, non-cyclic version of the alkyl moiety described above.

Straight chain alkenyl is a non-branched, non-cyclic version of the alkenyl moiety described above.

In certain aspects, the ionizable cationic lipids of this disclosure have a structure of the formula M5:

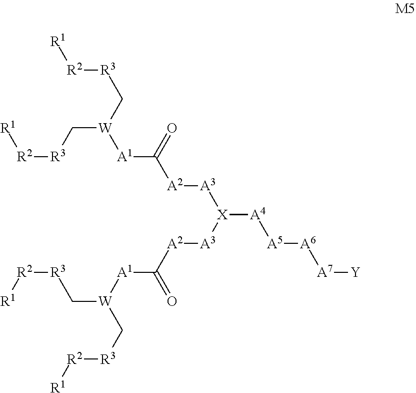

wherein:
each R¹ is independently selected from a C₇-C₁₁ alkyl or a C₇-C₁₁ alkenyl,
A¹ is (CH₂)₁₋₂,
A² is O,
A³ is (CH₂)₁₋₅, wherein A³ is not CH₂ if X is N,
X is N, CH, or C—CH₃,
A⁴ is CH₂, C═O, NH, NCH₃, or O,
A⁵ is absent, O, S, NH, or NCH₃ if A⁴ is C═O, or A⁵ is C═O if A⁴ is not C═O,
A⁶ is O, S, NH, NCH₃ or (CH₂)₀₋₂,
A⁷ is (CH₂)₀₋₆ wherein if A⁶ is O, S, NH, NCH₃, A⁷ is (CH₂)₂₋₄,
Y is

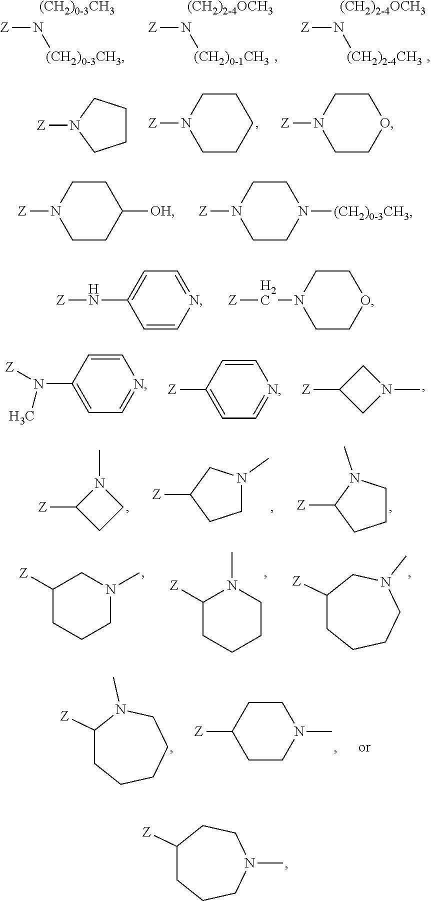

wherein Z is a bond; and
R² is O, R³ is C═O and W is CH or N, or R² is C═O, R³ is O and W is CH;
   wherein A⁶ and A⁷ are not both (CH₂)₀ unless A⁵ is C═0;
   wherein
a), A¹ is CH₂, A³ is (CH₂)₂₋₅, X is N, A⁴ is C═O, A⁵ is O, S, NH, NCH₃, A⁶ is (CH₂)₁₋₂, A⁷ is (CH₂)₁₋₄, or
b) A¹ is CH₂, A³ is (CH₂)₁₋₄, X is CH, A⁴ is CH₂, NH, NCH₃, O, A⁵ is C═O, A⁶ is O, NH, NCH₃, A⁷ is (CH)₂₋₆, or
c) A¹ is (CH₂)₂, A³ is (CH₂)₁₋₄, X is C—CH₃, A⁴ is C═O, A⁵ is O, NH, NCH₃, A⁶ is (CH₂)₁₋₂, A⁷ is (CH₂)₁₋₄, or
d) A¹ is CH₂, A³ is (CH₂)₂₋₅, X is N, A⁴ is C═O, A⁵ is absent, A⁶ is (CH₂)₀, A⁷ is (CH₂)₀, and Y is

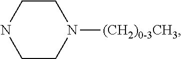

or
e) A¹ is CH₂, A³ is (CH₂)₁₋₅, X is CH, A⁴ is CH₂, NH, NCH₃ or O, A⁵ is C═O, A⁶ is (CH₂)₀, A⁷ is (CH₂)₀, and Y is

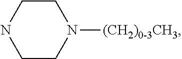

or
f) A¹ is (CH₂)₂, A³ is (CH₂)₁₋₅, X is CCH₃, A⁴ is C═O, A⁵ is absent, A⁶ is (CH₂)₀, A⁷ is (CH₂)₀, and Y is

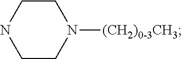

wherein
the number of contiguous atoms present in a span:

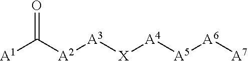

is in the range from 7-17.

As used herein, when a subscript has a value of “0”, the group is absent. For example, when A⁶ is (CH₂)₀, A⁶ is absent.

In certain embodiments of formula M5, R² is O, R³ is C═O and W is CH or N. For example, in certain embodiments of formula M5, R² is O, R³ is C═O and W is CH.

In certain embodiments of formula M5, R² is C═O, R³ is O and W is CH.

In certain embodiments of formula M5, A¹ is CH₂, A³ is (CH₂)₂₋₅, X is N, A⁴ is C═O, A⁵ is O, S, NH, NCH₃, A⁶ is (CH₂)₁₋₂, and A⁷ is (CH₂)₁₋₄. For example, in certain embodiments A¹ is CH₂, A³ is (CH₂)₂₋₅, X is N, A⁴ is C═O, A⁵ is O, A⁶ is (CH₂)₁₋₂, and A⁷ is (CH₂)₁₋₄.

In certain embodiments of formula M5, A¹ is CH₂, A³ is (CH₂)₁₋₄, X is CH, A⁴ is CH₂, NH, NCH₃, O, A⁵ is C═O, A⁶ is O, NH, NCH₃, or CH₂, and A⁷ is (CH)₂₋₆. In certain embodiments as described herein, A¹ is CH₂, A³ is (CH₂)₁₋₄, X is CH, A⁴ is NH, A⁵ is C═O, A⁶ is O, NH, NCH₃, or CH₂, and A⁷ is (CH)₂₋₆. For example, in certain embodiments of formula M5, A¹ is CH₂, A³ is (CH₂)₁₋₄, X is CH, A⁴ is NH, A⁵ is C═O, A⁶ is O, and A⁷ is (CH)₂₋₆. For example, in certain embodiments of formula M5, A¹ is CH₂, A³ is (CH₂)₁₋₄, X is CH, A⁴ is NH, A⁵ is C═O, A⁶ is CH₂, and A⁷ is (CH)₂₋₆. In certain embodiments as described herein, A¹ is CH₂, A³ is (CH₂)₁₋₄, X is CH, A⁴ is CH₂, A⁵ is C═O, A⁶ is O, NH, NCH₃, or CH₂, or A⁷ is (CH)₂₋₆. For example, in certain embodiments of formula M5, A¹ is CH₂, A³ is (CH₂)₁₋₄, X is CH, A⁴ is CH₂, A⁵ is C═O, A⁶ is O, A⁷ is (CH)₂₋₆. In certain embodiments as described herein, A¹ is CH₂, A³ is (CH₂)₁₋₄, X is CH, A⁴ is O, A⁵ is C═O, A⁶ is O, NH, NCH₃, or CH₂, and A⁷ is (CH)₂₋₆. For example, in certain embodiments of formula M5, A¹ is CH₂, A³ is (CH₂)₁₋₄, X is CH, A⁴ is O, A⁵ is C═O, A⁶ is CH₂, and A⁷ is (CH)₂₋₆. In certain embodiments as described herein, A¹ is CH₂, A³ is (CH₂)₁₋₄, X is CH, A⁴ is NCH₃, A⁵ is C═O, A⁶ is O, NH, NCH₃, or CH₂, and A⁷ is (CH)₂₋₆. For example, in certain embodiments of formula M5, A¹ is CH₂, A³ is (CH₂)₁₋₄, X is CH, A⁴ is NCH₃, A⁵ is C═O, A⁶ is CH₂, and A⁷ is (CH)₂₋₆.

In certain embodiments of formula M5, A¹ is (CH₂)₂, A³ is (CH₂)₁₋₄, X is C—CH₃, A⁴ is C═O, A⁵ is O, NH, NCH₃, A⁶ is (CH₂)₁₋₂, or A⁷ is (CH₂)₁₋₄. For example, in certain embodiments, A¹ is (CH₂)₂, A³ is (CH₂)₁₋₄, X is C—CH₃, A⁴ is C═O, A⁵ is O, A⁶ is (CH₂)₁₋₂, or A⁷ is (CH₂)₁₋₄.

In certain embodiments of formula M5, the number of contiguous connective atoms present in a span:

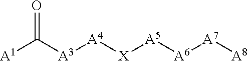

is in the range from 7-17. For example, in certain embodiments, the number of contiguous connective atoms present in a span:

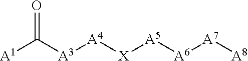

is in the range of 7-11 or 7-10. In certain embodiments, the number of contiguous connective atoms present in a span:

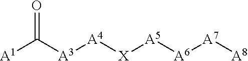

is in the range of 10-17 (e.g., in the range of 10-16, or 10-14, or 10-12). For example, in certain embodiments, the number of contiguous connective atoms present in a span:

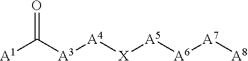

is 10. For example, in certain embodiments, the number of contiguous connective atoms present in a span:

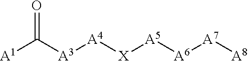

is 7. The present inventors have found that changing the number of contiguous connective atoms present in each span can allow for tuning of the pKa of the cationic lipid.

In some embodiments of formula M5, Y is

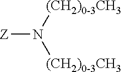

and Z is a bond. In some embodiments of formula M5, Y is

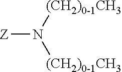

and Z is a bond. For example, in some embodiments of formula M5, Y is

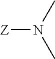

and Z is a bond.

In some embodiments of formula M5, Y is

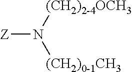

and Z is a bond. For example, on some embodiments of formula M5, Y is

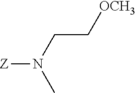

and Z is a bond.

In some embodiments of formula M5, Y is

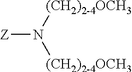

and Z is a bond. For example, in some embodiments of formula M5, Y is

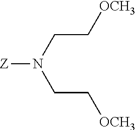

and Z is a bond.

In some embodiments of formula M5, Y is

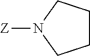

and Z is a bond.

In some embodiments of formula M5, Y is

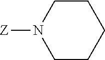

and Z is a bond.

In some embodiments of formula M5, Y is

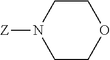

and Z is a bond.

In some embodiments of formula M5, Y is

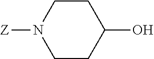

and Z is a bond.

In some embodiment of formula M5, Y is

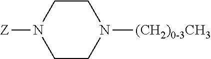

and Z is a bond.

In some embodiments of formula M5, Y is

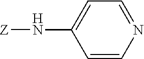

and Z is a bond.

In some embodiments of formula M5, Y is

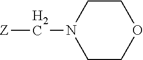

and Z is a bond.

In some embodiments of formula M5, Y is

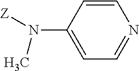

and Z is a bond.

In some embodiments of formula M5, Y is

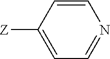

and Z is a bond.

In some embodiments of formula M5, Y is

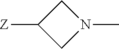

and Z is a bond.

In some embodiments of formula M5, Y is

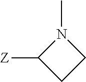

and Z is a bond.

In some embodiments of formula M5, Y is

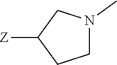

and Z is a bond.

In some embodiments of formula M5, Y is

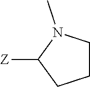

and Z is a bond.

In some embodiments of formula M5, Y is

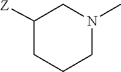

and Z is a bond.

In some embodiments of formula M5, Y is

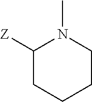

and Z is a bond.

In some embodiments of formula M5, Y is

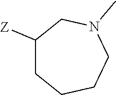

and Z is a bond.

In some embodiments of formula M5, Y is

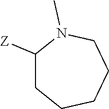

and Z is a bond.

In some embodiments of formula M5, Y is

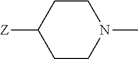

and Z is a bond.

In some embodiments of formula M5, Y is

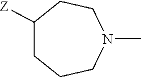

and Z is a bond.

In some embodiments as described herein, the ionizable cationic lipids have a structure of Formula 1:

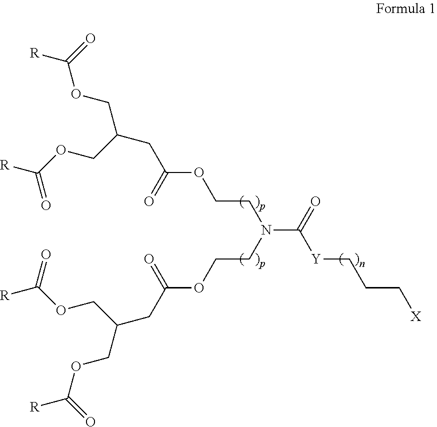

wherein:
Y is O, NH, N—CH₃, or CH₂,
n is an integer from 0 to 4,
X is

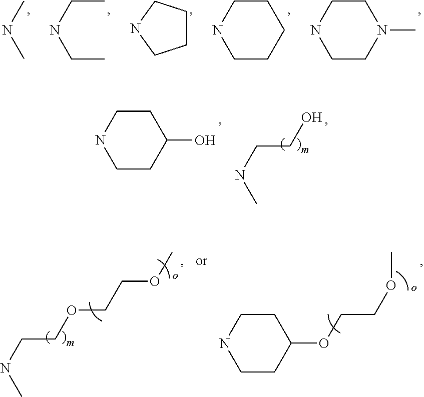

m is an integer from 1 to 3,
is an integer from 1 to 4,
p is an integer from 1 to 4,
wherein when p=1:
   each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain;
wherein when p=2:
   each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₈ to C₁₆ aryl-alkyl in which the aryl is phenyl or naphthalenyl and is positioned at either end or within the alkyl chain;
wherein when p=3:
   each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain; and
wherein when p=4:
   each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain.

In some embodiments, the ionizable cationic lipid has the structure CICL:

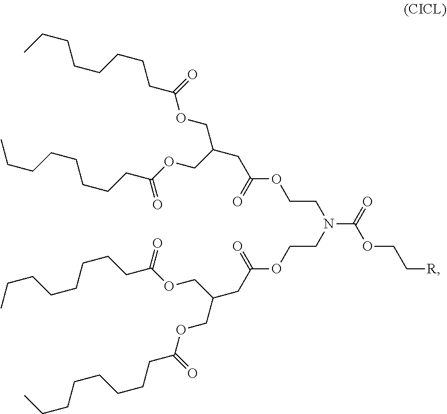

wherein R is

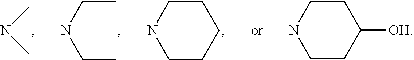

In certain embodiments, the ionizable cationic lipid of CICL is referred to as CICL1 when R is

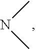

that is

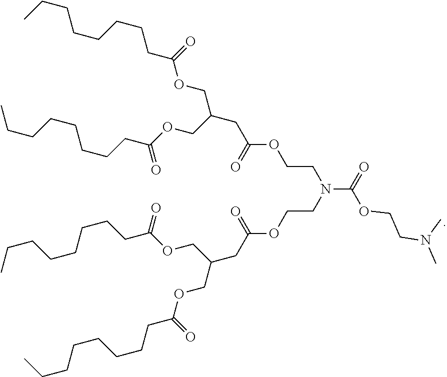 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN(C)C]

In certain embodiments the ionizable cationic lipid of CICL is referred to as CICL2 when R is

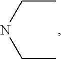

that is

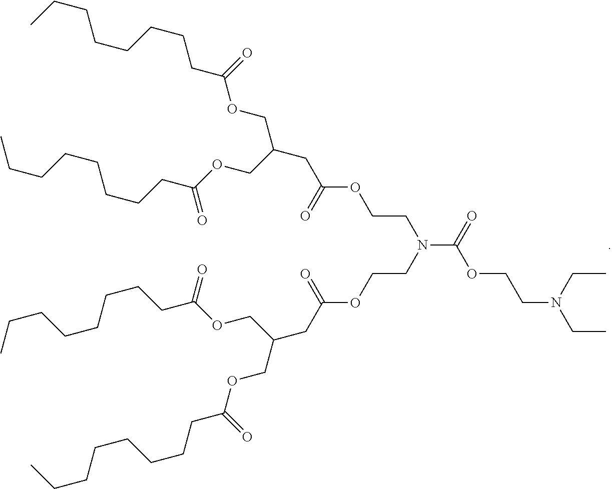 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN(CC)CC]

In certain embodiments, the ionizable cationic lipid of CICL is referred to as CICL3 when R is

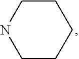

that is

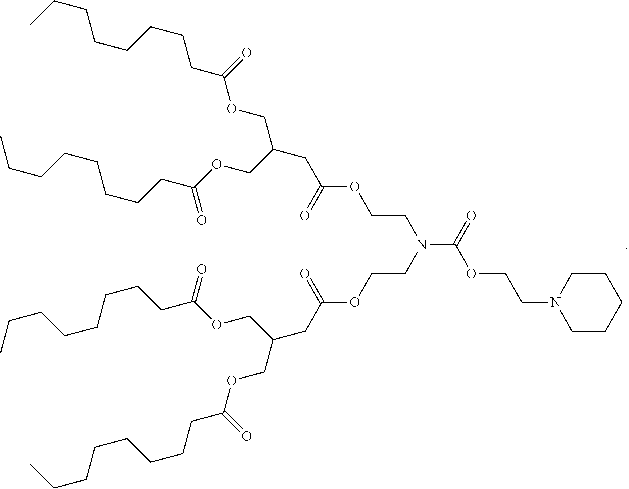 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN1CCCCC1]

In certain embodiments, the ionizable cationic lipid of CICL is referred to as CICL4 when R is

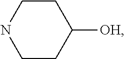

that is

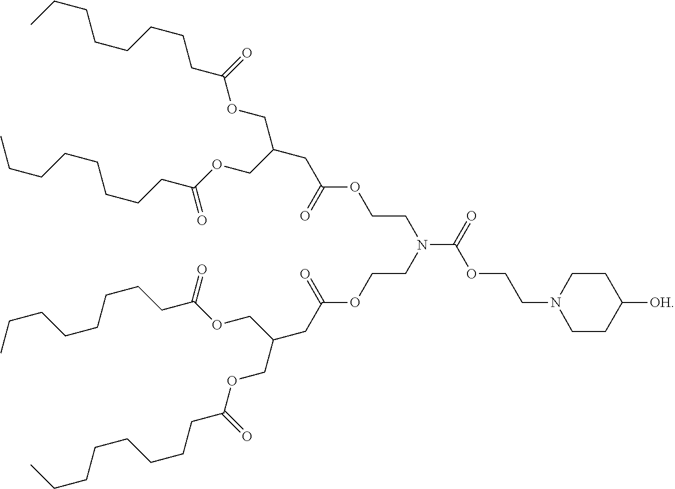 [SMILES: CCCCCCCCC(=O)OCC(COC(=O)CCCCCCCC)CC(=O)OCCN(CCOC(=O)CC(COC(=O)CCCCCCCC)COC(=O)CCCCCCCC)C(=O)OCCN1CCC(O)CC1]

In some embodiments, the ionizable cationic lipid has the structure CICL-IE.

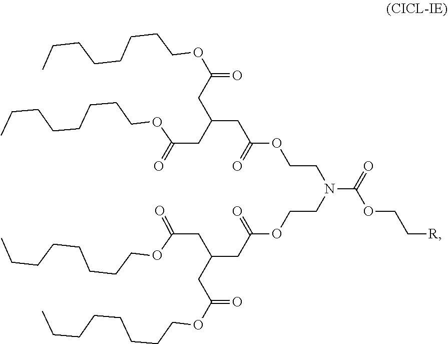

wherein R is

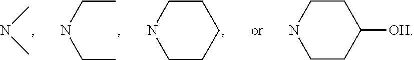

In certain embodiments, the ionizable cationic lipid of CICL-IE is referred to as CICL250 when R is

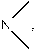

that is

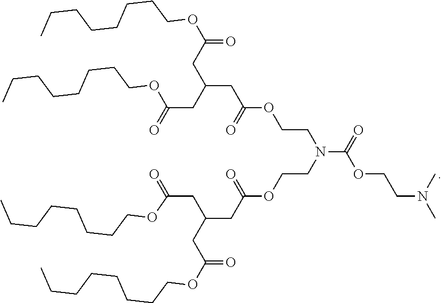 [SMILES: CCCCCCCCOC(=O)CC(CC(=O)OCCCCCCCC)CC(=O)OCCN(CCOC(=O)CC(CC(=O)OCCCCCCCC)CC(=O)OCCCCCCCC)C(=O)OCCN(C)C]

In certain embodiments, the ionizable cationic lipid of CICL-IE is referred to as CICL250.2 when R is

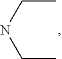

that is

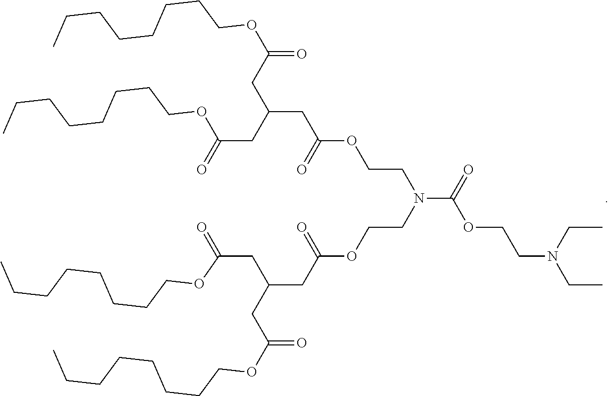 [SMILES: CCCCCCCCOC(=O)CC(CC(=O)OCCCCCCCC)CC(=O)OCCN(CCOC(=O)CC(CC(=O)OCCCCCCCC)CC(=O)OCCCCCCCC)C(=O)OCCN(CC)CC]

In certain embodiments, the ionizable cationic lipid of CICL-IE is referred to as CICL250.3 when R is

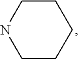

that is

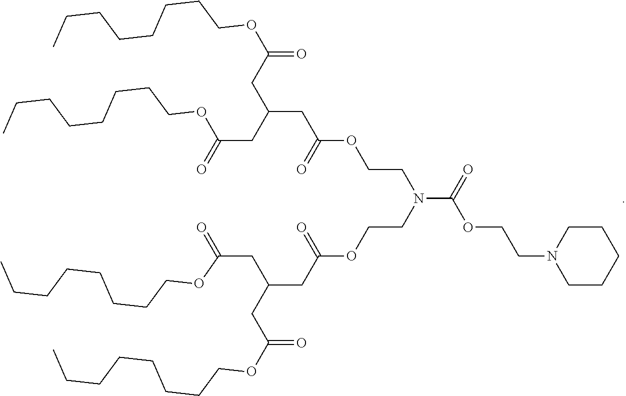 [SMILES: CCCCCCCCOC(=O)CC(CC(=O)OCCCCCCCC)CC(=O)OCCN(CCOC(=O)CC(CC(=O)OCCCCCCCC)CC(=O)OCCCCCCCC)C(=O)OCCN1CCCCC1]

In certain embodiments, the ionizable cationic lipid of CICL-IE is referred to as CICL250.4 when R is

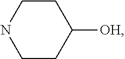

that is

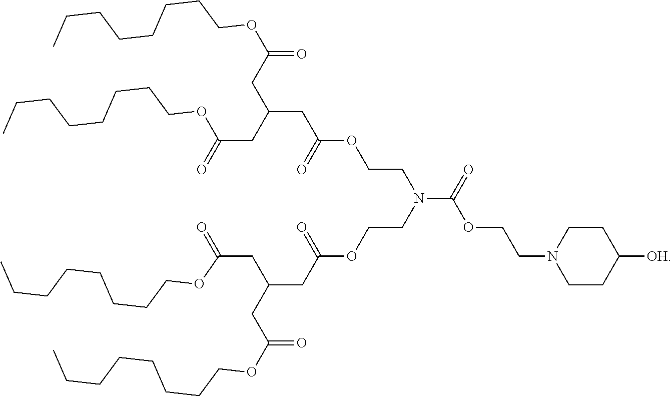 [SMILES: CCCCCCCCOC(=O)CC(CC(=O)OCCCCCCCC)CC(=O)OCCN(CCOC(=O)CC(CC(=O)OCCCCCCCC)CC(=O)OCCCCCCCC)C(=O)OCCN1CCC(O)CC1]

In certain aspects, the constrained ionizable cationic lipids of this disclosure have a structure of the formula M6:

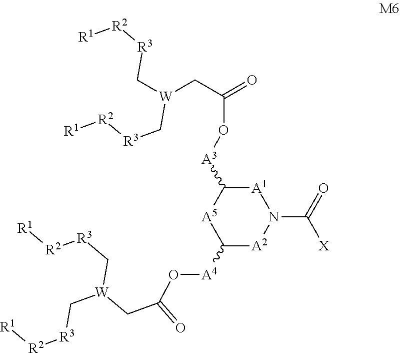

wherein X is

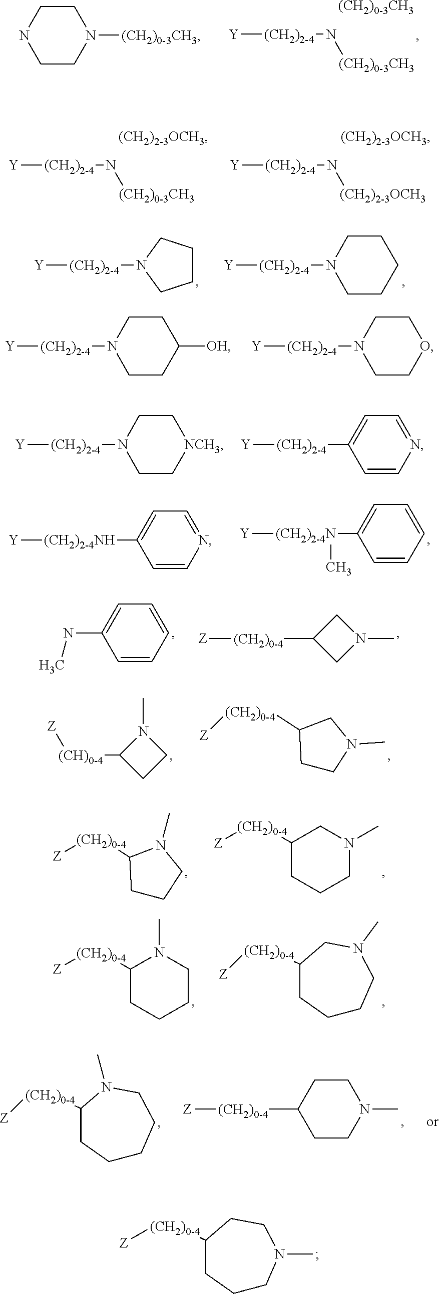

and
Y is O, S, NH, or NCH₃;
Z is O, NH, or NCH₃;
R² is O, R³ is C═O and W is CH or N, or R² is C═O, R³ is O and W is CH; and
each R¹ is independently selected from a C₇-C₁₁ alkyl or a C₇-C₁₁ alkenyl;
each A¹, A², A³, and A⁴ is independently selected from (CH₂)₀ and (CH₂)₁,
A⁵ is selected from (CH₂)₀₋₄, CH═CH, and CH₂—CH═CH—CH₂; and
a wavy bond indicate that any relative or absolute stereo-configuration of the corresponding ring atom, or a mixture of stereo-configurations, can be assumed.

As used herein, when a subscript has a value of “0”, the group is absent. For example, when A¹ is (CH₂)₀, A¹ is absent.

In certain embodiments of formula M6, R² is O, R³ is C═O and W is CH or N. For example, in certain embodiments of formula M6, R² is O, R³ is C═O and W is CH.

In certain embodiments of formula M6, R² is C═O, R³ is O and W is CH.

In various embodiments of M6, A¹ through A⁴ are chosen so that there are only two main chain atoms between the ring nitrogen and each nearest ester oxygen in the nearest tail group.

In certain embodiments of M6, A¹ is (CH₂)₀, A² is (CH₂)₀, A³ is (CH₂)₁, A⁴ is (CH₂)₁, and A⁵ is (CH₂)₁₋₄ or CH₂—CH═CH—CH₂.

In certain embodiments of M6, A¹ is (CH₂)₀, A² is (CH₂)₁, A³ is (CH₂)₁, A⁴ is (CH₂)₀, and A⁵ is (CH₂)₁.

In certain embodiments of M6, A¹ is (CH₂)₁, A² is (CH₂)₁, A³ is (CH₂)₀, A⁴ is (CH₂)₀, and A⁵ is (CH₂)₀.

In certain embodiments of M6, A¹ is (CH₂)₁, A² is (CH₂)₁, A³ is (CH₂)₀, A⁴ is (CH₂)₀, and A⁵ is (CH₂)₁.

In certain embodiments of M6, A¹ is (CH₂)₁, A² is (CH₂)₁, A³ is (CH₂)₀, A⁴ is (CH₂)₀, and A⁵ is (CH₂)₂ or CH═CH.

In some embodiments of formula M6 as described herein, X is

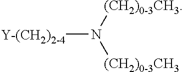

For example, in some embodiments of formula M6, X is

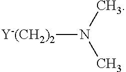

In some embodiments of formula M6 as described herein, X is

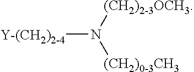

In some embodiments of formula M6 as described herein, X is

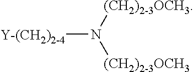

In some embodiments of formula M6 as described herein, X is

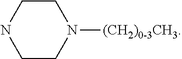

In some embodiments X is

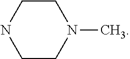

In some embodiments of formula M6 as described herein, X is

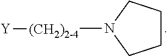

In some embodiments of formula M6 as described herein, X is

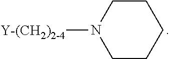

In some embodiments of formula M6 as described herein, X is

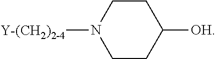

In some embodiments of formula M6 as described herein, X is

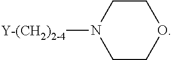

In some embodiments of formula M6 as described herein, X is

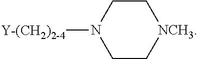

In some embodiments of formula M6 as described herein, X is

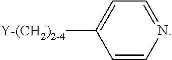

In some embodiments of formula M6 as described herein, X is

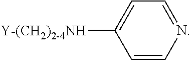

In some embodiments of formula M6 as described herein, X is

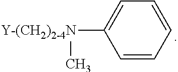

In some embodiments of formula M6 as described herein, X is

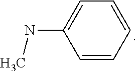

In some embodiments of formula M6, X is

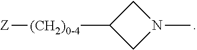

In some embodiments of formula M6, X is

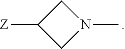

In some embodiments of formula M6, X is

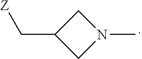

In some embodiments of formula M6, X is

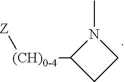

In some embodiments of formula M6, X is

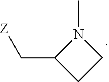

In some embodiments of formula M6, X is

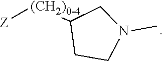

In some embodiments of formula M6, X is

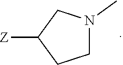

In some embodiments of formula M6, X is

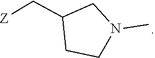

In some embodiments of formula M6, X is

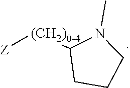

In some embodiments of formula M6, X is

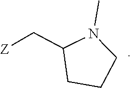

In some embodiments of formula M6, X is

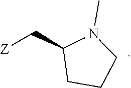

In some embodiments of formula M6, X is

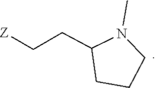

In some embodiments of formula M6, X is

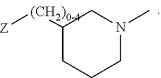

In some embodiments of formula M6, X is

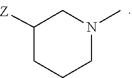

In some embodiments of formula M6, X is

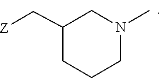

In some embodiments of formula M6, X is

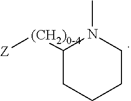

In some embodiments of formula M6, X is

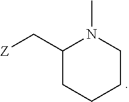

In some embodiments of formula M6, X is

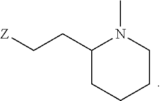

In some embodiments of formula M6, X is

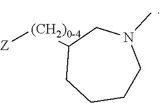

In some embodiments of formula M6, X is

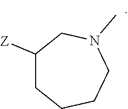

In some embodiments of formula M6, X is

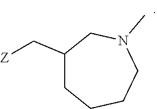

In some embodiments of formula M6, X is

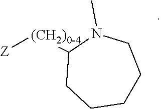

In some embodiments of formula M6, X is

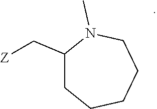

In some embodiments of formula M6, X is

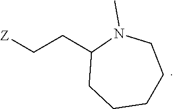

In some embodiments of formula M6, X is

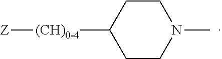

In some embodiments of formula M6, X is

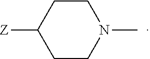

In some embodiments of formula M6, X is

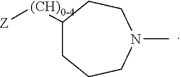

In some embodiments of formula M6, X is

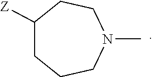

As described above, in some embodiments of formula M6, Y may be selected from O, S, NH, or NCH₃. In some embodiments of formula M6, Y is O. In some other embodiments of formula M6, Y is S.

In some embodiments of formula M6, X is

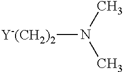

and Y is O. In some embodiments of formula M6, X is

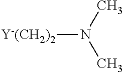

### and Y is S.

As described above, Z can be selected from O, NH, or NCH₃. In some embodiments, Z is O.

In some embodiments of formula M6, X is

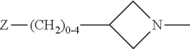

and Z is O. In some embodiments of formula M6, X is is

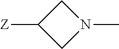

and Z is O. In some embodiments of formula M6, X is

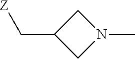

### and Z is O.

In some embodiments of formula M6, X is

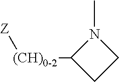

and Z is O. In some embodiments of formula M6, X is

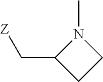

### and Z is O.

In some embodiments of formula M6, X is

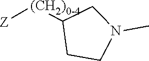

and Z is O. In some embodiments of formula M6, X is

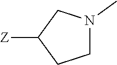

and Z is O. In some embodiments of formula M6, X is

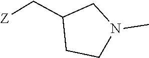

### and Z is O.

In some embodiments of formula M6, X is

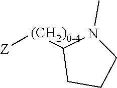

and Z is O. In some embodiments of formula M6, X is

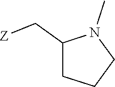

and Z is O. In some embodiments of formula M6, X is

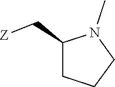

and Z is O. In some embodiments of formula M6, X is

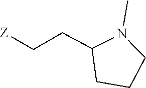

### and Z is O.

In some embodiments of formula M6, X is

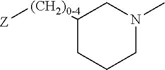

and Z is O. In some embodiments of formula M6, X is

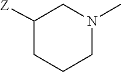

and Z is O. In some embodiments of formula M6, X is

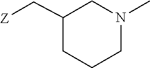

### and Z is O.

In some embodiments of formula M6, X is

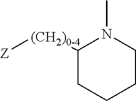

and Z is O. In some embodiments of formula M6, X is

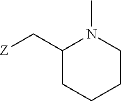

and Z is O. In some embodiments of formula M6, X is

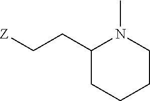

### and Z is O.

In some embodiments of formula M6, X is

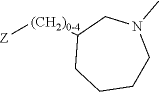

and Z is O. In some embodiments of formula M6, X is

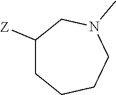

and Z is O. In some embodiments of formula M6, X is

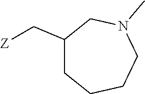

### and Z is O.

In some embodiments of formula M6, X is

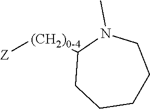

and Z is O. In some embodiments of formula M6, X is

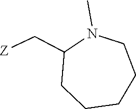

and Y is O. In some embodiments of formula M6, X is

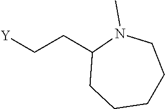

### and Z is O.

In some embodiments of formula M6, X is

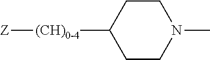

and Z is O. In some embodiments of formula M6, X is

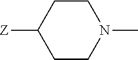

### and Z is O.

In some embodiments of formula M6, X is

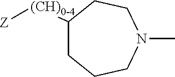

and Z is O. In some embodiments of formula M6, X is

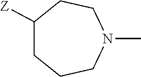

### and Z is O.

As described above, for both formula M5 and M6, each R¹ is independently selected from C₇-C₁₁ alkyl or C₇-C₁₁ alkenyl. In some embodiments of formula M5 and/or M6, each R¹ is independently selected from C₇-C₁₁ alkyl, e.g., C₇-C₁₀ alkyl, or C₇-C₉ alkyl. In certain embodiments of formula M5 and/or M6, each R¹ is independently selected from a linear C₇-C₁₁ alkyl, e.g., a linear C₇-C₁₀ alkyl, or a linear C₇-C₉ alkyl. In some embodiments of formula M5 and/or M6 as described herein, each R¹ is independently selected from (CH₂)₆₋₈CH₃. In some of these and other embodiments, R¹ is (CH₂)₇CH₃. In some embodiments of formula M5 and/or M6, each R¹ is independently selected from a linear C₇-C₁₁ alkenyl, e.g., a linear C₇-C₁₀ alkenyl, or a linear C₇-C₉ alkenyl. For example, in some embodiments of formula M5 and/or M6, each R¹ is a linear C₈ alkenyl. In certain other embodiments of formula M5 and/or M6, each R¹ is independently selected from a branched C₇-C₁₁ alkyl, e.g., C₇-C₁₀ alkyl, or C₇-C₉ alkyl. For example, in some embodiments of formula M5 and/or M6, each R¹ is a branched C₈ alkyl. In certain embodiments of formula M5 and/or M6, each R¹ is independently selected from a branched C₇-C₁₁ alkenyl, e.g., C₇-C₁₀ alkenyl, or C₇-C₉ alkenyl. For example, in some embodiments of formula M5 and/or M6, each R¹ is a branched C₈ alkenyl. In some embodiments of formula M5 and/or M6, wherein R¹ is a branched alkyl or alkenyl, the branch point is positioned so that ester carbonyls are not in an α position relative to the branch point, for example they are in a R position relative to the branch point.

In certain embodiments of formula M5 and/or M6 as described herein, each R¹ is the same. In certain embodiments of formula M5 and/or M6, each R¹ nearest a common branch point is the same, but those nearest a first common branch point differ from those nearest a second common branch point. In certain embodiments of formula M5 and/or M6, each R¹ nearest a common branch point is different but the pair of R¹s nearest a first common branch point is the same the pair nearest a second common branch point.

In certain embodiments of formula M6, the ionizable cationic lipid is substantially enantiomerically pure (e.g., at least about 80%, at least about 85%, at least about 90%, at least about 95%, at least about 96%, at least about 97%, at least about 98%, or at least about 99%). In certain embodiments of formula M6, the ionizable cationic lipid is a racemic mixture. In certain embodiments of formula M6, the ionizable cationic lipid is a mixture of two or more stereoisomers. In certain embodiments of formula M6, at least two of the two or more stereoisomers are diastereomers. In certain embodiments of formula M6, at least two of the two or more stereoisomers are enantiomers.

In some embodiments as described herein, the ionizable cationic lipids have a structure of Formula 2:

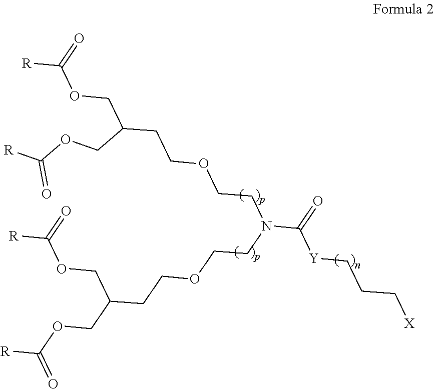

wherein:
Y is O, NH, N—CH₃, or CH₂,
n is an integer from 0 to 4,
X is

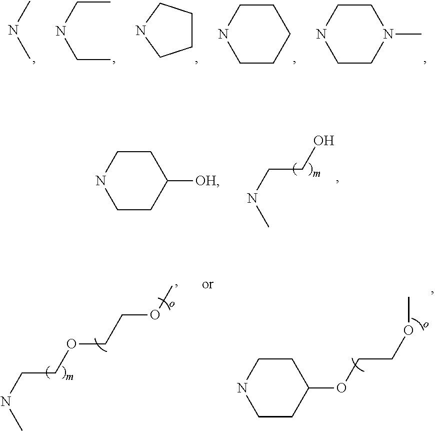

m is an integer from 1 to 3,
is an integer from 1 to 4,
p is an integer from 1 to 4,
wherein when p=1:
   each R is independently C₆ to C₁₆ straight-chain alkyl; C₆ to C₁₆ branched alkyl; C₆ to C₁₆ straight-chain alkenyl; C₆ to C₁₆ branched alkenyl; C₉ to C₁₆ cycloalkyl-alkyl in which the cycloalkyl is C₃ to C₈ cycloalkyl positioned at either end or within the alkyl chain; or C₈ to C₁₈ aryl-alkyl in which the aryl is phenyl or naphthalenyl and is positioned at either end or within the alkyl chain;,
wherein when p=2:
   each R is independently C₆ to C₁₄ straight-chain alkyl; C₆ to C₁₄ straight-chain alkenyl; C₆ to C₁₄ branched alkyl; C₆ to C₁₄ branched alkenyl; C₉ to C₁₄ cycloalkyl-alkyl in which the cycloalkyl is C₃ to C₈ cycloalkyl positioned at the either end or within the alkyl chain; or C₈ to C₁₆ aryl-alkyl in which the aryl is phenyl or naphthalenyl and is positioned at either end or within the alkyl chain,
wherein when p=3:
   each R is independently C₆ to C₁₂ straight-chain alkyl; C₆ to C₁₂ straight-chain alkenyl; C₆ to C₁₂ branched alkyl; C₆ to C₁₂ branched alkenyl; C₉ to C₁₂ cycloalkyl-alkyl in which the cycloalkyl is C₃ to C₈ cycloalkyl positioned at either end or within the alkyl chain; or C₈ to C₁₄ aryl-alkyl in which the aryl is phenyl or naphthalenyl and is positioned at the either end or within the alkyl chain; and
wherein when p=4:
   each R is independently C₆ to C₁₀ straight-chain alkyl; C₆ to C₁₀ straight-chain alkenyl; C₆ to C₁₀ branched alkyl; C₆ to C₁₀ branched alkenyl; C₉ to C₁₀ cycloalkyl-alkyl in which the cycloalkyl is C₃ to C₈ cycloalkyl positioned at either end or within the alkyl; or C₈ to C₁₂ aryl-alky in which the aryl is phenyl or naphthalenyl and is positioned at the either end or within the alkyl chain.

In some embodiments as described herein, the ionizable cationic lipids have a structure of Formula 3:

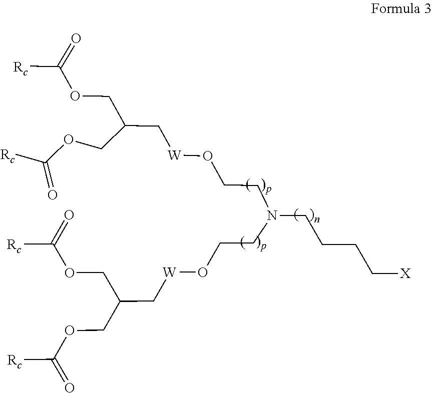

wherein:
W is C═O or CH₂,
n is an integer from 0 to 4,
X is

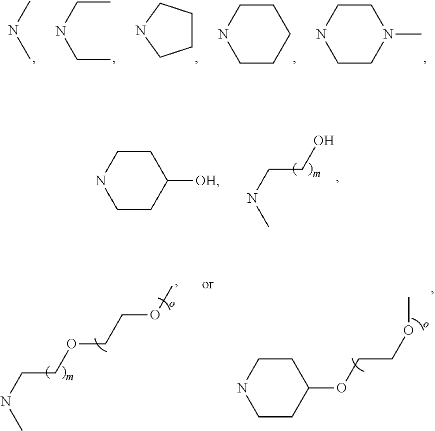

m is an integer from 1 to 3,
is an integer from 1 to 4,
p is an integer from 1 to 4,
wherein when p=1:
   each R^{c} is independently C₈ to C₁₈ straight-chain alkyl; C₈ to C₁₈ straight-chain alkenyl; C₈ to C₁₈ branched alkyl; C₈ to C₁₈ branched alkenyl; C₁₁ to C₁₈ cycloalkyl-alkyl in which the cycloalkyl is C₃ to C₈ cycloalkyl positioned at either end or within the alkyl chain; or C₁₀ to C₂₀ aryl-alkyl in which the aryl is phenyl or naphthalenyl and is positioned at either end or within the alkyl chain,
wherein when p=2:
   each R^{c} is independently C₈ to C₁₆ straight-chain alkyl; C₈ to C₁₆ straight-chain alkenyl; C₈ to C₁₆ branched alkyl; C₈ to C₁₆ branched alkenyl; C₁₁ to C₁₆ cycloalkyl-alkyl in which the cycloalkyl is C₃ to C₈ cycloalkyl positioned at the either end or within the alkyl chain; or C₁₀ to C₁₈ aryl-alkyl in which the aryl is phenyl or naphthalenyl and is positioned at either end or within the alkyl chain,
wherein when p=3:
   each R^{c} is independently C₈ to C₁₄ straight-chain alkyl; C₈ to C₁₄ straight-chain alkenyl; C₈ to C₁₄ branched alkyl; C₈ to C₁₄ branched alkenyl; C₁₁ to C₁₄ cycloalkyl-alkyl in which the cycloalkyl is C₃ to C₈ cycloalkyl positioned at either end or within the alkyl chain; or C₁₀ to C₁₆ aryl-alkyl in which the aryl is phenyl or naphthalenyl and is positioned at the either end or within the alkyl chain, and
wherein when p=4:
   each R^{c} is independently C₈ to C₁₂ straight-chain alkyl; C₈ to C₁₂ straight-chain alkenyl; C₈ to C₁₂ branched alkyl; C₈ to C₁₂ branched alkenyl; C₁₁ to C₁₂ cycloalkyl-alkyl in which the cycloalkyl is C₃ to C₈ cycloalkyl positioned at either end or within the alkyl; or C₁₀ to C₁₄ aryl-alky in which the aryl is phenyl or naphthalenyl and is positioned at the either end or within the alkyl chain.
   R^{c} in the text and R_{c} in the chemical structure are equivalent.

Ionizable cationic lipids of this disclosure have a branched structure to give the lipid a conical rather than cylindrical shape and such structure helps promote endosomolytic activity. The greater the endosomolytic activity, the more efficient is release of the biologically active payload (e.g., one or more species of nucleic acid molecules).

Ionizable cationic lipids as described herein, can be useful as a component of lipid nanoparticles for delivering nucleic acids, including DNA, mRNA, or siRNA into cells. The ionizable cationic lipids may have a c-pKa (calculated pKa) in the range of from about 6, 7, or 8 to about 9, 10, or 11. For example, in various embodiments as described herein, the ionizable cationic lipids have a c-pKa ranging from about 6 to about 10, about 7 to about 10, about 8 to about 10, about 8 to about 9, 6 to 10, 7 to 10, 8 to 10, or 8 to 9. In certain embodiments, the ionizable cationic lipids have a c-pKa ranging from about 8.4 to about 8.7 or 8.4 to 8.7. The ionizable cationic lipids as described herein may have cLogD ranging from about 9 to about 18, for example, ranging from about 10 to about 18, or about 10 to about 16, to about 10 to about 14, or about 11 to about 18, or about 11 to about 15, or about 11 to about 14. The ionizable cationic lipids as described herein may have cLogD ranging from 9 to 18, for example, ranging from 10 to 18, or 10 to 16, to 10 to 14, or 11 to 18, or 11 to 15, or 11 to 14. In certain embodiments, the ionizable cationic lipids have a cLogD ranging from about 13.6 to about 14.4 or from 13.6 to 14.4. In certain embodiments, the ionizable cationic lipids as described herein may have a c-pKa ranging from about 8 to about 11 or from 8 to 11 and a cLogD ranging from about 9 to about 18 or from 9 to 18. For example, in certain embodiments, the ionizable cationic lipids have a c-pKa ranging from about 8.4 to about 8.7 or from 8.4 to 8.7 and cLogD ranging from about 13.6 to about 14.4 or from 13.6 to 14.4. These ranges can lead to a measured pKa in the LNP ranging from about 6 to about 7 or from 6 to 7, which facilitates ionization in an endosome after delivery into a cell.

In some embodiments, somewhat greater basicity may be desirable and can be obtained from ionizable cationic lipids with c-pKa and cLogD in the ranges disclosed herein. In some embodiments, cLogD of ionizable cationic lipids of this disclosure is about 9, about 10, about 11, about 12, about 13, about 14, about 15, about 16, about 17, about 18, or in a range bound by any pair of these values. Lipid design also accounts for potential biodegradability pathways of target lipids, such as by way of esterases in plasma, liver, and other tissues. Another consideration in lipid design is the fate of fragments of ionizable lipids resulting from degradation, such as after esterase cleavage(s). Preferably, the resulting fragments are rapidly cleared from the body without the need for hepatic oxidative metabolism.

The synthesis of lipids having the structure of M5, CICL, CICL-IE, or M6 is described in US Patent Application Nos. 63/632,931 (some M6), 63/632,937 (some M5) 63/632,940 (CICL-IE, some M5), and 63/632,944 (some M6), and US Patent Application Publication No. 2023/0320995 (CICL) each of which is incorporated by reference in its entirety for all that it teaches about the synthesis of such lipids, as well as particular subgenera and individual species.

Further ionizable cationic lipids and LNP compositions comprising them can be found in WO 2017/049245, WO 2022/112855, WO2013/185116, WO2015074085, WO2016,081029, WO2017/117530, WO2018/118102, WO2022/235935, WO 2023/086514, WO2024/044728, WO2023/196931, WO2023/044333A1, WO2013089151, WO2023/183616, WO2013/065825, WO2013/089152, WO2015/186770, WO2022/166213, WO2023/045366, WO2019/131580, WO 2005/007196, WO 2006/053430, WO 2007/086883, WO 2009/129387, WO 2010/048536, U.S. Pat. Nos. 9,868,692, 10,435,616 11,246,933, 11,382,979, 8,058,069, 8,492,359, 8,722,082, 8,822,668, 9,364,435, 9,408,914, 9,504,651, 10,526,284, 10,961,188, 11,141,378, and 11,241,493, each of which in incorporated by reference herein for all that it teaches about cationic ionizable lipids, LNPs incorporating them, and nucleic acid delivery mediated by such LNPs that is not inconsistent with the present disclosure. In certain embodiments, a tLNP comprises as its targeting moiety an antibody or antigen binding portion thereof that comprises a humanized antigen binding domains of CT8 of this disclosure and further comprises a cationic ionizable lipid from any one of WO 2017/049245, WO 2022/112855, WO 2005/007196, WO 2006/053430, WO 2007/086883, WO 2009/129387, WO 2010/048536, U.S. Pat. Nos. 9,868,692, 10,435,616 11,246,933, 11,382,979, 8,058,069, 8,492,359, 8,822,668, 9,364,435, 9.504,651, 11,141,378, and 11,241,493.

In some embodiments, an LNP or tLNP comprises about 35 mol % to about 65 mol %, about 40 mol % to about 62 mol %, or about 54 mol % to about 60 mol % ionizable cationic lipid. In some embodiments, the lipid composition is at least 40 mol % and/or does not exceed 62 mol % ionizable cationic lipid. In certain embodiments, an LNP of tLNP comprises about 54 mol %, about 58 mol %, or about 62 mol % ionizable cationic lipid, or is a range bound by any pair of these values. In further embodiments an LNP comprises 35 mol % to 65 mol %, 40 mol % to 62 mol %, or 54 mol % to 60 mol % ionizable cationic lipid. In still further embodiments, an LNP has at least 40 mol % or does not exceed 62 mol % ionizable cationic lipid. In certain embodiments, an LNP comprises 54 mol %, 58 mol %, or 62 mol % ionizable cationic lipid, or is a range bound by any pair of these values.

### Phospholipids

As described above, in various embodiments, the LNPs and tLNPs include a phospholipid. As would be understood by the person or ordinary skill in the art, phospholipids are amphiphilic molecules. Due to the amphiphilic nature of phospholipids, these molecules are known to form bilayers and by including them in the LNPs and tLNPs, as described herein, they can provide membrane formation, stability, and rigidity. As used herein, phospholipids include a hydrophilic head group, including a functionalized phosphate group, and two hydrophobic tail groups derived from fatty acids. For example, in various embodiments as described herein, the phospholipids include a phosphate group functionalized with ethanolamine, choline, glycerol, serine, or inositol. As described above, the phospholipid includes two hydrophobic tail groups derived from fatty acids. These hydrophobic tail groups can be derived from unsaturated or saturated fatty acids. For example, the hydrophobic tail groups can be derived from a C12-C20 fatty acid.

With respect to LNPs or tLNPs of this disclosure, in various embodiments, the phospholipid comprises dimyristoylphosphatidyl glycerol (DMPG), dimyristoylphosphatidyl choline (DMPC), dipalmitoyl phosphatidylcholine (DPPC), distearoylphosphatidylcholine (DSPC), distearoyl-glycero-phosphate (18:0 PA, DSGP), dioleoylphosphatidyl ethanolamine (DOPE), dioleoyl-glycero-phosphate (18:1 PA, DOGP), or diarachidoylphosphotidylcholine (DAPC), or a combination thereof. In various embodiments, the phospholipid is dioleoylphosphatidyl ethanolamine (DOPE), dimyristoylphosphatidyl choline (DMPC), distearoylphosphatidylcholine (DSPC), dimyristoylphosphatidyl glycerol (DMPG), dipalmitoyl phosphatidylcholine (DPPC), or 1,2-diarachidoyl-sn-glycero-3-phosphocholine (DAPC). In some embodiments, the phospholipid is distearoylphosphatidylcholine (DSPC). Phospholipids can contribute to formation of a membrane, whether monolayer, bilayer, or multi-layer, surrounding the core of the LNP or tLNP. Additionally, phospholipids such as DSPC, DMPC, DPPC, DAPC impart stability and rigidity to membrane structure. Phospholipids, such as DOPE, impart fusogenicity. Further phospholipids, such as DMPG, which attains negative charge at physiologic pH, facilitates charge modulation. Thus, phospholipids constitute means for facilitating membrane formation, means for imparting membrane stability and rigidity, means for imparting fusogenicity, and means for charge modulation. Some embodiments specifically include one or more of the above phospholipids while other embodiments specifically exclude one or more of the above phospholipids.

In some embodiments, an LNP or tLNP has about 7 mol % to about 13 mol % phospholipid, about 7 mol % to about 10 mol % phospholipid, or about 10 mol % to about 13 mol % phospholipid. In certain embodiments, an LNP has about 7 mol %, about 10 mol %, or about 13 mol % phospholipid. In certain instances, the phospholipid is DSPC. In certain instances, the phospholipid is DAPC.

### Sterols

In certain embodiments, the disclosed LNP and tLNP comprise a sterol. Sterol refers to a subgroup of steroids that contain at least one hydroxyl (OH) group. More specifically, a gonane derivative with an OH group substituted for an H at position 3, or said differently, but equivalently, a steroid with an OH group substituted for an H at position 3. Examples of sterols include, without limitation, cholesterol, ergosterol, β-sitosterol, stigmasterol, stigmastanol, 20-hydroxycholesterol, 22-hydroxycholesterol, and the like. With respect to LNPs or tLNPs of this disclosure, in various embodiments, the sterol is cholesterol, 20-hydroxycholesterol, 20(S)-hydroxycholesterol, 22-hydroxycholesterol, or a phytosterol or combinations thereof. In further embodiments, the phytosterol comprises campesterol, sitosterol, or stigmasterol, or combinations thereof. In certain embodiments, the cholesterol is not animal-sourced but is obtained by synthesis using a plant sterol as a starting point. LNPs incorporating C-24 alkyl (such as methyl or ethyl) phytosterols have been reported to provide enhanced gene transfection. The length of the alkyl tail, the flexibility of the sterol ring, and polarity related to a retained C-3—OH group are important to obtaining high transfection efficiency. While β-sitosterol and stigmasterol performed well, vitamin D2, D3 and calcipotriol, (analogs lacking intact body of cholesterol) and betulin, lupeol ursolic acid and olenolic acid (comprising a 5th ring) should be avoided. Sterols serve to fill space between other lipids in the LNP or tLNP and influence LNP or tLNP shape. Sterols also control fluidity of lipid compositions, reducing temperature dependence. Thus, sterols such as cholesterol, ergosterol, 20-hydroxycholesterol, 22-hydroxycholesterol, campesterol, fucosterol, β-sitosterol, and stigmasterol constitute means for controlling LNP shape and fluidity or sterol means for increasing transfection efficiency. Some embodiments specifically include one or more of the above sterols while other embodiments specifically exclude one or more of the above sterols. In designing a lipid composition for a LNP or tLNP, in some embodiments, sterol content can be chosen to compensate for different amounts of other types of lipids, for example, ionizable cationic lipid or phospholipid.

In some embodiments, an LNP or tLNP has about 27 mol % or about 30 mol % to about 50 mol % sterol, or about 30 mol % to about 38 mol % sterol. In certain embodiments, an LNP or tLNP has about 30.5 mol %, about 33.5 mol %, or about 37.5 mol % sterol. In certain embodiments, an LNP or tLNP has 27 mol % or 30 mol % to 50 mol % sterol or 30 mol % to 38 mol % sterol. In further embodiments, an LNP or tLNP has 30.5 mol %, 33.5 mol %, or 37.5 mol % sterol. In certain instances, the sterol is cholesterol. In certain embodiments, the sterol is a mixture of sterols, for example, cholesterol and β-sitosterol or cholesterol and 20-hydroxycholesterol. In some instances, the sterol component is about 25 mol % 20-hydroxycholesterol and about 75 mol % cholesterol. In some instances, the sterol component is about 25 mol % β-sitosterol and about 75 mol % cholesterol. In some instances, the sterol component is about 50 mol % β-sitosterol and about 50 mol % cholesterol. In some instances, a sterol component is 25 mol % 20-hydroxycholesterol and 75 mol % cholesterol. In further instances, a sterol component is 25 mol % β-sitosterol and 75 mol % cholesterol. In still further instances, a sterol component is 50 mol % β-sitosterol and 50 mol % cholesterol.

### Co-Lipids

With respect to the LNP or the tLNP, in some embodiments, the co-lipid is absent or comprises an ionizable lipid. In some embodiments the ionizable lipid is cholesterol hemisuccinate (CHEMS). In some embodiments, the co-lipid is a charged lipid, such as a quaternary ammonium headgroup-containing lipid. In some instances, the quaternary ammonium headgroup-containing lipid comprises 1,2-dioleoyl-3-trimethylammonium propane (DOTAP), N-(1-(2,3-dioleyloxy)propyl)-N,N,N-trimethylammonium chloride (DOTMA), or 3β-(N—(N′,N′-Dimethylaminoethane)carbamoyl)cholesterol (DC-Chol), or combinations thereof. In addition to the chloride salts of the quaternary ammonium headgroup containing lipids, further instances include bromide, mesylate, and tosylate salts.

### PEG-Lipids

With respect to a LNP or tLNP of this disclosure, a PEG-lipid is a lipid conjugated to a polyethylene glycol (PEG). In some embodiments as described herein, the PEG-lipid is a C14-C20 lipid conjugated with a PEG. For example, in various embodiments as described herein, the PEG-lipid is a C14-C20 lipid conjugated with a PEG, or a C14-C18 lipid conjugated with a PEG, or a C14-C16 lipid conjugated with a PEG. In certain embodiments as described herein, the PEG-lipid is a fatty acid conjugated with a PEG. The fatty acid of the PEG-lipid can have a variety of chain lengths. For each, in some embodiments, the PEG-lipid is a fatty acid conjugated with PEG, wherein the fatty acid chain length is in the range of C14-C20 (e.g., in the range of C14-C18, or C14-C16). PEG-lipids with fatty acid chain lengths less than C14 are too rapidly lost from the LNP or tLNP while those with chain lengths greater than C20 are prone to difficulties with formulation.

PEG can be made in a large range of sizes. In certain embodiments, the PEG of the disclosed LNP and tLNP is PEG-1000 to PEG-5000. It is to be understood that polyethylene preparations of these sizes are polydisperse and that the nominal size indicates an approximate average molecular weight of the distribution. Taking the molecular weight of an individual repeating unit of (OCH2CH2)ₙ to be 44, a PEG molecule with n=22 would have a molecular weight of 986, with n=45 a molecular weight of 1998, and with n=113 a molecular weight of 4990. n≈22 to 113 is used to represent PEG-lipids incorporating PEG moieties in the range of PEG-1000 to PEG-5000 such as PEG-1000, PEG-1500, PEG-2000, PEG-2500, PEG-3000, PEG-3500, PEG-4000, PEG-4500, and PEG-5000, although some molecules from preparations at the average molecular weight boundaries will have an n outside that range. For individual preparations n≈22 is used to represent PEG-lipids incorporating PEG moieties from PEG-1000, n≈45 is used to represent PEG-lipids incorporating PEG moieties from PEG-2000 n≈67 is used to represent PEG-lipids incorporating PEG moieties from PEG-3000, n≈90 is used to represent PEG-lipids incorporating PEG moieties from PEG-4000, n≈113 is used to represent PEG-lipids incorporating PEG moieties from PEG-5000. Some embodiments incorporate PEG moieties in a range bounded by any pair of the foregoing values of n or average molecular weight. In some embodiments of the PEG-lipid, a PEG is of 500-5000 or 1000-5000 Da molecular weight (MW). For example, in some embodiments, the PEG of the PEG-lipid has a molecular weight in the range of 1500-5000 Da or 2000-5000 Da. In some embodiments as described herein, the PEG-lipid has a molecular weight in the range of 500-4000 Da, or 500-3000 Da, or 1000-4000 Da, or 1000-3000, or 1000-2500, or 1500-4000, or 1500-3000, or 1500-2500 Da. In some embodiments, the PEG moiety is PEG-500, PEG-1000, PEG-1500, PEG-2000, PEG-2500, PEG-3000, PEG-3500, PEG-4000, PEG-4500, and PEG-5000. In some embodiments, the PEG unit has a MW of 2000 Da (sometime abbreviated as PEG(2 k)). Some embodiments incorporate PEG moieties of PEG-1000, PEG-2000, or PEG-5000. In some instances, the PEG moiety is PEG-2000. Certain embodiments comprise a DSG-PEG, for example, DSG-PEG-2000. Certain embodiments comprise a DSPE-PEG, for example, DSPE-PEG-2000. Certain embodiments comprise both DSG-PEG-2000 and/or DSPE-PEG2000.

Common PEG-lipids fall into two classes diacyl glycerols and diacyl phospholipids. Examples of diacyl glycerol PEG-lipids include DMG-PEG (1,2-dimyristoyl-glycero-3-methoxypolyethylene glycol), DPG-PEG (1,2-dipalmitoyl-glycero-3-methoxypolyethylene glycol), DSG-PEG (1,2-distearoyl-glycero-3-methoxypolyethylene glycol), and DOG-PEG (1,2-dioleoyl-glycero-3-methoxypolyethylene glycol). Examples of diacyl phospholipids include DMPE-PEG (1,2-dimyristoyl-glycero-3-phosphoethanolamine-3-methoxypolyethylene glycol), DPPE-PEG (1,2-dipalmitoyl-glycero-3-phosphoethanolamine-3-methoxypolyethylene glycol), DSPE-PEG (1,2-distearoyl-glycero-3-phosphoethanolamine-3-methoxypolyethylene glycol), and DOPE-PEG (1,2-dioleoyl-glycero-3-phosphoethanolamine-3-methoxypolyethylene glycol).

In some embodiments, the MW2000 PEG-lipid (e.g., a PEG-lipid comprising a PEG of a molecular weight of 2000 Da) comprises DMG-PEG2000 (1,2-dimyristoyl-glycero-3-methoxypolyethylene glycol-2000), DPG-PEG2000 (1,2-dipalmitoyl-glycero-3-methoxypolyethylene glycol-2000), DSG-PEG2000 (1,2-distearoyl-glycero-3-methoxypolyethylene glycol-2000), DOG-PEG2000 (1,2-dioleoyl-glycero-3-methoxypolyethylene glycol-2000), DMPE-PEG200 (1,2-dimyristoyl-glycero-3-phosphoethanolamine-3-methoxypolyethylene glycol-2000), DPPE-PEG2000 (1,2-dipalmitoyl-glycero-3-phosphoethanolamine-3-methoxypolyethylene glycol-2000), DSPE-PEG2000 (1,2-distearoyl-glycero-3-phosphoethanolamine-3-methoxypolyethylene glycol-2000), DOPE-PEG2000 (1,2-dioleoyl-glycero-3-phosphoethanolamine-3-methoxypolyethylene glycol-2000), or combinations thereof. In some embodiments, the PEG unit has a MW of 2000 Da. In some embodiments, the MW2000 PEG-lipid comprises DMrG-PEG2000 (1,2-dimyristoyl-rac-glycero-3-methoxypolyethylene glycol-2000), DPrG-PEG2000 (1,2-dipalmitoyl-rac-glycero-3-methoxypolyethylene glycol-2000), DSrG-PEG2000 (1,2-distearoyl-rac-glycero-3-methoxypolyethylene glycol-2000), DorG-PEG2000 (1,2-dioleoyl-glycero-3-methoxypolyethylene-rac-glycol-2000), DMPEr-PEG200 (1,2-dimyristoyl-rac-glycero-3-phosphoethanolamine-3-methoxypolyethylene glycol-2000), DPPEr-PEG2000 (1,2-dipalmitoyl-rac-glycero-3-phosphoethanolamine-3-methoxypolyethylene glycol-2000), DSPEr-PEG2000 (1,2-distearoyl-rac-glycero-3-phosphoethanolamine-3-methoxypolyethylene glycol-2000), DOPEr-PEG2000 (1,2-dioleoyl-rac-glycero-3-phosphoethanolamine-3-methoxypolyethylene glycol-2000), or combinations thereof. The glycerol in these lipids is chiral. Thus, in some embodiments, the PEG-lipid is racemic. Alternatively, optically pure antipodes of the glycerol portion can be employed, that is, the glycerol portion is homochiral. As used herein with respect to glycerol moieties, optically pure means ≥95% of a single enantiomer (D or L). In some embodiments, the enantiomeric excess is ≥98%. In some embodiments, the enantiomeric excess is ≥99%. Additional PEG-lipids, including achiral PEG-lipids built on a symmetric dihydroxyacetone scaffold, a symmetric 2-(hydroxymethyl)butane-1,4-diol, or a symmetric glycerol scaffold, are disclosed in U.S. Provisional Application No. 63/362,502, filed on Apr. 5, 2022, and PCT/US2023/017648 application filed on Apr. 5, 2023 (WO 2023/196445), both entitled PEG-Lipids and Lipid Nanoparticles, which are incorporated by reference in their entirety.

The above PEG-lipid examples are presented as methoxypolyethylene glycols, but the terminus need not necessarily be methoxyl. With respect to any of the PEG-lipids that have not been functionalized, in alternative embodiments, the PEG moiety of the PEG lipids can terminate with a methoxyl, a benzyloxyl, a 4-methoxybenzyloxyl, or a hydroxyl group (that is, an alcohol). The terminal hydroxyl facilitates functionalization. The methoxyl, benzyloxyl, and 4-methoxybenzyloxyl groups are advantageously provided for PEG-lipid that will be used as a component of the LNP without functionalization. However, all four of these alternatives are useful as the (non-functionalized) PEG-lipid component of LNPs. The 4-methoxybenzyloxyl group, often used as a protecting group during synthesis of the PEG-lipid, is readily removed to generate the corresponding hydroxyl group. Thus, the 4-methoxybenzyloxyl group offers a convenient path to the alcohol when it is not synthesized directly. The alcohol is useful for being functionalized, prior to incorporation of the PEG-lipid into a LNP, so that a binding moiety, for example an engineered F(ab′) binding moiety or a humanized anti-CD8α binding moiety as disclosed herein, can be conjugated to it as a targeting moiety for the LNP (making it a tLNP). As used herein, the terminus of the PEG moiety, and similar constructions, refers to the end of the PEG moiety that is not attached to the lipid.

A PEG-moiety provides a hydrophilic surface on the LNP, inhibiting aggregation or merging of LNP, thus contributing to their stability and reducing polydispersity, i.e. reducing the heterogeneity of a dispersion of LNPs. Additionally, a PEG moiety can impede binding by the LNP, including binding to plasma proteins. These plasma proteins include apoE which is understood to mediate uptake of LNP by the liver so that inhibition of binding can lead to an increase in the proportion of LNP reaching other tissues. These plasma proteins also include opsonins so that inhibition of binding reduces recognition by the reticuloendothelial system. The PEG-moiety can also be functionalized to serve as an attachment point for a targeting moiety. Conjugating a binding moiety, for example an engineered F(ab′) binding moiety or a humanized anti-CD8α binding moiety as disclosed herein, to the PEG-moiety enables a tLNP to avoid the liver and bind to a target tissue or cell type, greatly increasing the proportion of LNP that reaches the targeted tissue or cell type. PEG-lipid can thus serve as means for inhibiting LNP binding, and PEG-lipid conjugated to an anti-CD8 can serve as means for LNP-targeting to CD8+ cells.

As used herein, the term “functionalized PEG-lipid” and similar constructions refer generally to both the unreacted and reacted entities. The lipid composition of a LNP can be described referencing the reactive species even after conjugation has taken place (forming a tLNP). For example, a lipid composition can be described as comprising DSPE-PEG-maleimide and can be said to further comprise an anti-CD8 binding moiety or an engineered F(ab′) binding moiety without explicitly noting that upon reaction to form the conjugate the maleimide will have been converted to a succinimide (or hydrolyzed succinimide). Similarly, if the reactive group is bromomaleimide, after conjugation it will be maleimide. These differences of chemical nomenclature for the unreacted and reacted species are to be implicitly understood even when not explicitly stated. Certain embodiments comprise a DSG-PEG, for example, DSG-PEG-2000. Certain embodiments comprise a functionalized DSPE-PEG, for example, functionalized DSPE-PEG-2000. Certain embodiments comprise both DSG-PEG-2000 and functionalized DSPE-PEG-2000. In some instances, the functionalized PEG-lipid is functionalized with a maleimide moiety, for example, DSPE-PEG-2000-MAL.

In certain aspects, the LNP comprises one or more PEG-lipids and/or functionalized PEG-lipids; when both a functionalized and unfunctionalized PEG-lipid, the PEG-lipid present they can be the same or different; and one or more ionizable cationic lipids; the LNP can further comprise a phospholipid, a sterol, a co-lipid, or any combination thereof. The term “functionalized PEG-lipid” refers to a PEG-lipid in which the PEG moiety has been derivatized with a chemically reactive group that can be used for conjugating a targeting moiety to the PEG-lipid. The functionalized PEG-lipid can be reacted with an anti-CD8 binding moiety or an engineered F(ab′) binding moiety so that the anti-CD8 binding moiety or the engineered F(ab′) binding moiety is conjugated to the PEG portion of the lipid. The conjugated anti-CD8 binding moiety can thus serve as a targeting moiety for the LNP to CD8+ cells constituting a tLNP. In some embodiments, the anti-CD8 binding moiety or the engineered F(ab′) binding moiety is conjugated to the functionalized PEG-lipid after an LNP comprising the functionalized PEG-lipid is formed. In other embodiments, the anti-CD8 binding moiety or the engineered F(ab′) binding moiety is conjugated to the PEG-lipid and then the conjugate is inserted into a previously formed LNP.

In certain embodiments, the LNP is a tLNP comprising one or more functionalized PEG-lipids that has been conjugated to an anti-CD8 binding moiety or an engineered F(ab′) binding moiety. In certain embodiments, the tLNP also comprises PEG-lipids not functionalized or conjugated with binding moiety. In some embodiments, the functionalization is a maleimide. In some embodiments the functionalization is a bromomaleimide or bromomaleimide amide, alkynylamide, or alkynylimide moiety at the terminal hydroxyl end of the PEG moiety. In some embodiments, the anti-CD8 binding moiety comprises an anti-CD8α antibody or anti-CD8α binding portion thereof, for example, an engineered F(ab′), as disclosed herein. In some embodiments, the binding moiety is a polypeptide comprising a binding domain and an N- or C-terminal extension comprising an accessible thiol group. In some embodiments, the conjugation linkage comprises a reaction product of a thiol in the anti-CD8 binding moiety or the engineered F(ab′) binding moiety with a functionalized PEG-lipid. In some embodiments, the functionalization is a maleimide, azide, alkyne, dibenzocyclooctyne (DBCO), bromomaleimide or bromomaleimide amide, alkynylamide, or alkynylimide. In some embodiments, the anti-CD8 binding moiety comprises an anti-CD8 antibody or anti-CD8 binding portion thereof. In some embodiments, the anti-CD8 binding moiety is a polypeptide comprising a binding domain and an N- or C-terminal extension comprising an accessible thiol group, for example, an engineered F(ab′), as disclosed herein.

In certain embodiments, the PEG-lipid and/or functionalized PEG-lipid comprises a scaffold selected from Formula S1, Formula S2, Formula S3, or Formula S4:

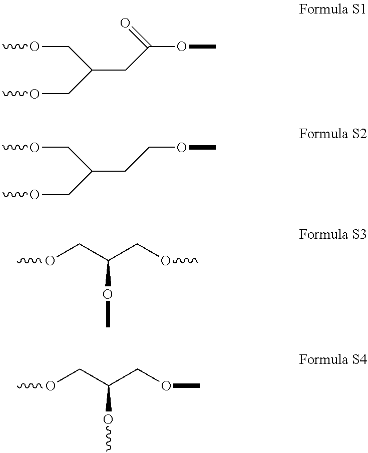

wherein [symbol] represents the points of ester connection with a fatty acid, and [symbol] represents the point of ester (S1) or ether (S2, S3, and S4) formation with the PEG moiety. In some embodiments, the fatty acid esters are C₁₄-C₂₀ straight-chain alkyl fatty acids. In some embodiments, the PEG moiety is functionalized and the fatty acid esters are C₁₆-C₂₀ straight-chain alkyl fatty acids. For example, the straight-chain alkyl fatty acid is C₁₄, C₁₅, C₁₆, C₁₇, C₁₈, C₁₉, or C₂₀. In some embodiments, the fatty acid esters are C₁₄-C₂₀ symmetric branched-chain alkyl fatty acids. For example, the branched-chain alkyl fatty acid is C₁₄, C₁₅, C₁₆, C₁₇, C₁₈, C₁₉, or C₂₀. By symmetric it is meant that each alkyl branch has the same number of carbons. In some embodiments, the branch is at the 3, 4, 5, 6, or 7 position of the fatty acid ester. The synthesis and use of PEG-lipids built on scaffolds S1-S4 is disclosed in WO2023/196445A1 which is incorporated by reference for all that it teaches about PEG-lipids and their use.

Some embodiments of the disclosed ionizable cationic lipids have head groups with small (<250 Da) PEG moieties. These lipids are not what is meant by the term PEG-lipid as used herein. These small PEG moieties are generally too small to impede binding to a similar extent as the larger PEG moieties of the PEG-lipids disclosed above, though they will impact the lipophilicity of ionizable cationic lipid. Moreover, the PEG-lipids are understood to be primarily located in an exterior facing lamella whereas much of the ionizable cationic lipid is in the interior of the LNP.

In certain embodiments, a functionalized PEG-lipid of a LNP or tLNP or this disclosure comprises one or more fatty acid tails, each that is no shorter than C16 nor longer than C20 for straight-chain fatty acids. For branched chain fatty acids, tails no shorter than C14 fatty acids nor longer than C20 are acceptable. In some embodiments, fatty acid tails are C16. In some embodiments, the fatty acid tails are C18. In some embodiments, the functionalized PEG-lipid comprises a dipalmitoyl lipid. In some embodiments, the functionalized PEG-lipid comprises a distearoyl lipid. The fatty acid tails serve as means to anchor the PEG-lipid in the tLNP to reduce or eliminate shedding of the PEG-lipid from the tLNP. This is a useful property for the PEG-lipid whether or not it is functionalized but has greater significance for the functionalized PEG-lipid as it will have a targeting moiety attached to it and the targeting function could be impaired if the PEG-lipid (with the conjugated anti-CD8α binding moiety, such as an antibody) were shed from the tLNP.

In some embodiments, an LNP or tLNP comprises about 0.5 mol % to about 3 mol % or 0.5 mol % to 3 mol % PEG-lipid comprising functionalized and non-functionalized PEG-lipid. In certain embodiments, an LNP or tLNP comprises DSG-PEG. In other embodiments, an LNP or tLNP comprises DMG-PEG or DPG-PEG. In certain embodiments, an LNP or tLNP comprises DSPE-PEG. In some embodiments, the functionalized and non-functionalized PEG-lipids are not the same PEG-lipid, for example, the non-functionalized PEG-lipid can be a diacylglycerol and the functionalized PEG-lipid a diacyl phospholipid. tLNP with such mixtures have reduced expression in the liver, possibly due to reduced uptake. In certain embodiments the functionalized PEG-lipid is DSPE-PEG and the non-functionalized PEG-lipid is DSG-PEG. In some embodiments, an LNP or tLNP comprises about 0.4 mol % to about 2.9 mol % or about 0.9 mol % to about 1.4 mol % non-functionalized PEG lipid. In certain embodiments, an LNP or tLNP comprises about 1.4 mol % or 1.4 mol % non-functionalized PEG lipid. In some embodiments, an LNP or tLNP comprises about 0.1 mol % to about 0.3 mol % or 0.1 mol % to 0.3 mol % functionalized lipid. In some instances, the functionalized lipid is DSPE-PEG. In certain instances, an LNP or tLNP comprises about 0.1 mol %, about 0.2 mol %, or about 0.3 mol % DSPE-PEG. In certain instances, an LNP or tLNP comprises 0.1 mol %, 0.2 mol %, or 0.3 mol % DSPE-PEG. In certain instances, the functionalized PEG-lipid is conjugated to an anti-CD8α binding moiety, or an engineered F(ab′), as disclosed herein. As used herein, the phrase “is conjugated to” and similar constructions are meant to convey a state of being, that is, a structure, and not a process, unless context dictates otherwise.

### Conjugation

Any suitable chemistry can be used to conjugate the anti-CD8α binding moiety to the PEG of the PEG-lipid, including maleimide (see Parhiz et al., Journal of Controlled Release 291:106-115, 2018) and click (see Kolb et al., Angewandte Chemie International Edition 40(11):2004-2021, 2001; and Evans, Australian Journal of Chemistry 60(6):384-395, 2007) chemistries. Reagents for such reactions include lipid-PEG-maleimide, lipid-PEG-cysteine, lipid-PEG-alkyne, lipid-PEG-dibenzocyclooctyne (DBCO), and lipid-PEG-azide. Further conjugations reactions make use of lipid-PEG-bromomaleimide, lipid-PEG-alkylnoic amide, lipid-PEG-alkynoic imide, and lipid-PEG-alkyne reactions, as disclosed in PCT/US23/17648 entitled PEG-Lipids and Lipid Nanoparticles, which is incorporated by reference for all that it teaches about conjugation chemistry and alternative PEG-lipids. On the anti-CD8α binding moiety side of the reaction one can use an existing cysteine sulfhydryl, or derivatize the protein by adding a sulfur containing carboxylic acid, for example, to the epsilon amino of a lysine to react with maleimide, bromomaleimide, (collectively, “a maleimide”), alkylnoic amide, or alkynoic imide. Alternatively, one can add an alkyne to a sulfhydryl or an epsilon amino of a lysine to participate in a click chemistry reaction.

To modify an epsilon amino of an anti-CD8α binding moiety lysine to react with a maleimide functionalized PEG-lipid the anti-CD8α binding moiety (e.g., an antibody) can be reacted with N-succinimidyl S-acetylthioacetate (SATA). SATA is then deprotected, for example, using 0.5 M hydroxylamine followed by removal of the unreacted components by G-25 Sephadex Quick Spin Protein columns (Roche Applied Science, Indianapolis, IN). The reactive sulfhydryl group on the anti-CD8α binding moiety is then conjugated to maleimide moieties on LNPs of the disclosure using thioether conjugation chemistry. Purification can be performed using Sepharose CL-4B gel filtration columns (Sigma-Aldrich). tLNPs (LNPs conjugated with a targeting antibody) can be stored frozen at −80° C. until needed. Others have conjugated antibody to free functionalized PEG-lipid and then incorporated the conjugated lipid into pre-formed LNP. However, incorporating functionalized PEG-lipid into an LNP during formation of the LNP and subsequently conjugating an anti-CD8α binding moiety to the functionalized PEG-lipid in the LNP was found to be more controllable and produces more consistent results.

There are also several approaches to site-specific conjugation. Particularly but not exclusively suitable for truncated forms of antibody, C-terminal extensions of native or artificial sequences containing a particularly accessible cysteine residue are commonly used. Partial reduction of cystine bonds in an antibody, for example, with tris(2-carboxy)phosphine (TCEP), can also generate thiol groups for conjugation which can be site-specific under defined conditions with an amenable antibody fragment. Potential cysteine residues, particularly in an F(ab′), that can be reduced with TCEP to conjugate to LNP are shown in Table 17. Cysteine, glutathione (GSH), mercaptoethylamine (MEA), and dithiobutylamine (DTBA) could also be used instead of TCEP for reduction. Use of the latter two is described in (Crivianu-Gaita et al., Biochem Biphys Rep. 2: 23-28, (2015)). With sufficient control of conditions, β-mercaptoethanol and dithiothreitol (DTT) could also be used. The various engineered F(ab′) constructs disclosed herein are capable of forming F(ab′)₂, at least to some degree. However, they are referred to throughout as F(ab′) consistent with their use as non-dimerized molecules to be conjugated to LNPs and serve as the targeting moiety of the tLNPs so formed.

Alternatively, the C-terminal extension can contain a sortase A substrate sequence, LPXTG (SEQ ID NO: 197) (where X is any amino acid) which can then be functionalized in a reaction catalyzed by sortase A and conjugated to the PEG-lipid, including through click chemistry reactions (see, for example, Moliner-Morro et al., 2020, Biomolecules 10(12):1661, which is incorporated by reference herein for all that it teaches regarding antibody conjugations mediated by the sortase A reaction and/or click chemistry). The use of click chemistry for the conjugation of a targeting moiety, such as various forms of antibody, is disclosed, for example, in WO2024/102,770 which is incorporated by reference in its entirety for all that it teaches about the conjugation of targeting moieties to LNPs that is not inconsistent with this disclosure.

For whole antibody and other forms comprising an Fc region, site-specific conjugation to either (or both) of two specific lysine residues (Lys248 and Lys288) can be accomplished without any change to or extension of the native antibody sequence by use of one of the AJICAP® reagents (see, for example, Matsuda et al., 2021, Molecular Pharmaceutics 18:4058-4066; Fujii et al., 2023, Bioconjugate Chemistry 34(4):728-738 [https://doi.org/10.1021/acs.bioconjchem.3c00040], and WO2019/240287 which are incorporated by reference herein for all that they teach regarding conjugation of antibodies with AJICAP reagents). AJICAP reagents are modified affinity peptides that bind to specific loci on the Fc and react with an adjacent lysine residue to form an affinity peptide conjugate of the antibody. The peptide is then cleaved with base to leave behind a thiol-functionalized lysine residue which can then undergo conjugation through maleimide or haloamide reactions, for example). Functionalization with azide or dibenzocyclooctyne (DBCO) for conjugation by click chemistry is also possible. This and similar technology are further described in US20200190165 (corresponding to WO2018199337), US20210139549 (corresponding to WO2019/240287) and US20230248842 (corresponding to WO2020184944) which are incorporated by reference in their entirety for all that they teach about such modified affinity peptides and their use.

The term “affinity peptide” refers to a peptide with capacity to bind specifically and with high affinity to other molecules. In certain embodiments, the affinity peptide binds to specific loci on the Fc region of an antibody. In certain embodiments, the affinity peptide is modified with chemically reactive groups that allow it to form a covalent bond with an adjacent amino acid residue in the antibody, for example, a particular lysine residue such as Lys248 or Lys288 in IgG1.

Accordingly, in some embodiments the anti-CD8α binding moiety is conjugated to the PEG moiety of the PEG-lipid through a thiol modified lysine residue. In some embodiments the conjugation is through a cysteine residue in a native or added antibody sequence. In such embodiments, whether using a thoilated lysine or the thiol of a cysteine, the thiol in the antibody can be conjugated to a maleimide group of a maleimide-modified PEG-lipid in the LNP utilized a maleimide-thiol reaction. In other embodiments, the conjugation is through a sortase A substrate sequence. In still other embodiments, the conjugation is through a specific lysine residue (Lys248 or Lys288) in the Fc region. In certain embodiments of such embodiments, the humanized anti-CD8 antibody is linked to the LNP using N-succinimidyl S-acetylthioacetate (SATA)-maleimide conjugation chemistry to form targeting LNP (tLNP). The antibody is beneficially first modified with SATA to introduce sulfhydryl groups at accessible lysine residues allowing conjugation to maleimide. (Some lysine residues may be buried in the interior of the protein and thus inaccessible to the SATA reagent.) Diabodies and F(ab′)₂ can be conjugated by first partially reducing cystine bonds in the antibody with tris(2-carboxy)phosphine (TCEP) to generate thiol groups for conjugation through the maleimide moieties of the LNP.

### Nucleic Acid Molecules

In certain embodiments, the disclosed LNP and tLNP comprise a payload comprising or consisting of one or more species of nucleic acid molecule. In some embodiments, the LNP or tLNP payload comprises only one nucleic acid species while in other embodiments the LNP or tLNP payload comprises multiple nucleic acid species, for example, 2, 3, or 4 nucleic acid species. For example, in embodiments in which the payload comprises a nucleic acid encoding a CAR or immune cell engager (ICE), the payload can comprise or consist of 1) a single nucleic acid species encoding a single species of CAR or ICE, 2) a single nucleic acid species encoding 2 or more species of CAR or ICE (or a mixture of CAR and ICE) such as a pursued antigen, 3) a single nucleic acid species encoding 2 or more species of CAR or ICE (or a mixture of CAR and ICE) such as a bicistronic or multicistronic mRNA in which at least one CAR and/or ICE has specificity for a different pursued antigen than the other(s), 4) two or more nucleic acid species encoding 2 or more species of CAR or ICE (or a mixture of CAR and ICE) in which each CAR and/or ICE has specificity for a same pursued antigen, or 5) two or more nucleic acid species encoding 2 or more species of CAR or ICE (or a mixture of CAR and ICE) in which at least one CAR and/or ICE has specificity for a different target antigen than the other(s). When two or more CAR and/or ICE have specificity for a same target antigen, they can have specificity for same or different epitopes of the same pursued antigen. Further variations will be apparent to one of skill in the art (e.g., multiple bi- or multicistronic nucleic acids, nucleic acids encoding a TCR and the like). The nucleic acid can be RNA or DNA. The nucleic acid can be multicistronic, for example, bicistronic.

In some embodiments, the nucleic acid molecule is an mRNA, a self-replicating RNA, a circular RNA, a siRNA, a miRNA, DNA, a gene editing component (for example, a guide RNA, a tracr RNA, a sgRNA), a gene writing component, an mRNA encoding a gene or base editing protein, a zinc-finger nuclease, a TALEN, a CRISPR nuclease, such as Cas9, a DNA molecule to be inserted or serve as a template for repair), and the like, or a combination thereof. In some embodiments, the nucleic acid comprises small interfering RNA (siRNA), microRNA (miRNA), antisense oligonucleotide (ASO). In some embodiments, the nucleic acid comprises a self-replicating RNA or a circular RNA. In some embodiments, the mRNA encodes a reprogramming agent or comprises or encodes a conditioning agent. In some embodiments, the mRNA (linear, circular, or self-replicating) comprises an miRNA binding site. In some embodiments, an mRNA encodes a chimeric antigen receptor (CAR). In other embodiments, an mRNA encodes a gene-editing or base-editing or gene writing protein. In some embodiments, a nucleic acid is a guide RNA. In some embodiments, an LNP or tLNP comprises both a gene- or base-editing or gene writing protein-encoding mRNA and one or more guide RNAs. CRISPR nucleases can have altered activity, for example, modifying the nuclease so that it is a nickase instead of making double-strand cuts or so that it binds the sequence specified by the guide RNA but has no enzymatic activity. Base-editing proteins are often fusion proteins comprising a deaminase domain and a sequence-specific DNA binding domain (such as an inactive CRISPR nuclease).

In some embodiments, the reprogramming agent comprises an immune receptor (for example, a chimeric antigen receptor or a T cell receptor) or an immune cell engager (for example, a bispecific T cell engager (BiTE), a bispecific killer cell engager (BiKE), a trispecific kill cell engager (TriKE), a dual affinity retargeting antibody (DART), a TRIDENT (linking two DART units or a DART unit and a Fab domain), a macrophage engager (e.g., BiME), an innate cell engager, and the like).

In some embodiments, the nucleic acid is an RNA, for example, mRNA, and the RNA comprises at least one modified nucleoside. In some embodiments, the modified nucleoside is pseudouridine, N1-methylpseudouridine, 5-methylcytosine, 5-methyluridine, N6-methyladenosine, 2′-O-methyluridine, or 2-thiouridine. In certain embodiments, all of the uridines are substituted with a modified nucleoside. Further disclosure of modified nucleosides and their use can be found in U.S. Pat. No. 8,278,036 which is incorporated herein by reference for those teachings.

In some embodiments, the reprogramming agent encodes or is a gene/genome editing component. In some embodiments, the gene/genome editing component is a guide RNA for an RNA-directed nuclease or other nucleic acid editing enzyme, clustered regularly interspaced short palindromic repeat RNA (crisprRNA), a trans-activating clustered regularly interspaced short palindromic repeat RNA (tracrRNA). In some embodiments, the gene/genome editing component is a nucleic acid-encoded enzyme, such as RNA-guided nuclease, a gene or base editing protein, a zinc finger nuclease (ZFN), a transcription activator-like effector nuclease (TALEN), a meganuclease, a transposase, or a CRISPR nuclease (e.g., Cas9 or Cas 12, etc.). In some embodiments, the gene/genome editing component is DNA to be inserted or that serves as a template in gene or genome editing for example a template for repair of a double-strand break.

In some embodiments comprising multiple agents, the nucleic acid can be multicistronic. In other embodiments comprising multiple agents or components, each agent or component is encoded or contained is a separate nucleic acid species. In some embodiments involving multiple payload nucleic acid species, two or more nucleic acid species are packaged together in a single LNP species. In other embodiments, a subset of the payload nucleic acid species to be delivered, (e.g., a single nucleic acid species) is packaged in one LNP or tLNP species while another subset of the nucleic acid species is packaged in another LNP or tLNP species. The different (t)LNP species can differ by only the payload they contain. The different (t)LNP species can be combined in a single formulation or pharmaceutical composition for administration.

In any of the aforementioned embodiments, a nucleic acid molecule payload of a LNP or tLNP of this disclosure encodes a CAR, TCR, or ICE that is specific for a particular antigen, such as B cell maturation agent (BCMA)^{†‡}, CA9^{†‡}, CD1, CD2*^{†‡}, CD3*^{†‡}, CD4*^{†‡}, CD5^{†‡}, CD7^{†‡}, CD11b^{‡}, CD14^{†‡}, CD16, CD19*^{†‡}, CD20 (MS4A1)*^{†‡}, CD22*^{†‡}, CD23*^{†‡}, CD25^{†‡}, CD26*^{‡}, CD27*^{†‡}, CD28*^{†‡}, CD30 (TNFRSF8)*^{†‡}, CD32*, CD33*^{†‡}, CD38*^{†‡}, CD39^{‡}, CD40*^{†‡}, CD40L (CD154)*^{†‡}, CD44*^{‡}, CD45^{†‡}, CD45^{†‡}, CD56 (NCAM1)*^{†‡}, CD64*^{‡}, CD62^{†‡}, CD68, CD69^{‡}, CD70*^{†‡}, CD73^{†‡}, CD80*^{‡}, CD83^{‡}, CD86*^{‡}, CD95^{‡}, CD103^{‡}, CD119^{‡}, CD126^{‡}, CD133^{‡}, CD137 (41BB)^{†‡}, CD138 (SDC1)*^{‡}, CD150^{‡}, CD153^{‡}, CD161^{‡}, CD166^{‡}, CD174, CD183 (CXCR3)^{‡}, CD185 (CXCR5)^{‡}, CD223 (LAG-3)*^{†‡}, CD254^{‡}, CD267 (TACI)^{‡}, CD274 (PD-L1)*^{†‡}, CD275^{‡}, CD276 (B7-H3)^{†‡}, ADAM12^{‡}, CTLA-4*^{†}*^{†}, DEC205, OX40^{†}, PD-1*^{†‡}, GITR^{†}, TIM-3*^{†‡}, FasL*^{‡}, IL18R1, ICOS (CD278)^{‡}, leu-12, TCR^{†}, TLR1, TLR2^{†‡}, TLR3*^{‡}, TLR4^{†‡}, TLR6, TREM2^{‡}, NKG2^{‡}, CCR, CCR1 (CD191)^{‡}, CCR2 (CD192)*^{†‡}, CCR4(CD194)*^{†‡}, CCR6(CD196)^{‡}, CCR7^{‡}, low affinity IL-2 receptor^{†‡}, IL-7 receptor^{‡}, IL-12 receptor^{‡}, IL-15 receptor^{‡}, IL-18 receptor^{‡}, and IL-21 receptor^{‡}CEACAM5*^{†‡}, CLL1^{‡}, CSPG4*^{‡}, Kappa*, Lambda*, FCRL5^{†‡}, GPRC5D^{†‡}, CTSK, PD-1 (CD279)T^{†‡}, CD319 (SLAMF7)*^{†‡}, CD248 (TEM1)^{‡}, ULBP1, ULBP2; CD319 (SLAMF7)*^{†‡}, GPRC5D^{†‡}, Claudin 6 (CLDN6), Claudin 18.2 (CLDN18.2), GD2*^{†‡}, HER2*^{†‡}, ITGA11, EGFR*^{†‡}, EGFRvIII*, CD276 (B7H3)^{†‡}, PSMA*^{†‡}, PSCA^{‡}, CAIX (CA9)^{†‡}, CD171 (L1-CAM)*^{‡}, CEA*^{‡}, CSPG4*^{‡}, DLL3, EPHA2*^{‡}, FAP*^{†‡}, LRRC15^{†‡}, FOLR1*^{†‡}, IL-13Rα*^{†‡}, Mesothelin (MSLN)*^{†‡}, MUC1*^{†‡}, MUC16^{†‡}, Nectin-4^{†‡}, NOX4, SGCD, SYNDIG1, CDH11^{‡}, PLPP4, SLC24A2, PDGFRB*^{‡}, THY1^{‡}, ANTXR1^{‡}, GAS1, CALHM5, SDC1EPCAM*^{†‡}, ERBB2*^{‡}, FOLH1, GPC3*^{†‡}, GPNMB*^{‡}, IL1RAP^{†‡}, IL3RA*^{‡}, IL13RA2 (IL13Rα2)*^{‡}, KDR (VEGFR2)*^{‡}, CD171 (L1CAM)*^{‡}, MET*^{‡}, TROP2*^{†‡}, and ROR1^{†‡}.

### Tolerability

Conventional LNPs deliver primarily to the liver. Liver toxicity has been the major dose limiting parameter observed with LNP-containing pharmaceuticals. For example, ONPATTRO®, comprising the ionizable lipid MC3, has a NOAEL (no observed adverse effect level) of only 0.3 mg/kg for multiple dosing in rats. A benchmark LNP comprising the ionizable cationic lipid ALC-0315, used in the SARS-CoV-2 vaccine COMIRNATY®, caused elevated levels of liver enzymes and acute phase proteins at single doses of ≥1 mg/kg in the rat. Merely attaching an antibody to the benchmark LNP partially reverses that elevation and the reversal is greater if the antibody directs the LNP to some other tissue (that is, a tLNP). However, use of a highly biodegradable ionizable cationic lipid, CICL-1, the catabolism of which should be similar to those disclosed herein, reduced delivery to the liver and associated liver enzyme and acute phase protein levels to a greater extent for LNP, antibody-conjugated LNP, and tLNP

Method of Making LNPs or tLNPs

In some aspects, the present disclosure provides a method of making a LNP or tLNP comprising mixing of an aqueous solution of a nucleic acid (or other negatively charged payload) and an alcoholic solution of the lipids in proportions disclosed herein. In particular embodiments, the mixing is rapid.

The aqueous solution is buffered at pH of about 3 to about 5, for example, without limitation, with citrate or acetate. In various embodiments, the alcohol can be ethanol, isopropanol, t-butanol, or a combination thereof. In some embodiments, the rapid mixing is accomplished by pumping the two solutions through a T-junction or with an impinging jet mixer. Microfluidic mixing through a staggered herringbone mixer (SHM) or a hydrodynamic mixer (microfluidic hydrodynamic focusing), microfluidic bifurcating mixers, and microfluidic baffle mixers can also be used. After the LNPs are formed they are diluted with buffer, for example phosphate, HEPES, or Tris, in a pH range of 6 to 8.5 to reduce the alcohol (ethanol) concentration, The diluted LNPs are purified either by dialysis or ultrafiltration or diafiltration using tangential flow filtration (TFF) against a buffer in a pH range of 6 to 8.5 (for example, phosphate, HEPES, or Tris) to remove the alcohol. Alternatively, one can use size exclusion chromatography. Once the alcohol is completely removed the buffer is exchanged with like buffer containing a cryoprotectant (for example, glycerol or a sugar such as sucrose, trehalose, or mannose). The LNPs are concentrated to a desired concentrated, followed by 0.2 μm filtration through, for example, a polyethersulfone (PES) or modified PES filter and filled into glass vials, stoppered, capped, and stored frozen. In alternative embodiments, a lyoprotectant is used and the LNP lyophilized for storage instead of as a frozen liquid. Further methodologies for making LNP can be found, for example, in U.S. Patent Application Publication Nos. US 2020/0297634, US 2013/0115274, and International Patent Application Publication No. WO 2017/048770, each of which is incorporated by reference for all that they teach about the production of LNP.

Some aspects are a method of making a tLNP comprising rapid mixing of an aqueous solution of a nucleic acid (or other negatively charged payload) and an alcoholic solution of the lipids as disclosed for LNP. In some embodiments, the lipid mixture includes functionalized PEG-lipid, for later conjugation to a targeting moiety. As used herein, functionalized PEG-lipid refers to a PEG-lipid in which the PEG moiety has been derivatized with a chemically reactive group (such as, maleimide, N-hydroxysuccinimide (NHS) ester, Cys, azide, alkyne, and the like) that can be used for conjugating a targeting moiety to the PEG-lipid, and thus, to the LNP comprising the PEG-lipid. In other embodiments, the functionalized PEG-lipid is inserted into and LNP subsequent to initial formation of an LNP from other components. In either type of embodiment, the targeting moiety is conjugated to functionalized PEG-lipid after the functionalized PEG-lipid containing LNP is formed. Protocols for conjugation can be found, for example, in Parhiz et al. 2018, J. Controlled Release 291:106-115, and Tombacz et al., 2021, Molecular Therapy 29(11):3293-3304, each of which is incorporated by reference for all that it teaches about conjugation of PEG-lipids to binding moieties. Alternatively, the targeting moiety can be conjugated to the PEG-lipid prior to insertion into pre-formed LNP.

In certain embodiments of the preparation methods of tLNP, the method comprises:
i) forming an initial LNP by mixing all components of the tLNP, in proportions disclosed herein, except for the one or more functionalized PEG-lipids and the one or more targeting moieties;
ii) forming a pre-conjugation tLNP by mixing the initial LNP with the one or more functionalized PEG-lipids; and
iii) forming the tLNP by conjugating the pre-conjugation tLNP with the one or more targeting moieties.

In certain embodiments of the preparation methods of tLNP, the method comprises:
i) forming a pre-conjugation tLNP by mixing all components of the tLNP, in proportions disclosed herein, including the one or more functionalized PEG-lipids, except for the one or more targeting moieties; and
ii) forming the tLNP by conjugating the pre-conjugation tLNP with the one or more targeting moieties.

In certain embodiments of the preparation methods of tLNP, the method comprises:
i) forming one or more conjugated functionalized PEG-lipids by conjugating the one or more functionalized PEG-lipids with the one or more targeting moieties; and
ii) forming the tLNP by mixing all components of the tLNP, in proportions disclosed herein, including the one or more conjugated functionalized PEG-lipids.

In certain embodiments of the preparation methods of tLNP, the method comprises:
i) forming one or more conjugated functionalized PEG-lipids by conjugating the one or more functionalized PEG-lipids with the one or more targeting moieties;
ii) forming an LNP by mixing all components of the tLNP, except the one or more conjugated functionalized PEG-lipids; and
iii) forming the tLNP by mixing the initial LNP with the one or more conjugated functionalized PEG-lipids.

After the conjugation the tLNPs are purified by dialysis, tangential flow filtration, or size exclusion chromatography, and stored, as disclosed above for LNPs.

The encapsulation efficiency of the nucleic acid by the LNP or tLNP is typically determined with a nucleic acid binding fluorescent dye added to intact and lysed aliquots of the final LNP or tLNP preparation to determine the amounts of unencapsulated and total nucleic acid, respectively. Encapsulation efficiency is typically expressed as a percentage and calculated as 100×(T-U)/T where T is the total amount of nucleic acid and U is the amount of unencapsulated nucleic acid. In various embodiments, the encapsulation efficiency is ≥80%, ≥85%, ≥90%, or ≥95%

Anti-CD8 tLNPs

The instant disclosure contemplates any of the aforementioned embodiments of anti-CD8α binders being conjugated to tLNP formulation disclosed herein. For example, in certain aspects, a targeted lipid nanoparticle (tLNP) comprises: (a) a lipid formulation (e.g., any listed in Table 14) comprising an ionizable cationic lipid (such as CICL or a variant thereof of this disclosure), a phospholipid, a sterol, a functionalized PEG-lipid, and a non-functionalized PEG-lipid, and (b) a humanized anti-CD8α antibody or antigen binding fragment thereof conjugated to the lipid, wherein the humanized anti-CD8α antibody or antigen binding fragment thereof comprises: (a)(i) a VH-CDR1 comprising the amino acid sequence of SEQ ID NO: 2, a VH-CDR2 comprising the amino acid sequence of SEQ ID NO: 3, and a VH-CDR3 comprising the amino acid sequence SEQ ID NO: 4; (ii) a VH-CDR1 comprising the amino acid sequence of SEQ ID NO: 2, a VH-CDR2 comprising the amino acid sequence of SEQ ID NO: 58, and a VH-CDR3 comprising the amino acid sequence SEQ ID NO: 4; (iii) a VH-CDR1 comprising the amino acid sequence of SEQ ID NO: 2, a VH-CDR2 comprising the amino acid sequence of SEQ ID NO: 59, and a VH-CDR3 comprising the amino acid sequence SEQ ID NO: 4; or (iv) a VH-CDR1 comprising the amino acid sequence of SEQ ID NO: 2, a VH-CDR2 comprising the amino acid sequence of SEQ ID NO: 60, and a VH-CDR3 comprising the amino acid sequence SEQ ID NO: 4; and (b) a VL-CDR1 comprising the amino acid sequence SEQ ID NO: 6, a VL-CDR2 comprising the amino acid sequence SEQ ID NO: 7; and a VL-CDR3 comprising the amino acid sequence SEQ ID NO: 8.

In further embodiments, a targeted lipid nanoparticle (tLNP) comprises: (a) a lipid formulation (e.g., any listed in Table 14) comprising an ionizable cationic lipid (such as CICL or a variant thereof of this disclosure), a phospholipid, a sterol, a functionalized PEG-lipid, and a non-functionalized PEG-lipid, and (b) an anti-CD8α antibody or antigen binding fragment thereof conjugated to the lipid, wherein the anti-CD8α antibody or antigen binding fragment thereof comprises a heavy chain variable region (VH) comprising an amino acid sequence that is at least 90% up to 100% identical to the amino acid sequence of SEQ ID NO: 10, 11, 12, 13, 14, 27, 28, 29, 35, or 36 and wherein the VH-CDR1 comprises the amino acid sequence of SEQ ID NO: 2, the VH-CDR2 comprises the amino acid sequence of SEQ ID NO: 3, 58, 59, or 60, and the VH-CDR3 comprises the amino acid sequence of SEQ ID NO: 4; and a light chain variable region (VL) comprising an amino acid sequence that is at least 90% up to 100% identical to the amino acid sequence of SEQ ID NO: 16, 17, 18, or 39, and wherein the VL-CDR1 comprises the amino acid sequence of SEQ ID NO: 6, the VL-CDR2 comprises the amino acid sequence of SEQ ID NO: 7, and the VL-CDR3 comprises the amino acid sequence of SEQ ID NO: 8.

As used herein, “LNP formulation” or “tLNP formulation” refers to the complete respective composition (e.g., all the lipids that comprise an LNP or all the lipids together with the targeting moiety that comprise a tLNP, each optionally encompassing a payload such as a nucleic acid molecule) and further including a buffer, carrier, solvent or other excipient. In some embodiments, humanized anti-CD8α antibodies or antigen binding fragments thereof of this disclosure, or targeted LNP (tLNP) of this disclosure are conjugated to such anti-CD8α antibodies or antigen binding fragments, which anti-CD8α binders or CD8-targeted tLNPs can be formulated with a pharmaceutically acceptable carrier, excipient, or stabilizer, as compositions or pharmaceutical compositions.

In certain embodiments, such compositions are suitable for administration to a human or non-human animal via one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations can also contain compatible solid or liquid fillers, diluents, or encapsulating substances, which are suitable for administration into a human. Other contemplated carriers, excipients, and/or additives, which can be utilized in the formulations described herein include, for example, antimicrobial agents, antioxidants, antistatic agents, lipids, protein excipients such as serum albumin, gelatin, casein, salt-forming counterions such as sodium, and the like. These and additional pharmaceutical carriers, excipients, and/or additives suitable for use in the formulations described herein are known in the art, for example, as listed in “Remington: The Science & Practice of Pharmacy,” 23rd ed., Lippincott Williams & Wilkins, (2005), and in the “Physician's Desk Reference,” 71st ed., Medical Economics, Montvale, N.J. (2005). Pharmaceutically acceptable carriers can be selected that are suitable for the mode of administration, solubility, and/or stability desired or required.

In some embodiments, the herein disclosed humanized anti-CD8α antibody or antigen binding fragments thereof can be delivered through various routes of administration, such as intradermal, intramuscular, intraperitoneal, intravenous, subcutaneous, or epidural. Administration can be local or systemic. The mode of administration can be left to the discretion of the practitioner and depends in part upon the site of the medical condition. In most instances, administration results in the release of the humanized anti-CD8α antibody or polypeptides comprising the antigen binding domain thereof described herein into the bloodstream.

In other embodiments, CD8-targeted tLNPs conjugated to humanized anti-CD8α antibody antigen binding fragments thereof of this disclosure are administered parenterally, such as by intravenous infusion. Other embodiments make use of other routes of administration, including subcutaneous, intraperitoneal, intranodal, and intratumoral. In most instances, administration results in binding of the tLNP to a CD8-positive cell (e.g., T cells) and the release of the payload (such as nucleic acid molecule like RNA) encapsulated by the tLNP into the cell.

Methods of Using Anti-CD8 tLNP to Deliver a Payload into a Cell

In some aspects, disclosed herein are methods of delivering a nucleic acid (or other negatively charged payload) into a cell expressing CD8+ (CD8+ cell) comprising contacting the CD8+ cell with a CD8-targeted tLNP of any of the foregoing aspects. Various embodiments of the methods of delivering a payload to a CD8+ cell are limited to one or another. Each of the various genera, subgenera, and or species of LNP or tLNP disclosed herein including those based on the inclusion or exclusion of particular lipids, particular lipid compositions, and/or particular payloads can be used in defining the scope of the methods of delivering a payload to a CD8+ cell. In some embodiments the contacting takes place ex vivo. In some embodiments, the contacting takes place in vivo. In some instances, the in vivo contacting comprises intravenous, intramuscular, subcutaneous, intranodal or intralymphatic administration. In further instances, transfection of hepatocytes is reduced as compared to tLNPs comprising a conventional ionizable cationic lipid, such as ALC-0315 (Table 14). In some embodiments, an LNP or tLNP is administered 1-3 times a week for 1, 2, 3, or 4 weeks. In some embodiments, toxicity is confined (or largely confined) to grades of 0 or 1 or 2, as discussed above.

The herein disclosed LNP and tLNP compositions and formulations have reduced toxicity as compared to widely used prior LNP compositions such as those containing ALC-0315. In various embodiments the toxicity can be described as an observable toxicity, a substantial toxicity, a severe toxicity, or an acceptable toxicity, or a dose-limiting toxicity (such as but not limited to a maximum tolerated dose (MTD)). By an observable toxicity it is meant that while a change is observed the effect is negligible or mild. By substantial toxicity it is meant that there is a negative impact on the patient's overall health or quality of life. In some instances, a substantial toxicity may be mitigated or resolved with other ongoing medical intervention. By a severe toxicity it is meant that the effect requires acute medical intervention and/or dose reduction or suspension of treatment. The acceptability of a toxicity will be influenced by the particular disease being treated and its severity and the availability of mitigating medical intervention. In some embodiments, toxicity is confined (or largely confined) to an observable toxicity. In some embodiments, toxicity is confined (or largely confined) to grades of 0 or 1 or 2.

In some embodiments, the payload is a nucleic acid and the method of delivering is a method of transfecting a CD8+ cell. In some embodiments, the nucleic acid payload comprises an mRNA, circular RNA, self-amplifying RNA, or guide RNA. Nucleic acid structures and especially mRNA structures, as well as individual RNA molecules encoding particular polypeptides, that are well-adapted to delivery by LNP or tLNP are disclosed in U.S. application Ser. No. 18/934,237 (Atty Docket No. 23-1871-US) filed on Nov. 1, 2024, each of which is incorporated by reference for all that it teaches about nucleic acid payloads for in vivo transfection and their design.

In some embodiments, the payload comprises a nucleic acid encoding an immune receptor or immune cell engager and the method of delivering is also a method of reprogramming an immune cell expressing CD8+ surface molecule. In some embodiments, the payload comprises a nucleic acid that encodes, or is, a BRM and the method of delivering is also a method of providing a conditioning agent. In various embodiments, the BRM or conditioning agent is a gamma chain receptor cytokine such as IL-2, IL-7, IL-15, IL-15/15Ralpha, IL-21; an immune modulating cytokine such as IL-12, IL-18; a chemokine such as RANTES, IP10, MIG; or another BRM such as Flt3, GM-CSF, and G-CSF.

In some embodiments, the payload comprises a nucleic acid encoding a gene/genome editing enzyme and/or a guide RNA or other component of a gene/genome editing system and the method of delivering is also a method of reprogramming a cell. In some instances, the cell is an immune cell expressing CD8+ surface molecule. In some instances, the cell is an hematopoietic stem cells (HSC). In some instances, the cell is an mesenchymal stem cells (MSC). In certain embodiments comprising delivering the payload into an immune cell, the anti-CD8 binding moiety binds to a lymphocyte CD8+ surface molecule.

In certain embodiments comprising delivering the payload into an immune cell, the anti-CD8 tLNP binds to CD8+ expressing lymphocyte.

### Methods of Treatment

Anti-CD8 binders and tLNP conjugated to such anti-CD8 binders of the present disclosure are useful for treating disease (e.g., CBD1032, CBD1033, CBD1035, CBD1037, CBD1039, CBD1047, CBD1049, or like and conjugates of such binders to LNPs). Those anti-CD8 binders and CD8-specific tLNPs disclosed herein offer a targeted approach to drug delivery strategies. Accordingly, certain embodiments provide a method of treating a disease (or the symptoms thereof) comprising administering to a mammal (e.g., a human) in need thereof a therapeutically effective amount of anti-CD8 binders or CD8-specific tLNPs or compositions comprising the same.

“Treat” and/or “treating” refer to any indicia of success in the treatment or amelioration of a disease or condition. Treating can include, for example, reducing, delaying, or alleviating the severity of one or more symptoms of the disease or condition, or it can include reducing the frequency with which symptoms of a disease, defect, disorder, or adverse condition or the like, are experienced by a patient. Treat can be used herein to refer to a method that results in some level of treatment or amelioration of the disease or condition and can contemplate a range of results directed to that end, including prevention of the condition entirely.

“Prevent”, “preventing” or the like refer to the prevention of the disease or condition, e.g., autoimmune antibody production, in a patient. For example, if an individual at risk of developing autoimmune flare ups or other related symptoms is treated with the methods of this disclosure and does not later develop autoimmune-related flare-ups or other related symptoms, then the disease has been prevented, at least over a period of time, in that individual. Preventing can also refer to preventing re-occurrence of a disease or condition in a patient that has previously been treated for the disease or condition, e.g., by preventing relapse.

A therapeutically effective amount (also referred to as an effective amount) can be the amount of a composition comprising an anti-CD8 binder or CD8-specific tLNP sufficient to provide a beneficial effect or to otherwise reduce a detrimental non-beneficial event to the individual to whom the composition is administered. A therapeutically effective dose can be a dose that produces one or more desired or desirable (e.g., beneficial) effects for which it is administered, such administration occurring one or more times over a given period of time. An exact dose can depend on the purpose of the treatment and can be ascertainable by one skilled in the art using known techniques and the teachings provided herein.

The anti-CD8 binders or CD8-specific tLNPs of this disclosure that can be used in therapy can be formulated and dosages established in a fashion consistent with good medical practice taking into account the disease or condition to be treated, the condition of the individual patient, the site of delivery of the composition, the method of administration, and other factors known to practitioners. The compositions can be prepared according to the description of preparation described herein.

Compositions can be used in the methods described herein and can be administered to a subject in need thereof using a technique known to one of ordinary skill in the art which can be suitable as a therapy for the disease or condition affecting the subject. One of ordinary skill in the art would understand that the amount, duration, and frequency of administration of a pharmaceutical composition to a subject in need thereof depends on several factors including, for example, the health of the subject, the specific disease or condition of the patient, the grade or level of a specific disease or condition of the patient, the additional treatments the subject is receiving or has received, or the like.

The anti-CD8 binders or CD8-specific tLNPs, compositions, and methods of this disclosure are useful in the treatment or prevention of disease, such as autoimmune disorders (e.g., idiopathic inflammatory myopathies, such as antisynthetase syndrome), and cancer as single agents. Alternatively, the anti-CD8 binders or CD8-specific tLNPs, compositions, and methods of this disclosure may be used in combination therapies with second therapeutic agents for treating or preventing diseases, such as autoimmune disorders, and cancer.

In certain aspects, this disclosure provides methods of treating a disease or disorder comprising administering an anti-CD8 binder or CD8-specific tLNP of this disclosure to a subject in need thereof. In some embodiments, a subject is a human. In some embodiments, an antibody or tLNP of this disclosure is administered systemically. In some embodiments, an antibody or tLNP of this disclosure is administered by intravenous or subcutaneous infusion or injection. In some embodiments, an antibody or tLNP of this disclosure is administered locally. In some embodiments, an antibody or tLNP of this disclosure is administered by intraperitoneal or intralesional infusion injection. Certain embodiments of the LNPs and tLNPs disclosed herein are capable of treating a disease or disorder as set forth in paragraphs [00307]-[00312].

In some embodiments, the disease or disorder is an autoimmune disease. Examples of autoimmune disease include, without limitation, myocarditis, acute idiopathic thrombocytopenic purpura, chronic idiopathic thrombocytopenic purpura, dermatomyositis, Sydenham's chorea, myasthenia gravis, systemic lupus erythematosus, fibrosing alveolitis, multiple sclerosis, rheumatic fever, polyglandular syndromes, agranulocytosis, autoimmune hemolytic anemias, bullous pemphigoid, Wegener's granulomatosis, membranous nephropathy, amyotrophic lateral sclerosis, tabes dorsalis, giant cell arteritis/polymyalgia, pernicious anemia, rapidly progressive glomerulonephritis, IgA nephropathy, polyarteritis nodosa, ankylosing spondylitis, allergic responses, insulin-resistant diabetes, psoriasis, diabetes mellitus, Addison's disease, Grave's disease, diabetes, endometriosis, celiac disease, Crohn's disease, Henoch-Schonlein purpura, ulcerative colitis, Goodpasture's syndrome, thromboangitisubiterans, Sjögren's syndrome, aplastic anemia, rheumatoid arthritis, sarcoidosis, scleritis, a T cell-mediated autoimmunity or a B cell-mediated autoimmunity, a B cell-mediated (antibody-mediated) autoimmune disease, necrotizing myopathy, chronic inflammatory demyelinating polyneuropathy (CIDP), neuromyelitis optica (NMO) myositis, neuromyelitis optica spectrum disorders, pemphigus vulgaris, systemic sclerosis, antisynthetase syndrome (idiopathic inflammatory myopathy), lupus nephritis, membranous nephropathy, Fanconi anemia, and vasculitis.

In some embodiments, the autoimmune disease is a T cell-mediated autoimmunity or a B cell-mediated autoimmunity. In some instances, the B cell-mediated autoimmune disease is myositis (such as anti-synthetase myositis), lupus nephritis, membranous nephropathy, systemic lupus erythematosus, anti-neutrophilic cytoplasmic antibody (ANCA) vasculitis, neuromyelitis optica spectrum disorder (NMOSD), myasthenia gravis, pemphigus vulgaris, rheumatoid arthritis, dermatomyositis, immune mediated necrotizing myopathy (IMNM), anti-synthetase syndrome, polymyositis, systemic sclerosis, diffuse cutaneous systemic sclerosis, limited cutaneous systemic sclerosis, anti-synthetase syndrome (idiopathic inflammatory myopathy), stiff person syndrome, myeloid oligodendrocyte glycoprotein autoantibody associated disease (MOGAD), amyloid light-chain amyloidosis, multiple sclerosis, relapsing-remitting multiple sclerosis, secondary progressive multiple sclerosis, primary progressive multiple sclerosis, non-active secondary progressive multiple sclerosis, Sjörgen's syndrome, IgA nephropathy, IgG4-related disease, or Fanconi anemia. In certain embodiments, the B cell-mediated autoimmune disease is myositis, lupus nephritis, membranous neuropathy, scleroderma, systemic lupus erythematosus, myasthenia gravis, ANCA vasculitis, multiple sclerosis, or pemphigus vulgaris. In certain embodiments, the B cell-mediated autoimmune disease is myositis, lupus nephritis, membranous neuropathy, or scleroderma. In certain embodiments, the B cell-mediated autoimmune disease is myositis. In some instances, the myositis is anti-synthetase myositis. In certain embodiments, the B cell-mediated autoimmune disease is systemic lupus erythematosus, myasthenia gravis, ANCA vasculitis, multiple sclerosis, or pemphigus vulgaris.

In some embodiments, the disease or disorder is rejection of an allogeneic organ or tissue graft. Pre-existing antibodies and/or B cells, in their role as antigen presenting cells, can facilitate rapid immune rejection through known mechanisms hence depleting a large number of B cells can help prevent allograft rejection.

In some embodiments, the disease or disorder is a cancer. Examples of cancers include, without limitation, carcinomas, sarcomas, and hematologic cancers. In some embodiments, the hematologic cancer is a lymphoma, leukemia, or myeloma. In some instances, the hematologic cancer is a B lineage or T lineage cancer. In some instances, the B lineage cancer is multiple myeloma, diffuse large B cell lymphoma, acute myeloid leukemia, Mantle Cell lymphoma, follicular lymphoma, B acute lymphoblastic leukemia, chronic lymphocytic leukemia, or myelodysplastic syndrome. In some embodiments, the cancer is a sarcoma. In some embodiments, the cancer is a carcinoma, such as breast cancer, colon cancer, ovarian cancer, lung cancer, testicular cancer, or pancreatic cancer. In some embodiments, the cancer is melanoma.

In some embodiments, the disease or disorder is a genetic disease or disorder such as a monogenic genetic disease. In some instances, the genetic disease or disorder is a hemoglobinopathy, for example, sickle cell disease or β-thalassemia.

In some embodiments, the disease or disorder is a fibrotic disease or disorder. In some instances, the fibrotic disease is cardiac fibrosis, arthritis, idiopathic pulmonary fibrosis, and nonalcoholic steatohepatitis (also known as metabolic dysfunction-associated steatohepatitis). In other instances, the disorder involves tumor-associated fibroblasts.

Methods of Treatment of tLNP Comprising a Nucleic Acid Encoding a Chimeric Antigen Receptor (CAR)

In some embodiments, a tLNP of this disclosure comprises a nucleic acid encoding a chimeric antigen receptor (CAR). The receptors are chimeric because they combine both antigen-binding and T cell activating functions into a single receptor. There are five generations of CARs that are commonly recognized. “First generation” CARs for use in the invention comprise an antigen binding domain, for example, a single-chain variable fragment (scFv) or VHH, fused to a transmembrane domain, which is fused to a cytoplasmic/intracellular domain of the T cell receptor chain. “First generation” CARs typically have the intracellular signaling (or activation) domain from the CD3ζ chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation. Use of a CD3ζ intracellular signaling domain in which one or two of the three ITAM motifs has been disrupted can modulate the balance of effector and memory programs (Feucht et al., 2019 Nat Med 25(1):82-88). The intracellular signaling domains of CD3ε or the low affinity receptor for IgG, FcyRIIIA (CD16A) can be used as alternatives to CD3ζ. In some embodiments, the intracellular signaling domain of CD3P comprises the sequence KNRKAKAKPVTRGAGAGGRQRGQNKERPPPVPNPDYEPIRKGQRDLYSGLNQRRI (SEQ ID NO: 115). In some embodiments, the intracellular signaling domain of FcγRIIIA (CD16A) comprises the sequence of FcγRIIIA: KTNIRSSTRDWKDHKFKWRKDPQDK (SEQ ID NO: 116). In some embodiments, these intracellular signaling domains constitute means for signaling or means for activation.

“Second-generation” CARs for use in the invention comprise an antigen binding domain, for example, a scFv or VHH, fused to a transmembrane domain, which is fused to an intracellular signaling domain capable of activating T cells and a co-stimulatory domain designed to augment T cell potency and persistence (Sadelain et al., 2013, Cancer Discov. 3:388-398). CAR design can therefore combine antigen recognition with signal transduction, two functions that are physiologically borne by two separate complexes, the TCR heterodimer and the CD3 complex.

“Second generation” CARs include an intracellular domain from various co-stimulatory molecules, for example, CD28, 4-1BB, ICOS, OX40, CD27, and the like, in the cytoplasmic tail of the CAR to provide additional signals to the cell. “Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of CAR-T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., 2012, Oncoimmunol. 1(9):1577-1583). In some embodiments, these costimulatory domains constitute means for costimulation.

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2RP.

Further variations on the basic CAR structure and sources for the various domains are described in Zabel et al., Immunol Lett 2019 212:53-69 which is incorporated by reference for all that it teaches about CAR structure and functional domains thereof to the extent it is consistent with this disclosure.

### a) Signal Peptide

In certain embodiments, the CAR can comprise a signal peptide at the N-terminus. Non-limiting examples of signal peptides include CD8α signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 1 below.

TABLE 1
Exemplary sequences of signal peptides

| SEQ ID NO: | Sequence | Description |
|---|---|---|
| 117 | MALPVTALLLP | CD8α signal peptide |
| | LALLLHAARP | |
| 118 | METDTLLLWVL | IgK signal peptide |
| | LLWVPGSTG | |
| 119 | MLLLVTSLLLC | GMCSFR-α (CSF2RA) |
| | ELPHPAFLLIP | signal peptide |

### b) Extracellular Binding Domain

A CAR comprises an extracellular binding domain, also referred to as a binder or binding moiety. In certain embodiments, the extracellular binding domain can comprise one or more antibodies specific to one pursued antigen or multiple pursued antigens. The antibody can be an antibody fragment, for example, an scFv, or a single-domain antibody fragment, for example, a VHH. In certain embodiments, the scFv can comprise a heavy chain variable region (V_{H}) and a light chain variable region (V_{L}) of an antibody connected by a linker. The V_{H} and the V_{L} can be connected in either order, i.e., V_{H}-linker-V_{L} or V_{L}-linker-V_{H}. Non-limiting examples of linkers include the Whitlow linker, (G₄S)n (SEQ ID NO: 123, n can be a positive integer, e.g., 1, 2, 3, 4, 5, 6, etc.), and variants thereof. In certain embodiments, the antigen can be an antigen that is exclusively or preferentially expressed on tumor cells, or an antigen that is characteristic of an autoimmune or inflammatory disease.

Exemplary pursued antigens against which a CAR, TCR, or ICE can have specificity include, but are not limited to, B cell maturation agent (BCMA)^{†‡}, CA9^{†‡}, CD4^{†‡}, CD5^{†‡}, CD19*^{†‡}, CD20 (MS4A1)*^{†‡}, CD22*^{†‡}, FCRL5^{†‡}, GPRC5D^{†‡}, CD23*^{†‡}, CD30 (TNFRSF8)*^{†‡}, CD33*^{†‡}, CD38*^{†‡}, CD44*^{‡}, CD70*^{†‡}, CD133^{†}, CD174, CD274 (PD-L1)*^{†‡}, CD276 (B7-H3)^{†‡}, CEACAM5*^{†‡}, CLL1^{‡}, CSPG4*^{‡}, Kappa*, , Lambda*, NCAM1 (CD56)*^{‡}, PD-1 (CD279)^{†‡}, ROR1^{†‡}, CD138 (SDC1)*^{‡}, CD319 (SLAMF7)*^{†‡}, CD248 (TEM1)^{‡}, ULBP1, and ULBP2 (associated with leukemias); CD319 (SLAMF7)*^{†‡}, CD38*^{†‡}, CD138^{†‡}, GPRC5D^{†‡}, CD267 (TACI)^{‡}, and BCMA^{†‡} (associated with myelomas); and Claudin 6 (CLDN6), Claudin 18.2 (CLDN18.2), GD2*^{†‡}, HER2*^{†‡}, EGFR*^{†‡}, EGFRvIII*, CD276 (B7H3)^{†‡}, PSMA*^{†‡}, PSCA^{‡}, CAIX (CA9)^{†‡}, CD171 (L1-CAM)*^{‡}, CEA*^{‡}, CSPG4*^{‡}, DLL3, EPHA2*^{‡}, FAP*^{†‡}, LRRC15^{†‡}, FOLR1*^{†‡}, IL-13Rα*^{†‡}, Mesothelin (MSLN)*^{†‡}, MUC1*^{†‡}, MUC16*^{†‡}, EPCAM*^{†‡}, ERBB2*^{‡}, FOLH1, GPC3*^{†‡}, GPNMB*^{‡}, IL1RAP^{†‡}, IL3RA*^{‡}, IL13RA2 (IL13Rα2)*^{‡}, KDR (VEGFR2)*^{‡}, CD171 (L1CAM)*^{‡}, MET*^{‡}, TROP2*^{†‡}, and ROR1^{†‡} (associated with solid tumors). Antigens associated with B cell leukemias can also be useful for B cell depletion in non-oncologic applications, however, CD19 (present on pro-B cells, pre-B cells, immature, naïve, germinal center, and memory B cells, and short-lived plasmablasts (sometime referred to as short-lived plasma cells)) and BCMA (present on memory B cells, short-lived plasmablasts, and long-lived plasma cells) are of particularly interest. (* indicates that exemplary antibodies with the indicated specificity from which a binding moiety could be derived can be found in U.S. Pat. No. 11,326,182B2 Table 9 or 10. ^{†} indicates that exemplary antibodies with the indicated specificity from which a binding moiety could be derived can be found in Wilkinson & Hale, 2022. Both references cited and incorporated by reference above. ^{‡} indicates that exemplary antibodies with the indicated specificity from which a binding moiety could be derived can be found in the Therapeutic Antibody Database (TABS) at tabs.craic.com). Other suitable antibodies can be found in Appendix A. Many of these pursued antigens are themselves receptors that could bind to their ligand if expressed on an immune cell. Accordingly, in some embodiments, the extracellular binding domain of the CAR comprises a ligand of a receptor expressed on the target cell. In still further embodiments, the extracellular binding domain of the CAR comprises a ligand binding domain of a receptor for a ligand expressed on the target cell. In any of these embodiments, the extracellular binding domain of the CAR can be codon-optimized for expression in a host cell or have variant sequences to increase functions of the extracellular binding domain. The advantages of the aspects and embodiments disclosed herein are independent of the specificity of the binding moiety. As such, the disclosed aspects and embodiments are generally agnostic to binding specificity. In certain embodiments, a particular binding specificity can be required. A more extensive discussion of antibodies recognizing many of the individual antigens listed above can be found in WIPO Publication WO2024040195A1 and U.S. patent application Ser. No. 18/731,223 which are each incorporated by reference for all that they teach about antibodies and related molecules that can be used to provide binding moieties recognizing target antigens.

### c) Hinge Domain

In certain embodiments, the CAR can comprise a hinge domain, also referred to as a spacer. The terms “hinge” and “spacer” can be used interchangeably in this disclosure. Non-limiting examples of hinge domains include CD8α hinge domain, CD28 hinge domain, IgG4 hinge domain, IgG4 hinge-CH₂—CH₃ domain, and variants thereof, the amino acid sequences of which are provided in Table 2 below.

TABLE 2
Exemplary sequences of hinge domains

| SEQ ID NO: | Sequence | Description |
|---|---|---|
| 120 | TTTPAPRPPTPAPTIAS | CD8α hinge domain |
| | QPLSLRPEACRPAAGGA | |
| | VHTRGLDFACD | |
| 121 | IEVMYPPPYLDNEKSNG | CD28 hinge domain |
| | TIIHVKGKHLCPSPLFP | |
| | GPSKP | |
| 122 | ESKYGPPCPPCP | IgG4 hinge domain |
| 124 | ESKYGPPCPPCPAPEFL | IgG4 hinge-CH2-CH3 |
| | GGPSVFLFPPKPKDTLM | domain |
| | ISRTPEVTCVVVDVSQE | |
| | DPEVQFNWYVDGVEVHN | |
| | AKTKPREEQFNSTYRVV | |
| | SVLTVLHQDWLNGKEYK | |
| | CKVSNKGLPSSIEKTIS | |
| | KAKGQPREPQVYTLPPS | |
| | QEEMTKNQVSLTCLVKG | |
| | FYPSDIAVEWESNGQPE | |
| | NNYKTTPPVLDSDGSFF | |
| | LYSRLTVDKSRWQEGNV | |
| | FSCSVMHEALHNHYTQK | |
| | SLSLSLGK | |

### d) Transmembrane Domain

In certain embodiments, the CAR can comprise a transmembrane domain. In other embodiments, the transmembrane domain can comprise a transmembrane region of CD3ζ, CDFε, CD3γ, CD3δ, CD4, CD5, CD8α, CD8β, CD9, CD16, CD22, CD28, CD32, CD33, CD34, CD37, CD40, CD45, CD64, CD8β, CD86, OX40/CD134, 4-1BB/CD137, CD40L/CD154, FAS, Fcε-RIγ, FGFR21B, TCRα, TCRβ, or VEGFR2, or a functional variant thereof, including the human versions of each of these sequences. Table 3 provides the amino acid sequences of a few exemplary transmembrane domains.

TABLE 3
Exemplary sequences of
transmembrane domains

| SEQ | | |
|---|---|---|
| ID | | |
| NO: | Sequence | Description |
| 125 | IYIWAPLAGTCG | CD8α transmembrane |
| | VLLLSLVITLYC | domain |
| 126 | FWVLVVVGGVLA | CD28 transmembrane |
| | CYSLLVTVAFII | domain |
| | FWV | |

### e) Intracellular Domain

In certain embodiments, the CAR can comprise an intracellular signaling domain. The various generations of CARs have including an intracellular domain that provides an activating or stimulatory function, such as from CD3ζ, CD3ε, or CD16A. The 2^{nd} and 3^{rd} generation CARs added one or more intracellular domains, respectively, to provide co-stimulatory function, such as from CD28 or 4-1BB among many others. In certain embodiments, the intracellular signaling domain can comprise one or more signaling domains selected from B7-1/CD8β, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFRSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4/CD134, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM⁻¹, LAG-3, TCL1A, TCL1B, CRTAM, DAP12, Dectin-1/CLEC7A, DPPIV/CD26, EphB6, TIM⁻¹/KIM⁻¹/HAVCR, TIM-4, TSLP, TSLP R, lymphocyte function associated antigen-1 (LFA-1), NKG2C, CD3ζ, an immunoreceptor tyrosine-based activation motif (ITAM), a ligand that specifically binds with CD83, and a functional variant thereof including the human versions of each of these domains. In some embodiments, the intracellular signaling domain comprises one or more signaling domains selected from a CD3ζ domain, an ITAM, a CD28 domain, 4-1in domain, or a functional variant thereof. Table 4 provides amino acid sequences for a few exemplary intracellular signaling domains. 4-1in, also known as CD137, transmits a potent costimulatory signal to T cells, promoting differentiation and enhancing long-term survival of T lymphocytes. CD28 is another co-stimulatory molecule on T cells. CD3 zeta (ζ) associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In certain embodiments, as in the case of tisagenlecleucel as described below, the CD3ζ signaling domain of SEQ ID NO: 129 can have a mutation, e.g., a glutamine (Q) to lysine (K) mutation, at amino acid position 14 (see SEQ ID NO: 130).

TABLE 4
Exemplary sequences of intracellular signaling domains

| SEQ ID NO: | Sequence | Description |
|---|---|---|
| 127 | KRGRKKLLYIFKQPFMRPVQTTQEED | 4-1BB signaling domain |
| | GCSCRFPEEEEGGCEL | |
| 128 | RSKRSRLLHSDYMNMTPRRPGPTRK | CD28 signaling domain |
| | HYQPYAPPRDFAAYRS | |
| 129 | RVKFSRSADAPAYQQGQNQLYNELN | CD3ζ signaling domain |
| | LGRREEYDVLDKRRGRDPEMGGKPR | |
| | RKNPQEGLYNELQKDKMAEAYSEIG | |
| | MKGERRRGKGHDGLYQGLSTATKD | |
| | TYDALHMQALPPR | |
| 130 | RVKFSRSADAPAYKQGQNQLYNELN | CD3ζ signaling domain (with Q |
| | LGRREEYDVLDKRRGRDPEMGGKPR | to K mutation at position 14) |
| | RKNPQEGLYNELQKDKMAEAYSEIG | |
| | MKGERRRGKGHDGLYQGLSTATKD | |
| | TYDALHMQALPPR | |

### f) Exemplary CAR Constructs

In certain embodiments, CARs are used to treat a disease or condition associated with a pursued cell that expresses the antigen pursued by the CAR as described in the uses and methods of treatment disclosed herein. For example, in some embodiments, an anti-CD19 or anti-CD20 or anti-BCMA CAR can be used to pursue and treat B cell malignancies or B cell-mediated autoimmune conditions or diseases. In other embodiments, an anti-FAP CAR can be used to pursue and treat solid tumors or fibrosis (e.g., cardiac fibrosis, cancer-associated fibroblasts). Examples of CARs that can be used in accordance with the embodiments described herein include to those disclosed in U.S. Pat. No. 7,446,190 (anti-CD19), U.S. Pat. No. 10,287,350 (anti-CD19), US2021/0363245 (anti-CD19 and anti-CD20), U.S. Pat. No. 10,543,263 (anti-CD22), U.S. Pat. No. 10,426,797 (anti-CD33), U.S. Pat. No. 10,844,128 (anti-CD123), U.S. Pat. No. 10,428,141 (anti-ROR1), and US2021/0087295 (anti-FAP), each of which is incorporated by reference for all that it teaches about CAR structure and function generically and with respect to the CAR's antigenic specificity and pursued indications to the extent that it is not inconsistent with this disclosure.

In certain embodiments, binding domains from antibodies can be used to construct a CAR to pursue and treat solid tumors or fibrosis. Exemplary binding domains can be obtained from antibodies, such as anti-LRRC15 (WO 2021/102332), anti-FAP (US 2012/0128591; US 2012/0128591; US 2012/0128591; US 2003/0103968, U.S. Pat. No. 6,455,677; US 2009/0304718; US 2009/0304718; US 2012/0258119); anti-ADAM12 (WO 2015/028027; WO 2020/191293); and anti-ITGA11 (WO 2008/075038; US 2011/0256061). Other antibodies that can be used to construct CARs to pursue and treat solid tumors or fibrosis include anti-CTSK, anti-NOX4, anti-SGCD, anti-SYNDIG1, anti-CDH11, anti-PLPP4, anti-SLC24A2, anti-PDGFRB, anti-THY1, anti-ANTXR1, anti-GAS1, anti-CALHM5, anti-COL11A1, anti-COL1A2, anti-FBN1, anti-COL10A1, anti-COL3A1, anti-COL5A2, anti-COL1A1, anti-COL8A2, anti-COL6A3, anti-GLT8D2, anti-SULF1, anti-COL12A1, anti-GXYLT2, anti-NID2, anti-THBS2, anti-COL5A1, anti-FN1, anti-COL6A1, anti-C₃orf80.

An mRNA disclosed herein encoding a CAR includes both the mature CAR and a signal peptide. A mature CAR minimally comprises an antigen binding domain, a transmembrane domain, and an intracellular signaling domain. In some embodiments, a CAR further comprises one or more co-stimulatory domains in the intracellular portion of the CAR. In some embodiments, a CAR further comprises an extracellular hinge or extension domain between the transmembrane domain and the antigen binding domain; this domain can be derived from the same protein as the transmembrane domain. In some embodiments, a CAR can comprise multiple antigen binding domains. In certain embodiments of the mRNA disclosed herein, the CAR is an anti-CD19 CAR, an anti-CD20 CAR, an anti-BCMA CAR, or an anti-FAP CAR.

### i) Anti-CD19 CAR

In certain embodiments, two CAR configurations are used for anti-CD19 CAR: CAR1 and CAR2. CAR1 mRNAs encode an amino acid sequence consisting of the following domains in N- to C-terminal order: CD8α signal peptide (SP), anti-CD19 scFv derived from mAb 47G4 (light chain variable domain, VL; linker, L; heavy chain variable domain, VH; 47G4 is disclosed in US2010/0104509), CD8α hinge, CD8α transmembrane domain (TM), CD28 costimulatory domain (co-stim), and CD3ζ signaling domain (stim). The CAR1 amino acid sequence is originally disclosed in U.S. Pat. No. 10,287,350 (WO2015/187528) as SEQ ID NO: 199, from which the CAR1 amino acid sequence and its synthesis are incorporated herein by reference. The amino acid sequence of the mature CAR1 protein (i.e., without a signal peptide) is provided as SEQ ID NO: 198. The incorporation of the CD8α hinge and transmembrane domains in CAR1 helps reduce cytokine release syndrome (cytokine storm) in comparison to similar anti-CD19 CAR molecules that instead incorporate a CD28 hinge and transmembrane domain but in vivo CARs can benefit from the stronger signal provided by the CD28 hinge and transmembrane domain.

In certain embodiments comprising an anti-CD19 CAR, the anti-CD19 CAR comprises an anti-CD19 binding domain. Some embodiments of an anti-CD19 CAR comprising an anti-CD19 binding domain further comprise a CD28 hinge, transmembrane, and co-stimulatory domains, and a CD3ζ signaling domain. Some embodiments of an anti-CD19 CAR comprising an anti-CD19 binding domain further comprise a hinge and transmembrane domain from CD8α, a CD28 costimulatory domain, and a CD3ζ chain signaling domain. In certain embodiments, an anti-CD19 binding domain comprises a 47G4 scFv. In certain embodiments, a CAR-T cell comprising an anti-CD19 CAR comprising CD28 hinge, transmembrane, and co-stimulatory domains exhibits more pursued cell killing than a CAR-T cell comprising an anti-CD19 CAR comprising CD8α hinge and transmembrane domains, and a CD28 co-stimulatory domain.

In certain embodiments, the CAR2 mRNAs that are used encode an amino acid sequence (SEQ ID NO: 201) consisting of the following domains in N- to C-terminal order: CD8α signal peptide (SP), anti-CD19 scFv derived from mAb 47G4 (light chain variable domain, VL; linker, L; heavy chain variable domain, VH), CD28 hinge, CD28 transmembrane (TM), CD28 co-stimulatory domain (co-stim), and CD3ζ signaling domain (stim). The amino acid sequence of the immature CAR2 protein (i.e., with a signal peptide) is disclosed in Genbank: QHQ73565.1 and provided as SEQ ID NO: 201. Combining the 47G4 scFv as well as the CD28 hinge and transmembrane domains provides CAR2 an advantage for transient in vivo transfection as opposed to the traditional CAR-T cell comprising an integrated DNA sequence encoding CAR. CAR2 is expressed at a higher level than CAR1 from mRNAs using the same UTRs and codon optimization method and the T cells expressing CAR2 eliminate more CD19+ cells.

Further examples of anti-CD 19 CARs include those incorporating a CD 19 binding moiety derived from the mouse antibody FMC63. FMC63 and the derived scFv have been described in Nicholson et al., 1997, Mol. Immun. 34(16-17):1157-1165 and PCT Application Publication Nos. WO 2018/213337 and WO 2015/187528, the entire contents of each of which are incorporated by reference herein for all that they teach about anti-CD 19 CARs and their use.

TABLE 5
Exemplary sequences of anti-CD19 scFv and components

| SEQ ID NO: | Amino Acid Sequence | Description |
|---|---|---|
| 131 | DIQMTQTTSSLSASLGDRVTISCRAS | Anti-CD19 FMC63 scFv |
| | QDISKYLNWYQQKPDGTVKLLIYHT | entire sequence, with |
| | SRLHSGVPSRFSGSGSGTDYSLTISN | Whitlow linker |
| | LEQEDIATYFCQQGNTLPYTFGGGT | |
| | KLEITGSTSGSGKPGSGEGSTKGEVK | |
| | LQESGPGLVAPSQSLSVTCTVSGVSL | |
| | PDYGVSWIRQPPRKGLEWLGVIWGS | |
| | ETTYYNSALKSRLTIIKDNSKSQVFL | |
| | KMNSLQTDDTAIYYCAKHYYYGGS | |
| | YAMDYWGQGTSVTVSS | |
| 132 | DIQMTQTTSSLSASLGDRVTISCRAS | Anti-CD19 FMC63 scFv |
| | QDISKYLNWYQQKPDGTVKLLIYHT | light chain variable region |
| | SRLHSGVPSRFSGSGSGTDYSLTISN | |
| | LEQEDIATYFCQQGNTLPYTFGGGT | |
| | KLEIT | |
| 133 | QDISKY | Anti-CD19 FMC63 scFv |
| | | light chain CDR1 |
| 134 | HTS | Anti-CD19 FMC63 scFv |
| | | light chain CDR2 |
| 135 | QQGNTLPYT | Anti-CD19 FMC63 scFv |
| | | light chain CDR3 |
| 136 | GSTSGSGKPGSGEGSTKG | Whitlow linker |
| 137 | EVKLQESGPGLVAPSQSLSVTCTVS | Anti-CD19 FMC63 scFv |
| | GVSLPDYGVSWIRQPPRKGLEWLGV | heavy chain variable |
| | IWGSETTYYNSALKSRLTIIKDNSK | region |
| | SQVFLKMNSLQTDDTAIYYCAKHY | |
| | YYGGSYAMDYWGQGTSVTVSS | |
| 138 | GVSLPDYG | Anti-CD19 FMC63 scFv |
| | | heavy chain CDR1 |
| 139 | IWGSETT | Anti-CD19 FMC63 scFv |
| | | heavy chain CDR2 |
| 140 | AKHYYYGGSYAMDY | Anti-CD19 FMC63 scFv |
| | | heavy chain CDR3 |
| 141 | DIQMTQTTSSLSASLGDRVTISCRAS | Anti-CD19 FMC63 scFv |
| | QDISKYLNWYQQKPDGTVKLLIYHT | entire sequence, with |
| | SRLHSGVPSRFSGSGSGTDYSLTISN | 3xG₄S linker |
| | LEQEDIATYFCQQGNTLPYTFGGGT | |
| | KLEITGGGGSGGGGSGGGGSEVKLQ | |
| | ESGPGLVAPSQSLSVTCTVSGVSLPD | |
| | YGVSWIRQPPRKGLEWLGVIWGSET | |
| | TYYNSALKSRLTIIKDNSKSQVFLK | |
| | MNSLQTDDTAIYYCAKHYYYGGSY | |
| | AMDYWGQGTSVTVSS | |
| 142 | GGGGSGGGGSGGGGS | 3xG₄S linker |

In some instances, the anti-CD19 CAR is the CAR found in tisagenlecleucel (Vairy et al., 2018, Drug Des Devel Ther. 12: 3885-3898), lisocabtagene maraleucel, or axicabtagene ciloleucel and brexucabtagene autoleucel (Cappell et al., 2023, Nat Rev Clin Oncol 20: 359-371) which use the same CAR. The entire contents of each of foregoing references in this paragraph are incorporated by reference for all that they teach about the design, structure, and activity of anti-CD 19 CARs.

TABLE 6
Exemplary sequences of CD19 CARs*

| SEQ | | |
|---|---|---|
| ID | | |
| NO: | Sequence | Description |
| 143 | atggccttaccagtgaccgccttgctcctgccgct | Tisagenlecleucel |
| | ggccttgctgctccacgccgccaggccggacatcc | CD19 CAR |
| | agatgacacagactacatcctccctgtctgcctct | nucleotide |
| | ctgggagacagagtcaccatcagttgcagggcaag | sequence |
| | tcaggacattagtaaatatttaaattggtatcagc | |
| | agaaaccagatggaactgttaaactcctgatctac | |
| | catacatcaagattacactcaggagtcccatcaag | |
| | gttcagtggcagtgggtctggaacagattattctc | |
| | tcaccattagcaacctggagcaagaagatattgcc | |
| | acttacttttgccaacagggtaatacgcttccgta | |
| | cacgttcggaggggggaccaagctggagatcacag | |
| | gtggcggtggctcgggcggtggtgggtcgggtggc | |
| | ggcggatctgaggtgaaactgcaggagtcaggacc | |
| | tggcctggtggcgccctcacagagcctgtccgtca | |
| | catgcactgtctcaggggtctcattacccgactat | |
| | ggtgtaagctggattcgccagcctccacgaaaggg | |
| | tctggagtggctgggagtaatatggggtagtgaaa | |
| | ccacatactataattcagctctcaaatccagactg | |
| | accatcatcaaggacaactccaagagccaagtttt | |
| | cttaaaaatgaacagtctgcaaactgatgacacag | |
| | ccatttactactgtgccaaacattattactacggt | |
| | ggtagctatgctatggactactggggccaaggaac | |
| | ctcagtcaccgtctcctcaaccacgacgccagcgc | |
| | cgcgaccaccaacaccggcgcccaccatcgcgtcg | |
| | cagcccctgtccctgcgcccagaggcgtgccggcc | |
| | agcggcggggggcgcagtgcacacgagggggctgg | |
| | acttcgcctgtgatatctacatctgggcgcccttg | |
| | gccgggacttgtggggtccttctcctgtcactggt | |
| | tatcaccctttactgcaaacggggcagaaagaaac | |
| | tcctgtatatattcaaacaaccatttatgagacca | |
| | gtacaaactactcaagaggaagatggctgtagctg | |
| | ccgatttccagaagaagaagaaggaggatgtgaac | |
| | tgagagtgaagttcagcaggagcgcagacgccccc | |
| | gcgtacaagcagggccagaaccagctctataacga | |
| | gctcaatctaggacgaagagaggagtacgatgttt | |
| | tggacaagagacgtggccgggaccctgagatgggg | |
| | ggaaagccgagaaggaagaaccctcaggaaggcct | |
| | gtacaatgaactgcagaaagataagatggcggagg | |
| | cctacagtgagattgggatgaaaggcgagcgccgg | |
| | aggggcaaggggcacgatggcctttaccagggtct | |
| | cagtacagccaccaaggacacctacgacgcccttc | |
| | acatgcaggccctgccccctcgc | |
| 144 | MALPVTALLLPLALLLHAARPDIQMTQTTSSLSAS | Tisagenlecleucel |
| | LGDRVTISCRASQDISKYLNWYQQKPDGTVKLLIY | CD19 CAR amino |
| | HTSRLHSGVPSRFSGSGSGTDYSLTISNLEQEDIA | acid sequence |
| | TYFCQQGNTLPYTFGGGTKLEITGGGGSGGGGSGG | |
| | GGSEVKLQESGPGLVAPSQSLSVTCTVSGVSLPDY | |
| | GVSWIRQPPRKGLEWLGVIWGSETTYYNSALKSRL | |
| | TIIKDNSKSQVFLKMNSLQTDDTAIYYCAKHYYYG | |
| | GSYAMDYWGQGTSVTVSSTTTPAPRPPTPAPTIAS | |
| | QPLSLRPEACRPAAGGAVHTRGLDFACDIYIWAPL | |
| | AGTCGVLLLSLVITLYCKRGRKKLLYIFKQPFMRP | |
| | VQTTQEEDGCSCRFPEEEEGGCELRVKFSRSADAP | |
| | AYKQGQNQLYNELNLGRREEYDVLDKRRGRDPEMG | |
| | GKPRRKNPQEGLYNELQKDKMAEAYSEIGMKGERR | |
| | RGKGHDGLYQGLSTATKDTYDALHMQALPPR | |
| 145 | atgctgctgctggtgaccagcctgctgctgtgcga | Lisocabtagene |
| | gctgccccaccccgcctttctgctgatccccgaca | maraleucel CD19 |
| | tccagatgacccagaccacctccagcctgagcgcc | CAR nucleotide |
| | agcctgggcgaccgggtgaccatcagctgccgggc | sequence |
| | cagccaggacatcagcaagtacctgaactggtatc | |
| | agcagaagcccgacggcaccgtcaagctgctgatc | |
| | taccacaccagccggctgcacagcggcgtgcccag | |
| | ccggtttagcggcagcggctccggcaccgactaca | |
| | gcctgaccatctccaacctggaacaggaagatatc | |
| | gccacctacttttgccagcagggcaacacactgcc | |
| | ctacacctttggcggcggaacaaagctggaaatca | |
| | ccggcagcacctccggcagcggcaagcctggcagc | |
| | ggcgagggcagcaccaagggcgaggtgaagctgca | |
| | ggaaagcggccctggcctggtggcccccagccaga | |
| | gcctgagcgtgacctgcaccgtgagcggcgtgagc | |
| | ctgcccgactacggcgtgagctggatccggcagcc | |
| | ccccaggaagggcctggaatggctgggcgtgatct | |
| | ggggcagcgagaccacctactacaacagcgccctg | |
| | aagagccggctgaccatcatcaaggacaacagcaa | |
| | gagccaggtgttcctgaagatgaacagcctgcaga | |
| | ccgacgacaccgccatctactactgcgccaagcac | |
| | tactactacggcggcagctacgccatggactactg | |
| | gggccagggcaccagcgtgaccgtgagcagcgaat | |
| | ctaagtacggaccgccctgccccccttgccctatg | |
| | ttctgggtgctggtggtggtcggaggcgtgctggc | |
| | ctgctacagcctgctggtcaccgtggccttcatca | |
| | tcttttgggtgaaacggggcagaaagaaactcctg | |
| | tatatattcaaacaaccatttatgagaccagtaca | |
| | aactactcaagaggaagatggctgtagctgccgat | |
| | ttccagaagaagaagaaggaggatgtgaactgcgg | |
| | gtgaagttcagcagaagcgccgacgcccctgccta | |
| | ccagcagggccagaatcagctgtacaacgagctga | |
| | acctgggcagaagggaagagtacgacgtcctggat | |
| | aagcggagaggccgggaccctgagatgggcggcaa | |
| | gcctcggcggaagaacccccaggaaggcctgtata | |
| | acgaactgcagaaagacaagatggccgaggcctac | |
| | agcgagatcggcatgaagggcgagcggaggcgggg | |
| | caagggccacgacggcctgtatcagggcctgtcca | |
| | ccgccaccaaggatacctacgacgccctgcacatg | |
| | caggccctgcccccaagg | |
| 146 | MLLLVTSLLLCELPHPAFLLIPDIQMTQTTSSLSA | Lisocabtagene |
| | SLGDRVTISCRASQDISKYLNWYQQKPDGTVKLLI | maraleucel CD19 |
| | YHTSRLHSGVPSRFSGSGSGTDYSLTISNLEQEDI | CAR amino acid |
| | ATYFCQQGNTLPYTFGGGTKLEITGSTSGSGKPGS | sequence |
| | GEGSTKGEVKLQESGPGLVAPSQSLSVTCTVSGVS | |
| | LPDYGVSWIRQPPRKGLEWLGVIWGSETTYYNSAL | |
| | KSRLTIIKDNSKSQVFLKMNSLQTDDTAIYYCAKH | |
| | YYYGGSYAMDYWGQGTSVTVSSESKYGPPCPPCPM | |
| | FWVLVVVGGVLACYSLLVTVAFIIFWVKRGRKKLL | |
| | YIFKQPFMRPVQTTQEEDGCSCRFPEEEEGGCELR | |
| | VKFSRSADAPAYQQGQNQLYNELNLGRREEYDVLD | |
| | KRRGRDPEMGGKPRRKNPQEGLYNELQKDKMAEAY | |
| | SEIGMKGERRRGKGHDGLYQGLSTATKDTYDALHM | |
| | QALPPR | |
| 147 | atgcttctcctggtgacaagccttctgctctgtga | Axicabtagene |
| | gttaccacacccagcattcctcctgatcccagaca | ciloleucel CD19 |
| | tccagatgacacagactacatoctccctgtctgcc | CAR nucleotide |
| | tctctgggagacagagtcaccatcagttgcagggc | sequence |
| | aagtcaggacattagtaaatatttaaattggtatc | |
| | agcagaaaccagatggaactgttaaactcctgatc | |
| | taccatacatcaagattacactcaggagtcccatc | |
| | aaggttcagtggcagtgggtctggaacagattatt | |
| | ctctcaccattagcaacctggagcaagaagatatt | |
| | gccacttacttttgccaacagggtaatacgcttcc | |
| | gtacacgttcggaggggggactaagttggaaataa | |
| | caggctccacctctggatccggcaagcccggatct | |
| | ggcgagggatccaccaagggcgaggtgaaactgca | |
| | ggagtcaggacctggcctggtggcgccctcacaga | |
| | gcctgtccgtcacatgcactgtctcaggggtctca | |
| | ttacccgactatggtgtaagctggattcgccagcc | |
| | tccacgaaagggtctggagtggctgggagtaatat | |
| | ggggtagtgaaaccacatactataattcagctctc | |
| | aaatccagactgaccatcatcaaggacaactccaa | |
| | gagccaagttttcttaaaaatgaacagtctgcaaa | |
| | ctgatgacacagccatttactactgtgccaaacat | |
| | tattactacggtggtagctatgctatggactactg | |
| | gggtcaaggaacctcagtcaccgtctcctcagcgg | |
| | ccgcaattgaagttatgtatcctcctccttaccta | |
| | gacaatgagaagagcaatggaaccattatccatgt | |
| | gaaagggaaacacctttgtccaagtcccctatttc | |
| | ccggaccttctaagcccttttggg | |
| 148 | tgctggtggtggttgggggagtcctggcttgctat | Axicabtagene |
| | agcttgctagtaacagtggcctttattattttctg | ciloleucel CD19 |
| | ggtgaggagtaagaggagcaggctcctgcacagtg | CAR amino acid |
| | actacatgaacatgactccccgccgccccgggccc | sequence |
| | acccgcaagcattaccagccctatgccccaccacg | |
| | cgacttcgcagcctatcgctccagagtgaagttca | |
| | gcaggagcgcagacgcccccgcgtaccagcagggc | |
| | cagaaccagctctataacgagctcaatctaggacg | |
| | aagagaggagtacgatgttttggacaagagacgtg | |
| | gccgggaccctgagatggggggaaagccgagaagg | |
| | aagaaccctcaggaaggcctgtacaatgaactgca | |
| | gaaagataagatggcggaggcctacagtgagattg | |
| | ggatgaaaggcgagcgccggaggggcaaggggcac | |
| | gatggcctttaccagggtctcagtacagccaccaa | |
| | ggacacctacgacgcccttcacatgcaggccctgc | |
| | cccctcgcMLLLVTSLLLCELPHPAFLLIPDIQMT | |
| | QTTSSLSASLGDRVTISCRASQDISKYLNWYQQKP | |
| | DGTVKLLIYHTSRLHSGVPSRFSGSGSGTDYSLTI | |
| | SNLEQEDIATYFCQQGNTLPYTFGGGTKLEITGST | |
| | SGSGKPGSGEGSTKGEVKLQESGPGLVAPSQSLSV | |
| | TCTVSGVSLPDYGVSWIRQPPRKGLEWLGVIWGSE | |
| | TTYYNSALKSRLTIIKDNSKSQVFLKMNSLQTDDT | |
| | AIYYCAKHYYYGGSYAMDYWGQGTSVTVSSAAAIE | |
| | VMYPPPYLDNEKSNGTIIHVKGKHLCPSPLFPGPS | |
| | KPFWVLVVVGGVLACYSLLVTVAFIIFWVRSKRSR | |
| | LLHSDYMNMTPRRPGPTRKHYQPYAPPRDFAAYRS | |
| | RVKFSRSADAPAYQQGQNQLYNELNLGRREEYDVL | |
| | DKRRGRDPEMGGKPRRKNPQEGLYNELQKDKMAEA | |
| | YSEIGMKGERRRGKGHDGLYQGLSTATKDTYDALH | |
| | MQALPPR | |

*Nucleotide sequences are presented here (and throughout) as DNA sequences; t should be understood as “u” for corresponding RNA sequences.

TABLE 7
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB signaling domain | 997-1122 | 333-374 |
| CD3ζ signaling domain | 1123-1458 | 375-486 |

TABLE 8
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB signaling domain | 922-1047 | 308-349 |
| CD3ζ signaling domain | 1048-1383 | 350-461 |

TABLE 9
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 signaling domain | 1009-1131 | 337-377 |
| CD3ζ signaling domain | 1132-1467 | 378-489 |

CAR based on 47G4 are disclosed in U.S. Pat. No. 10,287,350 which is incorporated by reference herein for all that it teaches about anti-CD19 CARs and their use. In some embodiments, the extracellular binding domain of the CD19 CAR is derived from an antibody specific to CD19, including, for example, SJ25C₁ (Bejcek et al., 1995, Cancer Res. 55:2346-2351), HD37 (Pezutto et al., 1987, J. Immunol. 138(9):2793-2799), 4G7 (Meeker et al., 1984, Hybridoma 3:305-320), B43 (Bejcek et al., 1995 Cancer Res 55(11):2346-2351), BLY3 (Bejcek et al., 1995 Cancer Res 55(11):2346-2351), B4 (Freedman et al., 1987, Blood 70:418-427), B4 HB12b (Kansas & Tedder, 1991, J. Immunol. 147:4094-4102; Yazawa et al., 2005, Proc. Natl. Acad. Sci. USA 102:15178-15183; Herbst et al., J. Pharmacol. Exp. Ther. 335:213-222 (2010)), BU12 (Callard et al., 1992, J. Immunology, 148(10): 2983-2987), and CLB-CD19 (De Rie, 1989, Cell. Immunol. 118:368-381). In any of these embodiments, the extracellular binding domain of the CD19 CAR can comprise the V_{H}, the V_{L}, and/or one or more CDRs of any of the antibodies.

TABLE 10
Exemplary sequences of 47G4-based anti-CD19 CAR

| SEQ ID NO: | Sequence | Description |
|---|---|---|
| 117 | MALPVTALLLPLALLLHAARP | CD8 signal peptide |
| 149 | EIVLTQSPGTLSLSPGERATLSCRASQSVSS | CD19 antibody |
| | SYLAWYQQKPGQAPRLLIYGASSRATGIPD | (47G4 scFv) |
| | RFSGSGSGTDFTLTISRLEPEDFAVYYCQQ | |
| | YGSSRFTFGPGTKVDIKGSTSGSGKPGSGE | |
| | GSTKGQVQLVQSGAEVKKPGSSVKVSCKD | |
| | SGGTFSSYAISWVRQAPGQGLEWMGGIIPI | |
| | FGTTNYAQQFQGRVTITADESTSTAYMELS | |
| | SLRSEDTAVYYCAREAVAADWLDPWGQG | |
| | TLVTVSS | |
| 121 | IEVMYPPPYLDNEKSNGTIIHVKGKHLC | CD28 hinge |
| | PSPLFPGPSKP | |
| 126 | FWVLVVVGGVLACYSLLVTVAFIIFWV | CD28 |
| | | transmembrane |
| | | domain |
| 128 | RSKRSRLLHSDYMNMTPRRPGPTRKHYQP | CD28 cytoplasmic |
| | YAPPRDFAAYRS | (co-stim) |
| 129 | RVKFSRSADAPAYQQGQNQLYNELNLGRR | CD3ζ (stim) |
| | EEYDVLDKRRGRDPEMGGKPRRKNPQEGL | |
| | YNELQKDKMAEAYSEIGMKGERRRGKGH | |
| | DGLYQGLSTATKDTYDALHMQALPPR | |
| 150 | FVPVFLPAKPTTTPAPRPPTPAPTIASQ | CD8 hinge |
| | PLSLRPEACRPAAGGAVHTRGLDFACD | |
| 151 | IYIWAPLAGTCGVLLLSLVITLYCNHRN | CD8 |
| | | transmembrane |
| | | domain |

### ii) Anti-CD20 CAR

CD20 is an antigen found on the surface of B cells as early as the pro-B phase and progressively at increasing levels until B cell maturity, as well as on the cells of most B-cell neoplasms. CD20 positive cells are also sometimes found in cases of Hodgkin's disease, myeloma, and thymoma. Examples of anti-CD20 CARs include those incorporating a CD20 binding moiety derived from an antibody specific to CD20, including, for example, MB-106 (Fred Hutchinson Cancer Research Center, see Shadman et al., 2019, Blood 134(Suppl.1):3235), UCART20 (Cellectis, www.cellbiomedgroup.com), or C-CAR066 (Cellular Biomedicine Group, see Liang et al., 2021, J. Clin. Oncol. 39(15) suppl:2508) Leu16 and 2.1.2. In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker (See Wu et al., 2001, Protein Engineering. 14(12):1025-1033), such as CAR22 and CAR25 described herein. In some embodiments, the extracellular binding domain of the CD20 CAR comprises an scFv derived from the monoclonal antibody, 2.1.2, which comprises the heavy chain variable region (V_{H}) and the light chain variable region (V_{L}) of 2.1.2 connected by a linker, such as CAR7 described herein. Further antibodies that can provide an anti-CD20 binding domain include IF5, 1.5.3, rituximab, obinutuzumab, ibritumomab, ofatumumab, tositumumab, odronextamab, veltuzumab, ublituximab, and ocrelizumab.. The entire contents of each of foregoing references in this paragraph are incorporated by reference for all that they teach about the design, structure, and activity of anti-CD20 CARs.

In certain embodiments, CAR25 is provided herein as a CAR configuration used for anti-CD20 CAR. The CAR25 mRNA encodes an amino acid sequence consisting of the following domains in N- to C-terminal order: mouse Ig-kappa signal peptide (Igk sp), anti-CD20 scFv derived from the Leu16 mAb (light chain variable domain, VL; linker, L; heavy chain variable domain, VH), IgG4 hinge, CD28 transmembrane domain (TM), 4-1BB co-stimulatory domain (co-stim), and CD3ζ signaling domain (stim). The amino acid sequence of the mature CAR25 protein (i.e., without a signal peptide) is provided as SEQ ID NO: 19.

In certain embodiments comprising an anti-CD20 CAR, the anti-CD20 CAR comprises a Leu16 scFv. In some embodiments, the anti-CD20 CAR comprising a Leu16 scFv further comprises an IgG4 hinge, CD28 transmembrane domain, 4-1BB costimulation, and a CD3ζ signaling domain. Examples of such an anti-CD20 CAR include, without limitation, CAR25 (SEQ ID NO: 19, or with a signal peptide, SEQ ID NO: 20). In some embodiments, the anti-CD20 CAR comprising a Leu16 scFv further comprises an IgG4 hinge, CD28 transmembrane and costimulation domains, 4-1BB costimulation, and a CD3ζ signaling domain. Examples of such an anti-CD20 CAR include CAR22 (SEQ ID NO: 21), or with a signal peptide (SEQ ID NO: 22).

TABLE 11
Exemplary sequences of anti-CD20 scFv and components

| SEQ ID NO: | Amino Acid Sequence | Description |
|---|---|---|
| 152 | DIVLTQSPAILSASPGEKVTMTCRAS | Anti-CD20 Leu16 scFv |
| | SSVNYMDWYQKKPGSSPKPWIYAT | entire sequence, with |
| | SNLASGVPARFSGSGSGTSYSLTISR | Whitlow linker |
| | VEAEDAATYYCQQWSFNPPTFGGG | |
| | TKLEIKGSTSGSGKPGSGEGSTKGEV | |
| | QLQQSGAELVKPGASVKMSCKASG | |
| | YTFTSYNMHWVKQTPGQGLEWIGA | |
| | IYPGNGDTSYNQKFKGKATLTADKS | |
| | SSTAYMQLSSLTSEDSADYYCARSN | |
| | YYGSSYWFFDVWGAGTTVTVSS | |
| 153 | DIVLTQSPAILSASPGEKVTMTCRAS | Anti-CD20 Leu16 scFv |
| | SSVNYMDWYQKKPGSSPKPWIYAT | light chain variable region |
| | SNLASGVPARFSGSGSGTSYSLTISR | |
| | VEAEDAATYYCQQWSFNPPTFGGG | |
| | TKLEIK | |
| 154 | RASSSVNYMD | Anti-CD20 Leu16 scFv |
| | | light chain CDR1 |
| 155 | ATSNLAS | Anti-CD20 Leu16 scFv |
| | | light chain CDR2 |
| 156 | QQWSFNPPT | Anti-CD20 Leu16 scFv |
| | | light chain CDR3 |
| 157 | EVQLQQSGAELVKPGASVKMSCKA | Anti-CD20 Leu16 scFv |
| | SGYTFTSYNMHWVKQTPGQGLEWI | heavy chain |
| | GAIYPGNGDTSYNQKFKGKATLTA | |
| | DKSSSTAYMQLSSLTSEDSADYYC | |
| | ARSNYYGSSYWFFDVWGAGTTVTV | |
| | SS | |
| 158 | SYNMH | Anti-CD20 Leu16 scFv |
| | | heavy chain CDR1 |
| 159 | AIYPGNGDTSYNQKFKG | Anti-CD20 Leu16 scFv |
| | | heavy chain CDR2 |
| 202 | SNYYGSSYWFFDV | Anti-CD20 Leu16 scFv |
| | | heavy chain CDR3 |
| 203 | DIVLTQSPAILSASPGEKVTMTCRA | Anti-CD20 Leu16 scFv |
| | SSSVNYMDWYQKKPGSSPKPWIYAT | entire sequence, with |
| | SNLASGVPARFSGSGSGTSYSLTIS | (G3S)2-(G4S) linker |
| | RVEAEDAATYYCQQWSFNPPTFGGG | |
| | TKLEIKGSTSGGGSGGGSGGGGSSE | |
| | VQLQQSGAELVKPGASVKMSCKAS | |
| | GYTFTSYNMHWVKQTPGQGLEWIG | |
| | AIYPGNGDTSYNQKFKGKATLTAD | |
| | KSSSTAYMQLSSLTSEDSADYYCAR | |
| | SNYYGSSYWFFDVWGAGTTVTVSS | |
| 204 | GSTSGGGSGGGSGGGGSS | (G₃S)₂-(G₄S) linker |
| 205 | DIVMTQTPHSSPVTLGQPASISCRS | Anti-CD20 2.1.2 scFv |
| | SQSLVSRDGNTYLSWLQQRPGQPPR | entire sequence, with |
| | LLIYKISNRFSGVPNRFSGSGAGTD | (G₄S)₃ linker |
| | FTLKISRVKAEDVGVYYCMQATQFP | |
| | LTFGQGTRLEIKGGGGSGGGGSGGG | |
| | GSEVQLVQSGAEVKKPGESLKISCK | |
| | GSGYSFTSYWIGWVRQMPGKGLEWM | |
| | GIIYPGDSDTRYSPSFQGQVTISAD | |
| | KSISTAYLQWSSLKASDTAMYYCAR | |
| | QGDFWSGYGGMDVWGQGTTVTVSS | |
| 206 | DIVMTQTPHSSPVTLGQPASISCRS | Anti-CD20 2.1.2 scFv |
| | SQSLVSRDGNTYLSWLQQRPGQPPR | light chain variable region |
| | LLIYKISNRFSGVPNRFSGSGAGTD | |
| | FTLKISRVKAEDVGVYYCMQATQFP | |
| | LTFGQGTRLEIK | |
| 207 | RSSQSLVSRDGNTYLS | Anti-CD20 2.1.2 scFv |
| | | light chain CDR1 |
| 208 | KISNRFS | Anti-CD20 2.1.2 scFv |
| | | light chain CDR2 |
| 209 | MQATQFPLT | Anti-CD20 2.1.2 scFv |
| | | light chain CDR3 |
| 142 | GGGGSGGGGSGGGGS | (G₄S)₃ or 3x G₄S linker |
| 210 | EVQLVQSGAEVKKPGESLKISCKG | Anti-CD20 2.1.2 scFv |
| | SGYSFTSYWIGWVRQMPGKGLEWM | heavy chain variable |
| | GIIYPGDSDTRYSPSFQGQVTISA | region |
| | DKSISTAYLQWSSLKASDTAMYYC | |
| | ARQGDFWSGYGGMDVWGQGTTVTV | |
| | SS | |
| 211 | SYWIG | Anti-CD20 2.1.2 scFv |
| | | heavy chain CDR1 |
| 212 | IIYPGDSDTRYSPSFQG | Anti-CD20 2.1.2 scFv |
| | | heavy chain CDR2 |
| 213 | QGDFWSGYGGMDV | Anti-CD20 2.1.2 scFv |
| | | heavy chain CDR3 |

In certain embodiments comprising an anti-CD20 CAR, the anti-CD20 CAR comprises a 2.1.2 scFv. In some embodiments, the anti-CD20 CAR comprising a 2.1.2 scFv further comprises CD28 hinge, transmembrane, and costimulation domains and a CD3ζ signaling domain. Examples of such an anti-CD20 CAR include, without limitation, CAR7 (SEQ ID NO: 214), or with a signal peptide, SEQ ID NO: 215).

iii) Anti-BCMA CAR

In certain embodiments, the anti-CD8 tLNP encapsulates a nucleic acid encoding an anti-BCMA chimeric antigen receptor (CAR). BCMA is a tumor necrosis family receptor (TNFR) member expressed on cells of the B cell lineage, with the highest expression on terminally differentiated B cells or mature B lymphocytes. BCMA is involved in mediating survival of plasma cells for maintaining long-term humoral immunity. Expression of BCMA has been recently linked to a number of cancers, such as multiple myeloma, Hodgkin's and non-Hodgkin's lymphoma, various leukemias, and glioblastoma. Examples of anti-BCMA CARs include those incorporating a BCMA binding moiety derived from C11D5.3, a mouse monoclonal antibody as described in Carpenter et al., 2013, Clin. Cancer Res. 19(8):2048-2060. See also PCT Application Publication No. WO 2010/104949. In some embodiments, the extracellular binding domain of the BCMA CAR comprises an scFv derived from another mouse monoclonal antibody, C12A3.2, as described in Carpenter et al., 2013, Clin. Cancer Res. 19(8):2048-2060 and PCT Application Publication No. WO2010104949. In some embodiments, the extracellular binding domain of the BCMA CAR comprises an scFv derived from a mouse monoclonal antibody with high specificity to human BCMA, referred to as BB2121 in Friedman et al., 2018, Hum. Gene Ther. 29(5):585-601. See also, PCT Application Publication No. WO2012163805. In some embodiments, the extracellular binding domain of the BCMA CAR comprises single variable fragments of two heavy chains (VHH) that can bind to two epitopes of BCMA as described in Zhao et al., 2018, J. Hematol. Oncol. 11(1):141, also referred to as LCAR-B38M. See also, PCT Application Publication No. WO 2018/028647. In some embodiments, the extracellular binding domain of the BCMA CAR comprises a fully human heavy-chain variable domain (FHVH) as described in Lam et al., 2020, Nat. Commun. 11(1):283, also referred to as FHVH33. See also, PCT Application Publication No. WO 2019/006072. In some embodiments, the extracellular binding domain of the BCMA CAR comprises an scFv derived from CT103A (or CAR0085) as described in U.S. Pat. No. 11,026,975 B2. Further anti-BCMA CARs are disclosed in U.S. Patent Application Publication Nos. 2020/0246381 and 2020/0339699. The entire contents of each of foregoing references in this paragraph are incorporated by reference for all that they teach about the design, structure, and activity of anti-BCMA CARs.

TABLE 12
Exemplary sequences of anti-BCMA binder and components

| SEQ | | |
|---|---|---|
| ID NO: | Amino Acid Sequence | Description |
| 160 | DIVLTQSPASLAMSLGKRATISCRAS | Anti-BCMA C11D5.3 |
| | ESVSVIGAHLIHWYQQKPGQPPKLLI | scFv entire sequence, |
| | YLASNLETGVPARFSGSGSGTDFTLT | with Whitlow linker |
| | IDPVEEDDVAIYSCLQSRIFPRTFGG | |
| | GTKLEIKGSTSGSGKPGSGEGSTKG | |
| | QIQLVQSGPELKKPGETVKISCKASG | |
| | YTFTDYSINWVKRAPGKGLKWMG | |
| | WINTETREPAYAYDFRGRFAFSLETS | |
| | ASTAYLQINNLKYEDTATYFCALDY | |
| | SYAMDYWGQGTSVTVSS | |
| 161 | DIVLTQSPASLAMSLGKRATISCRAS | Anti-BCMA C11D5.3 |
| | ESVSVIGAHLIHWYQQKPGQPPKLLI | scFv light chain variable |
| | YLASNLETGVPARFSGSGSGTDFTLT | region |
| | IDPVEEDDVAIYSCLQSRIFPRTFGG | |
| | GTKLEIK | |
| 162 | RASESVSVIGAHLIH | Anti-BCMA C11D5.3 |
| | | scFv light chain CDR1 |
| 163 | LASNLET | Anti-BCMA C11D5.3 |
| | | scFv light chain CDR2 |
| 164 | LQSRIFPRT | Anti-BCMA C11D5.3 |
| | | scFv light chain CDR3 |
| 165 | QIQLVQSGPELKKPGETVKISCKASG | Anti-BCMA C11D5.3 |
| | YTFTDYSINWVKRAPGKGLKWMG | scFv heavy chain |
| | WINTETREPAYAYDFRGRFAFSLETS | variable region |
| | ASTAYLQINNLKYEDTATYFCALDY | |
| | SYAMDYWGQGTSVTVSS | |
| 166 | DYSIN | Anti-BCMA C11D5.3 |
| | | scFv heavy chain CDR1 |
| 167 | WINTETREPAYAYDFRG | Anti-BCMA C11D5.3 |
| | | scFv heavy chain CDR2 |
| 168 | DYSYAMDY | Anti-BCMA C11D5.3 |
| | | scFv heavy chain CDR3 |
| 169 | DIVLTQSPPSLAMSLGKRATISCRAS | Anti-BCMA C12A3.2 |
| | ESVTILGSHLIYWYQQKPGQPPTLLI | scFv entire sequence, |
| | QLASNVQTGVPARFSGSGSRTDFTL | with Whitlow linker |
| | TIDPVEEDDVAVYYCLQSRTIPRTFG | |
| | GGTKLEIKGSTSGSGKPGSGEGSTK | |
| | GQIQLVQSGPELKKPGETVKISCKAS | |
| | GYTFRHYSMNWVKQAPGKGLKWM | |
| | GRINTESGVPIYADDFKGRFAFSVET | |
| | SASTAYLVINNLKDEDTASYFCSND | |
| | YLYSLDFWGQGTALTVSS | |
| 170 | DIVLTQSPPSLAMSLGKRATISCRAS | Anti-BCMA C12A3.2 |
| | ESVTILGSHLIYWYQQKPGQPPTLLI | scFv light chain variable |
| | QLASNVQTGVPARFSGSGSRTDFTL | region |
| | TIDPVEEDDVAVYYCLQSRTIPRTFG | |
| | GGTKLEIK | |
| 171 | RASESVTILGSHLIY | Anti-BCMA C12A3.2 |
| | | scFv light chain CDR1 |
| 172 | LASNVQT | Anti-BCMA C12A3.2 |
| | | scFv light chain CDR2 |
| 173 | LQSRTIPRT | Anti-BCMA C12A3.2 |
| | | scFv light chain CDR3 |
| 174 | QIQLVQSGPELKKPGETVKISCKASG | Anti-BCMA C12A3.2 |
| | YTFRHYSMNWVKQAPGKGLKWMG | scFv heavy chain |
| | RINTESGVPIYADDFKGRFAFSVETS | variable region |
| | ASTAYLVINNLKDEDTASYFCSNDY | |
| | LYSLDFWGQGTALTVSS | |
| 175 | HYSMN | Anti-BCMA C12A3.2 |
| | | scFv heavy chain CDR1 |
| 176 | RINTESGVPIYADDFKG | Anti-BCMA C12A3.2 |
| | | scFv heavy chain CDR2 |
| 177 | DYLYSLDF | Anti-BCMA C12A3.2 |
| | | scFv heavy chain CDR3 |
| 178 | EVQLLESGGGLVQPGGSLRLSCAAS | Anti-BCMA FHVH33 |
| | GFTFSSYAMSWVRQAPGKGLEWVS | entire sequence |
| | SISGSGDYIYYADSVKGRFTISRDISK | |
| | NTLYLQMNSLRAEDTAVYYCAKEG | |
| | TGANSSLADYRGQGTLVTVSS | |
| 179 | GFTFSSYA | Anti-BCMA FHVH33 |
| | | CDR1 |
| 180 | ISGSGDYI | Anti-BCMA FHVH33 |
| | | CDR2 |
| 181 | AKEGTGANSSLADY | Anti-BCMA FHVH33 |
| | | CDR3 |
| 182 | DIQMTQSPSSLSASVGDRVTITCRAS | Anti-BCMA CT103A |
| | QSISSYLNWYQQKPGKAPKLLIYAA | scFv entire sequence, |
| | SSLQSGVPSRFSGSGSGTDFTLTISSL | with Whitlow linker |
| | QPEDFATYYCQQKYDLLTFGGGTK | |
| | VEIKGSTSGSGKPGSGEGSTKGQLQ | |
| | LQESGPGLVKPSETLSLTCTVSGGSI | |
| | SSSSYYWGWIRQPPGKGLEWIGSISY | |
| | SGSTYYNPSLKSRVTISVDTSKNQFS | |
| | LKLSSVTAADTAVYYCARDRGDTIL | |
| | DVWGQGTMVTVSS | |
| 183 | DIQMTQSPSSLSASVGDRVTITCRAS | Anti-BCMA CT103A |
| | QSISSYLNWYQQKPGKAPKLLIYAA | scFv light chain variable |
| | SSLQSGVPSRFSGSGSGTDFTLTISSL | region |
| | QPEDFATYYCQQKYDLLTFGGGTK | |
| | VEIK | |
| 184 | QSISSY | Anti-BCMA CT103A |
| | | scFv light chain CDR1 |
| 185 | AAS | Anti-BCMA CT103A |
| | | scFv light chain CDR2 |
| 186 | QQKYDLLT | Anti-BCMA CT103A |
| | | scFv light chain CDR3 |
| 187 | QLQLQESGPGLVKPSETLSLTCTVSG | Anti-BCMA CT103A |
| | GSISSSSYYWGWIRQPPGKGLEWIGS | scFv heavy chain |
| | ISYSGSTYYNPSLKSRVTISVDTSKN | variable region |
| | QFSLKLSSVTAADTAVYYCARDRG | |
| | DTILDVWGQGTMVTVSS | |
| 188 | GGSISSSSYY | Anti-BCMA CT103A |
| | | scFv heavy chain CDR1 |
| 189 | ISYSGST | Anti-BCMA CT103A |
| | | scFv heavy chain CDR2 |
| 190 | ARDRGDTILDV | Anti-BCMA CT103A |
| | | scFv heavy chain CDR3 |

### iv) Anti-FAP CAR

In certain embodiments comprising an anti-FAP CAR, the anti-FAP CAR comprises as scFv based on the antibody 4G5 (see WO2021/061708 and WO2021/061778). In some embodiments comprising an anti-FAP CAR comprising a scFv based on the antibody 4G5 further comprises a hinge and transmembrane from CD8, a 4-1BB co-stimulatory domain, and a CD3ζ signaling domain, Examples of an anti-FAP CARs include CARs disclosed in WO2021/061778.

TABLE 13
Exemplary sequences of anti-FAP 4G5-based CARs

| SEQ | | |
|---|---|---|
| ID NO: | Amino Acid Sequence | Description |
| 191 | MALPVTALLLPLALLLHAARPGS | Signal peptide (plus GS) |
| 192 | QVQLQQPGAELVKPGASVKLSCKA | 4G5 scFv VH N- |
| | SGYTITSYSLHWVKQRPGQGLEWIG | terminal |
| | EINPANGDHNFSEKFEIKATLTVDSS | |
| | SNTAFMQLSRLTSEDSAVYYCTRLD | |
| | DSRFHWYFDVWGAGTTVTVSSGGG | |
| | GSGGGGSGGGGSQIVLTQSPALMSA | |
| | SPGEKVTMTCTASSSVSYMYWYQQ | |
| | KPRSSPKPWIFLTSNLASGVPARFSG | |
| | RGSGTSFSLTISSMEAEDAATYYCQ | |
| | QWSGYPPITFGSGTKLEIK | |
| 193 | SGTTTPAPRPPTPAPTIASQPLSLRPE | CD8α hinge |
| | ACRPAAGGAVHTRGLDFACD | (preceded by SG spacer |
| 125 | IYIWAPLAGTCGVLLLSLVITLYC | CD8α transmembrane |
| | | domain |
| 127 | KRGRKKLLYIFKQPFMRPVQTTQEE | 4-1BB co-stimulatory |
| | DGCSCRFPEEEEGGCEL | domain |
| 130 | RVKFSRSADAPAYKQGQNQLYNEL | Cd3ζ signaling domain |
| | NLGRREEYDVLDKRRGRDPEMGGK | |
| | PRRKNPQEGLYNELQKDKMAEAYS | |
| | EIGMKGERRRGKGHDGLYQGLSTA | |
| | TKDTYDALHMQALPPR | |
| 194 | QIVLTQSPALMSASPGEKVTMTCTA | 4G5 scFv VL N-terminal |
| | SSSVSYMYWYQQKPRSSPKPWIFLT | |
| | SNLASGVPARFSGRGSGTSFSLTISS | |
| | MEAEDAATYYCQQWSGYPPITFGS | |
| | GTKLEIKGGGGSGGGGSGGGGSQV | |
| | QLQQPGAELVKPGASVKLSCKASG | |
| | YTITSYSLHWVKQRPGQGLEWIGEI | |
| | NPANGDHNFSEKFEIKATLTVDSSSN | |
| | TAFMQLSRLTSEDSAVYYCTRLDDS | |
| | RFHWYFDVWGAGTTVTVSS | |

In some embodiments, the anti-CD8 tLNP encapsulates a nucleic acid encoding an anti-GPRC5D chimeric antigen receptor (CAR). GPRC5D is a G protein-coupled receptor without known ligands and of unclear function in human tissue. However, this receptor is expressed in myeloma cell lines and in bone marrow plasma cells from patients with multiple myeloma. GPRC5D has been identified as an immunotherapeutic target in multiple myeloma and Hodgkin lymphomas. Examples of anti-GPRC5D CARs include those incorporating a GPRC5D binding moiety such as MCARH109 (Mailankody et al., N Engl J Med. 387(13): 1196-1206 (2022)), BMS-986393, or OriCAR-017 (Rodriguez-Otero et al., Blood Cancer J. 14(1): 24 (2024)). Examples of anti-GPRC5D CARs include those incorporating a GPRC5D binding moiety derived from an antibody specific to GPRC5D, for example, talquetamab (Pillarisetti et al., Blood 135:1232-43 (2020)), or forimtamig. In some embodiments, the extracellular binding domain of the anti-GPRC5D CAR comprises an scFv derived from a 6D9 Mouse antibody with specificity to human GPRC5D (see creative-biolabs.com/car-t/anti-gprc5d-6d9-h-41bb-cd3-car-pcdcar1-26380.htm). In some embodiments, the extracellular binding domain of the GPRC5D CAR comprises an scFv of anti-GPRC5D antibody linked to 4-1BB or CD28 costimulatory domain and CD3ζ signaling domain as described in Mailankody et al., N Engl J Med. 387(13): 1196-1206 (2022); creative-biolabs.com/car-t/anti-gprc5d-6d9-h-41bb-cd3-car-pcdcar1-26380.htm; and Rodriguez-Otero et al., Blood Cancer J. 14(1): 24 (2024). The entire contents of each of foregoing references in this paragraph are incorporated by reference for all that they teach about the design, structure, and activity of anti-GPRC5D CARs and anti-GPRC5D antibodies that can provide an antigen binding domain for a CAR or immune cell engager, and each example constitutes a means for binding GPRC5D. In any of the aforementioned tLNP embodiments, certain embodiments include tLNPs encapsulating a GPRC5D CAR payload encoded by RNA and having a T cell targeting moiety, such as an anti-CD8 antibody.

In some embodiments, the the anti-CD8 tLNP encapsulates a nucleic acid encoding an anti-FCRL5 chimeric antigen receptor (CAR). FCRL5 (Fc receptor-like 5), also known as FCRH5, BXMAS1, CD307, CD307E, and IRTA2, is a protein marker expressed on the surface of plasma cells in patients with multiple myeloma. Furthermore, contact with FCRL5 stimulates B-cell proliferation; thus, FCRL5 has been identified as an immunotherapeutic target for this disease. Examples of anti-FCRL5 CARS include those incorporating an FCRL5 binding moiety, such as those described in WO2016090337, WO2017096120, WO2022263855, and WO2024047558. In some embodiments, the extracellular binding domain of the anti-FCRL5 CAR comprises an scFv with specificity to FCRL5, such as ET200-31, ET200-39, ET200-69, ET200-104, ET200-105, ET200-109, or ET200-117. In some embodiments, the extracellular binding domain of the anti-FCRL5 CAR comprises an scFv derived from a mouse antibody with specificity to human FCRL5. Such antibodies include 7D11, F25, F56, and F119, as described in Polson et al., Int. Immunol., 18(9): 1363-1373 (2006); Franco et al., J. Immunol. 190(11): 5739-5746 (2013); Ise et al., Clin. Cancer Res. 11(1): 87-96 (2005); and Ise et al., Clin. Chem. Lab. Med. 44(5): 594-602 (2006), all of which are incorporated by reference herein. In some embodiments, the extracellular binding domain of the anti-FCRL5 CAR comprises a binding moiety derived from the antigen binding domain of an anti-FCRL5 antibody or nanobody, including cevostamab, 2A10H7, 307307, 2A10D6, 13G9, 10A8, 509f6, EPR27365-87, EPR26948-19, or EPR26948-67, or as disclosed in WO2016090337, WO2017096120, WO2022263855, or WO2024047558. In some embodiments, the extracellular binding domain of the anti-FCRL5 CAR comprises a binding moiety derived from an antibody-drug conjugate targeting FCRL5, such as those described in Elkins et al., Mol. Cancer Ther. 11(10): 2222-2232 (2012). In some embodiments, the extracellular binding domain of the anti-FCRL5 CAR is linked to a costimulatory domain, such as a 4-1BB or CD28 costimulatory domain, and a signaling domain, such as a CD3ζ signaling domain. The entire contents of each of the foregoing references in this paragraph are incorporated by reference for all that they teach about the design, structure, properties, and activity of anti-FCRL5 CARs and anti-FCRL5 antibodies that can provide an antigen binding domain for a CAR or immune cell engager. Each example constitutes a means for binding FCRL5. In any of the aforementioned tLNP embodiments, certain embodiments include tLNPs encapsulating a FCRL5 CAR payload encoded by RNA and having a T cell targeting moiety, such as an anti-CD8 antibody.

Each of the CARs with specificity for a particular antigen described herein constitute means for antigen recognition with respect to that antigen and collectively all of the CARs described herein constitute means for antigen recognition. The function can be alternatively stated as antigen recognition by an immune cell or antigen recognition by a T cell and the like.

In certain embodiments, on ORF can encode a gene-editing nuclease such as one encoding an RNA-guided nuclease, a gene or base editing protein, a prime editing protein, a Gene Writer protein (e.g., a modified or modularized non-long terminal repeat (LTR) retrotransposon), a retrotransposase, an RNA writer, a zinc finger nuclease (ZFN), a transcription activator-like effector nuclease (TALEN), a meganuclease, a transposase, a retrotransposon, a reverse transcriptase (e.g., M-HLV reverse transcriptase), a nickase or inactive nuclease (e.g., Cas9, nCas9, dCas9), a DNA recombinase, a CRISPR nuclease (e.g., Cas9, Cas12, Cas13, Cas3, CasMINI, Cas7-11, CasX), a DNA nickase, a Cas9 nickase (e.g., D10A or H840A), or any fusion or combination thereof. Genome-, gene-, and base-editing technology are reviewed in Anzalone et al., Nature Biotechnology 38:824-844, 2020, Sakuma, Gene and Genome Editing 3-4:100017, 2022, and Zhou et al., MedComm 3(3):e155, 2022, each of which is incorporated by reference for all that they teach about the components and uses of this technology to the extent that it does not conflict with the present disclosure.

In certain embodiments of any of the above aspects, the poly(A) sequence can have at least about 80 adenosine residues to about 130 or more adenosine residues. In some embodiments the poly(A) sequence has about 80 adenosine residues. In certain embodiments, the poly(A) sequence has about 90 adenosine residues. In certain embodiments, the poly(A) sequence has about 100 adenosine residues. In certain embodiments, the poly(A) sequence has about 110 adenosine residues. In certain embodiments, the poly(A) sequence has about 130 adenosine residuesEach of the CARs with specificity for a particular antigen described herein constitute means for antigen recognition with respect to that antigen and collectively all of the CARs described herein constitute means for antigen recognition. The function may be alternatively stated as antigen recognition by an immune cell or antigen recognition by a T cell and the like.

Each of the various genera, subgenera, and or species of LNP or tLNP disclosed herein including those based on the inclusion or exclusion of particular lipids, particular lipid compositions, particular payloads, and/or particular humanized anti-CD8 antibodies, can be used in defining the scope of embodiments of each of the methods of treatment.

Unless otherwise defined, all technical terms used herein have the same meaning as commonly understood by a person of ordinary skill in the art to which this disclosure belongs.

For the purposes of promoting an understanding of the principles of the disclosure, reference will now be made to embodiments and specific language will be used to describe the same. It will nevertheless be understood that no limitation of the scope of the disclosure was thereby intended, such alteration and further modifications of the disclosure as illustrated herein, being contemplated as would normally occur to one skilled in the art to which the disclosure relates.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

TABLE 14
LNP Compositions

| Composition | | |
|---|---|---|
| Code | Lipid Composition [Ratios] | N/P |
| BF1 | ALC-0315:DSPC:CHOL:DMG-PEG(2k):DSPE- | 6 |
| | PEG(2k)-MAL [50:10:38.5:1.4:0.1] | |
| F1 | CICL1:DSPC:CHOL:DMG-PEG(2k):DSPE- | 6 |
| | PEG(2k)-MAL [50:10:38.5:1.4:0.1] | |
| F2 | CICL1:DSPC:CHOL:DMG-PEG(2k):DSPE- | 3 |
| | PEG(2k)-MAL [50:10:38.5:1.3:0.2] | |
| F3 | CICL1:DSPC:CHOL:DMG-PEG(2k):DSPE- | 9 |
| | PEG(2k)-MAL [50:10:38.5:1.425:0.075] | |
| F4 | CICL1:DSPC:CHOL:DMG-PEG(2k):DSPE- | 6 |
| | PEG(2k)-MAL [42:10:46.5:1.4:0.1] | |
| F5 | CICL1:DSPC:CHOL:DMG-PEG(2k):DSPE- | 6 |
| | PEG(2k)-MAL [58:10:30.5:1.4:0.1] | |
| F6 | CICL1:DSPC:CHOL:DSG-PEG(2k):DSPE- | 6 |
| | PEG(2k)-MAL [35:10:53.5:1.4:0.1] | |
| F7 | CICL1:DSPC:CHOL:DSG-PEG(2k):DSPE- | 6 |
| | PEG(2k)-MAL [42:10:46.5:1.4:0.1] | |
| F8 | CICL1:DSPC:CHOL:DSG-PEG(2k):DSPE- | 6 |
| | PEG(2k)-MAL [50:10:38.5:1.4:0.1] | |
| F9 | CICL1:DSPC:CHOL:DSG-PEG(2k):DSPE- | 6 |
| | PEG(2k)-MAL [58:10:30.5:1.4:0.1] | |
| F10 | CICL1:DSPC:CHOL:DSG-PEG(2k):DSPE- | 6 |
| | PEG(2k)-MAL [62:10:26.5:1.4:0.1] | |
| F11 | CICL1:DSPC:CHOL:DMG-PEG(2k):DSPE- | 6 |
| | PEG(2k)-MAL [58:7:33.5:1.4:0.1] | |
| F12 | CICL1:DSPC:CHOL:DPG-PEG(2k):DSPE- | 6 |
| | PEG(2k)-MAL [58:7:33.5:1.4:0.1] | |
| F13 | CICL1:DSPC:CHOL:DSG-PEG(2k):DSPE- | 6 |
| | PEG(2k)-MAL [58:7:33.5:1.4:0.1] | |
| F14 | CICL1:DSPC:CHOL:DMG-PEG(2k):DSPE- | 6 |
| | PEG(2k)-MAL [58:7:34:0.9:0.1] | |
| F15 | CICL1:DSPC:CHOL:DSG-PEG(2k):DSPE- | 6 |
| | PEG(2k)-MAL [58:10:30:1.9:0.1] | |
| F16 | CICL1:DSPC:CHOL:DSG-PEG(2k):DSPE- | 6 |
| | PEG(2k)-MAL [50:10:39.5:0.4:0.1] | |
| F17 | CICL1:DSPC:CHOL:DSG-PEG(2k):DSPE- | 6 |
| | PEG(2k)-MAL [50:10:39:0.9:0.1] | |
| F18 | CICL1:DSPC:CHOL:DSG-PEG(2k):DSPE- | 6 |
| | PEG(2k)-MAL [50:10:38.5:1.4:0.1] | |
| F19 | CICL1:DSPC:CHOL:DSG-PEG(2k):DSPE- | 6 |
| | PEG(2k)-MAL [50:10:38:1.9:0.1] | |
| F20 | CICL1:DSPC:CHOL:DSG-PEG(2k):DSPE- | 6 |
| | PEG(2k)-MAL [50:10:37.5:2.4:0.1] | |
| F21 | CICL1:DSPC:CHOL:DSG-PEG(2k):DSPE- | 6 |
| | PEG(2k)-MAL [50:10:37:2.9:0.1] | |
| F22 | CICL1:DSPC:CHOL:DSG-PEG(2k):DSPE- | 6 |
| | PEG(2k)-MAL [58:10:31:0.9:0.1] | |
| F23 | CICL1:DSPC:CHOL:DSG-PEG(2k):DSPE- | 6 |
| | PEG(2k)-MAL [58:10:30:1.9:0.1] | |
| F24 | CICL1:DSPC:CHOL:DSG-PEG(2k):DSPE- | 6 |
| | PEG(2k)-MAL [58:10:29.5:2.4:0.1] | |
| F25 | CICL1:DSPC:CHOL:DSPE-PEG(0.75k):DSPE- | 6 |
| | PEG(2k)-MAL [58:10:30.5:1.4:0.1] | |
| F26 | CICL1:DSPC:CHOL:DSPE-PEG(1k):DSPE- | 6 |
| | PEG(2k)-MAL [58:10:30.5:1.4:0.1] | |
| F27 | CICL1:DSPC:CHOL:DMPE-PEG(1k):DSPE- | 6 |
| | PEG(2k)-MAL [58:10:30.5:1.4:0.1] | |
| F29 | CICL1:DSPC:CHOL:DSG-PEG(5k):DSPE- | 6 |
| | PEG(5k)-MAL [58:10:31.4:0.5:0.1] | |
| F30 | CICL1:DSPC:CHOL:DMG-PEG(2k):DSG- | 6 |
| | PEG(2k)-MAL [58:10:30.5:1.4:0.1] | |
| F31 | CICL1:DSPC:CHOL:DSG-PEG(2k):DSG- | 6 |
| | PEG(2k)-MAL [58:10:30.5:1.4:0.1] | |
| F32 | CICL1:DSPC:CHOL:DSPE-PEG(2k):DSPE- | 6 |
| | PEG(2k)-MAL [58:10:30.5:1.4:0.1] | |
| F33 | CICL1:DSPC:CHOL:DSG-PEG(2k):DSPE- | 6 |
| | PEG(5k)-MAL [58:10:30.5:1.4:0.1] | |
| F34 | CICL1:DSPC:CHOL:DSG-PEG(2k):DSPE- | 6 |
| | PEG(2k)-MAL [58:13:27.5:1.4:0.1] | |
| F35 | CICL1:DMPC:CHOL:DSG-PEG(2k):DSPE- | 6 |
| | PEG(2k)-MAL [58:10:30.5:1.4:0.1] | |
| F36 | CICL1:DPPC:CHOL:DSG-PEG(2k):DSPE- | 6 |
| | PEG(2k)-MAL [58:10:30.5:1.4:0.1] | |
| F37 | CICL1:DAPC:CHOL:DSG-PEG(2k):DSPE- | 6 |
| | PEG(2k)-MAL [58:10:30.5:1.4:0.1] | |
| F38 | CICL1:18:1 PA:CHOL:DSG-PEG(2k):DSPE- | 6 |
| | PEG(2k)-MAL [58:10:30.5:1.4:0.1] | |
| F40 | CICL1:DSPC:CHOL:20(S)- | 6 |
| | Hydroxycholesterol:DSG-PEG(2k):DSPE- | |
| | PEG(2k)-MAL [58:10:22.9:7.6:1.4:0.1] | |
| F41 | CICL1:DSPC:CHOL:β-Sitosterol:DSG- | 6 |
| | PEG(2k):DSPE-PEG(2k)-MAL | |
| | [58:10:22.9:7.6:1.4:0.1] | |
| F42 | CICL1:DSPC:CHOL:β-Sitosterol:DSG- | 6 |
| | PEG(2k):DSPE-PEG(2k)-MAL | |
| | [58:10:15.25:15.25:1.4:0.1] | |

### EXAMPLES

### Materials and Methods

### Generation of Humanized Andi-CD8α Binders/Antibodies

The sequence of the CT8 antibody (also referred to herein as CBD1017p) is disclosed herein. The VH and VL sequences were compared to a library of known human germline sequences from human VH genes and human VLkappa genes (IMGT® the international ImMunoGeneTics information system® www.imgt.org; founder and director: Marie-Paule Lefranc, Montpellier, France); databases used were IMGT human VH genes (F+ORF, 273 germline sequences) and IMGT human VLkappa genes (F+ORF, 74 germline sequences) as used by the NCBI IgBLAST program. The acceptor human germline was chosen from those closest in sequence to the parental antibody.

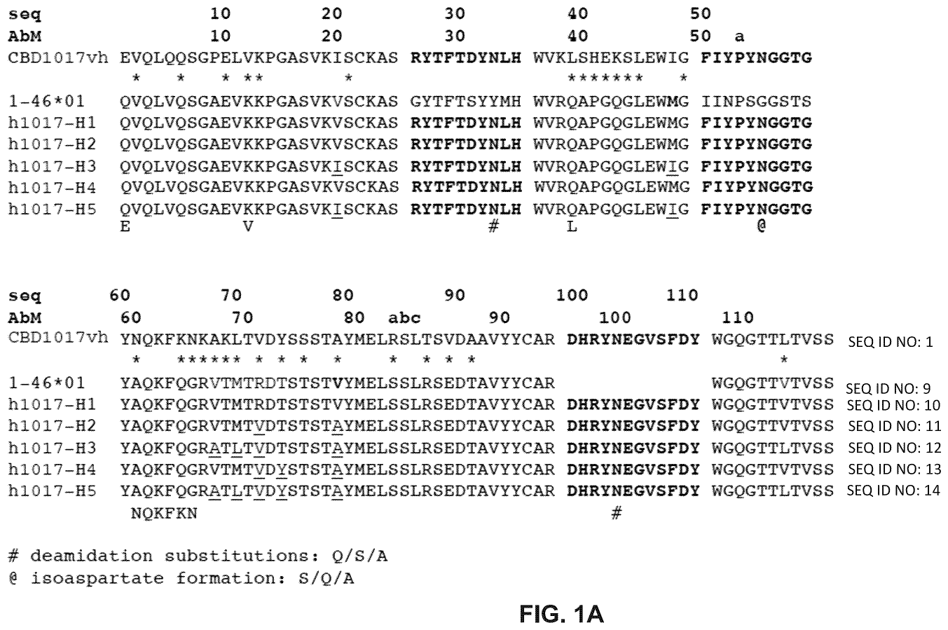

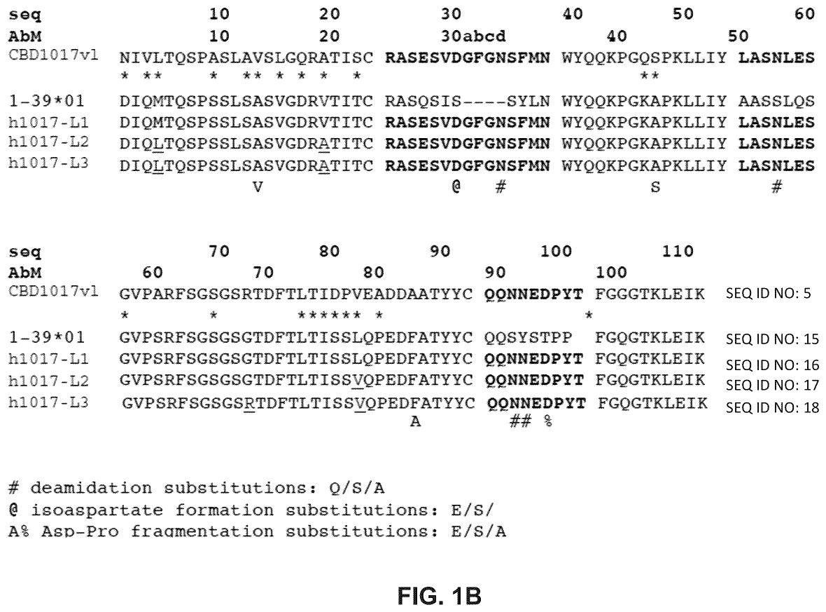

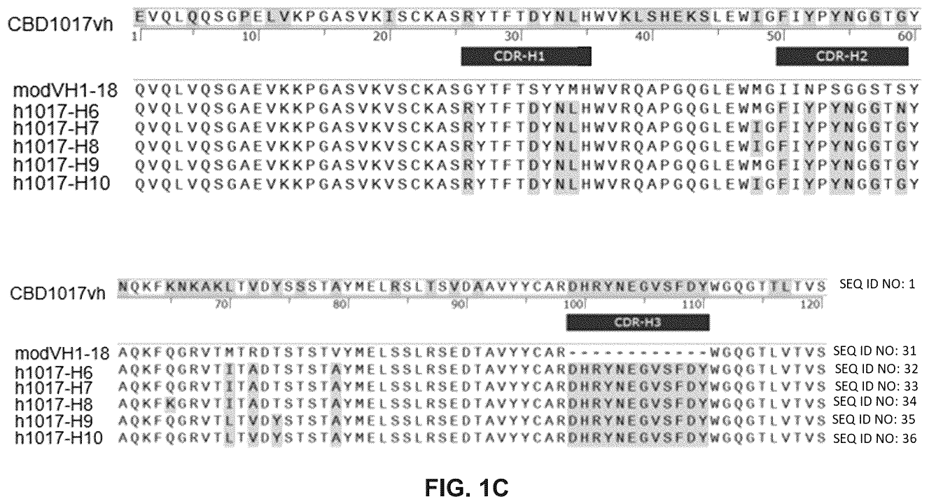

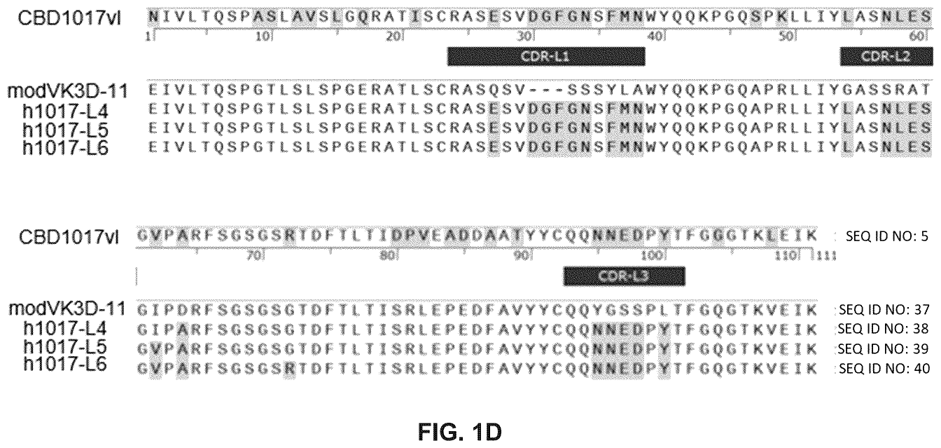

For VH, human germline IGHV1-46*01 was used as the acceptor sequence and the human heavy chain IGHJ6 (allele 1) joining region (J gene) was selected from human joining region sequences compiled at IMGT® the international ImMunoGeneTics information system® www.imgt.org (founder and director: Marie-Paule Lefranc, Montpellier, France) (see FIG. 1A).

For VL, human germline IGKV1-39*01 was used as the acceptor sequence and human light chain IGKJ2 (allele 1) joining region (J gene) was selected from human joining region sequences compiled at IMGT® the international ImMunoGeneTics information system® www.imgt.org (founder and director: Marie-Paule Lefranc, Montpellier, France) (see FIG. 1B).

CDRs were defined according to the AbM definition (see the website of Dr. Andrew C. R. Martin on bioinf website for a table comparing CDR definitions). Alteration of human germline framework positions (i.e., non-CDR residues in VH and VL) to the corresponding parental murine sequence is contemplated to optimize binding of the humanized antibody to CD8-specific antigens. Potential changes for each humanized sequence are noted in FIG. 1A and FIG. 1B.

CBD1017p VH and VL sequences also were compared to a library of known human germline sequences from human VH genes and human VL kappa genes using the IMGT/BlastSearch implementation online. IGHV1-18*01 was used as the heavy chain acceptor sequence and IGKV3D-11*02 was used as the human light chain acceptor sequence. Manual inspection using BioLuminate® modeling software online (Schrödinger Inc.) was used to arrive at a consensus modified germline sequence that was used as the starting point for humanization (FIG. 1C and FIG. 1D) positions contemplated.

### Binding Affinity (K_{D}) Measurement by Biolayer Interferometry Kinetic Assays

Biolayer interferometry kinetic assays were performed using a GatorBio Gator Plus BLI system. Kinetic buffer containing PBS plus 0.1% Tween 20 and 0.2% BSA was used for calculating the baseline. To measure the binding kinetics of anti-CD8α antibody fragment (Fab), streptavidin-immobilized SA-XT sensors (#160029, Gator Bio) were prehydrated in the kinetic buffer and loaded with 100 mM biotinylated CD8α recombinant protein (CDA-H82E3, Acro Biosystems) at a spin speed of 400 rpm to a threshold response of 10 nm. The sensors were then incubated in the kinetic buffer for 300 s to acquire a baseline measurement prior to each association. Each anti-CD8α Fab fragment was diluted into kinetic buffer via 2-fold dilution at concentrations ranging from 100 nM to 3.12 nM. The antigen-loaded sensors were incubated in the diluted solutions to capture Fab fragments for 300 s to record the association phase. Sensors were finally incubated in the kinetic buffer for 900 s to record the dissociation phase. The spin speed for all steps except the antigen loading step was 1000 rpm. The temperature of the performed assays was either 30° C. or 37° C. during the association and dissociation phases. Sensor data were baseline subtracted, and global curve fitting of the kinetic data from six different analyte concentrations at a 1:1 monovalent binding model was performed using GatorOne software (version v2.10) to determine kinetic rate constants (kₒₙ and k_{off}) and equilibrium dissociation constants (K_{D}) of the analyzed antibodies.

The binding kinetics of CBD1033 Fab and its parental CBD1017ch Fab against human CD8αα homodimer, human CD8αβ heterodimer, and cynomolgus macaque CD8ααhomodimer were assessed following the same method in BLI evaluation of humanized Fab variants. For evaluating the binding kinetics against cynomolgus macaque CD8αβ heterodimer protein, the antigen protein was directly coupled onto amine-reactive biosensor tips (Gator Bio) following the manufacturer protocol. Briefly, the sensors were activated for 300 s with a mixture of 1-ethyl-3-(3-dimethylaminopropyl)carbodiimide hydrochloride (EDC) (ThermoFisher Scientific) and 10 mM N-Hydroxysulfosuccinimide sodium salt (sulfo-NHS) (Sigma-Aldrich) in water. The cynomolgus macaque CD8αβ protein diluted to 100 mM in 10 mM sodium acetate buffer, pH 6.0, was allowed to be covalently captured for 300 s to an approximate threshold of 5 nm, and the sensors were subsequently deactivated with 1 M ethanolamine (ETA) (Sigma-Aldrich) solution, pH 8.5, for 300 s. The sensors were then rinsed in the kinetic buffer for 600 s. The rinse step was repeated for an additional 120 s for baseline calculation prior to the measurements of the binding kinetics. The antibody fragment was diluted into kinetic buffer via 3-fold dilution at concentrations ranging from 1000 nM to 1.37 nM. As above, the antigen-captured sensors were incubated in the diluted solutions to capture the Fab fragments for 1200 s to record the association phase. Sensors were subsequently incubated in the kinetic buffer for 1200 s to record the dissociation phase. The shaking speed for all steps was 1000 rpm and the temperature of the performed assays were at 30° C. The kinetic parameters were determined with 1:1 monovalent (Langmuir) binding model using the previous method.

To measure the binding kinetics of the biotin-conjugated Fab molecule, streptavidin-immobilized SA-XT sensors (#160029, Gator Bio) were prehydrated in the kinetic buffer and loaded with 50 mM biotinylated Fab sample CBD1033.37 or CBD1033.24 to a threshold response of 10 nm. The sensors were then briefly blocked with the kinetic buffer containing 50 mM biotin and 3% BSA for 60 s, and further incubated in the kinetic buffer for 120 s to achieve the steady baseline measurement prior to each association. Custom recombinant human CD8α mouse IgG2a Fc fusion protein was diluted into kinetic buffer via 2-fold dilution at concentrations ranging from 100 nM to 1.56 nM. The binder-loaded sensors were incubated in the diluted solutions to capture the CD8α protein for 1200 s to record the association phase. Sensors were finally incubated in the kinetic buffer for another 1200 s to record the dissociation phase. The spin speed for all steps was 1000 rpm. The temperature of the performed assays were at 37° C. during the association and dissociation. The sensor data were baseline subtracted, and global curve fitting of the kinetic data from six different analyte concentrations at a 1:1 monovalent binding model was performed using the GatorOne software (version v2.10) to determine the kinetic rate constants (kₒₙ and k_{off}) and equilibrium dissociation constants (K_{D}) of the analyzed binder.

### Binding Affinity (K_{D}) Measurement by Biolayer Interferometry Steady State Analysis

To assess affinity of whole antibodies, steady state analysis was used as the bivalent nature of both whole antibody and CD8α complicates kinetic analysis. 50 nM CD8α-His (that is, CD8α with a C-terminal oligohistidine tag; Acro) was immobilized on Ni-NTA sensor probes. Binding was analyzed with a three-fold antibody dilutions series: 60 nM, 20 nM, 6.67 nM, 2.22 nM, 0.74 nM, and 0.25 nM. The antigen-loaded sensors were incubated in the diluted solutions to capture the whole IgG antibody for 300 s to record the association phase. Sensors were incubated in the kinetic buffer for 900 s to record the dissociation phase. The spin speed was 1000 rpm for all steps except the antigen loading step which was 400 rpm to control density of the antigen. The temperature of the performed assays was at 30° C. during the association and dissociation phases. The sensor data were baseline subtracted and further analyzed in GatorOne software (version 2.10) to determine the equilibrium dissociation constants (K_{D}) of the antibodies by fitting the signals of the steady states to the 1:1 binding model.

### Binding Affinity (K_{D}) Measurement by Surface Plasmon Resonance Assays

Surface plasmon resonance (SPR) assays were performed using a Biacore 8K SPR instrument (Cytiva) in a running buffer containing 1×HBS-N(Cytiva) and 0.05% Tween-20 at 25° C. The CBD1033 antibody was captured by anti-human IgG (Fc) antibody (Cytiva) immobilized on a CM5 sensor chip (Cytiva) at a density within 40-50 and 80-100 response units (RU) for kinetic measurements with CD8αα and CD8αp, respectively. To measure the binding kinetics, serial two-fold dilutions of recombinant CD8 protein were prepared in the running buffer were injected into the flow cells at 30 L/min, with the concentration ranging from 200 to 6.25 nM (successive 1:2 dilutions). The association data was collected for 180 s followed by a 1200 s dissociation step. At the end of each binding cycle, the sensor surface was regenerated with a 3M MgCl₂ buffer. For cynomolgus macaque CD8αβ heterodimer, the binding kinetics were assessed with the same experimental parameters, except the concentration ranges from 400 to 12.5 nM. Sensorgrams were generated, and background-subtracted with the blank running buffer. The binding kinetic parameters were analyzed and determined with a standard 1:1 monovalent binding (Langmuir) model using BIAcore Evaluation software (Cytiva).

### Expression and Purification of Disulfide-Engineered F(Ab′)

Disulfide-engineered F(ab′) analogs were transiently expressed with an engineered CHO—K1 cell line (Wuxi Biologics) using a proprietary expression protocol. After 7 days, the culture supernatant was harvested by centrifugation and filtration. The F(ab′) analog were first captured from the filtered supernatant by affinity chromatography using KanCap™ G resin (Kaneka) and eluted with 50 mM citrate Buffer, pH 3.5. The antibody-containing eluate was buffer-exchanged to PBS pH 6.5 containing 10 mM EDTA by dialysis. The Fab molecules were then reduced by adding 5 mM 2-mercaptoethylamine (2-MEA) and incubated at room temperature for up to 90 minutes. The reduction process was monitored by taking a small sample at every 30-minute timepoint and checked for intact Fab purity by SDS-PAGE analysis. The reduced F(ab′) analogs were subsequently diluted with 20 mM NaAc, pH 5.0, captured by cation exchange chromatography using SP Sepharose High Performance resin (Cytiva) and eluted with a gradient of 0 to 1 M NaCl. The polished F(ab′) proteins were then dialyzed against 20 mM histidine-HCl, pH 5.5, 240 mM sucrose. The purities of the final F(ab′) analogs were assessed by SDS-PAGE, analytical SEC-HPLC and LC-MS analysis.

### EC₅₀ Measurement of Antigen CD8-Specific Binding

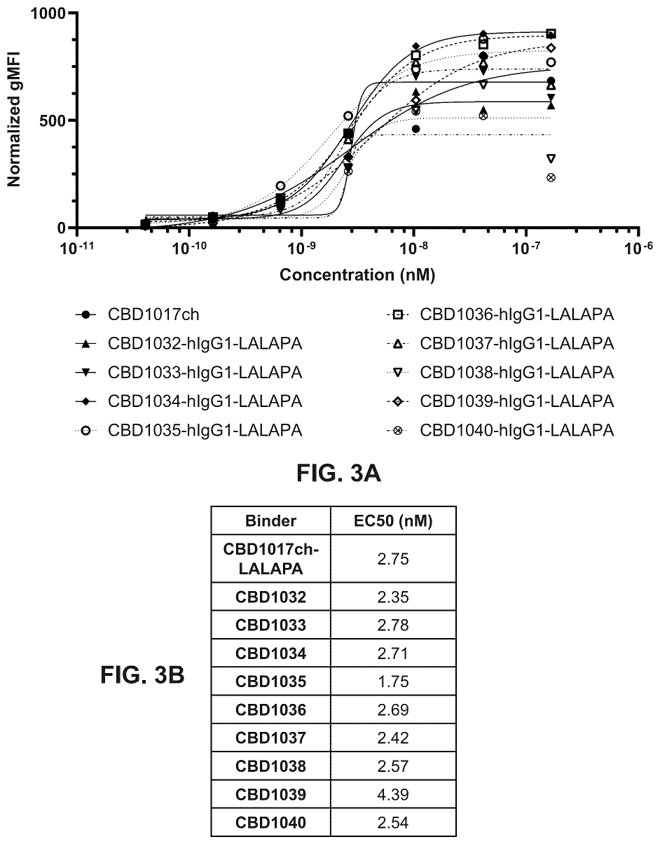

To obtain the results shown in FIG. 3A, that determined EC₅₀ values for antigen-specific binding, CD8-overexpressing HEK293T cells were dissociated in Versine solution (PBS buffer at pH 7.4 supplemented with 0.5 mM EDTA), and 2×10⁵ total cells were transferred to a V-bottom 96 well culture plate (Corning). Cells were washed twice with Cell Staining buffer (#420201, Biolegend). Each anti-CD8α antibody with human IgG1 isotype and Fc-silencing mutations L234A, L235A and P329A was diluted in four-fold antibody titration series from 100 μg/mL to 6.1 ng/ml into Cell Staining buffer. eFluor 780 Fixable Viability dye (eBioscience) was also included during antibody solutions at a 1:4000 dilution. Cells were stained with each diluted antibody solution for 30 minutes on ice. Cells were then washed three times with Cell Staining buffer and primary antibody binding was detected by staining with anti-human Fc BV421 conjugate antibody (#410704, Biolegend) at 1:100 dilution for 30 minutes on ice. After staining with the secondary detection antibody, cells were washed three times with Cell Staining buffer, fixed in FluoroFix buffer (Biolegend) and kept at 4° C. in the dark until analysis. Flow cytometry analysis on each sample was performed on an Agilent NovoCyte flow cytometer. Flow cytometry data was analyzed by Flowjo 10 (Becton, Dickinson & Company) to measure the geometric median fluorescence intensity (gMFI) of the on-cell binding from each antibody. The gMFI values were normalized with the gMFI measured in the sample stained with only the secondary detection antibody and plotted against the concentration of the added antibodies. The binding curves were constructed using the Graphpad Prism software, and EC₅₀ values were determined by non-linear curve fitting.

To obtain the results shown in FIG. 6, SupT1 (ATCC #CRL-1942) and HPB-ALL (DSMZ; ACC-483) were cultured in RPMI medium supplemented with 10% Fetal Bovine Serum (#97068-085; Avantor). To determine EC₅₀ values of cell binding from the anti-CD8α antibody, 2×10⁵ total cells were transferred to a V bottom 96-well culture plate (Corning). Cells were washed twice with Cell Staining buffer (#420201, Biolegend). The anti-CD8α antibody was diluted in four-fold antibody titration series from 60 μg/mL to 57 μg/mL into Cell Staining buffer. Cells were stained with each diluted antibody solution for 30 minutes on ice. Cells were then washed three times with Cell Staining buffer and primary antibody binding was detected by staining with anti-human Fc BV421 conjugate antibody (#410704, Biolegend) at 1:200 dilution for 30 minutes on ice. eFluor 780 Fixable Viability dye (eBioscience) at a 1:4000 dilution were also included in the detection antibody solutions. After staining with the secondary detection antibody, cells were washed three times with Cell Staining buffer, fixed in FluoroFix buffer (Biolegend) and kept at 4° C. in the dark until analysis. Flow cytometry analysis on each sample was performed on an Agilent NovoCyte flow cytometer. Flow cytometry data was analyzed by Flowjo 10 (Becton, Dickinson & Company) to measure the geometric median fluorescence intensity (gMFI) of the on-cell binding from each antibody. The gMFI values were normalized with the gMFI measured in the sample stained with only the secondary detection antibody and plotted against the concentration of the added antibodies. The binding curves were constructed using Graphpad Prism software, and EC₅₀ values were determined by non-linear curve fitting.

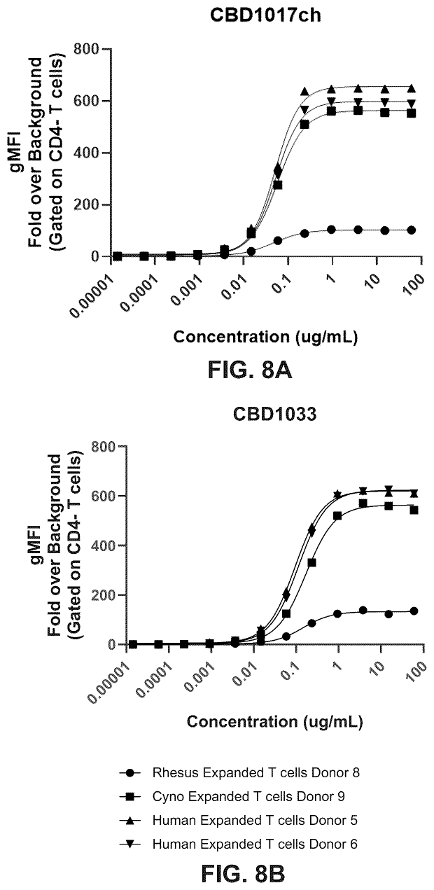

To obtain the results shown in FIG. 8A and FIG. 8B, 5×10⁴ expanded primary T cells (human, cynomolgus macaque or rhesus monkey) were thawed and transferred to a V bottom 96-well culture plate (Corning). Cells were washed twice with Cell Staining buffer (#420201, Biolegend). Each anti-CD8α antibody was diluted in four-fold antibody titration series from 60 μg/mL to 0.057 ng/mL into Cell Staining buffer. Cells were stained with each diluted antibody solution for 30 minutes on ice. Cells were then washed three times with Cell Staining buffer and primary antibody binding was detected by staining with anti-human Fc BV421 conjugate antibody (#410704, Biolegend) at 1:200 dilution for 30 minutes on ice. Anti-CD3 SP34-2 Alexa Fluor 700 conjugate antibody (#557917, BD Pharmagen) at 1:200 dilution, anti-CD4 OKT4 BV650 conjugate antibody (#317436, Biolegend) at 1:200 dilution, and eFluor 780 Fixable Viability dye (eBioscience) at a 1:5000 dilution were also included in the detection antibody solutions. After staining with the secondary detection antibody, cells were washed three times with Cell Staining buffer, fixed in eBioscience™ IC Fixation Buffer and kept at 4° C. in the dark until analysis. Flow cytometry analysis on each sample was performed on an Agilent NovoCyte flow cytometer. Flow cytometry data was analyzed by Flowjo 10 (Becton, Dickinson & Company) to measure the geometric median fluorescence intensity (gMFI) of the on-cell binding from each antibody. The gMFI values were normalized with the gMFI measured in the sample stained with only the secondary detection antibody and plotted against the concentration of the added antibodies. The binding curves were constructed using Graphpad Prism software, and EC₅₀ values were determined by non-linear curve fitting.

tLNP Formation

Initial LNPs were formed by mixing of an aqueous solution of mCherry mRNA and an ethanolic solution of the lipids in proportions CICL1:DSPC:CHOL:DSG-PEG(2 k):DSPE-PEG(2 k)-MAL [58:10:30.5:1.4:0.1](F9 composition referred to in Table 14). This is followed by stepwise phosphate and Tris buffer dilution and tangential flow filtration (TFF) purification.

Whole antibody was coupled to LNP via N-succinimidyl S-acetylthioacetate (SATA)-maleimide conjugation chemistry. The antibody was modified with SATA (Sigma-Aldrich) to introduce sulfhydryl groups at accessible lysine residues allowing conjugation to maleimide. SATA was deprotected using 0.5 M hydroxylamine followed by removal of the unreacted components by G-25 Sephadex Quick Spin Protein columns (Roche Applied Science, Indianapolis, IN). The reactive sulfhydryl group on the antibody was then conjugated to maleimide moieties on the LNPs using thioether conjugation chemistry. Conjugated tLNP (LNP conjugated with a targeting antibody) purification was performed using Sepharose CL-4B gel filtration columns (Sigma-Aldrich) or TFF (tangential flow filtration). tLNPs were frozen at −80° C. until use.

Diabodies and F(ab′)₂ were conjugated by first partially reducing cystine bonds in an antibody with tris(2-carboxy)phosphine (TCEP) to generate thiol groups for conjugation through the maleimide moieties of the LNP as described in the previous paragraph. Fab and Fab′ molecules engineered to have free thiols were similarly conjugated to maleimide moieties of the LNP.

### Conjugation Reaction Between Anti-CD8 F(Ab′) and Maleimide-PEG-Biotin (MPB)

Prior to the conjugation reaction, PBS pH 7.4, 10 mM EDTA was prepared as the conjugation buffer. Reduced F(ab′) analogs were buffer-exchanged into the conjugation buffer and diluted to 30 M concentration. The diluted F(ab′) solution was then reacted with 10-fold molar excess of EZ-Link™ Maleimide-PEG₁₁-Biotin (Thermo Fisher) with gentle shaking for 1 hour at room temperature. The reaction was subsequently quenched by adding 50-fold molar excess of N-acetylcysteine (Sigma Aldrich) and incubated with gentle shaking for another 1 hour at room temperature. The reaction mixture of Fab was buffer-exchanged into 20 mM Tris, 150 mM NaCl, pH 7.4 using a Zeba 7k MWCO column (Thermo Fisher). Biotin conjugation was verified by performing immunoblotting and detecting with HRP-conjugated streptavidin using a Protein Simple™ Jess system (Bio-Techne). The purities of the biotin conjugates were further analyzed by analytical SEC-HPLC, LC-MS, and peptide mapping analysis.

Transfection Rate and mCherry Expression Measurement

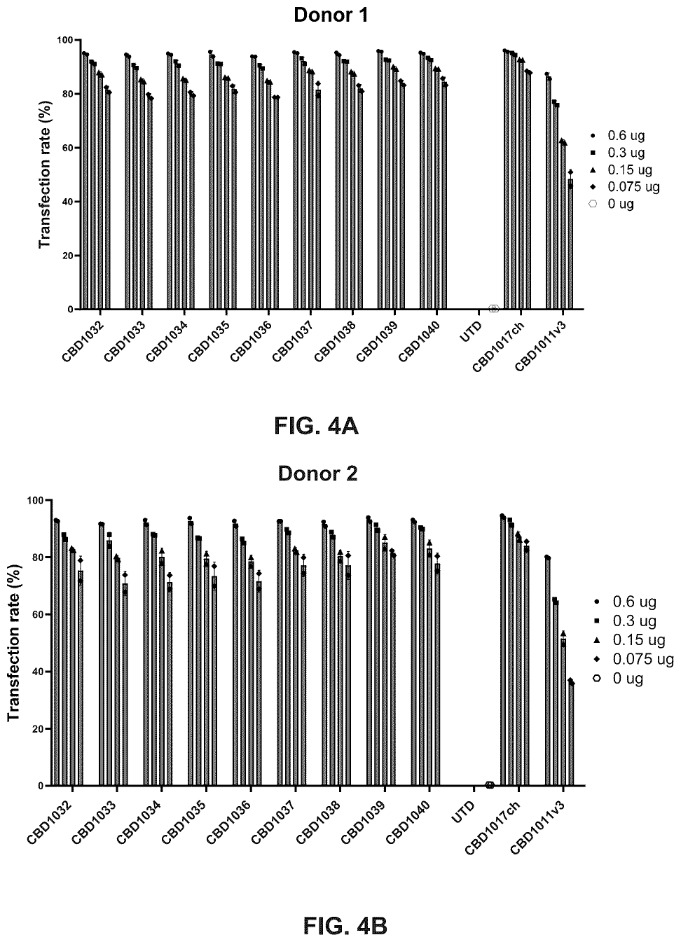

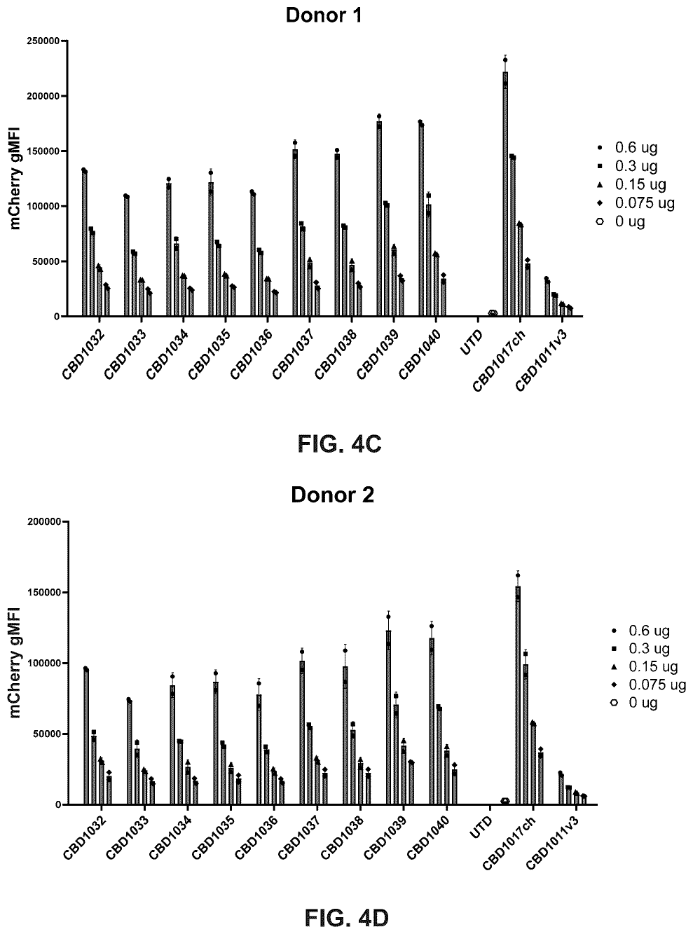

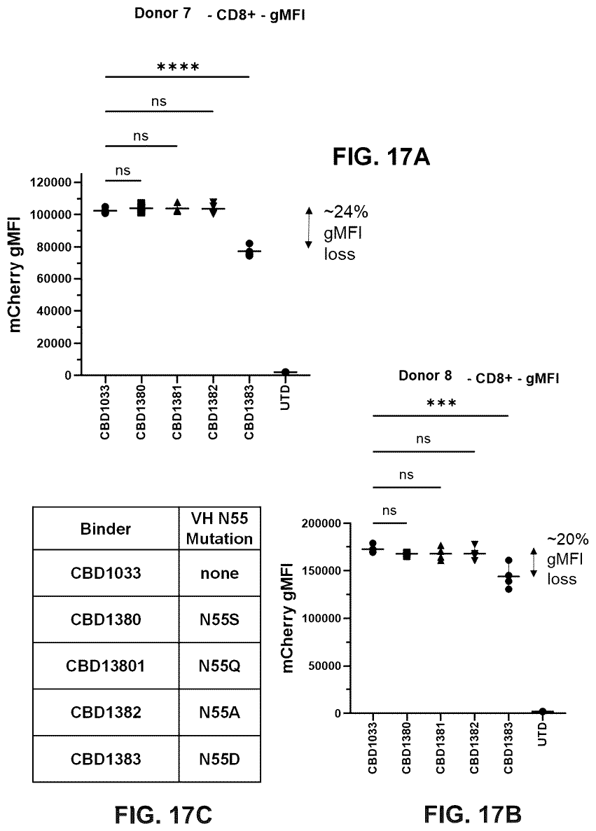

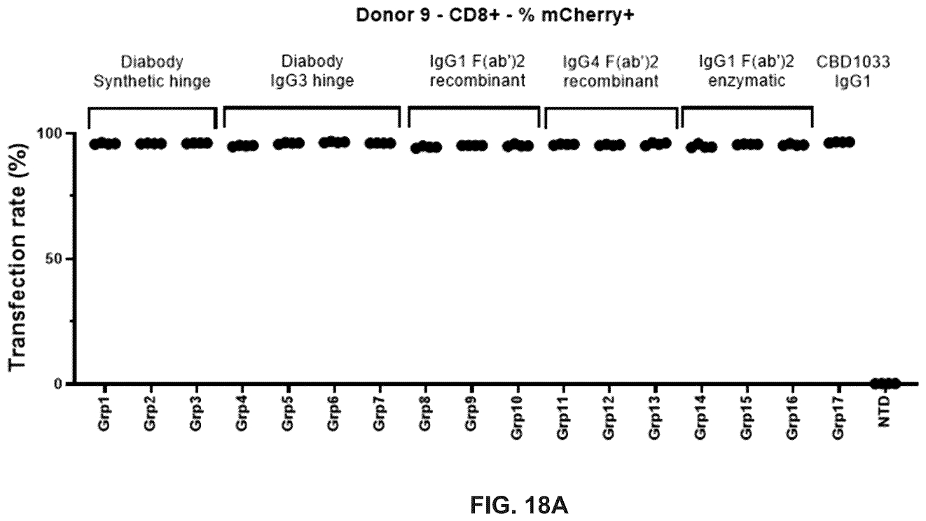

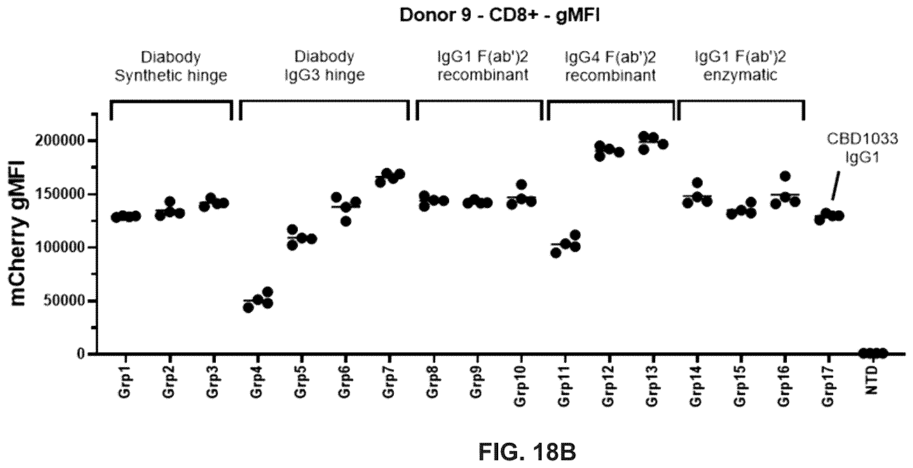

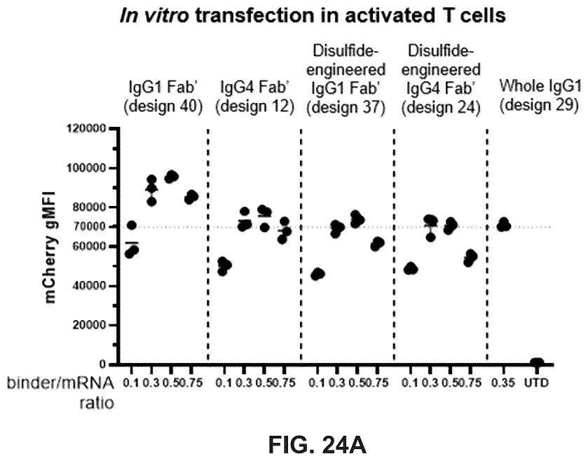

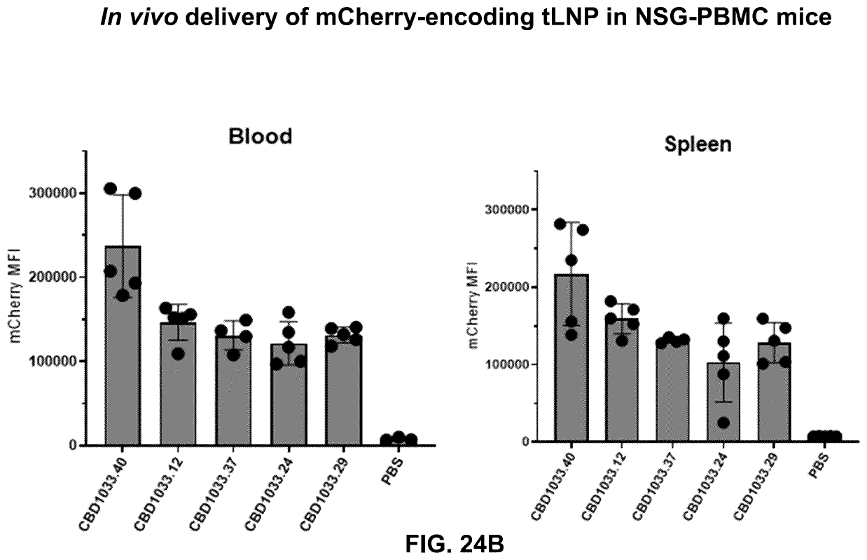

To obtain the results shown in FIG. 4A to FIG. 5C, FIG. 17A to FIG. 18B, and FIG. 24A, primary human T cells from different donors were isolated from leukapheresis samples using an Easysep Human T Cell Isolation kit (#17951 Stem Cell Technologies) and cryogenically stored in CryoStor CS10 freeze medium (#210102 Biolife Solutions). Complete T cell medium was prepared by supplementing 5% heat-inactivated human AB serum (#HP1022HI Valley Biomedical), 1% GlutaMAX (#35050061 Thermo Fisher), 1× Penicillin-Streptomycin (#15140122 Thermo Fisher), and 100 IU/mL human IL-2 (#202-IL-500 R&D) in CTS™ OpTmizer™ T-Cell Expansion serum-free medium (#A1048501 Thermo Fisher). The isolated T cells were thawed and resuspended in supplemented with Complete T cell medium at 1×10⁶ cells per mL. The cells were activated by adding Dynabeads® Human T-Activator CD3/CD28 magnetic beads at 1×10⁶ beads per mL and incubated under 5% CO₂ at 37° C. for 3 days. The mixture of T cells and activator beads were resuspended by pipetting, and the magnetic beads were carefully removed from the activated T cells using an Easysep magnet (#100-0821 Stem Cell Technologies). Isolated activated T cells were then resuspended in fresh Complete T cell medium at 1×10⁶ cells per mL. Prior to transfection, frozen formulated mCherry-encoded mRNA-encapsulated CD8α-targeted lipid nanoparticles (tLNPs) were thawed and reconstituted in Sterile Water for Injection at 100 μg mRNA/mL concentration. 2×10⁵ activated T cells were transferred to each well of U-bottom 96 well culture plates (Corning). These reconstituted tLNPs were added to the cells via 2-fold dilution at the amounts ranging from 0.6 μg to 0.075 μg. The transfected cells were incubated under 5% CO₂ at 37° C. for 1 hour before washing three times with Complete T cell medium to remove the tLNPs. The cells were further incubated under 5% CO₂ at 37° C. for 24 hours to allow for the expression of the mCherry-encoded mRNA.

To measure expression levels of mCherry, flow cytometry analysis was performed on the transfected cells. The cells were transferred to a V bottom 96-well culture plate (Corning) to prepare for antibody staining. Prior to antibody staining, an antibody solution mix was prepared by adding into Cell Staining buffer (#420201, Biolegend) an anti-CD3 FITC antibody conjugate (#556611, BD Biosciences) at 1:100 dilution, an anti-CD4 BV421 antibody conjugate (#317434, Biolegend) at 1:100 dilution, and eFluor 780 Fixable Viability dye (eBioscience) at a 1:4000 dilution. Cells were washed twice with Cell Staining buffer (#420201, Biolegend) and stained with the antibody solution mix for 30 minutes on ice. Cells were then washed three times with Cell Staining buffer and kept at 4° C. in the dark until analysis. Flow cytometry analysis on each sample was performed on an Agilent NovoCyte flow cytometer. Flow cytometry data was analyzed by Flowjo 10 (Becton, Dickinson & Company) to measure the percentage of mCherry-positive CD4-(CD8+) T cells and the geometric median fluorescence intensity (gMFI) of the mCherry expression in CD4− (CD8+) T cells.

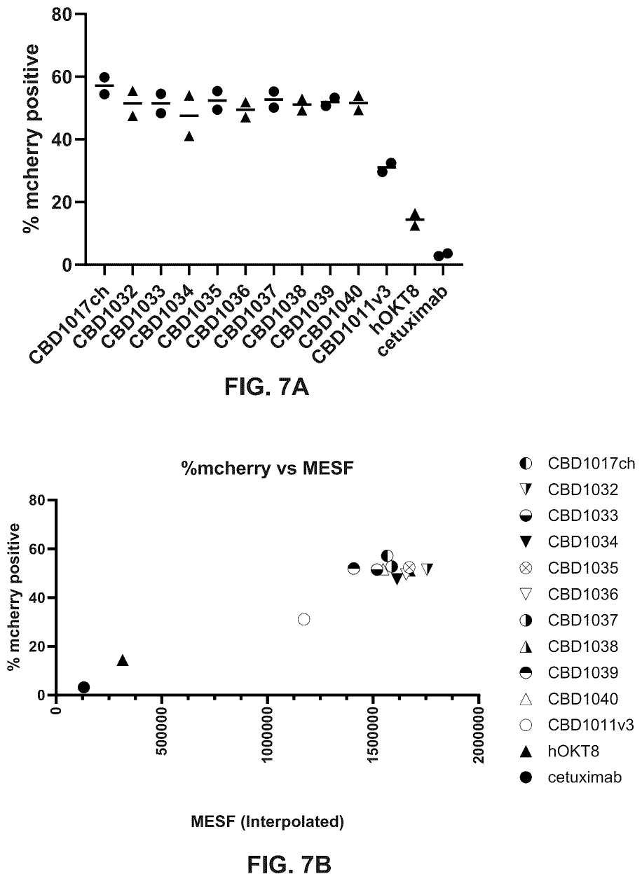

To obtain the results shown in FIG. 7A through FIG. 9B, non-human primate (NHP) peripheral blood mononuclear cells (PBMCs) from different donors were isolated from whole blood using density gradient separation. Blood was diluted in complete DPBS (DPBS with 2% FBS and 2 mM EDTA) and added to SepMate™ PBMC Isolation Tubes (StemCell Technologies, Catalog No. 85450) containing FiColl (100% for Rhesus Macaque blood, 90% for Cynomolgus Macaque blood). Tubes were centrifuged at 1,200×g for 20 min with the brake off. Plasma and PBMC layers were poured into a new 50 mL Falcon conical tube and centrifuged at 500×g for 7 min. Supernatants were removed and cell pellets resuspended into 2 mL of freezing medium. After counting, cells were stored at −80° C. for 24-48 h, then transferred to a cryobox and stored in liquid nitrogen.

Primary NHP T cells from different donors were isolated from frozen PBMCs using the EasySep NHP T Cell Isolation Kit (Catalog No. 19581 StemCell Technologies). Isolated T cells were pooled and cultured in Complete T cell medium (X-VIVO™ media (Lonza Catalog No. 02-053Q) with 100 IU/mL human IL-2 (R&D Systems Catalog No. 202-IL-500)). 100 μL of washed, activated beads (Miltenyi Biotec Catalog No. 130-092-919) were added to the culture containing isolated T cells. After 4-day incubation at 37° C. with 5% CO₂, activated T cells were harvested, de-beaded with the EasySep magnet (StemCell Technologies Catalog No. 100-0821), and washed in Complete T cell medium. Cells were diluted to 2×10⁶ cells/mL after counting and 100 μL were plated into a 96-well, round bottom plate. Frozen tLNPs were thawed and reconstituted in sterile water for injection (SWFI) according to their associated formulation handling instructions. 6 μL of reconstituted tLNPs were added to each well containing cells and incubated for 1 h at 37° C. with 5% CO₂. Transfected cells were washed three times with Complete T cell medium before an additional 24 h incubation at 37° C. with 5% CO₂.

After 24 h incubation, mCherry expression was assessed by flow cytometry. Cells were centrifuged and washed with 200 μL BD Stain Buffer (BD Biosciences Catalog No. 554656). Cells were stained with an antibody cocktail containing anti-CD3 V450, anti-CD4 (PerCP-Cy5.5), anti-CD8α (APC-H7) at 1:100 dilution for 30 min at 4° C. in the dark. Cells were then washed BD Stain Buffer until analysis. Flow cytometry sample acquisition and analysis on each sample were performed on an Agilent NovoCyte Flow Cytometer. Data reported were mCherry expression (%, geometric median fluorescence intensity (gMFI), and Molecules of Equivalent Soluble Fluorochrome (MESF)) in CD3+CD4-CD8+ T Cells and CD3+CD4+CD8− T Cells.

### In Vivo Experiments

NOD SCID gamma (NSG) mice (approximately 10 weeks old) were purchased from The Jackson Laboratory and acclimated for at least 5 days. Ten million human peripheral blood mononuclear cells (PBMCs) were injected intravenously via the tail vein. After 20 days of engraftment, mice were evaluated for frequency of human CD45+ cells in circulation and staged in groups with similar averages. The day after staging, groups of mice were injected intravenously with a single dose (5 μg/animal) human CD8-targeted tLNPs with mCherry mRNA payload. The anti-CD8α antibody was conjugated using N-succinimidyl S-acetylthioacetate (SATA) chemistry at three antibody-to-mRNA (w/w) densities (0.3, 0.5, and 1). Untreated mice were used as a negative control for flow cytometry purposes and were subjected to the same engraftment and staging protocol. Mice were sacrificed 24 hours after dosing to assess mCherry expression on T-cell subsets in the blood. Data is reported as a frequency of engineered cells based on mCherry expression of T cells (CD3+ cells), CD4 T cells (CD3+CD4+CD8−) and CD8 T cells (CD3+CD4-CD8+) respectively.

NOD-Prkdc^{em26Cd52}Il2rg^{em26Cd22}/NjuCrl (NCG) mice (approximately 9 weeks old) were purchased from Charles River laboratories and acclimated for at least 5 days. 10 million human peripheral blood mononuclear cells (PBMCs) were injected intravenously via the tail vein. After 10 days of engraftment, 500,000 Nalm6 tumor cells were injected intravenously via the tail vein. 7 days later, mice were evaluated for frequency of human CD45+ cells in circulation and staged in groups with similar averages. The day after staging, groups of mice were injected intravenously with a single dose, (10 μg/animal) human CD8-targeted tLNPs with mCherry mRNA payload. The anti-CD8 antibody (F(ab′) or whole antibody thiolated with the AJICAP process) was conjugated using SATA chemistry at an antibody to mRNA (w/w) density of 0.35 (full-length IgG) or 0.3 (Fab). Untreated mice were used as a negative control for flow cytometry purposes and were subjected to the same engraftment protocol. Mice were sacrificed 24 hours after dosing to assess mCherry expression on T-cell subsets in the blood. Data is reported as median fluorescence intensity (MFI) of mCherry+ T cells (CD3+ cells) in the blood and spleen, respectively.

### Affinity-Capture Self-Interaction Nanoparticle Spectroscopy (AC-SINS)

Gold nanoparticles were coated with capturing anti-human IgG Fc and with polyclonal nonspecific antibody. The anti-CD8α antibodies were then incubated with the particles for 2 h and the wavelengths of maximum absorbance were measured using a multimode microplate reader. The wavelength difference (Δλₘₐₓ) from the PBS sample were then calculated to determine the affinity-capture self-interaction nanoparticle spectroscopy (AC-SINS) score of each antibody. Alirocumab and the NEI variant of bococizumab were used as control antibodies with low propensity to self-interact, and bococizumab was used as a highly self-interacting antibody control in the AC-SINS assessment. Antibodies with Δλₘₐₓ values below a threshold of 5 were considered to have low propensity to self-interact and aggregate.

Tₘ and T_{agg} Determinations

Thermal stabilities of the anti-CD8α antibodies were assessed by incorporating intrinsic fluorescence and static light scattering (SLS) analyses on the Uncle instrument (Unchained Labs). The antibodies were diluted to 1.5 mg/mL, and analysis was performed on 9 μL of sample loaded in triplicate into the UNI sample holder. Thermal melt profiles were obtained using a linear heating ramp of 0.3° C./minute between 20° C. and 95° C. Thermal melting mid-point (Tₘ) was determined by the ratio of intrinsic fluorescence at 350/330 nm using differential scanning fluorimetry (DSF) and thermal aggregation (T_{agg}) was determined by SLS through UV acquisition at 266 nm. Data were collected and analyzed concurrently with Uncle software and Tₘ and T_{agg} values were directly exported.

Polyreactive ELISA Assay to Insulin and dsDNA

Insulin (5 g/mL; Sigma) and dsDNA (1 g/mL; Sigma) were coated onto 96-well high-binding ELISA microplates (Corning) at 50 μL per well overnight at 4° C. Plates were blocked with PBS with 0.5% BSA (blocking buffer) at room temperature (RT) for 1 h, followed by three washes with PBST (PBS with 0.1% Tween-20). 50 μL of 100 nM antibody in blocking buffer was added to each well and incubated at RT for 1 h, followed by six washes with 100 μL of PBS. 50 μL of diluted anti-human IgG-horseradish peroxidase (HRP) conjugate (Jackson ImmunoResearch) was added to the wells and incubated for 1 h followed by another six washes. Finally, 50 μL of 3, 3′, 5, 5′-Tetramethylbenzidine (TMB) substrate (Fisher Scientific) was added to each well to develop the detection signal. The reactions were stopped by adding 50 μL of 2 M sulfuric acid to each well. The absorbance was measured at 450 nm using a multimode microplate reader. Ustekinumab as the control antibody with low polyreactivity and bococizumab as a highly polyreactive antibody control were also included in the polyreactive ELISA assessment.

### Baculovirus Particle (BVP) Polyreactive Assay

50 μL baculovirus particles (BVP) stock (Curia) was diluted with an equal volume of 50 mM sodium carbonate pH 9.6 per well and incubated on 96-well high binding ELISA plates (Corning) at 4° C. overnight. Unbound BVPs were aspirated from the wells after overnight incubation. Plates were blocked with PBS containing 2% BSA (blocking buffer) at room temperature (RT) for 1 h, followed by three washes with PBS. Antibody solutions in blocking buffer at 4 different concentrations 150, 50, 16.7, and 5.6 g/mL were added in triplicates and incubated at RT for 1 h, followed by six washes with 100 μL of PBS. 50 μL of diluted anti-human IgG-HRP conjugate (Jackson ImmunoResearch) was added to the wells and incubated for 1 h followed by another six washes. Finally, 50 μL of TMB substrate (Fisher Scientific) was added to each well to develop the detection signal. The reactions were stopped by adding 50 μL of 2 M sulfuric acid to each well. Human IgG1 poly-specificity control antibody (#H1308; Medna Scientific) and human IgG4 isotype control antibody (#H1304; Medna Scientific) were also included in the BVP polyreactive assessment as controls. Absorbance was measured at 450 nm using a multimode microplate reader, and BVP scores were determined by normalizing the absorbance by control wells with no test antibody. Antibodies with BVP scores above 5 as the threshold were considered to be polyspecific.

### Library Screen to Confirm CD8α Specificity of Anti-CD8α Binders

Library Screen: Human cell membrane receptor proteome array binding assays were performed at Retrogenix (Charles River Laboratories). A pool containing 2 g/mL each of test antibodies CBD1017ch (chimeric antibody), CBD1033, CBD1035 and CBD1039 was screened for binding against fixed HEK293 cells expressing duplicate 6,105 human plasma membrane proteins, secreted and cell surface tethered human secreted proteins plus 400 human heterodimers arrayed across cell microarray slide sets (n=2 per slide set). All transfection efficiencies exceeded the minimum threshold. An AlexaFluor647 anti-hIgG Fc detection antibody was used. In total, 11 library interaction hits (duplicate spots) were identified by analyzing fluorescence (AF647 and ZsGreen1) on ImageQuant.

Confirmation Screen: Vectors encoding all 11 interactors identified in the library screen, and control vector encoding CD20, in duplicate, plus a control vector encoding EGFR in quadruplicate, were spotted on new slides and used to reverse transfect human HEK293 cells as before. All transfection efficiencies exceeded the minimum threshold. Identical fixed slides were treated with either 2 μg/mL of CBD1017ch, 2 μg/mL of CBD1033, 2 μg/mL of CBD1035, 2 μg/mL of CBD1039,1 μg/mL of Rituximab biosimilar (positive control), or no test molecule (secondary only; negative control) (n=2 slide per treatment). Slides were analyzed as above. Interactions were categorized as specific, or non-specific (i.e. interaction also observed with either a positive control or a negative control).

### High pH Stress Study on Deamidation

Accelerated High pH stress: Samples from chimeric anti-CD8α and the top 3 humanized clones CBD1033, CBD1035, and CBD1039 whole antibody were buffer exchanged to 50 mM Tris, 10 mM EDTA, pH 8.5, followed by incubation at 40° C. for 7 days. These high pH-stressed samples were compared to no stress samples, which were stored in the original buffer at pH 7.2, 4° C. for 7 days.

Peptide mapping: Samples were first denatured and treated with 5 mM dithiothreitol (DTT) and 10 mM Iodoacetamide (IAM) for 60 min at 4° C., followed by buffer exchange to 1M Urea in 0.1M Tris pH 7.4. Next, trypsin was added to the sample at enzyme:protein ratio 1:18 (w/w), and the mixture was incubated for 4 hours at room temperature. At the end of the incubation, the digestion was stopped by the addition of formic acid (1% v/v). The digested sample were then injected to LC-QTOF (Waters Acquity UPLC and Xevo G3 MS) that connected to XSelect CSH C18 2.1×150, 2.5 um, 130A (Part: 186006727). Mobile Phase A was 0.1% Difluoroacetic acid (DFA) in Water; Mobile Phase B was 0.1% DFA in acetonitrile. Flow Rate was set to 200 uL/min, and temperature was set to 40° C. Data were then analyzed by UNIFI Scientific Information System (Version 3.1.0.16) to determine all post-translational modifications on the antibody.

### Epitope Mapping by Crosslinking Reaction and Mass Spectrometry

The CBD1033 Fab molecule and human CD8αα homodimer protein (Acro Biosystems) were mixed with each other at a molar ratio of 1:2, and then a bis(sulfosuccinimidyl)suberate (BS3) cross-linker (CovalX) was added to cross-link the formed protein complex. The mixture was allowed to react at room temperature for 3 hours to form a Fab-antigen complex. The molecular weight of the reaction product was analyzed using an Autoflex II MALDI ToF/ToF mass spectrometer (Bruker) equipped with a HM4 interaction module (CovalX).

In order to identify specific sites of interaction between the Fab and the CD8ααhomodimer protein cross-linked peptide fragments were produced. The protein complex was incubated with bis(sulfosuccinimidyl)suberate (BS3) cross-linkers and subjected to multi-enzymatic cleavage. An equimolar mixture solution of deuterated and non-deuterated BS3 cross-linkers was added to a 1:2 mixture of CBD1033 Fab molecule and human CD8αα homodimer protein and subjected to a cross-linking reaction at room temperature for 3 hours. The reaction product was reduced, alkylated and digested separately with one of the four proteases: trypsin, chymotrypsin, elastase, and thermolysin. The produced fragments were analyzed by an Ultimate 3000-RSLC nano-liquid chromatography system (Thermo Scientific) and the Orbitrap Fusion LUMOS mass spectrometer (Thermo Scientific). The obtained mass spectrometry data were analyzed by Xquest and Stavrox softwares in order to detect the cross-linked peptide interface between the Fab molecule and human CD8αα homodimer. Crosslinked amino-acid positions were then mapped onto an existing structural model of human CD8αα homodimer (PDB: 1CD8).

### Competition Binning by BLI

Competition binding experiment were performed on the Gator Plus BLI system (Gator Bio). The kinetic buffer containing PBS plus 0.01% Tween 20 and 0.02% BSA was used for calculating the baseline. Binding competition was assessed on a panel of anti-CD8 antibodies consisting of CBD1033, TRX2, YTC182.20 (Novus Biologicals), OKT8 (BioXCell), SK1, and HIT8a (Biolegend). Streptavidin-immobilized SA-XT sensors (#160029, Gator Bio) were prehydrated in the kinetic buffer and loaded with 100 mM biotinylated CD8α recombinant protein (CDA-H82E3, Acro Biosystems) at spin speed 400 rpm to a threshold response of 4 nm. The sensors were further incubated twice in the kinetic buffer for 60 s to achieve the steady baseline measurement prior to binding competition. The antigen-loaded sensors were incubated in 200 nM of the first antibody in the antibody panel and allowed to reach saturation for at least 300 s to ensure occupation of all available binding sites. After another 60 s baseline step, the sensors were then incubated in 100 nM of the second antibody in the panel for 300s. The binding competition was analyzed using the GatorOne software (version v2.10). A heat map of the competition matrix was then generated based upon the threshold settings. Binding responses less than 0.6 nm were determined to be blocking, between 0.6 and 0.7 nm were intermediate blocking, while above 0.7 nm were not blocking. A blocking network plot was also generated by the software from the heat map. Epitope bins and their inter-connectivities are displayed in the plot as antibodies grouped with the same blocking profile compared to all others in the panel.

### Example 1: Generating Humanized Anti-CD8α Binders and Testing their Binding Affinities

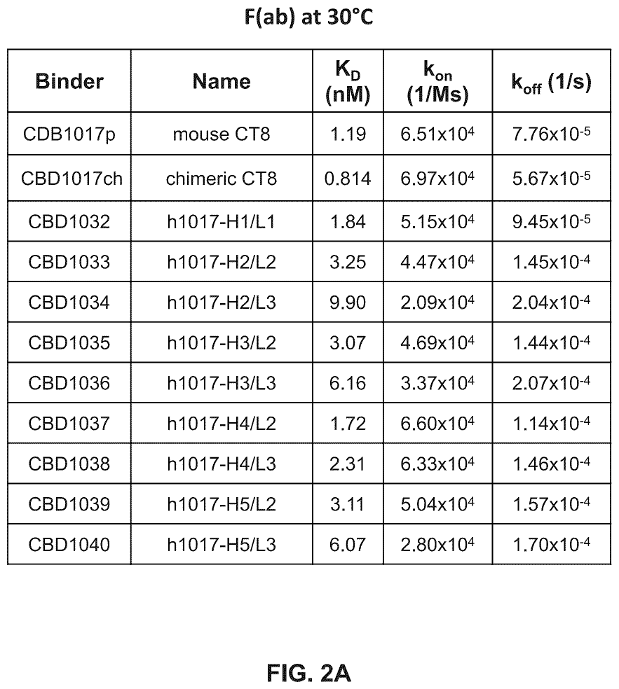

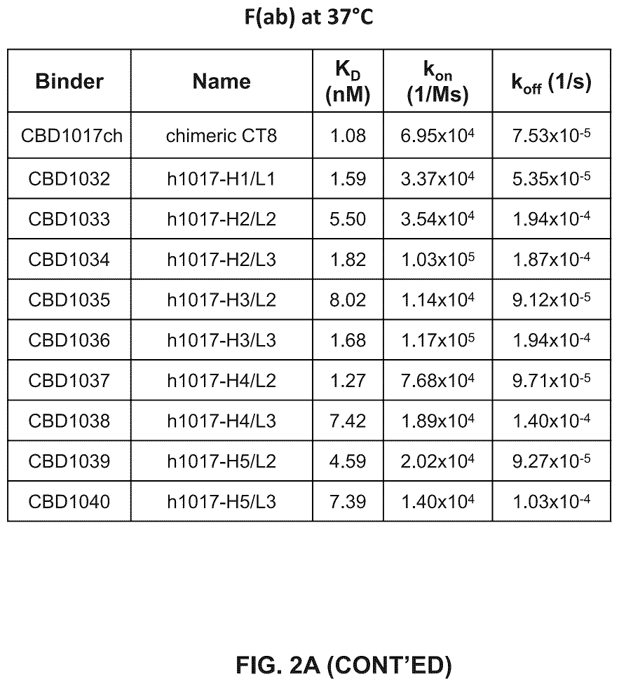

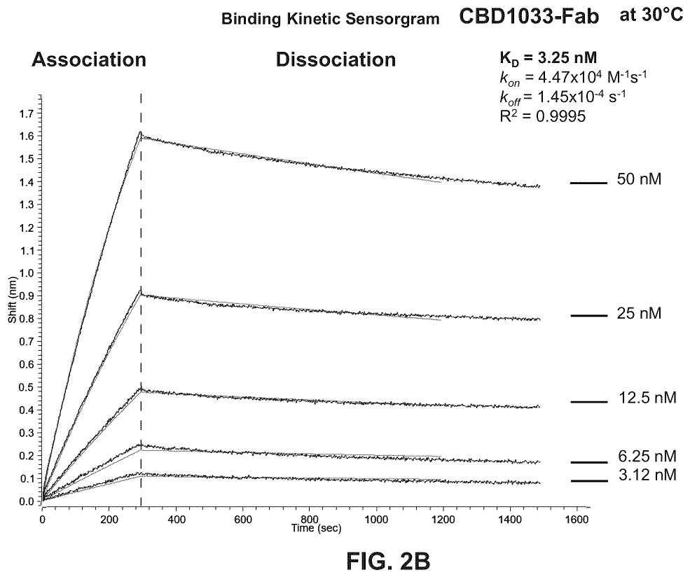

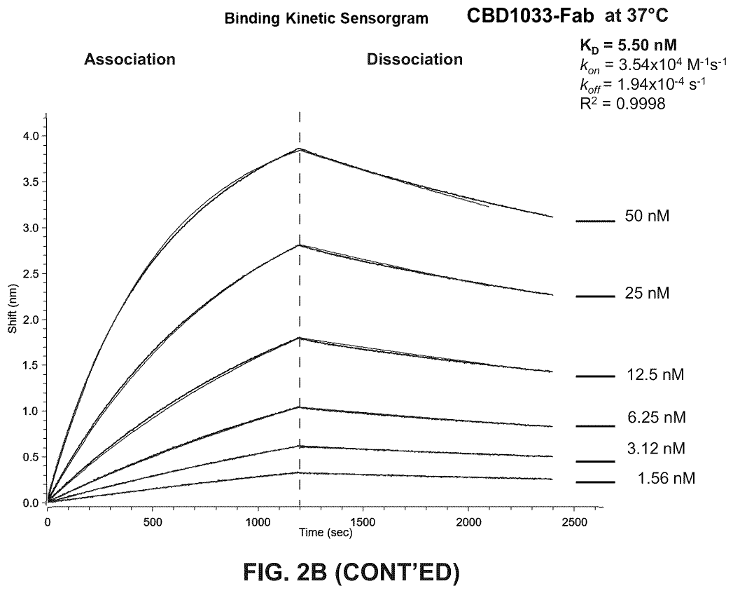

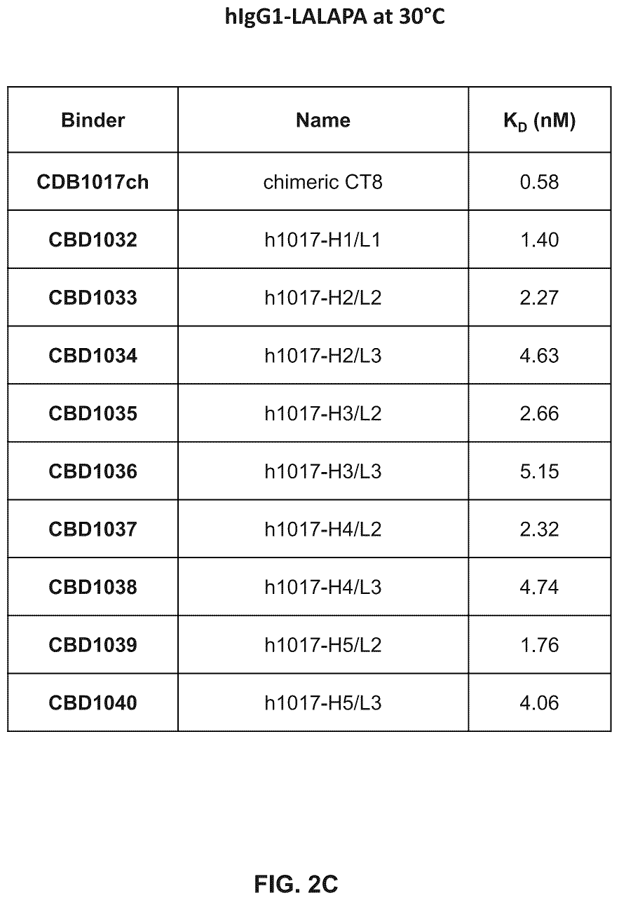

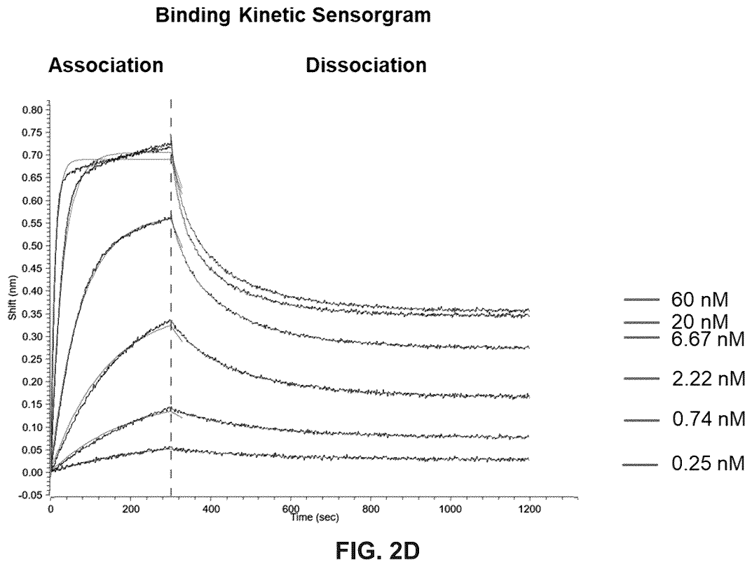

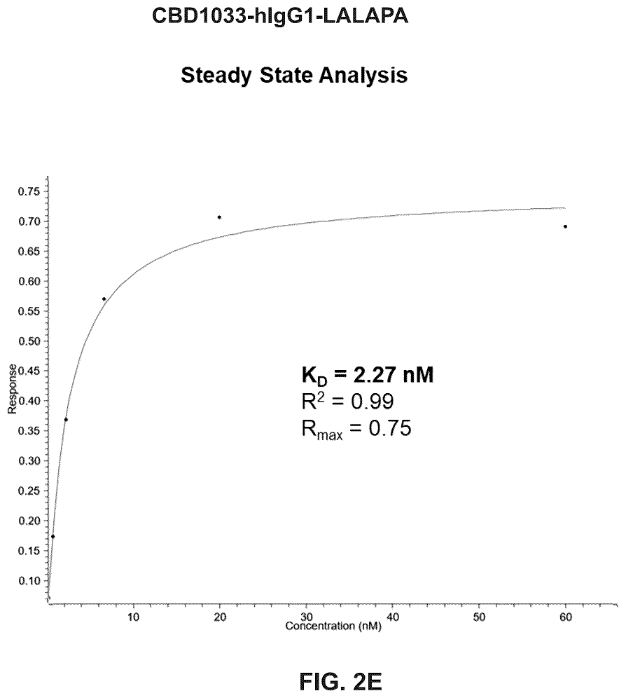

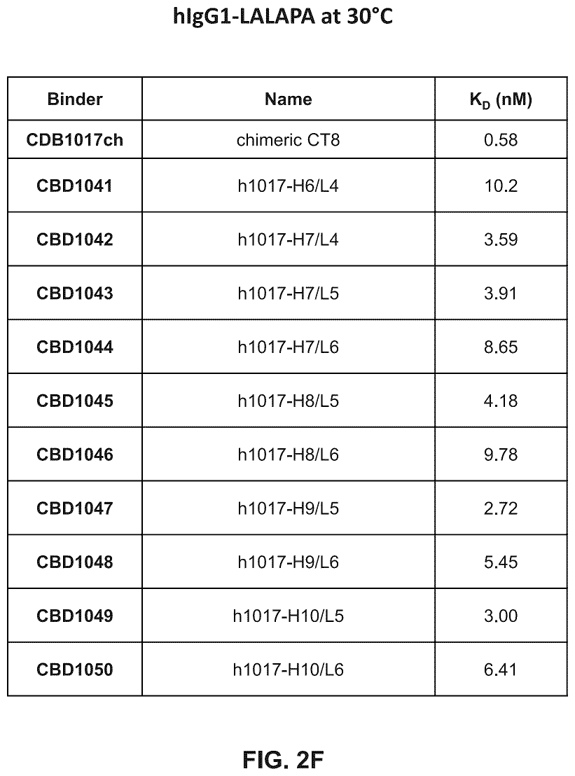

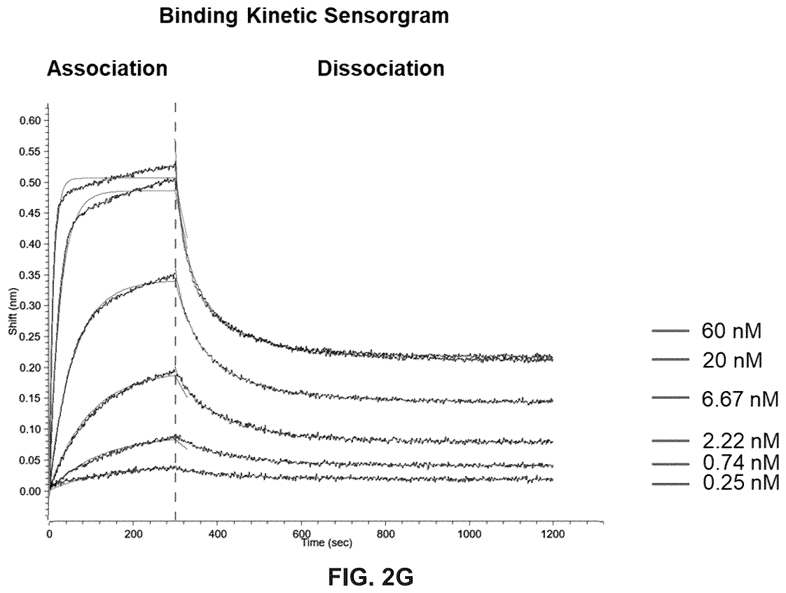

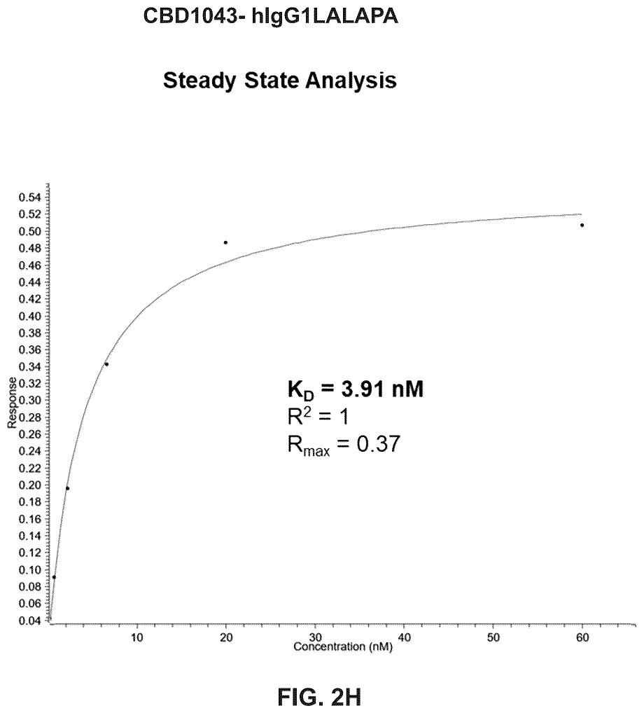

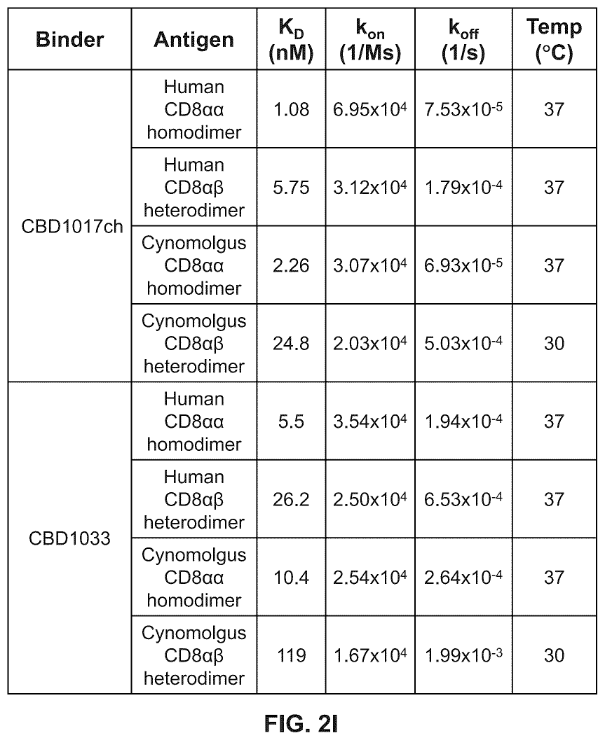

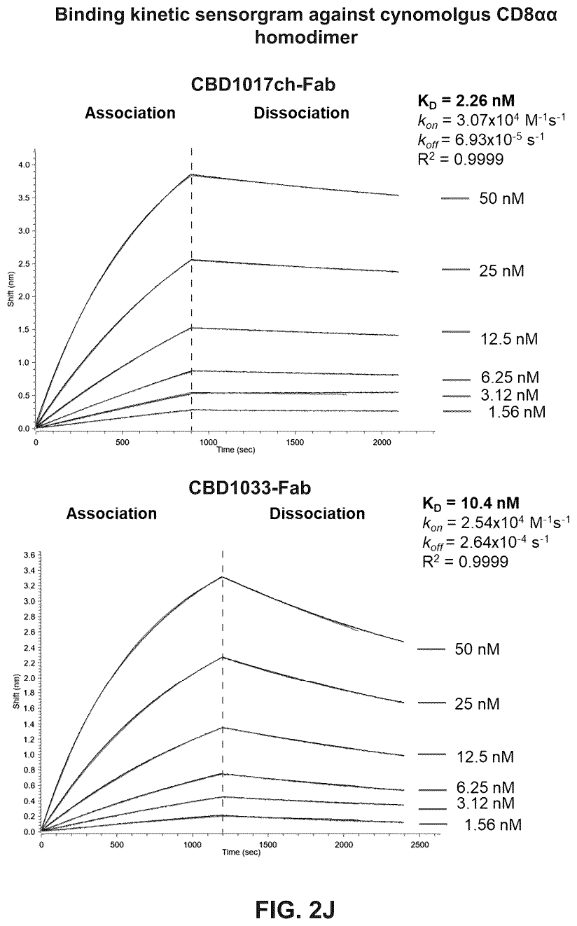

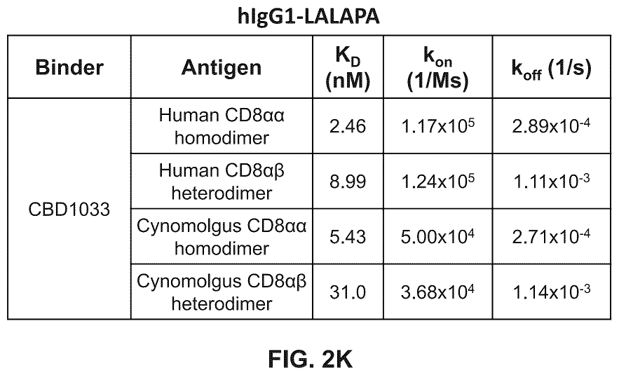

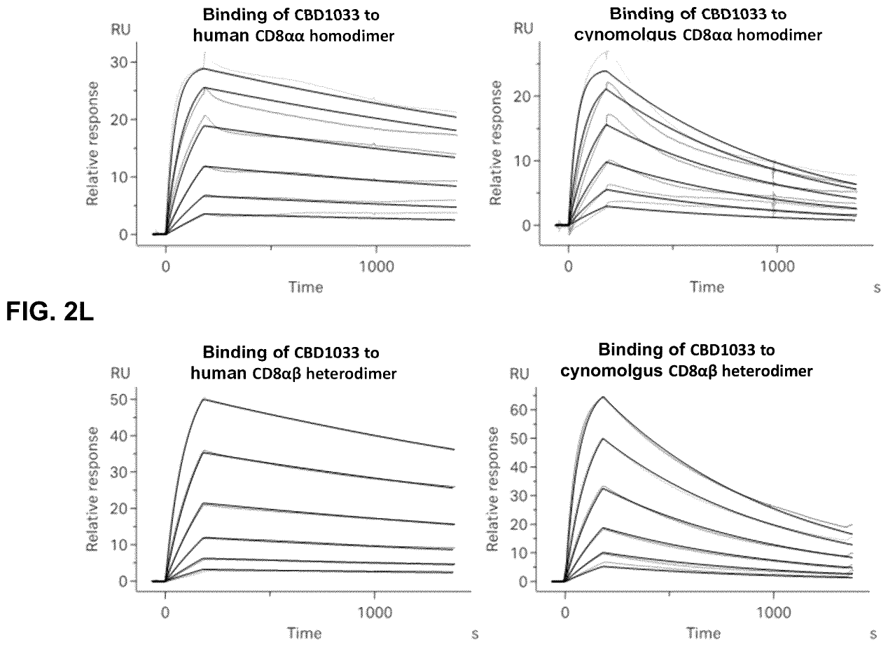

CDRs from mouse anti-human CD8α antibody CT8 were incorporated into human VH and VL germlines VH1-46*01 and VK1-39*01 respectively, and modified versions of VH1-18*01 and VK3D-11*02, respectively. One or more mouse back mutations were used in the framework regions of each humanized sequence, which have the potential to better maintain binding affinity or other properties of the parental antibody, as shown in FIG. 1A to FIG. 1D. The variable regions shown in FIG. 1A to FIG. 1D were paired in various combinations (anti-CD8α binders), as indicated in FIG. 2A, expressed as F(ab) fragments, and their binding constants (K_{D})determined by biolayer interferometry kinetic assays. All tested anti-CD8α binders showed acceptable K_{D} compared to the parental mouse (CBD1017p) and chimeric (CBD1017ch) anti-CD8 F(ab) fragments, with K_{D} determined by kinetic analysis and having values ranging from 1.72 to 9.90 nM, at both 30° C. and physiological temperature 37° C. (FIG. 2A and FIG. 2B). In addition, the anti-CD8α binders based on VH1-46/VK1-39 and VH1-18/VK3D-11 pair of germlines were also expressed as whole antibodies in human IgG1 isotype with Fc-silencing mutations L234A, L235A and P329A (hIgG1-LALAPA; SEQ ID NO: 43) to determine their K_{D} including by steady state assay (FIG. 2C and FIG. 2F). (Note that this amino acid numbering is commonly used in the literature, however, in UniProt entry P0DOX5, the numbering would be L236A, L237A, and P331A. The hIgG1 sequence used in constructing the whole antibodies also differed from UniProt entry P0DOX5 at two additional positions D358E and L360M, according to the UniProt numbering. These positions are commonly referred to as 356 and 358 in the scientific literature. The E and M allotype is considered potentially less immunogenic.) Although all tested binders showed acceptable K_{D}, as a group the binders based on VH1-46/VK1-39 germlines showed better binding capacity compared to binders based on VH1-18/VK3D-11 germlines even though there is substantial amino acid sequence overlap between the two groups. In general, the latter group required more mouse back mutation to achieve binding and so had greater potential to be immunogenic. Accordingly, only the former binders were used in the subsequent experiments.

The above results showed binding affinities to CD8αα homodimer but it was known that human T cells primarily express CD8αβ heterodimer. Thus, binding affinities of CBD1033 and its parental CBD1017ch in Fab or whole antibody versions were measured by BLI or SPR assays respectively, for human and cynomolgus macaque CD8αα homodimer and CD8aP heterodimer (FIG. 2I through FIG. 2L). All versions showed acceptable binding affinities, indicating that the same antibody to be used in human patients can be used in the experimental cynomolgus macaque model (FIG. 2I through FIG. 2L)..

The anti-CD8α binders were also expressed as whole antibodies in human IgG1 isotype with Fc-silencing mutations L234A, L235A and P329A (hIgG1-LALAPA; SEQ ID NO: 43) and used to target CD8⁺ cells to determine an EC₅₀ in a bivalent assay assessing their ability to bind CD8-overexpressing HEK cells. All of these anti-CD8α binders showed EC₅₀ comparable with each other and the parental anti-CD8 antibody, with values ranging from 1.75 to 4.39 nM (FIG. 3A and FIG. 3B). Humanized anti-CD8α CBD1033 Fc silenced human IgG1 antibody also showed binding to CD8 expressing lymphoma T cell lines with comparable EC₅₀ of 312 ng/ml (2.13 nM) and 274 ng/ml (1.87 nM) on SupT1 and HPB-ALL, respectively (FIG. 6). In summary, these experiments showed that the humanized anti-CD8α antigen binding domains of this disclosure retained affinity comparable to the parental antibody. The data further demonstrated that the humanized antigen binding domains in the context of bivalent antibody all provided comparable EC₅₀ for binding to cell surface CD8 over a range of expression levels.

### Example 2: Humanized Anti-CD8α Binders were Functional as Targeting Moieties when Conjugated to Lipid Nanoparticles (tLNP)

Humanized anti-CD8 antibodies as disclosed herein can be used to guide lipid nanoparticles containing therapeutic cargos (e.g., mRNA, small molecules) specifically to CD8α-expressing cells such as T cells. CD8α-targeted lipid nanoparticles encapsulating mRNA-encoding mCherry were generated with anti-CD8α antibodies of this disclosure as targeting moieties and used to transfect primary human T cells from different donors. The targeting moieties were in the form of IgG1 antibodies with silenced Fc incorporating the LALAPA mutations. At all amounts of tLNP, the number of mCherry positive cells (transfection efficiency) was at least 70% and was similar from one humanized anti-CD8 antibody to the next and to CBD1017ch a chimeric antibody comprising the variable regions of the donor antibody. Transfection efficiency was noticeably greater for the CD8-targeted tLNP than was obtained with a control CD5-targeted tLNP decorated with CBD1011v3 whole antibody (FIG. 4A to FIG. 4B). Expression level, observed as mCherry fluorescence intensity, was more variable but still generally comparable for the tLNP targeted with the humanized anti-CD8 whole antibodies if somewhat less than that obtained with tLNP decorated with chimeric anti-CD8α antibody (FIG. 4C to FIG. 4D). The expression level obtained with the CD5-targeted control tLNP was poor by comparison. tLNP targeted with CBD1033 whole antibody showed superior expression levels compared to tLNP decorated with a humanized anti-CD8 binder based on mouse anti-CD8 antibody from a different clone (hOKT8 variant 1 with VL and VH sequence as shown in Table 16 as SEQ ID NO: 229 and 230 respectively; taken from U.S. Ser. No. 11/254,744B2) (FIG. 5A to FIG. 5C). Taken together, these data showed that humanized anti-CD8α binders had the potential to be effective in tLNP therapeutic application and superior to another known humanized anti-CD8 antibody.

### Example 3: Cross-Species Binding Activity of Humanized Anti-CD8α Binders

The donor antibody had been previously reported to bind CD8α from human, cynomolgus macaque, and rhesus macaque. It is an advantageous property for an antibody to maintain its binding affinity in both human and other species, so that experimental data in non-human species can be obtained and be reliably translatable to humans. All humanized anti-CD8α binders tested as whole antibody showed comparable transfection rates and mCherry expression levels when used as the tLNP targeting moiety for transfection of cynomolgus macaque PBMC (FIG. 7A to FIG. 7B). The CD8-targeting moieties were in the form of IgG1 antibodies with silenced Fc incorporating the LALAPA mutations. The CD5-targeted control tLNP (CBD1011v3 whole antibody as the targeting moiety) produced substantially less transfection and a lower level of expression in comparison to the CD8-targeted tLNP. The performance of hOKT8-targeted tLNP was also poor by both measures as compared to tLNP bearing a CT8-derived binding moieyy (FIG. 7A to FIG. 7B). Cetuximab (anti-EGFR) as the tLNP targeting moiety was used as a negative control.

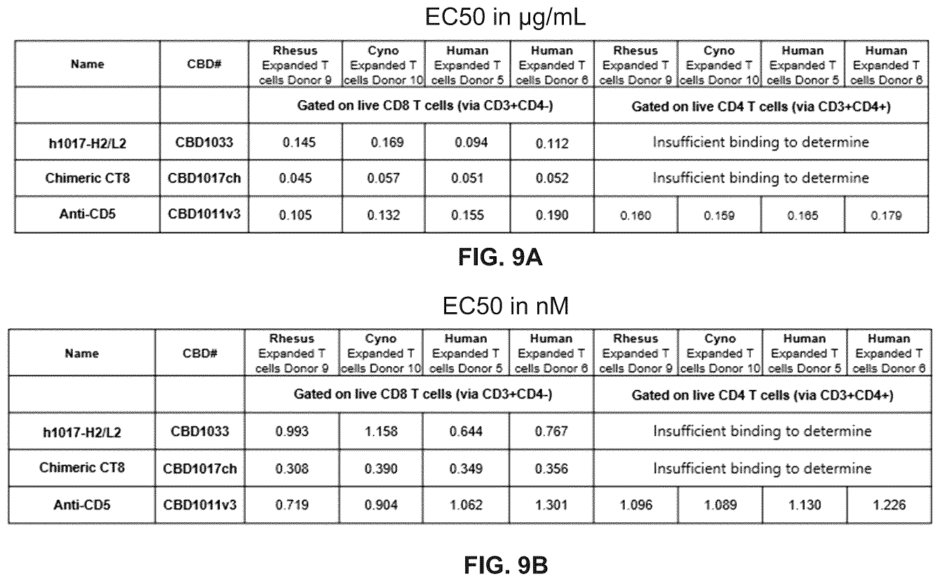

Binding EC₅₀ were determined on expanded T cells from human, cynomolgus macaque, and rhesus macaque for the chimeric CT8 antibody, CBD1017ch, and humanized CBD1033 whole antibody as well as the anti-CD5 CBD1011v3 whole antibody (FIG. 8A to FIG. 9B). CBD1033 whole antibody exhibited a similar EC₅₀ on both the human and non-human primate T cells, though somewhat less than the chimeric antibody with parental CD8 binding domain. Moreover, binding was specific to CD8 T cells and not CD4 T cells (FIG. 9A and FIG. 9B). As expected, the anti-CD5 antibody displayed similar EC₅₀ on both CD8+ and CD4+ T cells. These results indicated that the anti-CD8α binding antibodies disclosed herein all maintained advantageous cross-species binding affinities.

### Example 4: Anti-CD8α Binder in tLNP was Specific and Functional In Vivo

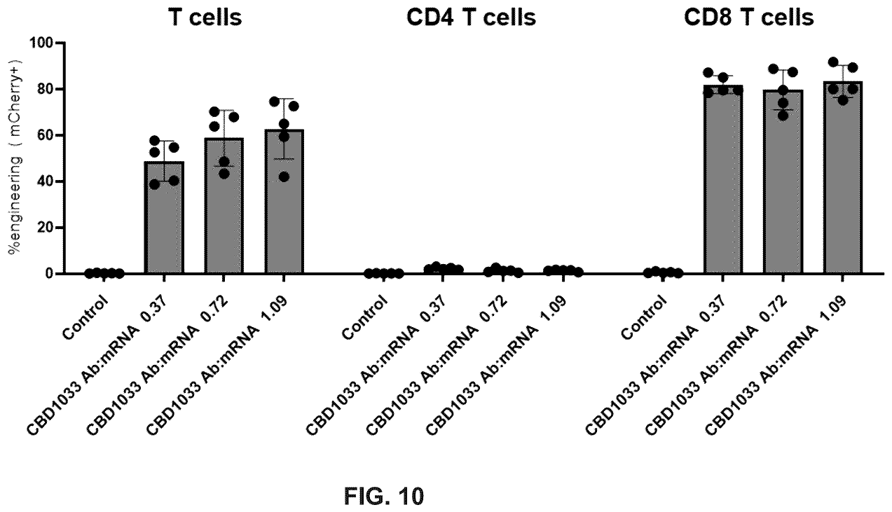

CBD1033 Fc silenced human IgG1 whole antibody, with the LALAPA mutations, was used as the targeting moiety on tLNP at various Ab:mRNA ratios (w/w) to deliver mRNA encoded mCherry to human CD8-expressing cells in NSG mice engrafted with human PBMCs. Ab:mRNA ratios (w/w) of 0.2-1 has previously been found to work well for whole antibody. In all antibody:mRNA densities tested, mCherry expression was detected in T cells (CD3+) and CD8 T cells (CD3+CD4−CD8+), but not in CD4 T cells (CD3+CD4+CD8−) (FIG. 10). The data demonstrated that humanized CBD1033 is effective in specifically guiding tLNP to CD8α-expressing targets in vivo.

### Example 5: Biophysical Properties of Anti-CD8α Binders

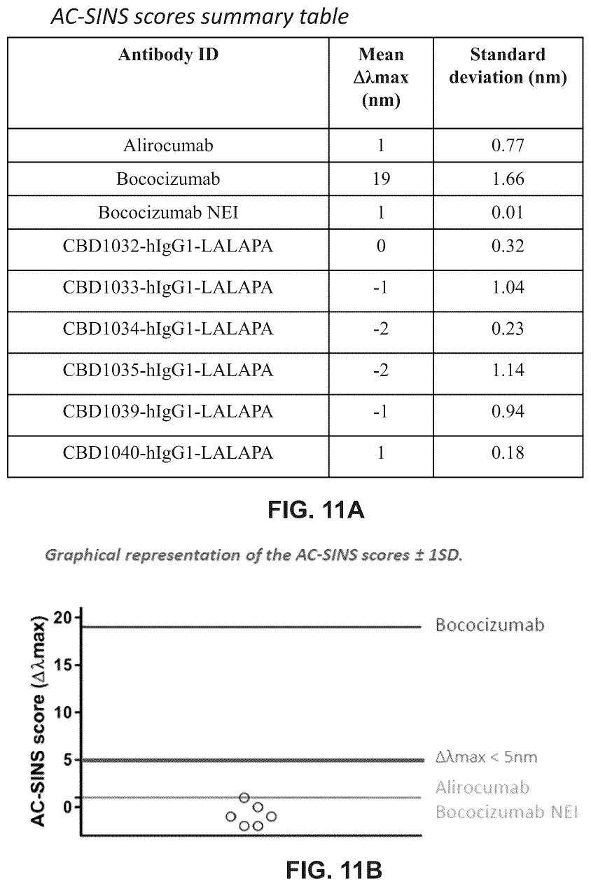

A set of experiments was carried out to characterize the biophysical properties of humanized anti-CD8α binder whole antibodies, important in determining their capacity for clinical development. Several studies in the art have suggested that certain monoclonal antibodies can interact non-specifically with themselves and other serum proteins leading to antibody aggregation, off-target effects, immunogenicity, and fast antibody clearance (Kelly et al., 2015, Mabs 7(4):770-777). Indeed, self-aggregation and polyreactivity are highly correlated with monoclonal antibody product failure (Dyson et al., 2020, MAbs 12(1):1829335). An affinity-capture self-interaction nanoparticle spectroscopy (AC-SINS) assay has been shown to predict viscosity, solubility issues, and in vivo clearance, which are helpful in predicting the antibody's potential development profile. FIG. 11A and FIG. 11B showed that 6 anti-CD8α binders (CBD1032-CBD1035 and CBD1039-CBD1040) had AC-SINS scores comparable to Alirocumab and Bococizumab NEI, which are known to have low propensity to self-interact. Bococizumab was used as a positive control of self-interaction. Although all six of the tested humanized CD8α binders had a low propensity for self-aggregation there was no reason to believe this would be the case apriori so this result, while fortunate, was not predictable.

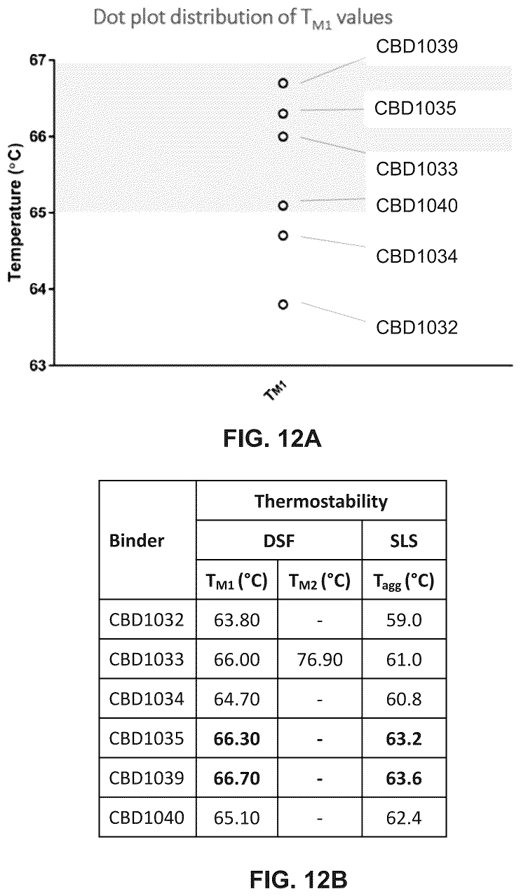

The same six humanized anti-CD8α binders were assessed for melting temperature (Tₘ) and thermal aggregation temperature (T_{agg}) as measured by differential scanning fluorimetry (DSF) and static light scattering (SLS), respectively. The threshold between poor developability and reasonable developability is generally considered to be 65° C. Of the six humanized CD8α binders tested (as whole antibody) CBD1033, CBD1035, CBD1039, and CBD1040 met this threshold but CBD1032 and CBD1034 fell below (FIG. 12A and FIG. 12B). The T_{agg} values were considered acceptable for all six humanized antibodies tested.

### Example 6: Comprehensive Non-Specific Binding Analysis of Humanized Anti-CD8A Binders

Many anti-CD8 antibodies have been developed for in vitro analytic and diagnostic uses where they may be exposed to a limited number and different array of antigens as compared to in vivo use. In developing an anti-CD8 antibody for use as a targeting moiety for a in vivo human therapeutic product it becomes more important that the antibody not recognize other antigens that can be encountered in the human body and that the humanization process not introduce such reactivity. For this reason, several of the disclosed humanized anti-CD8α binders were tested by three methods of increasing stringency for assessing polyreactivity.

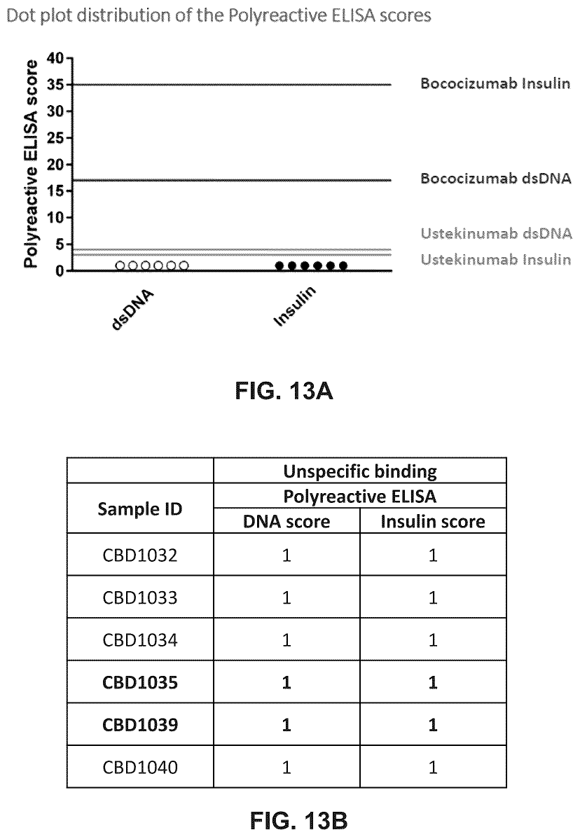

The first assessment tested for low non-specific binding to dsDNA and insulin by ELISA. CBD1032 to CBD1035 and CBD1039 to CBD1040 Fc silenced IgG1 whole antibodies, with the LALAPA mutations, were tested and displayed very low reactivity to the antigens with scores of 1 on the assay's polyreactivity scale, less than the low-reactive control ustekinumab (FIG. 13A and FIG. 13B). Thus, by this standard, none of the tested antibodies were polyreactive.

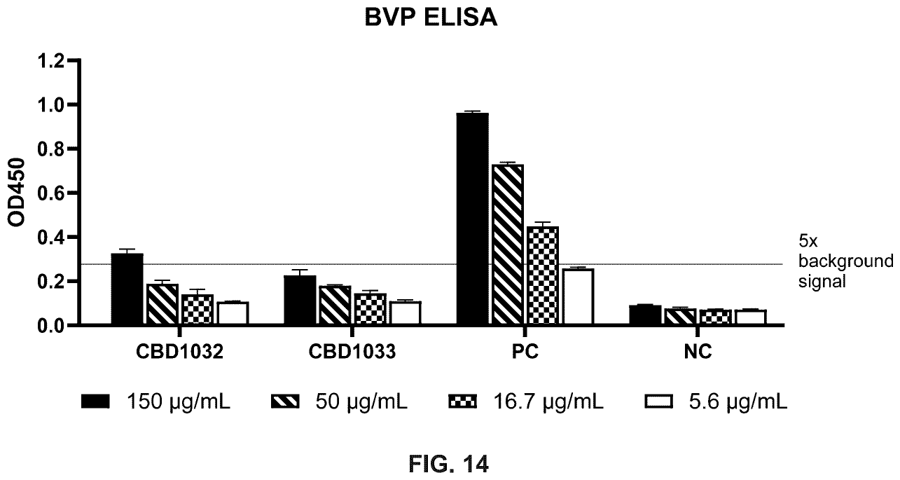

The second assessment tested for binding to baculovirus particles in an ELISA. Budded BV virions are stable nanoparticles that mimic infected cell surfaces, presenting a complex mixture of phospholipid, carbohydrate, glycoproteins, extracellular matrix, and nucleic acids, as well as the viral capsid, and binding to the particles has been found to be predictive of fast clearance of antibodies in cynomolgus macaques and humans (Hotzel et al., 2012, MAbs 4(6):753-760). CBD1032 antibody showed a small non-specific binding at high dose 150 μg/mL, whereas CBD1033 antibody did not (FIG. 14). Thus, by this standard, CBD1032 is polyreactive, if marginally so.

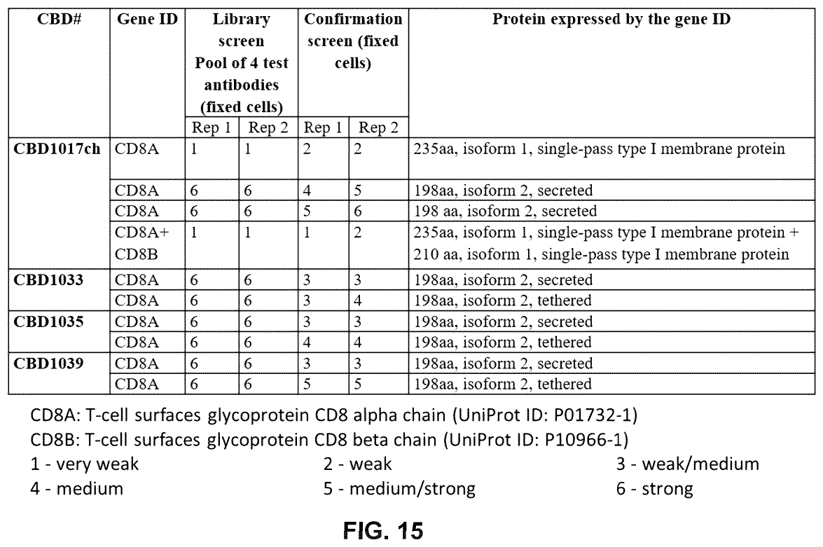

Finally, a more comprehensive test assessing binding to over 6000 human antigens including membrane proteins, heterodimers, secreted proteins, and tethered secreted proteins was performed. Chimeric CT8 (CBD1017ch) antibody showed significant specific interactions with the primary target CD8α, with 3 forms of the homodimer (plasma membrane bound single pass type I membrane protein, 198 amino acid secreted form, and a surface-tethered form of the soluble form), and with CD8α (Isoform 1) in combination with CD80 (the heterodimer) (FIG. 15). CBD1033, CBD1035, and CBD1039 all showed significant specific interactions with the primary target CD8α, with both the tethered secreted and the secreted forms of the soluble form. No additional significant specific interactions were identified for any of the test antibodies, indicating high specificity for the primary target. Thus, by this assessment CBD1033, CBD1035, and CBD1039 are not polyreactive and preserved the lack of cross-reactivity of the parental/donor antigen binding domain.

### Example 7: Assessment of Post-Translational Modifications

The amino acid asparagine (N) can be prone to deamidation depending on its environment within the protein and the conditions to which the protein is exposed. Aspartic acid, whether a native residue or generated by deamidation of asparagine, can form isoaspartic acid. This can be problematic for product uniformity and stability. More importantly, deamidation and isoaspartate formation can have functional consequences impacting antibody affinity especially if the asparagine is located within a CDR. CT8 has several N residues in CDRs and VH N55 in HCDR2 was particularly concerning. Therefore, the susceptibility of VH N55 to deamidation, the effect on binding affinity, and the acceptability of alternative amino acids was assessed.

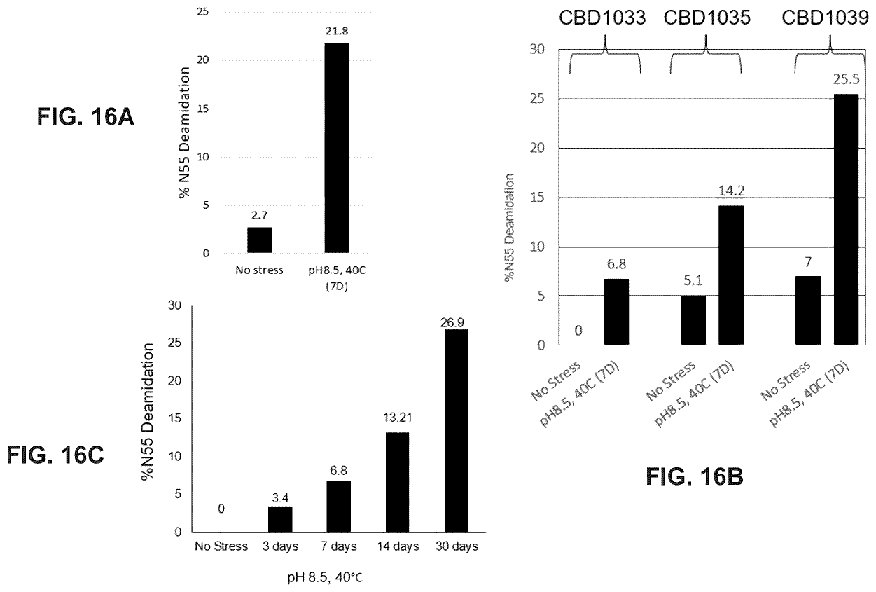

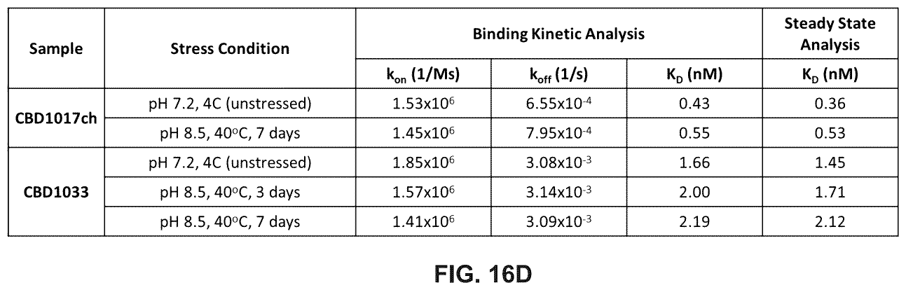

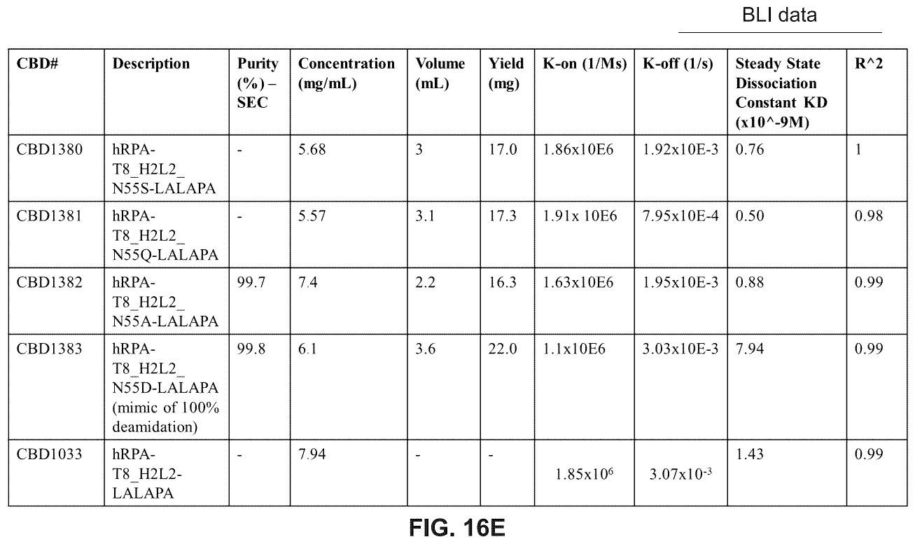

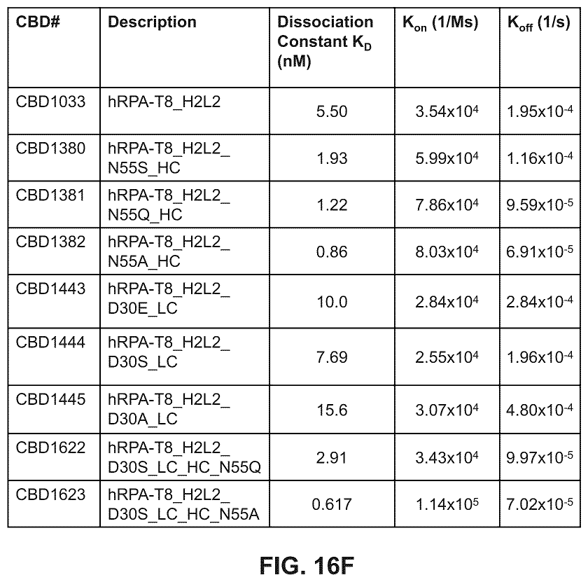

The chimeric antibody, CBD1017ch, was exposed to high stress conditions of pH 8.5 at 40° C. for seven days and compared to unstressed antibody (pH 7.2 at 4° C.). By peptide mapping it was determined that there was substantial VH N55 deamidation under high stress conditions and even a small amount of deamidation in the unstressed antibody (FIG. 16A). CBD1017ch and CBD1033 Fc silenced IgG1 whole antibody were then subjected to high stress and unstressed conditions for up to 7 days and affinity measurements were made, revealing a slight diminution of affinity in the stressed versus the unstressed samples. (FIG. 16D). Three humanized anti-CD8 binders CBD1033, CBD1035, and CBD1039 (in the form of Fc silenced IgG1 whole antibodies with the LALAPA mutations) were tested for deamidation under stressed and unstressed conditions. While all three of these antibodies exhibited varying degrees of VH N55 deamidation under stressed conditions, the CBD1033 antibody exhibited the least and there was no detectable deamidation detected for this antibody in the unstressed condition (FIG. 16B, see also FIG. 16C). Accordingly, if processing conditions avoiding high stress are used, CBD1033 could be manufactured without inducing appreciable deamidation. Alternatively or additionally, modifying other parameters such as choice of buffer or inclusion of stabilizing additives can reduce the risk of deamidation. Additionally, although CBD1035 and CBD1039 antibodies exhibited some deamidation under unstressed conditions, the amount was comparable to CBD1033 under stressed conditions so that only a slight and functionally negligible decrease in affinity would be expected if they were processed in a manner avoiding high stress conditions.

Four VH N55 mutants were made on a CBD1033 background: N55D (CBD1383) to mimic the effect of 100% deamidation and three to see if N55 could be replaced with a stable residue while maintaining binding affinity, N55S (CBD1380), N55Q (CBD1381), and N55A (CBD1382). Based on kinetic rate K_{D} determined with biolayer interferometry (BLI) on whole antibodies, the VH N55D mutant, 100% deamidation mimic had its affinity reduced by about 5.5-fold as compared to CBD1033 in which the mutation was made. Each of the other N substitution mutants had their affinity improved in the neighborhood or 2-fold to 5-fold compared to CBD1033 (FIG. 16E and FIG. 16F).

These VH N55 mutants, and CBD1033, were used as the targeting moiety on tLNP encapsulating mCherry mRNA to assess the ability of these mutants to mediate transfection of human T cells. In two donors, tLNP decorated with CBD1033 and the N55Q, N55S, and N55A mutants obtained robust and very similar levels of mCherry expression. The deamidation mimic N55D mutant still obtained substantial mCherry expression, although reduced by about 20-25% as compared to CBD1033, which was statistically significant (FIG. 17A and FIG. 17B). Nonetheless, this indicates the maximum functional reduction that would be expected from VH N55 deamidation.

The D30 residue in VL-CDR1 could form isoaspartate, another potential liability and was mutated to E, S, or A in individual constructs. Three D30 mutants on VL-CDR1 and two double mutations on N55 and D30 were made on a CBD1033 background: D30E (CBD1443), D30S (CBD1444), D30A (CBD1445), D30S and N55Q (CBD1622), and D30S and N55A (CBD1623). Based on the K_{D} values determined with kinetic rate analysis by biolayer interferometry (BLI) on Fab fragments, all the D30 mutants had acceptable binding affinities, albeit slightly reduced relative to CBD1033. The double mutations variants had their affinities improved 2-fold to 5-fold compared to CBD1033 (FIG. 16F).

### Example 8: Humanized Anti-CD8α Binders in tLNP for CD8+ T Cells Transfection

Expression of mCherry reporter delivered as an mRNA payload by tLNPs was measured to access the efficiency of delivery by tLNPs decorated with anti-CD8α CBD1033 targeting moieties in various binding formats and expression level of the delivered payload. In addition, targeting moiety density on the LNPs nanoparticle, expressed as the ratio of targeting moiety to mRNA (w/w), was varied as set out in Table 15 for each of the tLNP groups indicated in FIG. 18A and FIG. 18B. There was some correlation of higher antibody density with higher expression level. Antibody:mRNA ratio (w/w) of less than 0.2 was associated with reduced mCherry expression level (Table 15 and FIG. 18B). When insufficient TCEP concentration was used (resulting in lower partial reduction of cysteine bonds and fewer thiols for conjugation), incomplete conjugation of available targeting moiety was observed, which led to lower antibody density on the tLNPs and reduced expression of the encapsulated mCherry mRNA (FIG. 18B, Groups 4 and 5). FIG. 18A shows that transfection efficiency was insensitive to these parameters or the antibody format used.

TABLE 15
Targeting Moiety Density of tLNPs

| LNP | Targeting Moiety:mRNA |
|---|---|
| Group No. | Ratio (w/w) |
| 1 | 0.26 |
| 2 | 0.27 |
| 3 | 0.38 |
| 4 | 0.11 |
| 5 | 0.17 |
| 6 | 0.24 |
| 7 | 0.30 |
| 8 | 0.25 |
| 9 | 0.40 |
| 10 | 0.39 |
| 11 | 0.13 |
| 12 | 0.21 |
| 13 | 0.39 |
| 14 | 0.30 |
| 15 | 0.46 |
| 16 | 0.36 |

The transfection rate was uniform across different binder formats (FIG. 18A) showing that tLNP decorated with CBD1033 using any of these formats as the targeting moiety, including as a full-length antibody, can successfully transfect cells with similar efficiency and that neither antibody density nor reducing condition within the tested range were critical parameters. mCherry expression level showed some variation though most of the tLNP preparations achieved an expression level at least as high as the CBD1033 Fc silenced IgG1 whole antibody positive control. While tLNP targeting moiety (e.g., antibody) density within the tested range did not appear to have a consistent effect on payload expression as some preparations with relatively low antibody density produced high levels of expression (see for example group 12 in FIG. 18B), the poorest payload expression levels were associated with low targeting moiety density (FIG. 18B, groups 4 and 5).

### Example 9: Generating Disulfide Engineered Anti-CD8α F(Ab′) and Testing their Binding Affinities

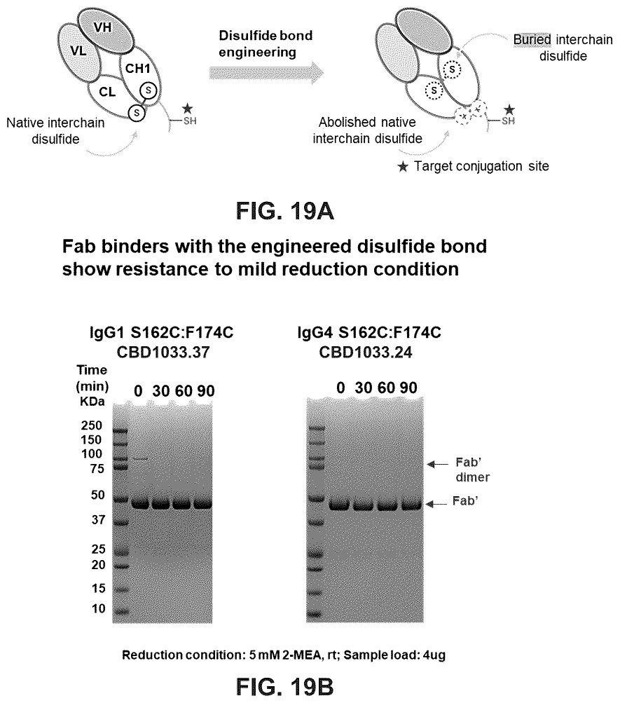

Humanized anti-CD8 F(ab′) analogs with either IgG1-derived or IgG4-derived constant regions, and C-Kappa domains contain a native disulfide bond formed by cysteine in position 233 of the hinge region (hinge-C233) in IgG1 or first heavy chain constant domain (CH1-C127) in IgG4 with cysteine in position 214 of kappa light chain constant domain (CK—C214). Several uses of Fab or F(ab′) analogs require the reduction of cysteine residues to facilitate chemical reactions with a free thiol group. However, a cysteine capping modification can occur due to disulfide bond formation between the engineered cysteine site for conjugation and thiol-containing metabolites cysteine or glutathione that are present in the expression medium during protein production. A chemical reduction step required to remove this modification also indiscriminately disrupts the above-mentioned native interchain disulfide bond. To increase resistance of the F(ab′) interchain disulfide bond to reduction and maintain the structural integrity of the molecule, the native disulfide bond was removed by mutating these cysteine residues to serine residues; and a new, less accessible disulfide bond was formed by mutations of CH1-F174 in IgG1 or IgG4 and CK—S162 to cysteine residues (FIG. 19A and FIG. 19B). This allows reduction of any disulfide that may form between two hinge region cysteine residues of two F(ab′) units (thus forming F(ab′)₂) without reducing the interchain disulfide bond between the constant domains of the heavy and light chains. The F(ab′) design also contained a C-terminal “CAA” (FIG. 19A) (see SEQ ID NOS: 85, 87, 90, 92, and 93) or “CP” see SEQ ID NOS: 95 and 97) motif for site-specific conjugation on the cysteine residue. Other designs further truncate the hinge region and retain hinge-C233 of IgG1 or CK—C214 of the kappa chain as the conjugation site (see SEQ ID NOS: 97 and 100). This can avoid loss of more C-terminal conjugations sites due to C-terminal clipping. The sequences and mutations of all disulfide engineered F(ab′) generated in this application are described in Table 17.

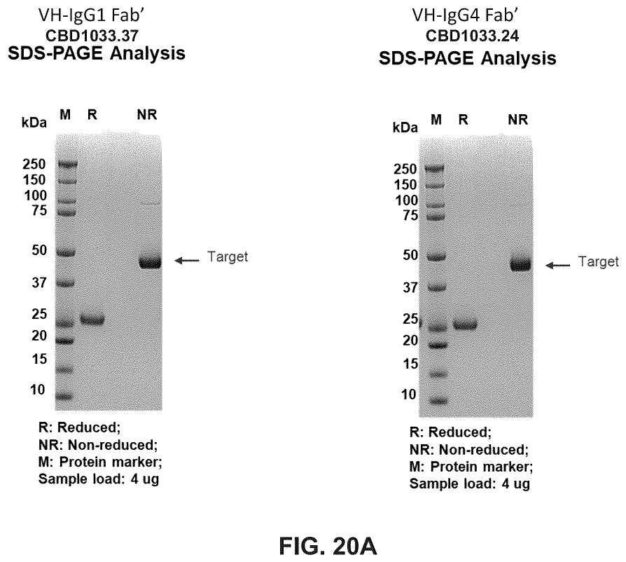

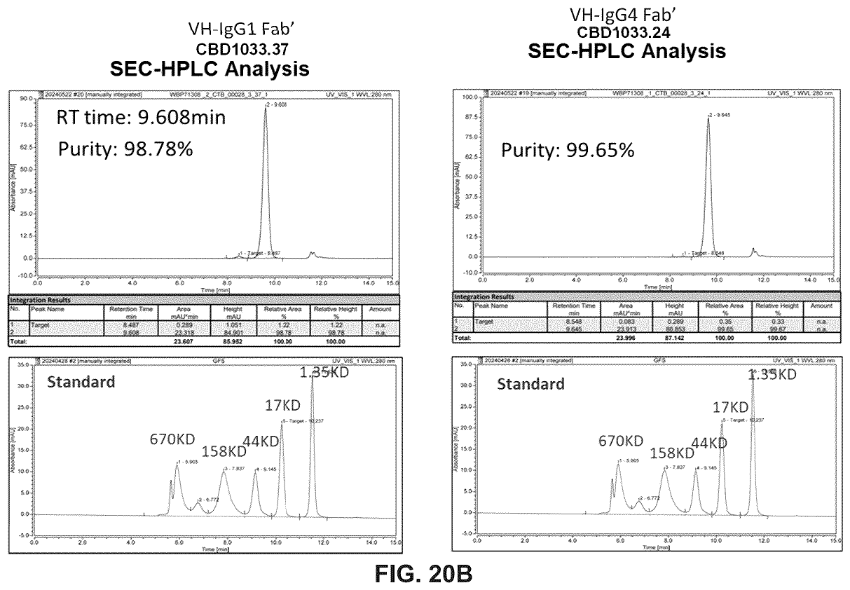

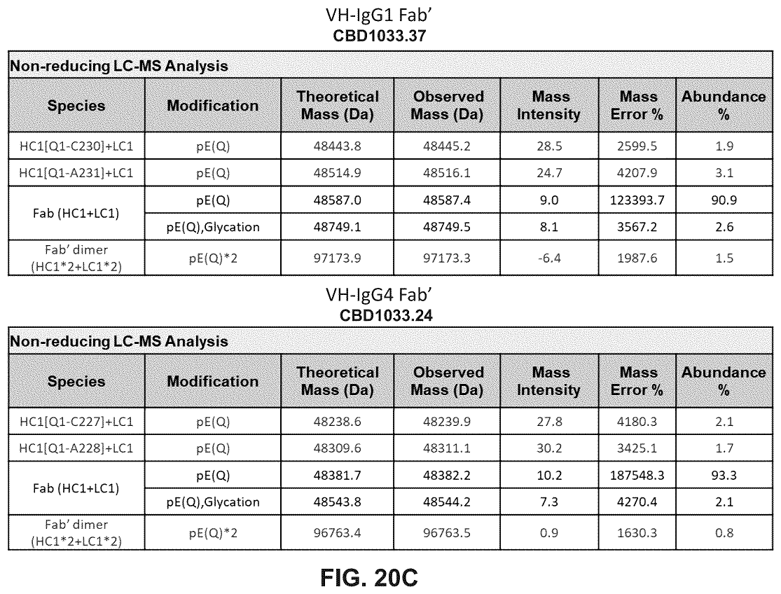

The disulfide-engineered humanized anti-CD8 F(ab′) analogs CBD1033.37 (IgG1-derived) and CBD1033.24 (IgG4-derived) were expressed and subjected to a three-step purification comprising affinity capture with KanCap G, reduction with 5 mM 2-mercaptoehtylamine (2-MEA) in PBS pH 6.5 and 10 mM EDTA for 1 hour at room temperature, and polish with cation exchange (SP—HP). The SDS-PAGE analysis and SEC-HPLC analysis confirmed main peaks corresponding to F(ab′) fraction with high purity of about 99%, indicating high quality purification (FIG. 20A and FIG. 20B). Non-reducing LC-MS analysis confirmed main peaks with 90% abundance corresponding to the correct mass of F(ab′) size, indicating intact interchain disulfide remained intact, the cysteine capping modification was successfully removed, and the engineered cysteine residue was available for conjugation (FIG. 20C).

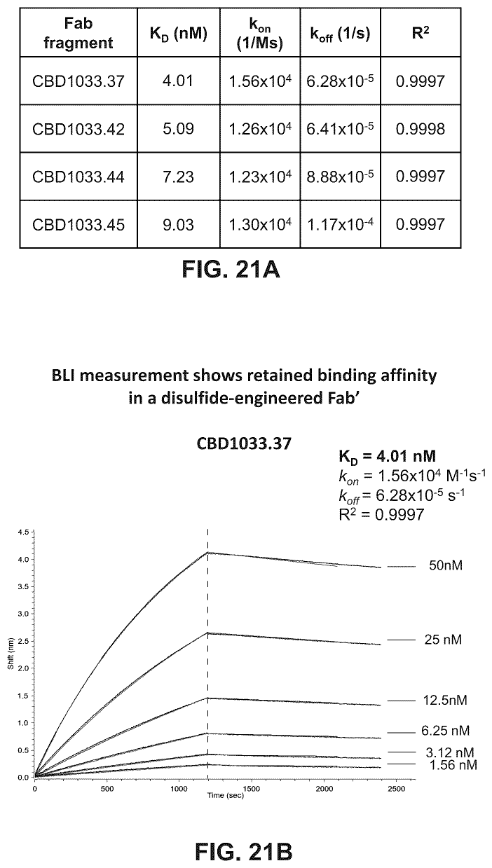

Four versions of disulfide engineered CBD1033 F(ab′)—CBD1033.37, CBD1033.42, CBD1033.44, and CBD1033.45 were generated with varied lengths depending on the truncation mutations (see Table 17 for description of the mutations in each version). All versions showed acceptable binding affinities in BLI assays (FIG. 21), indicating that the engineered disulfide did not interfere with the ability of these F(ab′) analogs to bind to CD8.

### Example 10: Conjugation of Anti-CD8 F(Ab′) with Maleimide-PEG and Testing their Binding Affinities

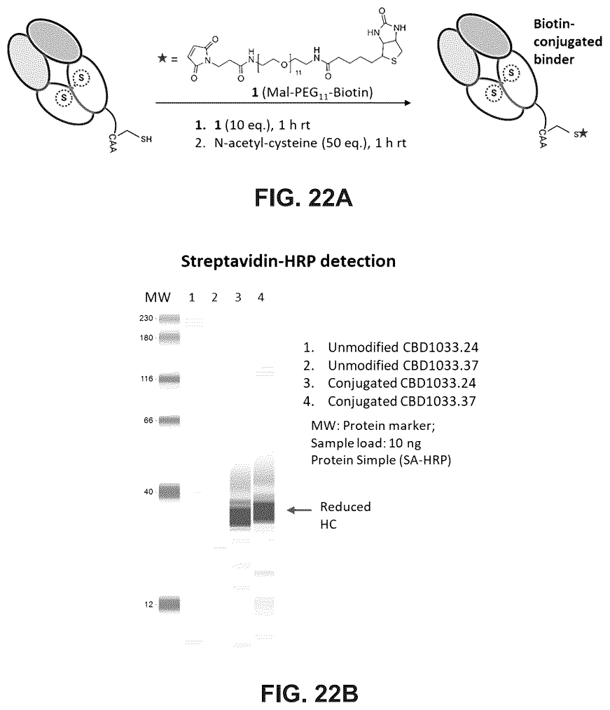

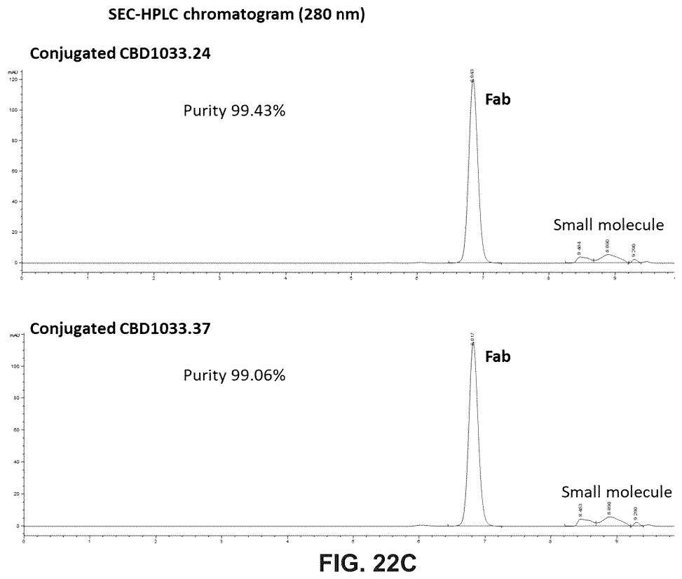

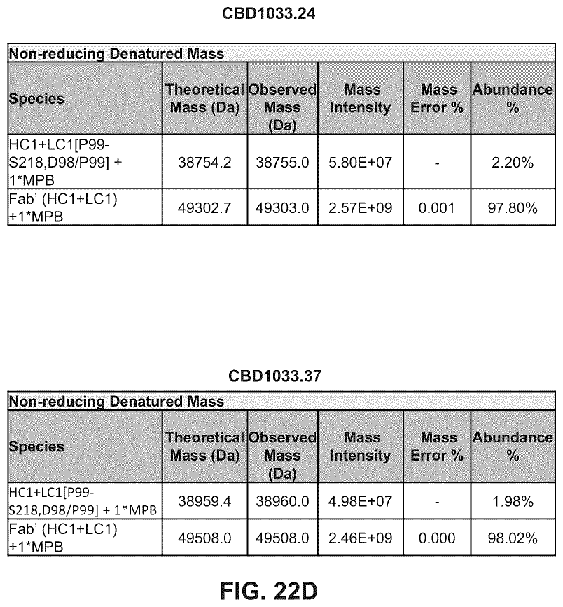

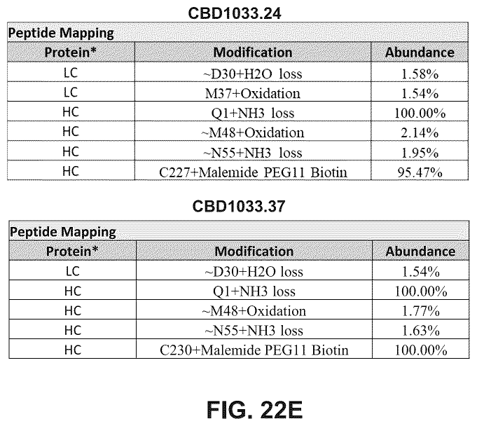

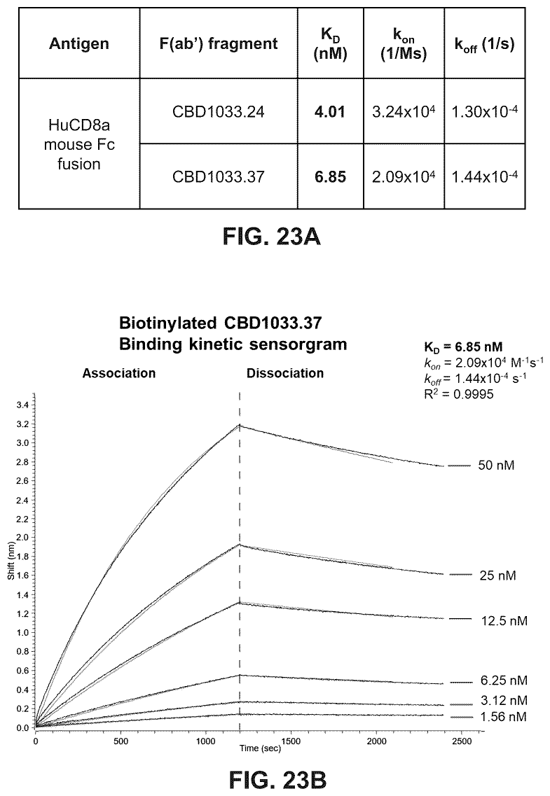

To test whether the conjugation reaction between the C-terminal cysteine on CAA motif and the maleimide-PEG lipid on LNP was robust and specific, maleimide-PEG-biotin-conjugated to anti-CD8 F(ab′) constructs. The biotin conjugation was assessed by streptavidin-horseradish peroxidase (HRP) immunoblotting (FIG. 22A and FIG. 22B). In SEC-HPLC studies of the conjugated molecules, the main peaks corresponded to F(ab′) fraction with over 99% purity, indicating no change in protein purity during conjugation (FIG. 22C). LC-MS analysis confirmed the correct mass of biotin conjugates with 98% abundance (FIG. 22D). Peptide mapping analysis showed 100% abundance for biotin conjugates at CH C230 (sequential numbering corresponding to C239 in Kabat numbering) for CBD1033.37 and >95% abundance of conjugates at CH C227 (sequential numbering corresponding to C239 in Kabat numbering) for CBD1033.24 (FIG. 22E). Together, the results showed that the conjugation reaction was efficient and specific to the desired cysteines), indicating that the engineered F(ab′) can be site-specifically conjugated to functionalized PEG-lipid in LNPs. The binding affinities of these engineered and maleimide-PEG-biotin conjugated F(ab′) analogs were also acceptable as confirmed by BLI assays (FIG. 23A and FIG. 23B).

### Example 11: Disulfide Engineered Anti-CD8α F(Ab′) were Functional as Targeting Moieties when Conjugated to Lipid Nanoparticles (tLNP)

To test whether the engineered anti-CD8α F(ab′) analogs maintained function as targeting moieties when conjugated to tLNP, CD8α-targeted lipid nanoparticles encapsulating mRNA-encoding mCherry were generated with anti-CD8α F(ab′) molecules having native or engineered disulfide bond. The mutations on the native disulfide anti-CD8α F(ab′) grafted on IgG1 (CBD1033.40) or IgG4 (CBD1033.12), and on the engineered disulfide anti-CD8α F(ab′) grafted on IgG1 (CBD1033.37) or IgG4 (CBD1033.24) are described in Table 17.

The in vitro transfection efficiency as measured by mCherry fluorescence level was comparable between the native disulfide F(ab′) and the engineered disulfide F(ab′) conjugated tLNPs. As a positive control, the whole anti-CD8 antibody with the same variable domains conjugated to the tLNPs produced the same transfection efficiency to the tested F(ab′) (FIG. 24A). Similarly, in vivo transfection efficiency assessed in the NCG mouse-human PBMC model, was also comparable among the tested F(ab′) and positive control IgG tLNPs, indicating that the humanized anti-CD8α F(ab′) with native disulfide or engineered disulfide bond retained their function as targeting moieties on tLNPs (FIG. 24B).

### Example 12: Assessment of the Effect of Disulfide Engineered Anti-CD8α F(Ab′) Analogs with Post-Translational Modification Liability-Engineered Mutations in Anti-CD8α Binding Domain on tLNP Delivery of mRNA-Encoded CAR

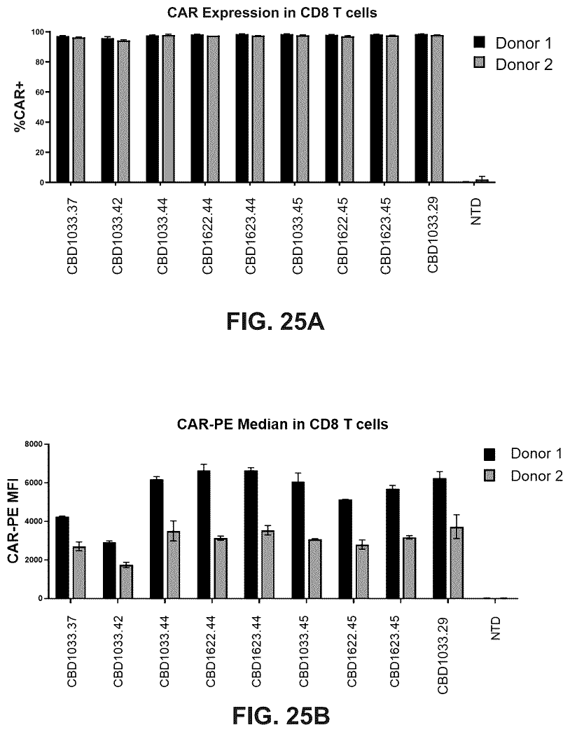

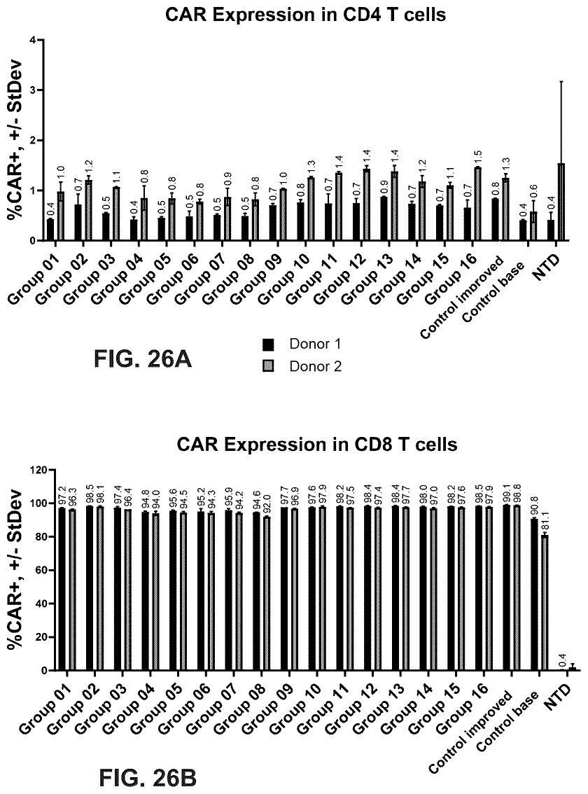

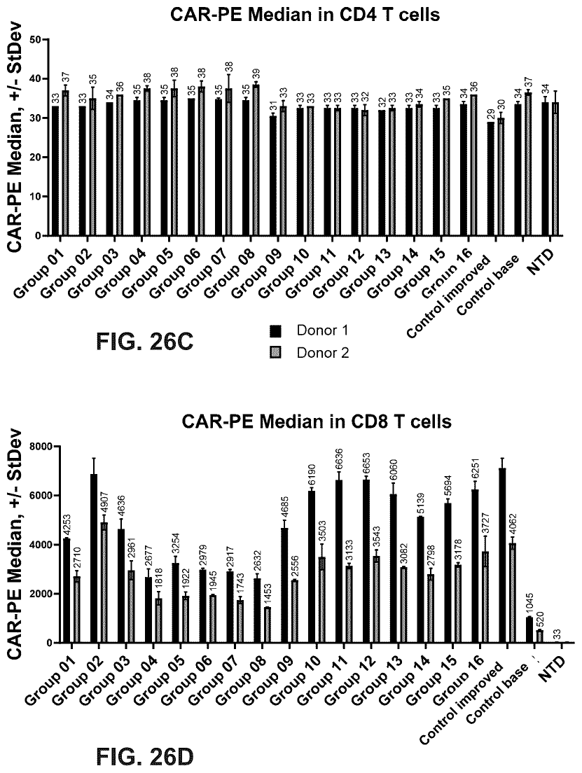

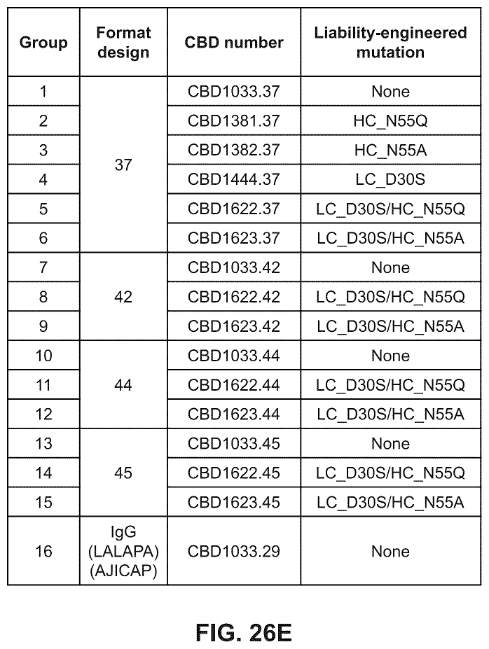

mRNA encoding a CAR delivered to CD8 expressing cells has promising therapeutic potential to treat various diseases. Thus, tLNPs conjugated to various designs of disulfide engineered anti-CD8 F(ab′) were tested for their abilities to deliver an mRNA-encoded CAR payload to CD8+ expanded T cells derived from human donors. Anti-CD8 F(ab′) variants were created to assess potential manufacturing liabilities in the VH and VL domains. Specific mutations were introduced to VH N55 and VL D30 to reduce deamidation and isomerization post-translational modifications of F(ab′) analogs. Additionally, a base construct known to mediate lower CAR expression than the improved mRNA construct and the improved mRNA construct were utilized as assay controls and encapsulated in tLNPs utilizing CBD1033.29 as their targeting moiety, a CBD1033.3 Fc-silenced IgG1 whole antibody thiolated by the AJICAP process and conjugated to LNP (control improved and control base in FIGS. 26A-26D; the former is the same as CBD1033.29 in FIGS. 25A-25B). As shown in FIG. 25A and FIG. 25B the in vitro transfection rates of tLNPs encapsulating CAR-encoding mRNA and targeted with F(ab′) analogs, and expression levels of the mRNA-encoded CAR, were comparable among different liability-engineered anti-CD8 binding domains (CBD1622 and CBD1623 vs CBD1033), among the disulfide-engineered F(ab′) designs (designs 0.37, 0.42, 0.44, and 0.45), and a positive control IgG with the same binding domain as the CBD1033 variants. However, while the 0.44 and 0.45 designs appear to behave consistently, the 0.37 and 0.42 designs of these liability engineered F(ab′) analogs sometimes exhibited reduced expression of the mRNA-encoded CAR (FIG. 26D) although transfection efficiency was comparable for all the tested constructs (FIG. 26B). As expected, there was minimal transfection and expression in CD4+ T cells (FIG. 26A and FIG. 26C). Taken together, these results showed that modifications of anti-CD8 binding domains, disulfide bond, and Fab′ lengths did not alter tLNP's targeting function and transfection efficiency. More importantly, monovalent anti-CD8 antibody in F(ab) or F(ab′) formats did not affect tLNP's function compared to bivalent anti-CD8 whole antibody controls.

### Example 13: Identification of the Epitope of CBD1033 and Cross-Competition Study with Other Anti-CD8 Binding Domains

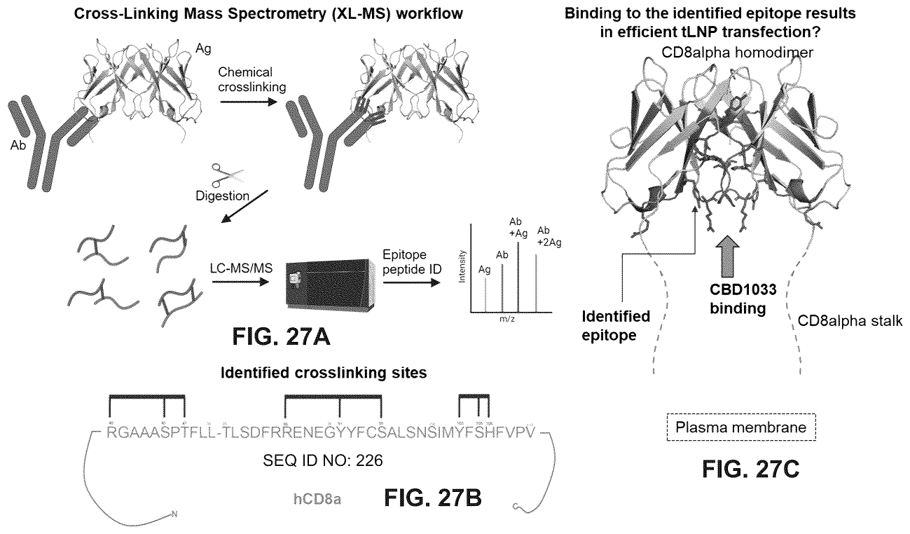

It was known that different antibodies could bind to different epitopes on the same target, which can influence the function of the antibodies in various contexts. To identify the epitope on CD8, CBD1033 Fab was cross-linked to CD8α homodimer protein. The complex was digested enzymatically and subjected to LC-MS analysis. The cross-linked amino acids were identified and mapped on the known structure of CD8α homodimer to identify the epitope bound by CBD1033 Fab (FIG. 27B). Amino acid residues 40, 45, 47, 86, 91, 95, 103, 105, and 106 of CD8α were crosslinked to the Fab indicating that CBD1033 interacted with the CC′ loop, C′ strand, turn before F strand, F strand, and G strand of CD8α (as predicted by AlphaFold2; see Srinivasan et al., 2024 Front. Immunol. 15:1412513, which is incorporated by reference for its teachings about the structure of CD8 and its interactions with mAbs.) Residues 40 and 45 are int the CC′ loop, residue 47 is in the C′ strand, residue 86 is in the turn before the F strand, residues 91 and 95 are in the F strand, and residues 103, 105, and 106 are in the G strand, The epitope is a structural (non-linear) epitope located on the membrane proximal portion of the CD8 ectodomain near the dimer interface above the hinge (or stalk) emerging from the cell membrane. (see FIG. 27C)

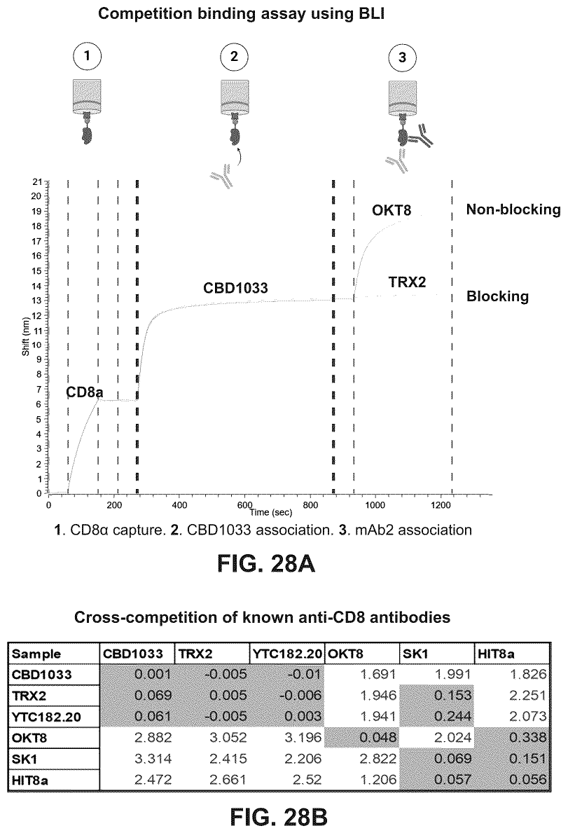

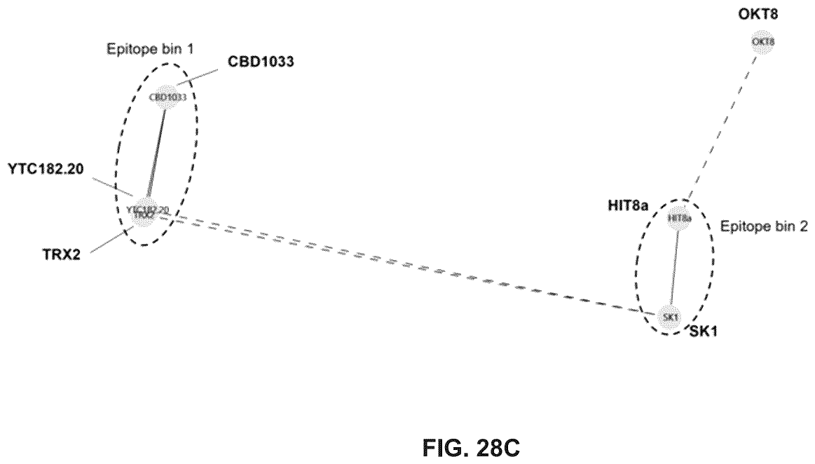

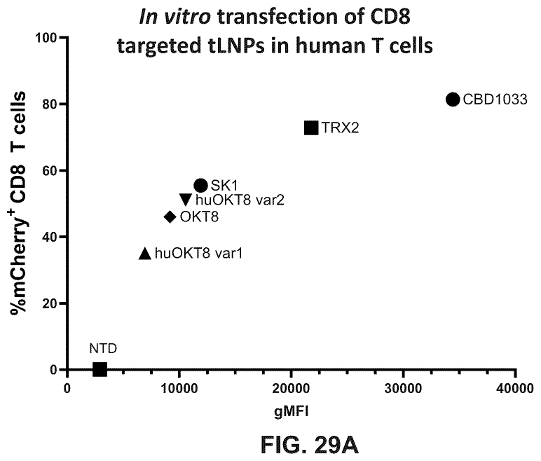

To test whether this epitope was also bound by other anti-CD8 antibodies, competition binding assays using BLI were performed (FIG. 28A). Binding of anti-CD8 antibodies TRX2, YTC182.20, OKT8, SK1, and HIT8a to CD8α homodimer was tested for competition with CBD1033 Fab (representing the antibody CT8). The results showed that TRX2 and YTC182.20 binding to CD8α were hindered by CBD1033 Fab binding while OKT8, SK1, and HIT8a were not affected by the pre-association of CBD1033 with CD8α (FIG. 28A). Cross-competition binding using pairs of antibodies showed that CBD1033, TRX2, and YTC182.20 blocked each other from binding to the CD8 target, while OKT8, SK1, and HIT8a did not (FIG. 28B). Based on these results, it was found that CBD1033, YTC182.20, and TRX2 share an overlapping epitope on CD8α that is distinct from the epitopes of the other antibodies tested (FIG. 28C).

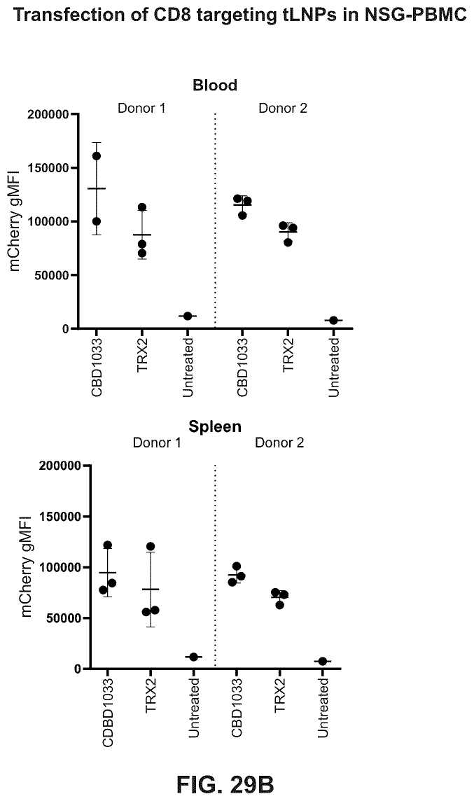

In transfection assays of activated human T cells in vitro, essentially as described above, CBD1033 and TRX2 conjugated tLNPs showed higher transfection efficiency than SK1 and OKT8 conjugated tLNPs (FIG. 29A). With in vivo transfection in NSG-PBMC mice, essentially as described above, CBD1033 and TRX2 conjugated tLNPs also showed comparable transfection efficiency in vivo (FIG. 29B), indicating that TRX2 can act efficiently as a targeting moiety on a CD8-targeted tLNP. These data further suggested that YTC182.20 or an antigen binding fragment thereof could also serve as an efficient CD8 targeting moiety.

TABLE 16
Sequence Listing of variable domains, full-length constant
regions, and full-length humanized anti-CD8α variants.

| | | | SEQ |
|---|---|---|---|
| | | | ID |
| Name | Note | Sequence | NO |
| CBD1017vh | VH of mouse | EVQLQQSGPELVKPGASVKISCKASRYTFTDY | 1 |
| | anti-CD8α | NLHWVKLSHEKSLEWIGFIYPYNGGTGYNQK | |
| | antibody clone | FKNKAKLTVDYSSSTAYMELRSLTSVDAAVY | |
| | CT8 | YCARDHRYNEGVSFDYWGQGTTLTVSS | |
| VH-CDR1 | VH-CDR1 of | RYTFTDYNLH | 2 |
| CBD1017 | mouse anti- | | |
| | CD8αantibody | | |
| | clone CT8 | | |
| VH-CDR2 | VH-CDR2 of | FIYPYNGGTG | 3 |
| CBD1017 | mouse anti- | | |
| | CD8α antibody | | |
| | clone CT8 | | |
| VH-CDR3 | VH-CDR3 of | DHRYNEGVSFDY | 4 |
| CBD1017 | mouse anti- | | |
| | CD8α antibody | | |
| | clone CT8 | | |
| CBD1017vl | VL of mouse | NIVLTQSPASLAVSLGQRATISCRASESVDGF | 5 |
| | anti-CD8α | GNSFMNWYQQKPGQSPKLLIYLASNLESGVP | |
| | antibody clone | ARFSGSGSRTDFTLTIDPVEADDAATYYCQQ | |
| | CT8 | NNEDPYTFGGGTKLEIK | |
| VL-CDR1 | VL-CDR1 of | RASESVDGFGNSFMN | 6 |
| CBD1017 | mouse anti- | | |
| | CD8α antibody | | |
| | clone CT8 | | |
| VL-CDR2 | VL-CDR2 of | LASNLES | 7 |
| CBD1017 | mouse anti- | | |
| | CD8α antibody | | |
| | clone CT8 | | |
| VL-CDR3 | VL-CDR3 of | QQNNEDPYT | 8 |
| CBD1017 | mouse anti- | | |
| | CD8α antibody | | |
| | clone CT8 | | |
| 1-46*01 | IgHV1- | QVQLVQSGAEVKKPGASVKVSCKASGYTFTS | 9 |
| | 46*01/ | YYMHWVRQAPGQGLEWMGIINPSGGSTSYA | |
| | IGHJ6*01 | QKFQGRVTMTRDTSTSTVYMELSSLRSEDTA | |
| | germline VH | VYYCARWGQGTTVTVSS | |
| h1017-H1 | Humanized | QVQLVQSGAEVKKPGASVKVSCKASRYTFTD | 10 |
| | CT8 VH | YNLHWVRQAPGQGLEWMGFIYPYNGGTGY | |
| | variant based | AQKFQGRVTMTRDTSTSTVYMELSSLRSEDT | |
| | on VH1-46 | AVYYCARDHRYNEGVSFDYWGQGTTVTVSS | |
| h1017-H2 | Humanized | QVQLVQSGAEVKKPGASVKVSCKASRYTFTD | 11 |
| | CT8 VH | YNLHWVRQAPGQGLEWMGFIYPYNGGTGY | |
| | variant based | AQKFQGRVTMTVDTSTSTAYMELSSLRSEDT | |
| | on VH1-46 | AVYYCARDHRYNEGVSFDYWGQGTTVTVSS | |
| h1017-H3 | Humanized | QVQLVQSGAEVKKPGASVKISCKASRYTFTD | 12 |
| | CT8 VH | YNLHWVRQAPGQGLEWIGFIYPYNGGTGYA | |
| | variant based | QKFQGRATLTVDTSTSTAYMELSSLRSEDTA | |
| | on VH1-46 | VYYCARDHRYNEGVSFDYWGQGTTLTVSS | |
| h1017-H4 | Humanized | QVQLVQSGAEVKKPGASVKVSCKASRYTFTD | 13 |
| | CT8 VH | YNLHWVRQAPGQGLEWMGFIYPYNGGTGY | |
| | variant based | AQKFQGRVTMTVDYSTSTAYMELSSLRSEDT | |
| | on VH1-46 | AVYYCARDHRYNEGVSFDYWGQGTTVTVSS | |
| h1017-H5 | Humanized | QVQLVQSGAEVKKPGASVKISCKASRYTFTD | 14 |
| | CT8 VH | YNLHWVRQAPGQGLEWIGFIYPYNGGTGYA | |
| | variant based | QKFQGRATLTVDYSTSTAYMELSSLRSEDTA | |
| | on VH1-46 | VYYCARDHRYNEGVSFDYWGQGTTLTVSS | |
| 1-39*01 | IgKV1- | DIQMTQSPSSLSASVGDRVTITCRASQSISSYL | 15 |
| | 39*01/ | NWYQQKPGKAPKLLIY | |
| | IGKJ2*01 | AASSLQSGVPSRFSGSGSGTDFTLTISSLQPED | |
| | germline VL | FATYYCQQSYSTPPFGQGTKLEIK | |
| h1017-L1 | Humanized | DIQMTQSPSSLSASVGDRVTITCRASESVDGF | 16 |
| | CT8 VL | GNSFMNWYQQKPGKAPKLLIYLASNLESGVP | |
| | variant based | SRFSGSGSGTDFTLTISSLQPEDFATYYCQQN | |
| | on VK1-39 | NEDPYTFGQGTKLEIK | |
| h1017-L2 | Humanized | DIQLTQSPSSLSASVGDRATITCRASESVDGFG | 17 |
| | CT8 VL | NSFMNWYQQKPGKAPKLLIYLASNLESGVPS | |
| | variant based | RFSGSGSGTDFTLTISSVQPEDFATYYCQQNN | |
| | on VK1-39 | EDPYTFGQGTKLEIK | |
| h1017-L3 | Humanized | DIQLTQSPSSLSASVGDRATITCRASESVDGFG | 18 |
| | CT8 VL | NSFMNWYQQKPGKAPKLLIYLASNLESGVPS | |
| | variant based | RFSGSGSRTDFTLTISSVQPEDFATYYCQQNN | |
| | on VK1-39 | EDPYTFGQGTKLEIK | |
| VH hOKT8 | VH of | EVQLVQSGAEVKKPGASVKVSCKASGFNIKD | 23 |
| | humanized | TYIHWVRQAPGQGLEWIGRIDPANDNTLYAS | |
| | mouse anti- | KFQGRATITADTSTSTAYLELSSLRSEDTAVY | |
| | CD8α antibody | YCGRGYGYYVFDHWGQGTLVTVSS | |
| | clone OKT8 | | |
| VL | VL of | DVQITQSPSSLSASVGDRVTITCRTSRSISQYL | 24 |
| hOKT8 | humanized | AWYQEKPGKTNKLLIYSGSTLQSGIPSRFSGS | |
| | mouse anti- | GSGTDFTLTISSLQPEDFATYYCQQVNEFPPTF | |
| | CD8α antibody | GQGTKVEIK | |
| | clone OKT8 | | |
| VH | VH of mouse | EVQLQQSGAELVKPGASVKLSCTASGFNIKD | 25 |
| OKT8 | anti-CD8α | TYIHFVRQRPEQGLEWIGRIDPANDNTLYASK | |
| | antibody clone | FQGKATITADTSSNTAYMHLCSLTSGDTAVY | |
| | OKT8 | YCGRGYGYYVFDHWGQGTTLTVSS | |
| VL | VL of mouse | DVQINQSPSFLAASPGETITINCRTSRSISQYLA | 26 |
| OKT8 | anti-CD8α | WYQEKPGKTNKLLIYSGSTLQSGIPSRFSGSG | |
| | antibody clone | SGTDFTLTISGLEPEDFAMYYCQQHNENPLTF | |
| | OKT8 | GAGTKLELK | |
| VH | N55S of | QVQLVQSGAEVKKPGASVKVSCKASRYTFTD | 27 |
| CBD1380 | h1017-H2 | YNLHWVRQAPGQGLEWMGFIYPYSGGTGYA | |
| | | QKFQGRVTMTVDTSTSTAYMELSSLRSEDTA | |
| | | VYYCARDHRYNEGVSFDYWGQGTTVTVSS | |
| VH | N55Q of | QVQLVQSGAEVKKPGASVKVSCKASRYTFTD | 28 |
| CBD1381 | h1017-H2 | YNLHWVRQAPGQGLEWMGFIYPYQGGTGY | |
| | | AQKFQGRVTMTVDTSTSTAYMELSSLRSEDT | |
| | | AVYYCARDHRYNEGVSFDYWGQGTTVTVSS | |
| VH | N55A of | QVQLVQSGAEVKKPGASVKVSCKASRYTFTD | 29 |
| CBD1382 | h1017-H2 | YNLHWVRQAPGQGLEWMGFIYPYAGGTGY | |
| | | AQKFQGRVTMTVDTSTSTAYMELSSLRSEDT | |
| | | AVYYCARDHRYNEGVSFDYWGQGTTVTVSS | |
| VH | N55D of | QVQLVQSGAEVKKPGASVKVSCKASRYTFTD | 30 |
| CBD1383 | h1017-H2 | YNLHWVRQAPGQGLEWMGFIYPYDGGTGY | |
| | | AQKFQGRVTMTVDTSTSTAYMELSSLRSEDT | |
| | | AVYYCARDHRYNEGVSFDYWGQGTTVTVSS | |
| Mod 1-18 | Modified | QVQLVQSGAEVKKPGASVKVSCKASGYTFTS | 31 |
| | IgHV1-18*01 | YYMHWVRQAPGQGLEWMGIINPSGGSTSYA | |
| | germline VH | QKFQGRVTMTRDTSTSTVYMELSSLRSEDTA | |
| | | VYYCARWGQGTLVTVS | |
| h1017-H6 | Humanized | QVQLVQSGAEVKKPGASVKVSCKASRYTFTD | 32 |
| | CT8 VH | YNLHWVRQAPGQGLEWMGFIYPYNGGTNY | |
| | variant based | AQKFQGRVTITADTSTSTAYMELSSLRSEDTA | |
| | on VH1-18 | VYYCARDHRYNEGVSFDYWGQGTLVTVSS | |
| h1017-H7 | Humanized | QVQLVQSGAEVKKPGASVKVSCKASRYTFTD | 33 |
| | CT8 VH | YNLHWVRQAPGQGLEWIGFIYPYNGGTGYA | |
| | variant based | QKFQGRVTITADTSTSTAYMELSSLRSEDTAV | |
| | on VH1-18 | YYCARDHRYNEGVSFDYWGQGTLVTVSS | |
| h1017-H8 | Humanized | QVQLVQSGAEVKKPGASVKVSCKASRYTFTD | 34 |
| | CT8 VH | YNLHWVRQAPGQGLEWIGFIYPYNGGTGYA | |
| | variant based | QKFKGRVTITADTSTSTAYMELSSLRSEDTAV | |
| | on VH1-18 | YYCARDHRYNEGVSFDYWGQGTLVTVSS | |
| h1017-H9 | Humanized | QVQLVQSGAEVKKPGASVKVSCKASRYTFTD | 35 |
| | CT8 VH | YNLHWVRQAPGQGLEWMGFIYPYNGGTGY | |
| | variant based | AQKFQGRVTLTVDYSTSTAYMELSSLRSEDT | |
| | on VH1-18 | AVYYCARDHRYNEGVSFDYWGQGTLVTVSS | |
| h1017-H10 | Humanized | QVQLVQSGAEVKKPGASVKVSCKASRYTFTD | 36 |
| | CT8 VH | YNLHWVRQAPGQGLEWIGFIYPYNGGTGYA | |
| | variant based | QKFQGRVTLTVDYSTSTAYMELSSLRSEDTA | |
| | on VH1-18 | VYYCARDHRYNEGVSFDYWGQGTLVTVSS | |
| Mod 3D-11 | Modified | EIVLTQSPGTLSLSPGERATLSCRASQSVSSSY | 37 |
| | IgKV3D- | LAWYQQKPGQAPRLLIYGASSRATGIPDRFSG | |
| | 11*01 | SGSGTDFTLTISRLEPEDFAVYYCQQYGSSPLT | |
| | germline VL | FGQGTKVEIK | |
| h1017-L4 | Humanized | EIVLTQSPGTLSLSPGERATLSCRASESVDGFG | 38 |
| | CT8 VL | NSFMNWYQQKPGQAPRLLIYLASNLESGIPA | |
| | variant based | RFSGSGSGTDFTLTISRLEPEDFAVYYCQQNN | |
| | on VK3D-11 | EDPYTFGQGTKVEIK | |
| h1017-L5 | Humanized | EIVLTQSPGTLSLSPGERATLSCRASESVDGFG | 39 |
| | CT8 VL | NSFMNWYQQKPGQAPRLLIYLASNLESGVPA | |
| | variant based | RFSGSGSGTDFTLTISRLEPEDFAVYYCQQNN | |
| | on VK3D-11 | EDPYTFGQGTKVEIK | |
| h1017-L6 | Humanized | EIVLTQSPGTLSLSPGERATLSCRASESVDGFG | 40 |
| | CT8 VL | NSFMNWYQQKPGQAPRLLIYLASNLESGVPA | |
| | variant based | RFSGSGSRTDFTLTISRLEPEDFAVYYCQQNN | |
| | on VK3D-11 | EDPYTFGQGTKVEIK | |
| IGKC_human | Kappa chain | RTVAAPSVFIFPPSDEQLKSGTASVVCLLNNF | 41 |
| | constant region | YPREAKVQWKVDNALQSGNSQESVTEQDSK | |
| | used in the | DSTYSLSSTLTLSKADYEKHKVYACEVTHQG | |
| | whole antibody | LSSPVTKSFNRGEC | |
| | constructs | | |
| | (UniProt | | |
| | P01834) | | |
| IGG1_human | Wild-type | ASTKGPSVFPLAPSSKSTSGGTAALGCLVKDY | 42 |
| | IgG1 constant | FPEPVTVSWNSGALTSGVHTFPAVLQSSGLYS | |
| | regions | LSSVVTVPSSSLGTQTYICNVNHKPSNTKVDK | |
| | (UniProt | KVEPKSCDKTHTCPPCPAPELLGGPSVFLFPP | |
| | P0DOX5) | KPKDTLMISRTPEVTCVVVDVSHEDPEVKFN | |
| | | WYVDGVEVHNAKTKPREEQYNSTYRVVSVL | |
| | | TVLHQDWLNGKEYKCKVSNKALPAPIEKTIS | |
| | | KAKGQPREPQVYTLPPSRDELTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPGK | |
| IGG1_human | Fc-silenced | ASTKGPSVFPLAPSSKSTSGGTAALGCLVKDY | 43 |
| LALAPA EM | IgG1 constant | FPEPVTVSWNSGALTSGVHTFPAVLQSSGLYS | |
| | regions used in | LSSVVTVPSSSLGTQTYICNVNHKPSNTKVDK | |
| | the whole | KVEPKSCDKTHTCPPCPAPEAAGGPSVFLFPP | |
| | antibody | KPKDTLMISRTPEVTCVVVDVSHEDPEVKFN | |
| | constructs | WYVDGVEVHNAKTKPREEQYNSTYRVVSVL | |
| | (with E358 and | TVLHQDWLNGKEYKCKVSNKALAAPIEKTIS | |
| | M360 allotypic | KAKGQPREPQVYTLPPSREEMTKNQVSLTCL | |
| | reversions) | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| IGG1_human | Fc-silenced | ASTKGPSVFPLAPSSKSTSGGTAALGCLVKDY | 44 |
| LALAPA DL | IgG1 constant | FPEPVTVSWNSGALTSGVHTFPAVLQSSGLYS | |
| | regions used in | LSSVVTVPSSSLGTQTYICNVNHKPSNTKVDK | |
| | the whole | KVEPKSCDKTHTCPPCPAPELLGGPSVFLFPP | |
| | antibody | KPKDTLMISRTPEVTCVVVDVSHEDPEVKFN | |
| | constructs | WYVDGVEVHNAKTKPREEQYNSTYRVVSVL | |
| | (with D358 | TVLHQDWLNGKEYKCKVSNKALPAPIEKTIS | |
| | and L360 | KAKGQPREPQVYTLPPSRDELTKNQVSLTCL | |
| | allotypic | VKGFYPSDIAVEWESNGQPENNYKTTPPVLD | |
| | residues) | SDGSFFLYSKLTVDKSRWQQGNVFSCSVMHE | |
| | | ALHNHYTQKSLSLSPGK | |
| VH-CDR1 | VH N33 | RYTFTDYX₁LH | 45 |
| N33var | degenerate | | |
| VH-CDR2 | VH N55 | FIYPYX₁GGTG | 46 |
| N55var1 | degenerate (set | | |
| | w/o D) | | |
| VH-CDR2 | VH N55 | FIYPYX₂GGTG | 47 |
| N55var1 | degenerate (set | | |
| | w/ D) | | |
| VH-CDR3 | VH N103 | DHRYX₁EGVSFDY; | 48 |
| N103var | degenerate | | |
| VL-CDR1 D30 | VL D30 and | RASESVX₃GFGX₁SFMN; | 49 |
| and N34var | N34 | | |
| | degenerate | | |
| VL-CDR2 | VL N57 | LASX₂LES | 50 |
| N57var | degenerate | | |
| VL-CDR3 N95, | VL N95, N96, | QQX₂X₂EX₃PYT | 51 |
| N96, and D98var | and D98 | | |
| | degenerate | | |
| h1017-1-46 (1) | Humanized | QVQLVQSGAEVKKPGASVKVSCKASRYTFT | 52 |
| | CT8 VH based | DYX₁LHWVRQAPGQGLEWMGFIYPYX₁GGT | |
| | on VH1-46 | GYAQKFQGRVTMTRDTSTSTVYMELSSLRSE | |
| | with CDR | DTAVYYCARDHRYX₁EGVSFDYWGQGTTVT | |
| | variants (1) | VSS | |
| h1017-1-46 (2) | Humanized | QVQLVQSGAEVKKPGASVKVSCKASRYTFT | 53 |
| | CT8 VH based | DYX₁LHWVRQAPGQGLEWMGFIYPYX₂GGT | |
| | on VH1-46 | GYAQKFQGRVTMTRDTSTSTVYMELSSLRSE | |
| | with CDR | DTAVYYCARDHRYX₁EGVSFDYWGQGTTVT | |
| | variants (2) | VSS | |
| h1017-1-39 | Humanized | DIQMTQSPSSLSASVGDRVTITCRASESVX₃GF | 54 |
| | CT8 VL based | GX₁SFMNWYQQKPGKAPKLLIYLASX₂LESG | |
| | on VK1-39 | VPSRFSGSGSGTDFTLTISSLQPEDFATYYCQQ | |
| | with CDR | X₂X₂EX₃PYTFGQGTKLEIK | |
| | variants | | |
| h1017-1-18 (1) | Humanized | QVQLVQSGAEVKKPGASVKVSCKASRYTFT | 55 |
| | CT8 VH based | DYX₁LHWVRQAPGQGLEWMGFIYPYX₁GGT | |
| | on VH1-18 | GYAQKFQGRVTMTRDTSTSTVYMELSSLRSE | |
| | with CDR | DTAVYYCARDHRYX₁EGVSFDYWGQGTLVT | |
| | variants | VS | |
| h1017-1-18 (2) | Humanized | QVQLVQSGAEVKKPGASVKVSCKASRYTFT | 56 |
| | CT8 VH based | DYX₁LHWVRQAPGQGLEWMGFIYPYX₂GGT | |
| | on VH1-18 | GYAQKFQGRVTMTRDTSTSTVYMELSSLRSE | |
| | with CDR | DTAVYYCARDHRYX₁EGVSFDYWGQGTLVT | |
| | variants | VS | |
| h1017-3D-11 | Humanized | EIVLTQSPGTLSLSPGERATLSCRASESVX₃GF | 57 |
| | CT8 VL based | GX₁SFMNWYQQKPGQAPRLLIYLASX₂LESGI | |
| | on VK3D-11 | PDRFSGSGSGTDFTLTISRLEPEDFAVYYCQQ | |
| | with CDR | X₂X₂EX₃PYTFGQGTKVEIK | |
| | variants | | |
| VH-CDR2v2 | Modified VH- | FIYPYSGGTG | 58 |
| CBD1017 N55S | CDR2 of | | |
| | mouse anti- | | |
| | CD8α clone | | |
| | CT8 | | |
| VH-CDR2v3 | Modified VH- | FIYPYQGGTG | 59 |
| CBD1017 N55Q | CDR2 of | | |
| | mouse anti- | | |
| | CD8α clone | | |
| | CT8 | | |
| VH-CDR2v4 | Modified VH- | FIYPYAGGTG | 60 |
| CBD1017 N55A | CDR2 of | | |
| | mouse anti- | | |
| | CD8α antibody | | |
| | clone CT8 | | |
| CBD1033 HC | h1017-H2 Fc- | QVQLVQSGAEVKKPGASVKVSCKASRYTFTD | 61 |
| | silenced IgG1 | YNLHWVRQAPGQGLEWMGFIYPYNGGTGY | |
| | | AQKFQGRVTMTVDTSTSTAYMELSSLRSEDT | |
| | | AVYYCARDHRYNEGVSFDYWGQGTTVTVSS | |
| | | ASTKGPSVFPLAPSSKSTSGGTAALGCLVKDY | |
| | | FPEPVTVSWNSGALTSGVHTFPAVLQSSGLYS | |
| | | LSSVVTVPSSSLGTQTYICNVNHKPSNTKVDK | |
| | | KVEPKSCDKTHTCPPCPAPEAAGGPSVFLFPP | |
| | | KPKDTLMISRTPEVTCVVVDVSHEDPEVKFN | |
| | | WYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1033 LC | h1017-L2 | DIQLTQSPSSLSASVGDRATITCRASESVDGFG | 62 |
| CBD1035 LC | Kappa chain | NSFMNWYQQKPGKAPKLLIYLASNLESGVPS | |
| CBD1037 LC | | RFSGSGSGTDFTLTISSVQPEDFATYYCQQNN | |
| CBD1039 LC | | EDPYTFGQGTKLEIKRTVAAPSVFIFPPSDEQL | |
| CBD1380 LC | | KSGTASVVCLLNNFYPREAKVQWKVDNALQ | |
| CBD1381 LC | | SGNSQESVTEQDSKDSTYSLSSTLTLSKADYE | |
| CBD1382 LC | | KHKVYACEVTHQGLSSPVTKSFNRGEC | |
| VL CBD1443 | D30E of | DIQLTQSPSSLSASVGDRATITCRASESVEGFG | 63 |
| VL CBD1446 | h1017-L2 | NSFMNWYQQKPGKAPKLLIYLASNLESGVPS | |
| | | RFSGSGSGTDFTLTISSVQPEDFATYYCQQNN | |
| | | EDPYTFGQGTKLEIK | |
| VL CBD1444 | D30S of | DIQLTQSPSSLSASVGDRATITCRASESVSGFG | 64 |
| VL CBD1447 | h1017-L2 | NSFMNWYQQKPGKAPKLLIYLASNLESGVPS | |
| VL CBD1575 | | RFSGSGSGTDFTLTISSVQPEDFATYYCQQNN | |
| VL CBD1576 | | EDPYTFGQGTKLEIK | |
| VL CBD1622 | | | |
| VL CBD1623 | | | |
| VL CBD1445 | D30A of | DIQLTQSPSSLSASVGDRATITCRASESVAGFG | 65 |
| VL CBD1448 | h1017-L2 | NSFMNWYQQKPGKAPKLLIYLASNLESGVPS | |
| | | RFSGSGSGTDFTLTISSVQPEDFATYYCQQNN | |
| | | EDPYTFGQGTKLEIK | |
| VH CBD1449 | N55S of | QVQLVQSGAEVKKPGASVKVSCKASRYTFTD | 66 |
| | h1017-H4 | YNLHWVRQAPGQGLEWMGFIYPYSGGTGYA | |
| | | QKFQGRVTMTVDYSTSTAYMELSSLRSEDTA | |
| | | VYYCARDHRYNEGVSFDYWGQGTTVTVSS | |
| VH CBD1450 | N55Q of | QVQLVQSGAEVKKPGASVKVSCKASRYTFTD | 67 |
| VH CBD1575 | h1017-H4 | YNLHWVRQAPGQGLEWMGFIYPYQGGTGY | |
| | | AQKFQGRVTMTVDYSTSTAYMELSSLRSEDT | |
| | | AVYYCARDHRYNEGVSFDYWGQGTTVTVSS | |
| VH CBD1451 | N55A of | QVQLVQSGAEVKKPGASVKVSCKASRYTFTD | 68 |
| VH CBD1576 | h1017-H4 | YNLHWVRQAPGQGLEWMGFIYPYAGGTGY | |
| | | AQKFQGRVTMTVDYSTSTAYMELSSLRSEDT | |
| | | AVYYCARDHRYNEGVSFDYWGQGTTVTVSS | |
| CBD1035 HC | h1017-H3 Fc- | QVQLVQSGAEVKKPGASVKISCKASRYTFTD | 69 |
| | silenced IgG1 | YNLHWVRQAPGQGLEWIGFIYPYNGGTGYA | |
| | | QKFQGRATLTVDTSTSTAYMELSSLRSEDTA | |
| | | VYYCARDHRYNEGVSFDYWGQGTTLTVSS | |
| | | ASTKGPSVFPLAPSSKSTSGGTAALGCLVKDY | |
| | | FPEPVTVSWNSGALTSGVHTFPAVLQSSGLYS | |
| | | LSSVVTVPSSSLGTQTYICNVNHKPSNTKVDK | |
| | | KVEPKSCDKTHTCPPCPAPEAAGGPSVFLFPP | |
| | | KPKDTLMISRTPEVTCVVVDVSHEDPEVKFN | |
| | | WYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1039 HC | h1017-H5 Fc- | QVQLVQSGAEVKKPGASVKISCKASRYTFTD | 70 |
| CBD1040 HC | silenced IgG1 | YNLHWVRQAPGQGLEWIGFIYPYNGGTGYA | |
| | | QKFQGRATLTVDYSTSTAYMELSSLRSEDTA | |
| | | VYYCARDHRYNEGVSFDYWGQGTTLTVSS | |
| | | ASTKGPSVFPLAPSSKSTSGGTAALGCLVKDY | |
| | | FPEPVTVSWNSGALTSGVHTFPAVLQSSGLYS | |
| | | LSSVVTVPSSSLGTQTYICNVNHKPSNTKVDK | |
| | | KVEPKSCDKTHTCPPCPAPEAAGGPSVFLFPP | |
| | | KPKDTLMISRTPEVTCVVVDVSHEDPEVKFN | |
| | | WYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1040 LC | h1017-L3 | DIQLTQSPSSLSASVGDRATITCRASESVDGFG | 71 |
| | Kappa chain | NSFMNWYQQKPGKAPKLLIYLASNLESGVPS | |
| | | RFSGSGSRTDFTLTISSVQPEDFATYYCQQNN | |
| | | EDPYTFGQGTKLEIKRTVAAPSVFIFPPSDEQL | |
| | | KSGTASVVCLLNNFYPREAKVQWKVDNALQ | |
| | | SGNSQESVTEQDSKDSTYSLSSTLTLSKADYE | |
| | | KHKVYACEVTHQGLSSPVTKSFNRGEC | |
| CBD1380 HC | N55S of | QVQLVQSGAEVKKPGASVKVSCKASRYTFTD | 72 |
| | h1017-H2 Fc- | YNLHWVRQAPGQGLEWMGFIYPYSGGTGYA | |
| | silenced IgG1 | QKFQGRVTMTVDTSTSTAYMELSSLRSEDTA | |
| | | VYYCARDHRYNEGVSFDYWGQGTTVTVSS | |
| | | ASTKGPSVFPLAPSSKSTSGGTAALGCLVKDY | |
| | | FPEPVTVSWNSGALTSGVHTFPAVLQSSGLYS | |
| | | LSSVVTVPSSSLGTQTYICNVNHKPSNTKVDK | |
| | | KVEPKSCDKTHTCPPCPAPEAAGGPSVFLFPP | |
| | | KPKDTLMISRTPEVTCVVVDVSHEDPEVKFN | |
| | | WYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1381 HC | N55Q of | QVQLVQSGAEVKKPGASVKVSCKASRYTFTD | 73 |
| | h1017-H2 Fc- | YNLHWVRQAPGQGLEWMGFIYPYQGGTGY | |
| | silenced IgG1 | AQKFQGRVTMTVDTSTSTAYMELSSLRSEDT | |
| | | AVYYCARDHRYNEGVSFDYWGQGTTVTVSS | |
| | | ASTKGPSVFPLAPSSKSTSGGTAALGCLVKDY | |
| | | FPEPVTVSWNSGALTSGVHTFPAVLQSSGLYS | |
| | | LSSVVTVPSSSLGTQTYICNVNHKPSNTKVDK | |
| | | KVEPKSCDKTHTCPPCPAPEAAGGPSVFLFPP | |
| | | KPKDTLMISRTPEVTCVVVDVSHEDPEVKFN | |
| | | WYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1382 HC | N55A of | QVQLVQSGAEVKKPGASVKVSCKASRYTFTD | 74 |
| | h1017-H2 Fc- | YNLHWVRQAPGQGLEWMGFIYPYAGGTGY | |
| | silenced IgG1 | AQKFQGRVTMTVDTSTSTAYMELSSLRSEDT | |
| | | AVYYCARDHRYNEGVSFDYWGQGTTVTVSS | |
| | | ASTKGPSVFPLAPSSKSTSGGTAALGCLVKDY | |
| | | FPEPVTVSWNSGALTSGVHTFPAVLQSSGLYS | |
| | | LSSVVTVPSSSLGTQTYICNVNHKPSNTKVDK | |
| | | KVEPKSCDKTHTCPPCPAPEAAGGPSVFLFPP | |
| | | KPKDTLMISRTPEVTCVVVDVSHEDPEVKFN | |
| | | WYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| CBD1037 HC | h1017-H4 Fc- | QVQLVQSGAEVKKPGASVKVSCKASRYTFTD | 75 |
| | silenced IgG1 | YNLHWVRQAPGQGLEWMGFIYPYNGGTGY | |
| | | AQKFQGRVTMTVDYSTSTAYMELSSLRSEDT | |
| | | AVYYCARDHRYNEGVSFDYWGQGTTVTVSS | |
| | | ASTKGPSVFPLAPSSKSTSGGTAALGCLVKDY | |
| | | FPEPVTVSWNSGALTSGVHTFPAVLQSSGLYS | |
| | | LSSVVTVPSSSLGTQTYICNVNHKPSNTKVDK | |
| | | KVEPKSCDKTHTCPPCPAPEAAGGPSVFLFPP | |
| | | KPKDTLMISRTPEVTCVVVDVSHEDPEVKFN | |
| | | WYVDGVEVHNAKTKPREEQYNSTYRVVSVL | |
| | | TVLHQDWLNGKEYKCKVSNKALAAPIEKTIS | |
| | | KAKGQPREPQVYTLPPSREEMTKNQVSLTCL | |
| | | VKGFYPSDIAVEWESNGQPENNYKTTPPVLD | |
| | | SDGSFFLYSKLTVDKSRWQQGNVFSCSVMHE | |
| | | ALHNHYTQKSLSLSPG | |
| VL-CDR1 | Modified VL- | RASESVSGFGNSFMN | 227 |
| CBD1017 D30S | CDR1 of | | |
| | mouse anti- | | |
| | CD8α clone | | |
| | CT8 | | |
| VL-CDR1 | Modified VL- | RASESVAGFGNSFMN | 228 |
| CBD1017 D30A | CDR1 of | | |
| | mouse anti- | | |
| | CD8α clone | | |
| | CT8 | | |
| hOKT8 variant 1 | humanized | DVQITQSPSSLSASVGDRVTITCRTSRSISQYL | 229 |
| VL | OKT8 variant | AWYQQKPGKVPKLLIYSGSTLQSGVPS | |
| | 1 VL | RFSGSGSGTDFTLTISSLQPEDVATYYCQQHN | |
| | | ENPLTFGGGTKVEIK | |
| hOKT8 variant 1 | humanized | EVOLVESGGGLVQPGGSLRLSCAASGFNIKDT | 230 |
| VH | OKT8 variant | YIHFVRQAPGKGLEWIGRIDPANDNTLY | |
| | 1 VH | ASKFQGKATISADTSKNTAYLQMNSLRAEDT | |
| | | AVYYCGRGYGYYVFDHWGQGTLVTVSS | |
| hOKT2 LC | humanized | DVQITQSPSSLSASVGDRVTITCRTSRSISQYL | 231 |
| | OKT8 variant | AWYQEKPGKTNKLLIYSGSTLQSGIPS | |
| | 2 LC | RFSGSGSGTDFTLTISSLQPEDFATYYCQQVN | |
| | | EFPPTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFNR | |
| | | GEC | |
| hOKT2 HC | humanized | EVQLVQSGAEVKKPGASVKVSCKASGFNIKD | 232 |
| | OKT8 variant | TYIHWVRQAPGQGLEWIGRIDPANDNTLY | |
| | 2 HC | ASKFQGRATITADTSTSTAYLELSSLRSEDTA | |
| | | VYYCGRGYGYYVFDHWGQGTLVTVSSAS | |
| | | TKGPSVFPLAPSSKSTSGGTAALGCLVKDYFP | |
| | | EPVTVSWNSGALTSGVHTFPAVLQSSGL | |
| | | YSLSSVVTVPSSSLGTQTYICNVNHKPSNTKV | |
| | | DKKVEPKSCDKTHTCPPCPAPEAAGGPS | |
| | | VFLFPPKPKDTLMISRTPEVTCVVVDVSHEDP | |
| | | EVKFNWYVDGVEVHNAKTKPREEQYNST | |
| | | YRVVSVLTVLHQDWLNGKEYKCKVSNKALG | |
| | | APIEKTISKAKGQPREPQVYTLPPSREEMT | |
| | | KNQVSLSCAVKGFYPSDIAVEWESNGQPENN | |
| | | YKTTPPVLDSDGSFFLVSKLTVDKSRWQQ | |
| | | GNVFSCSVMHEALHNHYTQKSLSLSPGK | |

HC: heavy chain.
LC: light chain.
CDR residues are Bold.
X is any amino acid.
X₁ is N, S, Q, or A.
X₂ is N, Q, D, S, or A.
X₃ is D, E, S, or A

In various embodiments, any light chain comprising one of the above light chain variable domains is paired with any of the heavy chains comprising one of the above heavy chain variable domains to denote a whole antibody. For example, a light chain having SEQ ID NO: 62 (a complete kappa chain), 63, 64, or 65 (light chain variable domains) can be paired with a heavy chain having SEQ ID NO: 61, 72, 73, or 74. For example, CBD1033.3 is a pairing of SEQ ID NOS: 61 and 62. Each assemblage of VL and CL paired with an assemblage of VH and CH from components described in Table 16 or elsewhere herein above constitutes a further embodiment. In further embodiments, the variable domains can be paired in various antibody fragments as disclosed herein including scFv, diabodies, minibodies, F(ab), F(ab′), or F(ab′)₂, such as the F(ab′) set out in Table 17 (below).

TABLE 17
Humanized anti-CD8a F(ab′) variants and classic and
engineered F(ab′) constant regions.
Variable domains are Bold.
Constant regions are in regular fonts.
C-terminal hinge residue modification is Bold Italic Underlined.
The mutation sites of native disulfide residues(to remove the
cysteine used in the native disulfide) are Italic Underlined.
The engineered disulfide residues (mutation to introduce a
new cysteine for interchain disulfide bond) are Bold Underlined.
The target conjugation sites are Underlined.
Kabat numbering is used to identify the position of substitutions
and other modifications of the amino acid sequences.
Kabat numbering for IgG CHI and hinge domains and corresponding
Eu numbering can be found at imgt.org/IMGTScientificChart/Numbering/Hu_IGHGnber.html
and for Cκ at imgt.org/IMGTScientificChart/Numbering/Hu_IGKCnber.html.
CH1: first heavy chain constant domain.
CH: constant region of F(ab′) including partial hinge sequence.
CK or Cκ: kappa light chain constant domain.

| Name | Notes | LC | HC |
|---|---|---|---|
| .6 IgG1 | Wildtype | | ASTKGPSVFPLAPSSKSTSG |
| F(ab′) CH | human IgG1 | | GTAALGCLVKDYFPEPVTVS |
| | F(ab′) | | WNSGALTSGVHTFPAVLQSS |
| | | | GLYSLSSVVTVPSSSLGTQT |
| | | | YICNVNHKPSNTKVDKKVEP |
| | | | KSCDKTHTCPPCPAPELLG |
| | | | (SEQ ID NO: 76) |
| CBD1033.6 | h1017-L2 | DIQLTQSPSSLSASVGDR | QVQLVQSGAEVKKPGASV |
| | h1017-H2 | ATITCRASESVDGFGNSF | KVSCKASRYTFTDYNLHW |
| | IgG1 F(ab′) | MNWYQQKPGKAPKLLIYL | VRQAPGQGLEWMGFIYPY |
| | Kappa | ASNLESGVPSRFSGSGSG | NGGTGYAQKFQGRVTMTV |
| | | TDFTLTISSVQPEDFATY | DTSTSTAYMELSSLRSED |
| | | YCQQNNEDPYTFGQGT | TAVYYCARDHRYNEGVSF |
| | | KLEIKRTVAAPSVFIF | DYWGQGTTVTVSSASTKG |
| | | PPSDEQLKSGTASVVC | PSVFPLAPSSKSTSGGTA |
| | | LLNNFYPREAKVQWKVD | ALGCLVKDYFPEPVTVSW |
| | | NALQSGNSQESVTEQDS | NSGALTSGVHTFPAVLQS |
| | | KDSTYSLSSTLTLSKADY | SGLYSLSSVVTVPSSSLG |
| | | EKHKVYACEVTHQGLSS | TQTYICNVNHKPSNTKVD |
| | | PVTKSFNRGEC (SEQ ID | KKVEPKSCDKTHTCPPCP |
| | | | APELLG (SEQ |
| | | NO: 77) | ID NO: 78) |
| .61 IgG4 | Wildtype | | ASTKGPSVFPLAPCSRSTSE |
| F(ab′) CH | human IgG4 | | STAALGCLVKDYFPEPVTVS |
| | F(ab′) | | WNSGALTSGVHTFPAVLQSS |
| | | | GLYSLSSVVTVPSSSLGTKT |
| | | | YTCNVDHKPSNTKVDKRVE |
| | | | SKYGPPCPPCPAPEFLG |
| | | | (SEQ ID NO: 79) |
| CBD1033.61 | h1017-L2 | DIQLTQSPSSLSASVGDR | QVQLVQSGAEVKKPGASV |
| | h1017-H2 | ATITCRASESVDGFGNS | KVSCKASRYTFTDYNLHW |
| | IgG4 F(ab′) | FMNWYQQKPGKAPKL | VRQAPGQGLEWMGFIYPY |
| | Kappa | LIYLASNLESGVPSRFSG | NGGTGYAQKFQGRVTMTV |
| | | SGSGTDFTLTISSVQPED | DTSTSTAYMELSSLRSED |
| | | FATYYCQQNNEDPYTF | TAVYYCARDHRYNEGVSF |
| | | GQGTKLEIKRTVAAPSV | DYWGQGTTVTVSSASTKG |
| | | FIFPPSDEQLKSGTASVVC | PSVFPLAPCSRSTSESTA |
| | | LLNNFYPREAKVQWKVD | ALGCLVKDYFPEPVTVSW |
| | | NALQSGNSQESVTEQDS | NSGALTSGVHTFPAVLQS |
| | | KDSTYSLSSTLTLSKADY | SGLYSLSSVVTVPSSSLG |
| | | EKHKVYACEVTHQGLSS | TKTYTCNVDHKPSNTKVD |
| | | PVTKSFNRGEC | KRVESKYGPPCPPCPAPE |
| | | (SEQ ID | FLG |
| | | NO: 77) | (SEQ ID NO: 80) |
| .9 Truncated | Wildtype | | ASTKGPSVFPLAPSSKSTSG |
| P245 IgG1 | human IgG1 | | GTAALGCLVKDYFPEPVTVS |
| F(ab′) CH | F(ab′) | | WNSGALTSGVHTFPAVLQSS |
| | Truncated at | | GLYSLSSVVTVPSSSLGTQT |
| | Pro245 | | YICNVNHKPSNTKVDKKVE |
| | | | PKSCDKTHTCPPCPAP |
| | | | (SEQ ID NO: 81) |
| CBD1033.9 | h1017-L2 | DIQLTQSPSSLSASVGDR | QVQLVQSGAEVKKPGASV |
| | h1017-H2 | ATITCRASESVDGFGNS | KVSCKASRYTFTDYNLHW |
| | Truncated | FMNWYQQKPGKAPKL | VRQAPGQGLEWMGFIYPY |
| | P245 IgG1 | LIYLASNLESGVPSRFSG | NGGTGYAQKFQGRVTMTV |
| | F(ab′) | SGSGTDFTLTISSVQPED | DTSTSTAYMELSSLRSED |
| | Kappa | FATYYCQQNNEDPYTF | TAVYYCARDHRYNEGVSF |
| | | GQGTKLEIKRTVAAPSV | DYWGQGTTVTVSSASTKG |
| | | FIFPPSDEQLKSGTASVVC | PSVFPLAPSSKSTSGGTA |
| | | LLNNFYPREAKVQWKVD | ALGCLVKDYFPEPVTVSW |
| | | NALQSGNSQESVTEQDS | NSGALTSGVHTFPAVLQS |
| | | KDSTYSLSSTLTLSKADY | SGLYSLSSVVTVPSSSLG |
| | | EKHKVYACEVTHQGLSS | TQTYICNVNHKPSNTKVD |
| | | PVTKSFNRGEC (SEQ ID | KKVEPKSCDKTHTCPPCP |
| | | NO: 77) | AP (SEQ ID NO: 82) |
| .10 | Wildtype | | ASTKGPSVFPLAPCSRSTSE |
| Truncated | human IgG4 | | STAALGCLVKDYFPEPVTVS |
| P245 IgG4 | F(ab′) | | WNSGALTSGVHTFPAVLQSS |
| F(ab′) CH | Truncated at | | GLYSLSSVVTVPSSSLGTKT |
| | Pro245 | | YTCNVDHKPSNTKVDKRVE |
| | | | SKYGPPCPPCPAP |
| | | | (SEQ ID NO: 83) |
| CBD1033.10 | h1017-L2 | DIQLTQSPSSLSASVGDR | QVQLVQSGAEVKKPGASV |
| | h1017-H2 | ATITCRASESVDGFGNS | KVSCKASRYTFTDYNLHW |
| | Truncated | FMNWYQQKPGKAPKL | VRQAPGQGLEWMGFIYPY |
| | P245 IgG4 | LIYLASNLESGVPSRFSG | NGGTGYAQKFQGRVTMTV |
| | F(ab′) | SGSGTDFTLTISSVQPED | DTSTSTAYMELSSLRSED |
| | Kappa | FATYYCQQNNEDPYTF | TAVYYCARDHRYNEGVSF |
| | | GQGTKLEIKRTVAAPSV | DYWGQGTTVTVSSASTKG |
| | | FIFPPSDEQLKSGTASVVC | PSVFPLAPCSRSTSESTA |
| | | LLNNFYPREAKVQWKVD | ALGCLVKDYFPEPVTVSW |
| | | NALQSGNSQESVTEQDS | NSGALTSGVHTFPAVLQS |
| | | KDSTYSLSSTLTLSKADY | SGLYSLSSVVTVPSSSLG |
| | | EKHKVYACEVTHQGLSS | TKTYTCNVDHKPSNTKVD |
| | | PVTKSFNRGEC (SEQ ID | KRVESKYGPPCPPCPAP |
| | | NO: 77) | (SEQ ID NO: 84) |
| .40 | IgG1 F(ab′) | | ASTKGPSVFPLAPSSKSTSG |
| Truncated | Truncated | | GTAALGCLVKDYFPEPVTVS |
| P241, P240A | P241 | | WNSGALTSGVHTFPAVLQSS |
| and P241A | P240A and | | GLYSLSSVVTVPSSSLGTQT |
| IgG1 F(ab′) | P241A | | YICNVNHKPSNTKVDKKVEP |
| CH | | | KSCDKTHTCAA (SEQ ID |
| | | | NO: 85) |
| CBD1033.40 | h1017-L2 | DIQLTQSPSSLSASVGDR | QVQLVQSGAEVKKPGASV |
| | h1017-H2 | ATITCRASESVDGFGNS | KVSCKASRYTFTDYNLHW |
| | Truncated | FMNWYQQKPGKAPKL | VRQAPGQGLEWMGFIYPY |
| | P241, | LIYLASNLESGVPSRFSG | NGGTGYAQKFQGRVTMTV |
| | P240A and | SGSGTDFTLTISSVQPED | DTSTSTAYMELSSLRSED |
| | P241A IgG1 | FATYYCQQNNEDPYTF | TAVYYCARDHRYNEGVSF |
| | F(ab′) | GQGTKLEIKRTVAAPSV | DYWGQGTTVTVSSASTKG |
| | Kappa | FIFPPSDEQLKSGTASVVC | PSVFPLAPSSKSTSGGTAAL |
| | | LLNNFYPREAKVQWKVD | GCLVKDYFPEPVTVSWNSG |
| | | NALQSGNSQESVTEQDS | ALTSGVHTFPAVLQSSGLYS |
| | | KDSTYSLSSTLTLSKADY | LSSVVTVPSSSLGTQTYICNV |
| | | EKHKVYACEVTHQGLSS | NHKPSNTKVDKKVEPKSCD |
| | | PVTKSFNRGEC | KTHTCAA |
| | | (SEQ ID NO: 77) | (SEQ ID NO: 86) |
| .12 | IgG4 F(ab′) | | ASTKGPSVFPLAPCSRSTSE |
| Truncated | Truncated | | STAALGCLVKDYFPEPVTVS |
| P241, P240A | P241 | | WNSGALTSGVHTFPAVLQSS |
| and P241A | P240A and | | GLYSLSSVVTVPSSSLGTKT |
| IgG4 F(ab′) | P241A | | YTCNVDHKPSNTKVDKRVE |
| CH | | | SKYGPPCAA |
| | | | (SEQ ID NO: 87) |
| CBD1033.12 | h1017-L2 | DIQLTQSPSSLSASVGDR | QVQLVQSGAEVKKPGASV |
| | h1017-H2 | ATITCRASESVDGFGNS | KVSCKASRYTFTDYNLHW |
| | Truncated | FMNWYQQKPGKAPKL | VRQAPGQGLEWMGFIYPY |
| | P241, | LIYLASNLESGVPSRFSG | NGGTGYAQKFQGRVTMTV |
| | P240A and | SGSGTDFTLTISSVQPED | DTSTSTAYMELSSLRSED |
| | P241A IgG4 | FATYYCQQNNEDPYTF | TAVYYCARDHRYNEGVSF |
| | F(ab′) | GQGTKLEIKRTVAAPSV | DYWGQGTTVTVSSASTKG |
| | Kappa | FIFPPSDEQLKSGTASVVC | PSVFPLAPCSRSTSESTA |
| | | LLNNFYPREAKVQWKVD | ALGCLVKDYFPEPVTVSW |
| | | NALQSGNSQESVTEQDS | NSGALTSGVHTFPAVLQS |
| | | KDSTYSLSSTLTLSKADY | SGLYSLSSVVTVPSSSLG |
| | | EKHKVYACEVTHQGLSS | TKTYTCNVDHKPSNTKVD |
| | | PVTKSFNRGEC (SEQ ID | KRVESKYGPPCAA |
| | | NO: 77) | (SEQ ID NO: 88) |
| S162C and | Kappa chain | RTVAAPSVFIFPPSDEQLK | |
| C214S | S162C | SGTASVVCLLNNFYPREA | |
| Kappa chain | C214S | KVQWKVDNALQSGNSQ | |
| | | ECVTEQDSKDSTYSLSST | |
| | | LTLSKADYEKHKVYACE | |
| | | VTHQGLSSPVTKSFNRGE | |
| | | S (SEQ ID NO: 89) | |
| .37 | IgG1 F(ab′) | | ASTKGPSVFPLAPSSKSTSGG |
| Truncated | Truncated | | TAALGCLVKDYFPEPVTVS |
| P241, P240A | P241 | | WNSGALTSGVHTCPAVLQS |
| and P241A, | P240A and | | SGLYSLSSVVTVPSSSLGTQT |
| F174C, | P241A | | YICNVNHKPSNTKVDKKVE |
| C233S IgG1 | F174C, | | PKSSDKTHTCAA (SEQ ID |
| F(ab′) CH | C233S | | NO: 90) |
| CBD1033.37 | h1017-L2 | DIQLTQSPSSLSASVGDR | QVQLVQSGAEVKKPGASV |
| | h1017-H2 | ATITCRASESVDGFGNS | KVSCKASRYTFTDYNLHW |
| | Truncated | FMNWYQQKPGKAPKL | VRQAPGQGLEWMGFIYPY |
| | P241, | LIYLASNLESGVPSRFSG | NGGTGYAQKFQGRVTMTV |
| | P240A and | SGSGTDFTLTISSVQPED | DTSTSTAYMELSSLRSED |
| | P241A IgG1 | FATYYCQQNNEDPYTF | TAVYYCARDHRYNEGVSF |
| | F(ab′) | GQGTKLEIKRTVAAPSV | DYWGQGTTVTVSSASTKG |
| | CK-S162C | FIFPPSDEQLKSGTASVVC | PSVFPLAPSSKSTSGGTAAL |
| | and CH1- | LLNNFYPREAKVQWKVD | GCLVKDYFPEPVTVSWNSG |
| | F174C | NALQSGNSQECVTEQDS | ALTSGVHTCPAVLQSSGLYS |
| | engineered | KDSTYSLSSTLTLSKADY | LSSVVTVPSSSLGTQTYICNV |
| | disulfide | EKHKVYACEVTHQGLSS | NHKPSNTKVDKKVEPKSSD |
| | bond | PVTKSFNRGES (SEQ ID | KTHTCAA (SEQ ID NO: 92) |
| | CK-C214S | NO: 91) | |
| | and CH1- | | |
| | C233S to | | |
| | abolish | | |
| | native | | |
| | disulfide | | |
| | bond | | |
| .24 | IgG4 F(ab′) | | ASTKGPSVFPLAPSSRSTSE |
| Truncated | Truncated | | STAALGCLVKDYFPEPVTVS |
| P241, P240A | P241 | | WNSGALTSGVHTCPAVLQSS |
| and P241A, | P240A and | | GLYSLSSVVTVPSSSLGTKT |
| F174C, | P241A | | YTCNVDHKPSNTKVDKRVE |
| C127S IgG4 | F174C, | | SKYGPPCAA |
| F(ab′) CH | C127S | | (SEQ ID NO: 93) |
| CBD1033.24 | h1017-L2 | DIQLTQSPSSLSASVGDR | QVQLVQSGAEVKKPGASV |
| | h1017-H2 | ATITCRASESVDGFGNS | KVSCKASRYTFTDYNLHW |
| | Truncated | FMNWYQQKPGKAPKL | VRQAPGQGLEWMGFIYPY |
| | P241, | LIYLASNLESGVPSRFSG | NGGTGYAQKFQGRVTMTV |
| | P240A and | SGSGTDFTLTISSVQPED | DTSTSTAYMELSSLRSED |
| | P241A, | FATYYCQQNNEDPYTF | TAVYYCARDHRYNEGVSF |
| | IgG4 F(ab′) | GQGTKLEIKRTVAAPSV | DYWGQGTTVTVSSASTKG |
| | CK-S162C | FIFPPSDEQLKSGTASVVC | PSVFPLAPSSRSTSESTA |
| | and CH1- | LLNNFYPREAKVQWKVD | ALGCLVKDYFPEPVTVSW |
| | F174C | NALQSGNSQECVTEQDS | NSGALTSGVHTCPAVLQS |
| | engineered | KDSTYSLSSTLTLSKADY | SGLYSLSSVVTVPSSSLG |
| | disulfide | EKHKVYACEVTHQGLSS | TKTYTCNVDHKPSNTKVD |
| | bond. | PVTKSFNRGES (SEQ ID | KRVESKYGPPCAA |
| | CK-C214S | NO: 91) | (SEQ ID NO: 94) |
| | and CH1- | | |
| | C127S to | | |
| | abolish | | |
| | native | | |
| | disulfide | | |
| | bond | | |
| .42 | IgG1 F(ab′) | | ASTKGPSVFPLAPSSKSTSG |
| Truncated | Truncated | | GTAALGCLVKDYFPEPVTVS |
| P240, | P240 | | WNSGALTSGVHTCPAVLQSS |
| F174C, | F174C, | | GLYSLSSVVTVPSSSLGTQT |
| C233S IgG1 | C233S | | YICNVNHKPSNTKVDKKVE |
| F(ab′) CH | | | PKSSDKTHTCP |
| | | | (SEQ ID NO: 95) |
| CBD1033.42 | h1017-L2 | DIQLTQSPSSLSASVGDR | QVQLVQSGAEVKKPGASV |
| | h1017-H2 | ATITCRASESVDGFGNS | KVSCKASRYTFTDYNLHW |
| | Truncated | FMNWYQQKPGKAPKL | VRQAPGQGLEWMGFIYPY |
| | P240 IgG1 | LIYLASNLESGVPSRFSG | NGGTGYAQKFQGRVTMTV |
| | F(ab′) | SGSGTDFTLTISSVQPED | DTSTSTAYMELSSLRSED |
| | CK-C214S | FATYYCQQNNEDPYTF | TAVYYCARDHRYNEGVSF |
| | and CH1- | GQGTKLEIKRTVAAPSV | DYWGQGTTVTVSSASTKG |
| | C233S to | FIFPPSDEQLKSGTASVVC | PSVFPLAPSSKSTSGGTAAL |
| | abolish | LLNNFYPREAKVQWKVD | GCLVKDYFPEPVTVSWNSG |
| | native | NALQSGNSQECVTEQDS | ALTSGVHTCPAVLQSSGLYS |
| | disulfide | KDSTYSLSSTLTLSKADY | LSSVVTVPSSSLGTQTYICNV |
| | bond | EKHKVYACEVTHQGLSS | NHKPSNTKVDKKVEPKSSD |
| | CK-S162C | PVTKSFNRGES (SEQ ID | KTHTCP (SEQ ID NO: 96) |
| | and CH1- | NO: 91) | |
| | F174C | | |
| | engineered | | |
| | disulfide | | |
| | bond. | | |
| .44 | IgG1 F(ab′) | | ASTKGPSVFPLAPSSKSTS |
| Truncated | Truncated at | | GGTAALGCLVKDYFPEPVT |
| T238, F174C | T238 | | VSWNSGALTSGVHTCPAVL |
| IgG1 F(ab′) | F174C | | QSSGLYSLSSVVTVPSSSL |
| CH | | | GTQTYICNVNHKPSNTKVD |
| | | | KKVEPKSCDKTHT |
| | | | (SEQ ID NO: 97) |
| CBD1033.44 | h1017-L2 | DIQLTQSPSSLSASVGDR | QVQLVQSGAEVKKPGASV |
| | h1017-H2 | ATITCRASESVDGFGNS | KVSCKASRYTFTDYNLHW |
| | Truncated | FMNWYQQKPGKAPKL | VRQAPGQGLEWMGFIYPY |
| | T238 IgG1 | LIYLASNLESGVPSRFSG | NGGTGYAQKFQGRVTMT |
| | F(ab′) | SGSGTDFTLTISSVQPED | VDTSTSTAYMELSSLRSED |
| | CK-C214S | FATYYCQQNNEDPYTF | TAVYYCARDHRYNEGVSF |
| | to abolish | GQGTKLEIKRTVAAPSV | DYWGQGTTVTVSSASTKG |
| | native | FIFPPSDEQLKSGTASVVC | PSVFPLAPSSKSTSGGTAAL |
| | disulfide | LLNNFYPREAKVQWKVD | GCLVKDYFPEPVTVSWNSG |
| | bond | NALQSGNSQECVTEQDS | ALTSGVHTCPAVLQSSGLYS |
| | CK-S162C | KDSTYSLSSTLTLSKADY | LSSVVTVPSSSLGTQTYICNV |
| | and CH1- | EKHKVYACEVTHQGLSS | NHKPSNTKVDKKVEPKSCD |
| | F174C | PVTKSFNRGES (SEQ ID | KTHT (SEQ ID NO: 98) |
| | engineered | NO: 91) | |
| | disulfide | | |
| | bond | | |
| .45 | IgG1 F(ab′) | | ASTKGPSVFPLAPSSKSTSG |
| Truncated | Truncated | | GTAALGCLVKDYFPEPVTVS |
| T238, | T238 | | WNSGALTSGVHTCPAVLQSS |
| F174C, | F174C, | | GLYSLSSVVTVPSSSLGTQT |
| C233S IgG1 | C233S | | YICNVNHKPSNTKVDKKVE |
| F(ab′) CH | | | PKSSDKTHT (SEQ ID NO: 99) |
| S162C | Kappa chain | RTVAAPSVFIFPPSDEQLK | |
| Kappa chain | S162C | SGTASVVCLLNNFYPREA | |
| | | KVQWKVDNALQSGNSQ | |
| | | ECVTEQDSKDSTYSLSST | |
| | | LTLSKADYEKHKVYACE | |
| | | VTHQGLSSPVTKSFNRGE | |
| | | C (SEQ ID NO: 100) | |
| CBD1033.45 | h1017-L2 | DIQLTQSPSSLSASVGDR | QVQLVQSGAEVKKPGASV |
| | h1017-H2 | ATITCRASESVDGFGNS | KVSCKASRYTFTDYNLHW |
| | Truncated | FMNWYQQKPGKAPKL | VRQAPGQGLEWMGFIYPY |
| | T238 IgG1 | LIYLASNLESGVPSRFSG | NGGTGYAQKFQGRVTMT |
| | F(ab′) | SGSGTDFTLTISSVQPED | VDTSTSTAYMELSSLRSED |
| | CH1-C233S | FATYYCQQNNEDPYTF | TAVYYCARDHRYNEGVSF |
| | to abolish | GQGTKLEIKRTVAAPSV | DYWGQGTTVTVSSASTKG |
| | native | FIFPPSDEQLKSGTASVVC | PSVFPLAPSSKSTSGGTAAL |
| | disulfide | LLNNFYPREAKVQWKVD | GCLVKDYFPEPVTVSWNSG |
| | bond | NALQSGNSQECVTEQDS | ALTSGVHTCPAVLQSSGLYS |
| | CK-S162C | KDSTYSLSSTLTLSKADY | LSSVVTVPSSSLGTQTYICNV |
| | and CH1- | EKHKVYACEVTHQGLSS | NHKPSNTKVDKKVEPKSSD |
| | F174C | PVTKSFNRGEC (SEQ ID | KTHT (SEQ ID NO: 102) |
| | engineered | NO: 101) | |
| | disulfide | | |
| | bond | | |
| .48 | IgG4 F(ab′) | | ASTKGPSVFPLAPSSRSTSES |
| Truncated | Truncated | | TAALGCLVKDYFPEPVTVS |
| C239, | C239 | | WNSGALTSGVHTCPAVLQS |
| F174C, | F174C, | | SGLYSLSSVVTVPSSSLGTKT |
| C127S IgG4 | C127S | | YTCNVDHKPSNTKVDKRVE |
| F(ab′) CH | | | SKYGPPC (SEQ ID NO: 103) |
| CBD1033.48 | h1017-L2 | DIQLTQSPSSLSASVGDR | QVQLVQSGAEVKKPGASV |
| | h1017-H2 | ATITCRASESVDGFGNS | KVSCKASRYTFTDYNLHW |
| | Truncated | FMNWYQQKPGKAPKL | VRQAPGQGLEWMGFIYPY |
| | C239 IgG4 | LIYLASNLESGVPSRFSG | NGGTGYAQKFQGRVTMT |
| | F(ab′) | SGSGTDFTLTISSVQPED | VDTSTSTAYMELSSLRSED |
| | CK-C214S | FATYYCQQNNEDPYTF | TAVYYCARDHRYNEGVSF |
| | and CH1- | GQGTKLEIKRTVAAPSV | DYWGQGTTVTVSSASTKG |
| | C127S to | FIFPPSDEQLKSGTASVVC | PSVFPLAPSSRSTSESTAALG |
| | abolish | LLNNFYPREAKVQWKVD | CLVKDYFPEPVTVSWNSGA |
| | native | NALQSGNSQECVTEQDS | LTSGVHTCPAVLQSSGLYSL |
| | disulfide | KDSTYSLSSTLTLSKADY | SSVVTVPSSSLGTKTYTCNV |
| | bond | EKHKVYACEVTHQGLSS | DHKPSNTKVDKRVESKYGP |
| | CK-S162C | PVTKSFNRGES (SEQ ID | PC (SEQ ID NO: 104) |
| | and CH1- | NO: 91) | |
| | F174C | | |
| | engineered | | |
| | disulfide | | |
| | bond. | | |
| CBD1381.37 | h1017-L2 | DIQLTQSPSSLSASVGDR | QVQLVQSGAEVKKPGASV |
| | N55Q of | ATITCRASESVDGFGNS | KVSCKASRYTFTDYNLHW |
| | h1017-H2 | FMNWYQQKPGKAPKL | VRQAPGQGLEWMGFIYPY |
| | Truncated | LIYLASNLESGVPSRFSG | QGGTGYAQKFQGRVTMTV |
| | P241, | SGSGTDFTLTISSVQPED | DTSTSTAYMELSSLRSED |
| | P240A and | FATYYCQQNNEDPYTF | TAVYYCARDHRYNEGVSF |
| | P241A IgG1 | GQGTKLEIKRTVAAPSV | DYWGQGTTVTVSSASTKG |
| | F(ab′) | FIFPPSDEQLKSGTASVVC | PSVFPLAPSSKSTSGGTAAL |
| | CK-C214S | LLNNFYPREAKVQWKVD | GCLVKDYFPEPVTVSWNSG |
| | and CH1- | NALQSGNSQECVTEQDS | ALTSGVHTCPAVLQSSGLYS |
| | C233S to | KDSTYSLSSTLTLSKADY | LSSVVTVPSSSLGTQTYICNV |
| | abolish | EKHKVYACEVTHQGLSS | NHKPSNTKVDKKVEPKSSD |
| | native | PVTKSFNRGES (SEQ ID | KTHTCAA |
| | disulfide | NO: 91) | (SEQ ID NO: 105) |
| | bond | | |
| | CK-S162C | | |
| | and CH1- | | |
| | F174C | | |
| | engineered | | |
| | disulfide | | |
| | bond | | |
| CBD1382.37 | h1017-L2 | DIQLTQSPSSLSASVGDR | QVQLVQSGAEVKKPGASV |
| | N55A of | ATITCRASESVDGFGNS | KVSCKASRYTFTDYNLHW |
| | h1017-H2 | FMNWYQQKPGKAPKL | VRQAPGQGLEWMGFIYPY |
| | Truncated | LIYLASNLESGVPSRFSG | AGGTGYAQKFQGRVTMTV |
| | P241, | SGSGTDFTLTISSVQPED | DTSTSTAYMELSSLRSED |
| | P240A and | FATYYCQQNNEDPYTF | TAVYYCARDHRYNEGVSF |
| | P241A IgG1 | GQGTKLEIKRTVAAPSV | DYWGQGTTVTVSSASTKG |
| | F(ab′) | FIFPPSDEQLKSGTASVVC | PSVFPLAPSSKSTSGGTAAL |
| | CK-C214S | LLNNFYPREAKVQWKVD | GCLVKDYFPEPVTVSWNSG |
| | and CH1- | NALQSGNSQECVTEQDS | ALTSGVHTCPAVLQSSGLYS |
| | C233S to | KDSTYSLSSTLTLSKADY | LSSVVTVPSSSLGTQTYICNV |
| | abolish | EKHKVYACEVTHQGLSS | NHKPSNTKVDKKVEPKSSD |
| | native | PVTKSFNRGES (SEQ ID | KTHTCAA |
| | disulfide | NO: 91) | (SEQ ID NO: 106) |
| | bond | | |
| | CK-S162C | | |
| | and CH1- | | |
| | F174C | | |
| | engineered | | |
| | disulfide | | |
| | bond | | |
| CBD1444.37 | D30S of | DIQLTQSPSSLSASVGDR | QVQLVQSGAEVKKPGASV |
| | h1017-L2 | ATITCRASESVSGFGNSF | KVSCKASRYTFTDYNLHW |
| | h1017-H2 | MNWYQQKPGKAPKLLI | VRQAPGQGLEWMGFIYPY |
| | Truncated | YLASNLESGVPSRFSGS | NGGTGYAQKFQGRVTMTV |
| | P241, | GSGTDFTLTISSVQPEDF | DTSTSTAYMELSSLRSED |
| | P240A and | ATYYCQQNNEDPYTFG | TAVYYCARDHRYNEGVSF |
| | P241A IgG1 | QGTKLEIKRTVAAPSVFI | DYWGQGTTVTVSSASTKG |
| | F(ab′) | FPPSDEQLKSGTASVVCL | PSVFPLAPSSKSTSGGTAAL |
| | CK-C214S | LNNFYPREAKVQWKVD | GCLVKDYFPEPVTVSWNSG |
| | and CH1- | NALQSGNSQECVTEQDS | ALTSGVHTCPAVLQSSGLYS |
| | C233S to | KDSTYSLSSTLTLSKADY | LSSVVTVPSSSLGTQTYICNV |
| | abolish | EKHKVYACEVTHQGLSS | NHKPSNTKVDKKVEPKSSD |
| | native | PVTKSFNRGES (SEQ ID | KTHTCAA |
| | disulfide | NO: 107) | (SEQ ID NO: 92) |
| | bond | | |
| | CK-S162C | | |
| | and CH1- | | |
| | F174C | | |
| | engineered | | |
| | disulfide | | |
| | bond | | |
| CBD1622.37 | D30S of | DIQLTQSPSSLSASVGDR | QVQLVQSGAEVKKPGASV |
| | h1017-L2 | ATITCRASESVSGFGNSF | KVSCKASRYTFTDYNLHW |
| | N55Q of | MNWYQQKPGKAPKLLI | VRQAPGQGLEWMGFIYPY |
| | h1017-H2 | YLASNLESGVPSRFSGS | QGGTGYAQKFQGRVTMTV |
| | Truncated | GSGTDFTLTISSVQPEDF | DTSTSTAYMELSSLRSED |
| | P241, | ATYYCQQNNEDPYTFG | TAVYYCARDHRYNEGVSF |
| | P240A and | QGTKLEIKRTVAAPSVFI | DYWGQGTTVTVSSASTKG |
| | P241A IgG1 | FPPSDEQLKSGTASVVCL | PSVFPLAPSSKSTSGGTAAL |
| | F(ab′) | LNNFYPREAKVQWKVD | GCLVKDYFPEPVTVSWNSG |
| | CK-C214S | NALQSGNSQECVTEQDS | ALTSGVHTCPAVLQSSGLYS |
| | and CH1- | KDSTYSLSSTLTLSKADY | LSSVVTVPSSSLGTQTYICNV |
| | C233S to | EKHKVYACEVTHQGLSS | NHKPSNTKVDKKVEPKSSD |
| | abolish | PVTKSFNRGES (SEQ ID | KTHTCAA |
| | native | NO: 107) | (SEQ ID NO: 105) |
| | disulfide | | |
| | bond | | |
| | CK-S162C | | |
| | and CH1- | | |
| | F174C | | |
| | engineered | | |
| | disulfide | | |
| | bond. | | |
| CBD1623.37 | D30S of | DIQLTQSPSSLSASVGDR | QVQLVQSGAEVKKPGASV |
| | h1017-L2 | ATITCRASESVSGFGNSF | KVSCKASRYTFTDYNLHW |
| | N55A of | MNWYQQKPGKAPKLLI | VRQAPGQGLEWMGFIYPY |
| | h1017-H2 | YLASNLESGVPSRFSGS | AGGTGYAQKFQGRVTMT |
| | Truncated | GSGTDFTLTISSVQPEDF | VDTSTSTAYMELSSLRSED |
| | P241, | ATYYCQQNNEDPYTFG | TAVYYCARDHRYNEGVSF |
| | P240A and | QGTKLEIKRTVAAPSVFI | DYWGQGTTVTVSSASTKG |
| | P241A IgG1 | FPPSDEQLKSGTASVVCL | PSVFPLAPSSKSTSGGTAAL |
| | F(ab′) | LNNFYPREAKVQWKVD | GCLVKDYFPEPVTVSWNSG |
| | CK-C214S | NALQSGNSQECVTEQDS | ALTSGVHTCPAVLQSSGLYS |
| | and CH1- | KDSTYSLSSTLTLSKADY | LSSVVTVPSSSLGTQTYICNV |
| | C233S to | EKHKVYACEVTHQGLSS | NHKPSNTKVDKKVEPKSSD |
| | abolish | PVTKSFNRGES (SEQ ID | KTHTCAA |
| | native | NO: 107) | (SEQ ID NO: 106) |
| | disulfide | | |
| | bond | | |
| | CK-S162C | | |
| | and CH1- | | |
| | F174C | | |
| | engineered | | |
| | disulfide | | |
| | bond | | |
| CBD1622.42 | D30S of | DIQLTQSPSSLSASVGDR | QVQLVQSGAEVKKPGASV |
| | h1017-L2 | ATITCRASESVSGFGNSF | KVSCKASRYTFTDYNLHW |
| | N55Q of | MNWYQQKPGKAPKLLI | VRQAPGQGLEWMGFIYPY |
| | h1017-H2 | YLASNLESGVPSRFSGS | QGGTGYAQKFQGRVTMT |
| | Truncated | GSGTDFTLTISSVQPEDF | VDTSTSTAYMELSSLRSED |
| | P240 IgG1 | ATYYCQQNNEDPYTFG | TAVYYCARDHRYNEGVSF |
| | F(ab′) | QGTKLEIKRTVAAPSVFI | DYWGQGTTVTVSSASTKG |
| | CK-C214S | FPPSDEQLKSGTASVVCL | PSVFPLAPSSKSTSGGTAAL |
| | and CH1- | LNNFYPREAKVQWKVD | GCLVKDYFPEPVTVSWNSG |
| | C233S to | NALQSGNSQECVTEQDS | ALTSGVHTCPAVLQSSGLYS |
| | abolish | KDSTYSLSSTLTLSKADY | LSSVVTVPSSSLGTQTYICNV |
| | native | EKHKVYACEVTHQGLSS | NHKPSNTKVDKKVEPKSSD |
| | disulfide | PVTKSFNRGES (SEQ ID | KTHTCP |
| | bond | NO: 107) | (SEQ ID NO: 108) |
| | CK-S162C | | |
| | and CH1- | | |
| | F174C | | |
| | engineered | | |
| | disulfide | | |
| | bond | | |
| CBD1623.42 | D30S of | DIQLTQSPSSLSASVGDR | QVQLVQSGAEVKKPGASV |
| | h1017-L2 | ATITCRASESVSGFGNSF | KVSCKASRYTFTDYNLHW |
| | N55A of | MNWYQQKPGKAPKLLI | VRQAPGQGLEWMGFIYPY |
| | h1017-H2 | YLASNLESGVPSRFSGS | AGGTGYAQKFQGRVTMT |
| | Truncated | GSGTDFTLTISSVQPEDF | VDTSTSTAYMELSSLRSED |
| | P240 IgG1 | ATYYCQQNNEDPYTFG | TAVYYCARDHRYNEGVSF |
| | F(ab′) | QGTKLEIKRTVAAPSVFI | DYWGQGTTVTVSSASTKG |
| | CK-C214S | FPPSDEQLKSGTASVVCL | PSVFPLAPSSKSTSGGTAAL |
| | and CH1- | LNNFYPREAKVQWKVD | GCLVKDYFPEPVTVSWNSG |
| | C233S to | NALQSGNSQECVTEQDS | ALTSGVHTCPAVLQSSGLYS |
| | abolish | KDSTYSLSSTLTLSKADY | LSSVVTVPSSSLGTQTYICNV |
| | native | EKHKVYACEVTHQGLSS | NHKPSNTKVDKKVEPKSSD |
| | disulfide | PVTKSFNRGES (SEQ ID | KTHTCP |
| | bond | NO: 107) | (SEQ ID NO: 109) |
| | CK-S162C | | |
| | and CH1- | | |
| | F174C | | |
| | engineered | | |
| | disulfide | | |
| | bond | | |
| CBD1622.44 | D30S of | DIQLTQSPSSLSASVGDR | QVQLVQSGAEVKKPGASV |
| | h1017-L2 | ATITCRASESVSGFGNSF | KVSCKASRYTFTDYNLHW |
| | N55Q of | MNWYQQKPGKAPKLLI | VRQAPGQGLEWMGFIYPY |
| | h1017-H2 | YLASNLESGVPSRFSGS | QGGTGYAQKFQGRVTMT |
| | Truncated | GSGTDFTLTISSVQPEDF | VDTSTSTAYMELSSLRSED |
| | T238 IgG1 | ATYYCQQNNEDPYTFG | TAVYYCARDHRYNEGVSF |
| | F(ab′) | QGTKLEIKRTVAAPSVFI | DYWGQGTTVTVSSASTKG |
| | CK-C214S | FPPSDEQLKSGTASVVCL | PSVFPLAPSSKSTSGGTAAL |
| | to abolish | LNNFYPREAKVQWKVD | GCLVKDYFPEPVTVSWNSG |
| | native | NALQSGNSQECVTEQDS | ALTSGVHTCPAVLQSSGLYS |
| | disulfide | KDSTYSLSSTLTLSKADY | LSSVVTVPSSSLGTQTYICNV |
| | bond | EKHKVYACEVTHQGLSS | NHKPSNTKVDKKVEPKSCD |
| | CK-S162C | PVTKSFNRGES (SEQ ID | KTHT |
| | and CH1- | NO: 107) | (SEQ ID NO: 110) |
| | F174C | | |
| | engineered | | |
| | disulfide | | |
| | bond. | | |
| CBD1623.44 | D30S of | DIQLTQSPSSLSASVGDR | QVQLVQSGAEVKKPGASV |
| | h1017-L2 | ATITCRASESVSGFGNSF | KVSCKASRYTFTDYNLHW |
| | N55A of | MNWYQQKPGKAPKLLI | VRQAPGQGLEWMGFIYPY |
| | h1017-H2 | YLASNLESGVPSRFSGS | AGGTGYAQKFQGRVTMT |
| | Truncated | GSGTDFTLTISSVQPEDF | VDTSTSTAYMELSSLRSED |
| | T238 IgG1 | ATYYCQQNNEDPYTFG | TAVYYCARDHRYNEGVSF |
| | F(ab′) | QGTKLEIKRTVAAPSVFI | DYWGQGTTVTVSSASTKG |
| | CK-C214S | FPPSDEQLKSGTASVVCL | PSVFPLAPSSKSTSGGTAAL |
| | to abolish | LNNFYPREAKVQWKVD | GCLVKDYFPEPVTVSWNSG |
| | native | NALQSGNSQECVTEQDS | ALTSGVHTCPAVLQSSGLYS |
| | disulfide | KDSTYSLSSTLTLSKADY | LSSVVTVPSSSLGTQTYICNV |
| | bond | EKHKVYACEVTHQGLSS | NHKPSNTKVDKKVEPKSCD |
| | CK-S162C | PVTKSFNRGES (SEQ ID | KTHT |
| | and CH1- | NO: 107) | (SEQ ID NO: 111) |
| | F174C | | |
| | engineered | | |
| | disulfide | | |
| | bond. | | |
| CBD1622.45 | D30S of | DIQLTQSPSSLSASVGDR | QVQLVQSGAEVKKPGASV |
| | h1017-L2 | ATITCRASESVSGFGNSF | KVSCKASRYTFTDYNLHW |
| | N55Q of | MNWYQQKPGKAPKLLI | VRQAPGQGLEWMGFIYPY |
| | h1017-H2 | YLASNLESGVPSRFSGS | QGGTGYAQKFQGRVTMT |
| | Truncated | GSGTDFTLTISSVQPEDF | VDTSTSTAYMELSSLRSED |
| | T238, | ATYYCQQNNEDPYTFG | TAVYYCARDHRYNEGVSF |
| | F174C, | QGTKLEIKRTVAAPSVFI | DYWGQGTTVTVSSASTKG |
| | C233S IgG1 | FPPSDEQLKSGTASVVCL | PSVFPLAPSSKSTSGGTAAL |
| | F(ab′) | LNNFYPREAKVQWKVD | GCLVKDYFPEPVTVSWNSG |
| | CH1-C233S | NALQSGNSQECVTEQDS | ALTSGVHTCPAVLQSSGLYS |
| | to abolish | KDSTYSLSSTLTLSKADY | LSSVVTVPSSSLGTQTYICNV |
| | native | EKHKVYACEVTHQGLSS | NHKPSNTKVDKKVEPKSSD |
| | disulfide | PVTKSFNRGEC (SEQ ID | KTHT |
| | bond | NO: 112) | (SEQ ID NO: 113) |
| | CK-S162C | | |
| | and CH1- | | |
| | F174C | | |
| | engineered | | |
| | disulfide | | |
| | bond | | |
| CBD1623.45 | D30S of | DIQLTQSPSSLSASVGDR | QVQLVQSGAEVKKPGASV |
| | h1017-L2 | ATITCRASESVSGFGNSF | KVSCKASRYTFTDYNLHW |
| | N55A of | MNWYQQKPGKAPKLLI | VRQAPGQGLEWMGFIYPY |
| | h1017-H2 | YLASNLESGVPSRFSGS | AGGTGYAQKFQGRVTMT |
| | Truncated | GSGTDFTLTISSVQPEDF | VDTSTSTAYMELSSLRSED |
| | T238 IgG1 | ATYYCQQNNEDPYTFG | TAVYYCARDHRYNEGVSF |
| | F(ab′) | QGTKLEIKRTVAAPSVFI | DYWGQGTTVTVSSASTKG |
| | CH1-C233S | FPPSDEQLKSGTASVVCL | PSVFPLAPSSKSTSGGTAAL |
| | to abolish | LNNFYPREAKVQWKVD | GCLVKDYFPEPVTVSWNSG |
| | native | NALQSGNSQECVTEQDS | ALTSGVHTCPAVLQSSGLYS |
| | disulfide | KDSTYSLSSTLTLSKADY | LSSVVTVPSSSLGTQTYICNV |
| | bond | EKHKVYACEVTHQGLSS | NHKPSNTKVDKKVEPKSSD |
| | CK-S162C | PVTKSFNRGEC (SEQ ID | KTHT |
| | and CH1- | NO: 112) | (SEQ ID NO: 114) |
| | F174C | | |
| | engineered | | |
| | disulfide | | |
| | bond | | |

TABLE 18
Summary of full-length and F(ab′) humanized anti-CD8α variants constructs.

| | | VH constant | | VL constant |
|---|---|---|---|---|
| Name | VH | region | VL | region |
| CBD1032 | h1017-H1 (SEQ | Fc-silenced | h1017-L1 (SEQ | Kappa chain |
| | ID NO: 10) | IgG1 (SEQ ID | ID NO: 16) | (SEQ ID NO: |
| | | NO: 43) | | 41) |
| CBD1033 | h1017-H2 (SEQ | Fc-silenced | h1017-L2 (SEQ | Kappa chain |
| | ID NO: 11) | IgG1 (SEQ ID | ID NO: 17) | (SEQ ID NO: |
| | | NO: 43) | | 41) |
| CBD1034 | h1017-H2 (SEQ | Fc-silenced | h1017-L3 (SEQ | Kappa chain |
| | ID NO: 11) | IgG1 (SEQ ID | ID NO: 18) | (SEQ ID NO: |
| | | NO: 43) | | 41) |
| CBD1035 | h1017-H3 (SEQ | Fc-silenced | h1017-L2 (SEQ | Kappa chain |
| | ID NO: 12) | IgG1 (SEQ ID | ID NO: 17) | (SEQ ID NO: |
| | | NO: 43) | | 41) |
| CBD1036 | h1017-H3 (SEQ | Fc-silenced | h1017-L3 (SEQ | Kappa chain |
| | ID NO: 12) | IgG1 (SEQ ID | ID NO: 18) | (SEQ ID NO: |
| | | NO: 43) | | 41) |
| CBD1037 | h1017-H4 (SEQ | Fc-silenced | h1017-L2 (SEQ | Kappa chain |
| | ID NO: 13) | IgG1 (SEQ ID | ID NO: 17) | (SEQ ID NO: |
| | | NO: 43) | | 41) |
| CBD1038 | h1017-H4 (SEQ | Fc-silenced | h1017-L3 (SEQ | Kappa chain |
| | ID NO: 13) | IgG1 (SEQ ID | ID NO: 18) | (SEQ ID NO: |
| | | NO: 43) | | 41) |
| CBD1039 | h1017-H5 (SEQ | Fc-silenced | h1017-L2 (SEQ | Kappa chain |
| | ID NO: 14) | IgG1 (SEQ ID | ID NO: 17) | (SEQ ID NO: |
| | | NO: 43) | | 41) |
| CBD1040 | h1017-H5 (SEQ | Fc-silenced | h1017-L3 (SEQ | Kappa chain |
| | ID NO: 14) | IgG1 (SEQ ID | ID NO: 18) | (SEQ ID NO: |
| | | NO: 43) | | 41) |
| CBD1041 | h1017-H6 (SEQ | Fc-silenced | h1017-L4 (SEQ | Kappa chain |
| | ID NO: 32) | IgG1 (SEQ ID | ID NO: 38) | (SEQ ID NO: |
| | | NO: 43) | | 41) |
| CBD1042 | h1017-H7 (SEQ | Fc-silenced | h1017-L4 (SEQ | Kappa chain |
| | ID NO: 33) | IgG1 (SEQ ID | ID NO: 38) | (SEQ ID NO: |
| | | NO: 43) | | 41) |
| CBD1043 | h1017-H7 (SEQ | Fc-silenced | h1017-L5 (SEQ | Kappa chain |
| | ID NO: 33) | IgG1 (SEQ ID | ID NO: 39) | (SEQ ID NO: |
| | | NO: 43) | | 41) |
| CBD1044 | h1017-H7 (SEQ | Fc-silenced | h1017-L6 (SEQ | Kappa chain |
| | ID NO: 33) | IgG1 (SEQ ID | ID NO: 40) | (SEQ ID NO: |
| | | NO: 43) | | 41) |
| CBD1045 | h1017-H8 (SEQ | Fc-silenced | h1017-L5 (SEQ | Kappa chain |
| | ID NO: 34) | IgG1 (SEQ ID | ID NO: 39) | (SEQ ID NO: |
| | | NO: 43) | | 41) |
| CBD1046 | h1017-H8 (SEQ | Fc-silenced | h1017-L6 (SEQ | Kappa chain |
| | ID NO: 34) | IgG1 (SEQ ID | ID NO: 40) | (SEQ ID NO: |
| | | NO: 43) | | 41) |
| CBD1047 | h1017-H9 (SEQ | Fc-silenced | h1017-L5 (SEQ | Kappa chain |
| | ID NO: 35) | IgG1 (SEQ ID | ID NO: 39) | (SEQ ID NO: |
| | | NO: 43) | | 41) |
| CBD1048 | h1017-H9 (SEQ | Fc-silenced | h1017-L6 (SEQ | Kappa chain |
| | ID NO: 35) | IgG1 (SEQ ID | ID NO: 40) | (SEQ ID NO: |
| | | NO: 43) | | 41) |
| CBD1049 | h1017-H10 | Fc-silenced | h1017-L5 (SEQ | Kappa chain |
| | (SEQ ID NO: | IgG1 (SEQ ID | ID NO: 39) | (SEQ ID NO: |
| | 36) | NO: 43) | | 41) |
| CBD1050 | h1017-H10 | Fc-silenced | h1017-L6 (SEQ | Kappa chain |
| | (SEQ ID NO: | IgG1 (SEQ ID | ID NO: 40) | (SEQ ID NO: |
| | 36) | NO: 43) | | 41) |
| CBD1128 | h1017-H1 (SEQ | Fc-silenced | h1017-L2 (SEQ | Kappa chain |
| | ID NO: 10) | IgG1 (SEQ ID | ID NO: 17) | (SEQ ID NO: |
| | | NO: 43) | | 41) |
| CBD1129 | h1017-H2 (SEQ | Fc-silenced | h1017-L1 (SEQ | Kappa chain |
| | ID NO: 11) | IgG1 (SEQ ID | ID NO: 16) | (SEQ ID NO: |
| | | NO: 43) | | 41) |
| CBD1380 | N55S of h1017- | Fc-silenced | h1017-L2 (SEQ | Kappa chain |
| | H2 (SEQ ID | IgG1 (SEQ ID | ID NO: 17) | (SEQ ID NO: |
| | NO: 27) | NO: 43) | | 41) |
| CBD1381 | N55Q of h1017- | Fc-silenced | h1017-L2 (SEQ | Kappa chain |
| | H2 (SEQ ID | IgG1 (SEQ ID | ID NO: 17) | (SEQ ID NO: |
| | NO: 28) | NO: 43) | | 41) |
| CBD1382 | N55A of h1017- | Fc-silenced | h1017-L2 (SEQ | Kappa chain |
| | H2 (SEQ ID | IgG1 (SEQ ID | ID NO: 17) | (SEQ ID NO: |
| | NO: 29) | NO: 43) | | 41) |
| CBD1383 | N55D of h1017- | Fc-silenced | h1017-L2 (SEQ | Kappa chain |
| | H2 (SEQ ID | IgG1 (SEQ ID | ID NO: 17) | (SEQ ID NO: |
| | NO: 30) | NO: 43) | | 41) |
| CBD1443 | h1017-H2 (SEQ | Fc-silenced | D30E of h1017- | Kappa chain |
| | ID NO: 11) | IgG1 (SEQ ID | L2 (SEQ ID NO: | (SEQ ID NO: |
| | | NO: 43) | 63) | 41) |
| CBD1444 | h1017-H2 (SEQ | Fc-silenced | D30S of h1017- | Kappa chain |
| | ID NO: 11) | IgG1 (SEQ ID | L2 (SEQ ID NO: | (SEQ ID NO: |
| | | NO: 43) | 64 | 41) |
| CBD1445 | h1017-H2 (SEQ | Fc-silenced | D30A of h1017- | Kappa chain |
| | ID NO: 11) | IgG1 (SEQ ID | L2 (SEQ ID NO: | (SEQ ID NO: |
| | | NO: 43) | 65) | 41) |
| CBD1446 | h1017-H4 (SEQ | Fc-silenced | D30E of h1017- | Kappa chain |
| | ID NO: 13) | IgG1 (SEQ ID | L2 (SEQ ID NO: | (SEQ ID NO: |
| | | NO: 43) | 63) | 41) |
| CBD1447 | h1017-H4 (SEQ | Fc-silenced | D30S of h1017- | Kappa chain |
| | ID NO: 13) | IgG1 (SEQ ID | L2 (SEQ ID NO: | (SEQ ID NO: |
| | | NO: 43) | 64) | 41) |
| CBD1448 | h1017-H4 (SEQ | Fc-silenced | D30A of h1017- | Kappa chain |
| | ID NO: 13) | IgG1 (SEQ ID | L2 (SEQ ID NO: | (SEQ ID NO: |
| | | NO: 43) | 65) | 41) |
| CBD1449 | N55S of h1017- | Fc-silenced | h1017-L2 (SEQ | Kappa chain |
| | H4 (SEQ ID | IgG1 (SEQ ID | ID NO: 17) | (SEQ ID NO: |
| | NO: 66) | NO: 43) | | 41) |
| CBD1450 | N55Q of h1017- | Fc-silenced | h1017-L2 (SEQ | Kappa chain |
| | H4 (SEQ ID | IgG1 (SEQ ID | ID NO: 17) | (SEQ ID NO: |
| | NO: 67) | NO: 43) | | 41) |
| CBD1451 | N55A of h1017- | Fc-silenced | h1017-L2 (SEQ | Kappa chain |
| | H4 (SEQ ID | IgG1 (SEQ ID | ID NO: 17) | (SEQ ID NO: |
| | NO: 68) | NO: 43) | | 41) |
| CBD1575 | N55Q of h1017- | Fc-silenced | D30S of h1017- | Kappa chain |
| | H4 (SEQ ID | IgG1 (SEQ ID | L2 (SEQ ID NO: | (SEQ ID NO: |
| | NO: 67) | NO: 43) | 64) | 41) |
| CBD1576 | N55A of h1017- | Fc-silenced | D30S of h1017- | Kappa chain |
| | H4 (SEQ ID | IgG1 (SEQ ID | L2 (SEQ ID NO: | (SEQ ID NO: |
| | NO: 68) | NO: 43) | 64) | 41) |
| CBD1622 | N55Q of h1017- | Fc-silenced | D30S of h1017- | Kappa chain |
| | H2 (SEQ ID | IgG1 (SEQ ID | L2 (SEQ ID NO: | (SEQ ID NO: |
| | NO: 28) | NO: 43) | 64) | 41) |
| CBD1623 | N55A of h1017- | Fc-silenced | D30S of h1017- | Kappa chain |
| | H2 (SEQ ID | IgG1 (SEQ ID | L2 (SEQ ID NO: | (SEQ ID NO: |
| | NO: 29) | NO: 43) | 64) | 41) |
| CBD1033.6 | h1017-H2 (SEQ | IgG1 F(ab′) | h1017-L2 (SEQ | Kappa chain |
| | ID NO: 11) | (SEQ ID NO: | ID NO: 17) | (SEQ ID NO: |
| | | 76) | | 41) |
| CBD1033.61 | h1017-H2 (SEQ | IgG4 F(ab′) | h1017-L2 (SEQ | Kappa chain |
| | ID NO: 11) | (SEQ ID NO: | ID NO: 17) | (SEQ ID NO: |
| | | 79) | | 41) |
| CBD1033.9 | h1017-H2 (SEQ | Truncated P245 | h1017-L2 (SEQ | Kappa chain |
| | ID NO: 11) | IgG1 F(ab′) | ID NO: 17) | (SEQ ID NO: |
| | | (SEQ ID NO: | | 41) |
| | | 81) | | |
| CBD1033.10 | h1017-H2 (SEQ | Truncated P245 | h1017-L2 (SEQ | Kappa chain |
| | ID NO: 11) | IgG4 F(ab′) | ID NO: 17) | (SEQ ID NO: |
| | | (SEQ ID NO: | | 41) |
| | | 83) | | |
| CBD1033.40 | h1017-H2 (SEQ | Truncated P241, | h1017-L2 (SEQ | Kappa chain |
| | ID NO: 11) | P240A and | ID NO: 17) | (SEQ ID NO: |
| | | P241A IgG1 | | 41) |
| | | F(ab′) (SEQ | | |
| | | ID NO: 85) | | |
| CBD1033.12 | h1017-H2 (SEQ | Truncated P241, | h1017-L2 (SEQ | Kappa chain |
| | ID NO: 11) | P240A and | ID NO: 17) | (SEQ ID NO: |
| | | P241A IgG4 | | 41) |
| | | F(ab′) (SEQ | | |
| | | ID NO: 87) | | |
| CBD1033.37 | h1017-H2 (SEQ | Truncated P241, | h1017-L2 (SEQ | S162C and |
| | ID NO: 11) | P240A and | ID NO: 17) | C214S Kappa |
| | | P241A, F174C, | | chain (SEQ |
| | | C233S IgG1 | | ID NO: 89) |
| | | F(ab′) (SEQ | | |
| | | ID NO: 90) | | |
| CBD1033.24 | h1017-H2 (SEQ | Truncated P241, | h1017-L2 (SEQ | S162C and |
| | ID NO: 11) | P240A and | ID NO: 17) | C214S Kappa |
| | | P241A, F174C, | | chain (SEQ |
| | | C127S IgG4 | | ID NO: 89) |
| | | F(ab′) (SEQ | | |
| | | ID NO: 93) | | |
| CBD1033.42 | h1017-H2 (SEQ | Truncated P240, | h1017-L2 (SEQ | S162C and |
| | ID NO: 11) | F174C, C233S | ID NO: 17) | C214S Kappa |
| | | IgG1 F(ab′) | | chain (SEQ |
| | | (SEQ ID NO: | | ID NO: 89) |
| | | 95) | | |
| CBD1033.44 | h1017-H2 (SEQ | Truncated T238, | h1017-L2 (SEQ | S162C and |
| | ID NO: 11) | F174C IgG1 | ID NO: 17) | C214S Kappa |
| | | F(ab′) (SEQ | | chain (SEQ |
| | | ID NO: 97) | | ID NO: 89) |
| CBD1033.45 | h1017-H2 (SEQ | Truncated T238, | h1017-L2 (SEQ | S162C Kappa |
| | ID NO: 11) | F174C, C233S | ID NO: 17) | chain (SEQ |
| | | IgG1 F(ab′) | | ID NO: 100) |
| | | (SEQ ID NO: | | |
| | | 99) | | |
| CBD1033.48 | h1017-H2 (SEQ | Truncated P239, | h1017-L2 (SEQ | S162C and |
| | ID NO: 11) | F174C, C127S | ID NO: 17) | C214S Kappa |
| | | IgG4 F(ab′) | | chain (SEQ |
| | | (SEQ ID NO: | | ID NO: 89) |
| | | 103) | | |
| CBD1381.37 | N55Q of h1017- | Truncated P241, | h1017-L2 (SEQ | S162C and |
| | H2 (SEQ ID | P240A and | ID NO: 17) | C214S Kappa |
| | NO: 28) | P241A, F174C, | | chain (SEQ |
| | | C233S IgG1 | | ID NO: 89) |
| | | F(ab′) (SEQ | | |
| | | ID NO: 90) | | |
| CBD1382.37 | N55A of h1017- | Truncated P241, | h1017-L2 (SEQ | S162C and |
| | H2 (SEQ ID | P240A and | ID NO: 17) | C214S Kappa |
| | NO: 29) | P241A, F174C, | | chain (SEQ |
| | | C233S IgG1 | | ID NO: 89) |
| | | F(ab′) (SEQ | | |
| | | ID NO: 90) | | |
| CBD1444.37 | h1017-H2 (SEQ | Truncated P241, | D30S of h1017- | S162C and |
| | ID NO: 11) | P240A and | L2 (SEQ ID NO: | C214S Kappa |
| | | P241A, F174C, | 64) | chain (SEQ |
| | | C233S IgG1 | | ID NO: 89) |
| | | F(ab′) (SEQ | | |
| | | ID NO: 90) | | |
| CBD1622.37 | N55Q of h1017- | Truncated P241, | D30S of h1017- | S162C and |
| | H2 (SEQ ID | P240A and | L2 (SEQ ID NO: | C214S Kappa |
| | NO: 28) | P241A, F174C, | 64) | chain (SEQ |
| | | C233S IgG1 | | ID NO: 89) |
| | | F(ab′) (SEQ | | |
| | | ID NO: 90) | | |
| CBD1623.37 | N55A of h1017- | Truncated P241, | D30S of h1017- | S162C and |
| | H2 (SEQ ID | P240A and | L2 (SEQ ID NO: | C214S Kappa |
| | NO: 29) | P241A, F174C, | 64) | chain (SEQ |
| | | C233S IgG1 | | ID NO: 89) |
| | | F(ab′) (SEQ | | |
| | | ID NO: 90) | | |
| CBD1622.42 | N55Q of h1017- | Truncated P240, | D30S of h1017- | S162C and |
| | H2 (SEQ ID | F174C, C233S | L2 (SEQ ID NO: | C214S Kappa |
| | NO: 28) | IgG1 F(ab′) | 64) | chain (SEQ |
| | | (SEQ ID NO: | | ID NO: 89) |
| | | 95) | | |
| CBD1623.42 | N55A of h1017- | Truncated P240, | D30S of h1017- | S162C and |
| | H2 (SEQ ID | F174C, C233S | L2 (SEQ ID NO: | C214S Kappa |
| | NO: 29) | IgG1 F(ab′) | 64) | chain (SEQ |
| | | (SEQ ID NO: | | ID NO: 89) |
| | | 95) | | |
| CBD1622.44 | N55Q of h1017- | Truncated T238, | D30S of h1017- | S162C and |
| | H2 (SEQ ID | F174C IgG1 | L2 (SEQ ID NO: | C214S Kappa |
| | NO: 28) | F(ab′) (SEQ | 64) | chain (SEQ |
| | | ID NO: 97) | | ID NO: 89) |
| CBD1623.44 | N55A of h1017- | Truncated T238, | D30S of h1017- | S162C and |
| | H2 (SEQ ID | F174C IgG1 | L2 (SEQ ID NO: | C214S Kappa |
| | NO: 29) | F(ab′) (SEQ | 64) | chain (SEQ |
| | | ID NO: 97) | | ID NO: 89) |
| CBD1622.45 | N55Q of h1017- | Truncated T238, | D30S of h1017- | S162C Kappa |
| | H2 (SEQ ID | F174C, C233S | L2 (SEQ ID NO: | chain (SEQ |
| | NO: 28) | IgG1 F(ab′) | 64) | ID NO: 100) |
| | | (SEQ ID NO: | | |
| | | 99) | | |
| CBD1623.45 | N55A of h1017- | Truncated T238, | D30S of h1017- | S162C Kappa |
| | H2 (SEQ ID | F174C, C233S | L2 (SEQ ID NO: | chain (SEQ |
| | NO: 29) | IgG1 F(ab′) | 64) | ID NO: 100) |
| | | (SEQ ID NO: | | |
| | | 99) | | |

TABLE 19
Sequences of TRX2 antibody. CDR sequences are based on IMGT numbering.

| | | | SEQ |
|---|---|---|---|
| CBD | | | ID |
| number | Description | Sequence | NO |
| CBD1145 | TRX2 heavy | QVQLVESGGGVVQPGRSLRLSCAASGFTFSDFGMNWVRQ | 216 |
| HC | chain | APGKGLEWVALIYYDGSNKFYADSVKGRFTISRDNSKNTL | |
| | | YLQMNSLRAEDTAVYYCAKPHYDGYYHFFDSWGQGTLVT | |
| | | VSSASTKGPSVFPLAPSSKSTSGGTAALGCLVKDYFPEP | |
| | | VTVSWNSGALTSGVHTFPAVLQSSGLYSLSSVVTVPSSS | |
| | | LGTQTYICNVNHKPSNTKVDKKVEPKSCDKTHTCPPCPA | |
| | | PEAAGGPSVFLFPPKPKDTLMISRTPEVTCVVVDVSHED | |
| | | PEVKFNWYVDGVEVHNAKTKPREEQYNSTYRVVSVLTVL | |
| | | HQDWLNGKEYKCKVSNKALAAPIEKTISKAKGQPREPQV | |
| | | YTLPPSREEMTKNQVSLTCLVKGFYPSDIAVEWESNGQP | |
| | | ENNYKTTPPVLDSDGSFFLYSKLTVDKSRWQQGNVFSCS | |
| | | VMHEALHNHYTQKSLSLSPG | |
| CBD1145 | TRX2 light | DIQMTQSPSSLSASVGDRVTITCKGSQDINNYLAWYQQK | 217 |
| LC | chain | PGKAPKLLIYNTDILHTGVPSRFSGSGSGTDFTFTISSL | |
| | | QPEDIATYYCYQYNNGYTFGQGTKVEIKRTVAAPSVFIF | |
| | | PPSDEQLKSGTASVVCLLNNFYPREAKVQWKVDNALQSG | |
| | | NSQESVTEQDSKDSTYSLSSTLTLSKADYEKHKVYACEV | |
| | | THQGLSSPVTKSFNRGEC | |
| CBD1145 | TRX2 | QVQLVESGGGVVQPGRSLRLSCAASGFTFSDFGMNWVRQ | 218 |
| VH | variable | APGKGLEWVALIYYDGSNKFYADSVKGRFTISRDNSKNT | |
| | heavy chain | LYLQMNSLRAEDTAVYYCAKPHYDGYYHFFDSWGQGTLV | |
| | | TVSS | |
| CBD1145 | TRX2 | DIQMTQSPSSLSASVGDRVTITCKGSQDINNYLAWYQQKP | 219 |
| VL | variable light | GKAPKLLIYNTDILHTGVPSRFSGSGSGTDFTFTISSLQP | |
| | chain | EDIATYYCYQYNNGYTFGQGTKVEIK | |
| | TRX2 | GFTFSDFG | 220 |
| | CDR_H1 | | |
| | TRX2 | IYYDGSNK | 221 |
| | CDR_H2 | | |
| | TRX2 | AKPHYDGYYHFFDS | 222 |
| | CDR_H3 | | |
| | TRX2 | KGSQDINNYLA | 223 |
| | CDR_L1 | | |
| | TRX2 | NTDILHT | 224 |
| | CDR_L2 | | |
| | TRX2 | YQYNNGYT | 225 |
| | CDR_L3 | | |

### Embodiments

Embodiment 1. An isolated antibody or antigen binding fragment thereof comprising a humanized immunoglobulin antigen binding domain that specifically binds to human CD8, comprising:
(a) a heavy chain variable region (VH) comprising an amino acid sequence that has at least 90% identity with the amino acid sequence of framework regions of SEQ ID NO: 9 or 31 wherein the VH comprises a heavy chain CDR1 (VH-CDR1) comprising the amino acid sequence RYTFTDYX₁LH (SEQ ID NO: 45), a VH-CDR2 comprising the amino acid sequence FIYPYX₁GGTG (SEQ ID NO: 46) or FIYPYX₂GGTG (SEQ ID NO: 47), and a VH-CDR3 having the amino acid sequence DHRYX₁EGVSFDY (SEQ ID NO: 48); and
(b) a light chain variable region (VL) comprising an amino acid sequence that has at least 90% identity with the amino acid sequence of framework regions of SEQ ID NO: 15 or 37, wherein the VL comprises a CDR1 (VL-CDR1) comprising the amino acid sequence RASESVX₃GFGX₁SFMN (SEQ ID NO: 49), VL-CDR2 comprising the amino acid sequence LASX₂LES (SEQ ID NO: 50), and a VL-CDR3 having the amino acid sequence QQX₂X₂EX₃PYT (SEQ ID NO: 51),
   wherein each X₁ is independently N, S, Q, or A; each X₂ is independently N, Q, D, S, or A; and each X₃ is independently D, E, S, or A.

Embodiment 2. The isolated antibody or antigen binding fragment thereof of embodiment 1, wherein X₁ of VH-CDR2 is S, Q, or A.

Embodiment 3. The isolated antibody or antigen binding fragment thereof of embodiment 1 or 2, wherein X₂ of VL-CDR1 is S or A.

Embodiment 4. The isolated antibody or antigen binding fragment thereof of embodiment 1 or 2, wherein the VH comprises the amino acid sequence of SEQ ID NO: 27, SEQ ID NO: 66, SEQ ID NO: 28, SEQ ID NO: 67, SEQ ID NO: 29, or SEQ ID NO: 68.

Embodiment 5. The isolated antibody or antigen binding fragment thereof of embodiments 1 and 3, wherein the VL comprises the amino acid sequence of SEQ ID NO: 64 or SEQ ID NO: 65.

Embodiment 6. The isolated antibody or antigen binding fragment thereof of embodiment 1, comprising:
(a) a human heavy chain variable region (VH) wherein the VH comprises a heavy chain CDR1 (VH-CDR1) comprising the amino acid sequence of SEQ ID NO:2, a VH-CDR2 comprising the amino acid sequence of SEQ ID NO: 3, 58, 59, or 60, a VH-CDR3 comprising the amino acid sequence of SEQ ID NO: 4; and
(b) a light chain variable region (VL) wherein the VL comprises a CDR1 (VL-CDR1) comprising the amino acid sequence of SEQ ID NO: 6, 227, or 228, a VL-CDR2 comprising the amino acid sequence of SEQ ID NO: 7, and a VL-CDR3 comprising the amino acid sequence of SEQ ID NO: 8.

Embodiment 7. The isolated antibody or antigen binding fragment thereof of embodiment 1 or 6, wherein:
(a) the VH comprising an amino acid sequence that has at least 90% identity with the amino acid sequence of SEQ ID NO: 10, SEQ ID NO: 11, SEQ ID NO: 12, SEQ ID NO: 13, or SEQ ID NO: 14, and wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4; and
(b) the VL comprising an amino acid sequence that has at least 90% identity with the amino acid sequence of SEQ ID NO: 16, SEQ ID NO: 17, SEQ ID NO: 18, and wherein the VL-CDR1 comprises the amino acid sequence of SEQ ID NO: 6, the VL-CDR2 comprises the amino acid sequence of SEQ ID NO: 7, and the VL-CDR3 comprises the amino acid sequence of SEQ ID NO: 8.

Embodiment 8. The isolated antibody or antigen binding fragment thereof of embodiment 1 or 6, wherein:
(a) the VH comprising an amino acid sequence that has at least 90% identity with the amino acid sequence of SEQ ID NO: 33, SEQ ID NO: 34, SEQ ID NO: 35, or SEQ ID NO: 36, and wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4; and
(b) the VL comprising an amino acid sequence that has at least 90% identity with the amino acid sequence of SEQ ID NO: 38, SEQ ID NO: 39, or SEQ ID NO: 40, and wherein the VL-CDR1 comprises the amino acid sequence of SEQ ID NO: 6, the VL-CDR2 comprises the amino acid sequence of SEQ ID NO: 7, and the VL-CDR3 comprises the amino acid sequence of SEQ ID NO: 8.

Embodiment 9. The isolated antibody or antigen binding fragment thereof of embodiment 1 or embodiment 6,
wherein the VH comprising an amino acid sequence that has at least 90% identity with the amino acid sequence of SEQ ID NO: 10, SEQ ID NO: 11, SEQ ID NO: 12, SEQ ID NO: 13, SEQ ID NO: 14, SEQ ID NO: 27, SEQ ID NO: 28, SEQ ID NO: 29, SEQ ID NO: 33, SEQ ID NO 34, SEQ ID NO: 35, SEQ ID NO: 36, SEQ ID NO: 66, SEQ ID NO: 67, or SEQ ID NO: 68, and
wherein VL comprising an amino acid sequence that has at least 90% identity with the amino acid sequence of SEQ ID NO: 16, SEQ ID NO: 17, SEQ ID NO: 18, SEQ ID NO: 38, SEQ ID NO: 39, SEQ ID NO: 40, SEQ ID NO: 64, or SEQ ID NO: 65.

Embodiment 10. The isolated antibody or antigen binding fragment thereof of embodiment 1, embodiment 6, or embodiment 9, wherein:
(a) the VH comprises the amino acid sequence of SEQ ID NO: 10, SEQ ID NO: 11, SEQ ID NO: 12, SEQ ID NO: 13, SEQ ID NO: 14, SEQ ID NO: 27, SEQ ID NO: 28, SEQ ID NO: 29, SEQ ID NO: 35, or SEQ ID NO: 36, and the VL comprises the amino acid sequence of SEQ ID NO: 16;
(b) the VH comprises the amino acid sequence of SEQ ID NO: 10, SEQ ID NO: 11, SEQ ID NO: 12, SEQ ID NO: 13, SEQ ID NO: 14, SEQ ID NO: 27, SEQ ID NO: 28, SEQ ID NO: 29, SEQ ID NO: 35, or SEQ ID NO: 36, and the VL comprises the amino acid sequence of SEQ ID NO: 17;
(c) the VH comprises the amino acid sequence of SEQ ID NO: 10, SEQ ID NO: 11, SEQ ID NO: 12, SEQ ID NO: 13, SEQ ID NO: 14, SEQ ID NO: 27, SEQ ID NO: 28, SEQ ID NO: 29, SEQ ID NO: 35, or SEQ ID NO: 36, and the VL comprises the amino acid sequence of SEQ ID NO: 18;
(d) the VH comprising the amino acid sequence of SEQ ID NO: 10, SEQ ID NO: 11, SEQ ID NO: 12, SEQ ID NO: 13, SEQ ID NO: 14, SEQ ID NO: 27, SEQ ID NO: 28, SEQ ID NO: 29, SEQ ID NO: 35, or SEQ ID NO: 36, and the VL comprises the amino acid sequence of SEQ ID NO: 39.

Embodiment 11. The isolated antibody or antigen binding fragment thereof of embodiment 1 or 6, wherein:
(a) the VH comprising an amino acid sequence that has at least 90% identity with the amino acid sequence of SEQ ID NO: 11, and wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4; and
(b) the VL comprising an amino acid sequence that has at least 90% identity with the amino acid sequence of SEQ ID NO: 17, and wherein the VL-CDR1 comprises the amino acid sequence of SEQ ID NO: 6, the VL-CDR2 comprises the amino acid sequence of SEQ ID NO: 7, and the VL-CDR3 comprises the amino acid sequence of SEQ ID NO: 8.

Embodiment 12. The isolated antibody or antigen binding fragment thereof of any one of embodiments 1-11, wherein VH and VL are joined in a scFv or diabody.

Embodiment 13. The isolated antibody or antigen binding fragment thereof of any one of embodiments 1-12, comprising a kappa, lambda, human IgG1, human IgG2, human IgG3, or human IgG4 constant region.

Embodiment 14. The isolated antibody or antigen binding fragment thereof of embodiment 13 comprising a silenced Fc region.

Embodiment 15. The isolated antibody of embodiment 14, wherein the silenced Fc region comprises SEQ ID NO: 43 or 44.

Embodiment 16. The isolated antibody of embodiment 15, comprising a heavy chain having the amino acid sequence of SEQ ID NO: 61.

Embodiment 17. The isolated antibody of embodiment 13 or 14 that is a whole antibody.

Embodiment 18. The isolated antibody or antigen binding fragment thereof of embodiment 13, wherein the kappa constant region has the amino acid sequence of SEQ ID NO: 41

Embodiment 19. The isolated antibody of embodiment 13, wherein the human IgG1 constant region has the amino acid sequence of SEQ ID NO: 42, SEQ ID NO: 43, or SEQ ID NO: 44.

Embodiment 20. The antigen binding fragment of embodiment 13 that is an F(ab), F(ab′), or F(ab′) analog.

Embodiment 21. The antigen binding fragment of embodiment 20 comprising a human IgG1 F(ab′) constant region having the amino acid sequence of SEQ ID NO: 76.

Embodiment 22. The antigen binding fragment of embodiment 21, wherein the F(ab′) heavy chain has the amino acid sequence of SEQ ID NO: 78.

Embodiment 23. The antigen binding fragment of embodiment 20, comprising a human IgG4 F(ab′) constant region having the amino acid sequence of SEQ ID NO: 79.

Embodiment 24. The antigen binding fragment of embodiment 23, wherein the F(ab′) heavy chain has the amino acid sequence of SEQ ID NO: 80.

Embodiment 25. The antigen binding fragment of any one of embodiments 20-24, comprising a kappa constant region having the amino acid sequence of SEQ ID NO: 41.

Embodiment 26. The antigen binding fragment of embodiment 20, wherein the F(ab′) analog comprising an IgG1 or IgG4 CH1 F174C substitution and a Cκ S162C substitution.

Embodiment 27. The antigen binding fragment of embodiment 26, wherein the F(ab′) analog further comprises a Cκ C214S substitution, a Cκ C214S substitution and a IgG1 hinge C233S substitution, or a Cκ C214S substitution and a IgG1 hinge truncation at T238.

Embodiment 28. The antigen binding fragment of embodiment 26, wherein the F(ab′) analog further comprises an IgG4 CH1 C127S substitution, or an IgG4 CH1 C127S substitution and a Cκ C214S substitution.

Embodiment 29. The antigen binding fragment of embodiment 20, wherein the IgG1 constant region has the amino acid sequence of SEQ ID NO: 81, SEQ ID NO: 85, SEQ ID NO: 90, SEQ ID NO: 95, SEQ ID NO: 97, or SEQ ID NO: 99.

Embodiment 30. The antigen binding fragment of embodiment 27 comprising a heavy chain having the amino acid sequence of SEQ ID NO: 78, SEQ ID NO: 82, SEQ ID NO: 86, SEQ ID NO: 92, SEQ ID NO: 96, SEQ ID NO: 98, SEQ ID NO: 102, SEQ ID NO: 105, SEQ ID NO: 106, SEQ ID NO: 108, SEQ ID NO: 109, SEQ ID NO: 110, SEQ ID NO: 111, SEQ ID NO: 113, or SEQ ID NO: 114.

Embodiment 31. The antigen binding fragment of embodiment 27 comprising a heavy chain having the amino acid sequence of SEQ ID NO: 92, SEQ ID NO: 98, or SEQ ID NO: 102.

Embodiment 32. The antigen binding fragment of embodiment 27 comprising a heavy chain having the amino acid sequence of SEQ ID NO: 98, SEQ ID NO: 102, SEQ ID NO: 110, SEQ ID NO: 111, SEQ ID NO: 113, or SEQ ID NO: 114.

Embodiment 33. The antigen binding fragment of embodiment 20, wherein the IgG4 constant region has the amino acid sequence of SEQ ID NO: 83, SEQ ID NO: 87, SEQ ID NO: 93, or SEQ ID NO: 103.

Embodiment 34. The antigen binding fragment of embodiment 33 comprising a heavy chain having the amino acid sequence of SEQ ID NO: 84, SEQ ID NO: 88, SEQ ID NO: 94, or SEQ ID NO: 104.

Embodiment 35. The antigen binding fragment of embodiment 26, wherein the Cκ has the amino acid sequence of SEQ ID NO: 89, or SEQ ID NO: 100.

Embodiment 36. The antigen binding fragment of embodiment 35 comprising a light chain having the amino acid sequence of SEQ ID NO: 91, SEQ ID NO: 101, SEQ ID NO: 107, or SEQ ID NO: 112.

Embodiment 37. The antigen binding fragment of embodiment 31 or embodiment 36, comprising:
(a) the heavy chain having the amino acid sequence of SEQ ID NO: 92 and the light chain having the amino acid sequence of SEQ ID NO: 91;
(b) the heavy chain having the amino acid sequence of SEQ ID NO: 98 and the light chain having the amino acid sequence of SEQ ID NO: 91; or
(c) the heavy chain having the amino acid sequence of SEQ ID NO: 102 and the light chain having the amino acid sequence of SEQ ID NO: 101.

Embodiment 38. The antigen binding fragment of embodiment 32 or embodiment 36, comprising:
(a) the heavy chain having the amino acid sequence of SEQ ID NO: 98 and the light chain having the amino acid sequence of SEQ ID NO: 91;
(b) the heavy chain having the amino acid sequence of SEQ ID NO: 102 and the light chain having the amino acid sequence of SEQ ID NO: 101;
(c) the heavy chain having the amino acid sequence of SEQ ID NO: 110 and the light chain having the amino acid sequence of SEQ ID NO: 107;
(d) the heavy chain having the amino acid sequence of SEQ ID NO: 113 and the light chain having the amino acid sequence of SEQ ID NO: 112;
(e) the heavy chain having the amino acid sequence of SEQ ID NO: 111 and the light chain having the amino acid sequence of SEQ ID NO: 107; or
(f) the heavy chain having the amino acid sequence of SEQ ID NO: 114 and the light chain having the amino acid sequence of SEQ ID NO: 112;

Embodiment 39. The isolated antibody or antigen binding fragment thereof of any one of embodiments 1-38, wherein the humanized antibody or antigen binding fragment thereof has a temperature of aggregation (T_{agg})≥60 □C and a melting temperature (T_{M}) of ≥65 □C.

Embodiment 40. The isolated antibody or antigen binding fragment thereof of any one of embodiments 1-39, wherein the humanized antibody or antigen binding fragment thereof has a low propensity for self-interaction.

Embodiment 41. The isolated antibody or antigen binding fragment thereof of any one of embodiments 1-40, wherein the humanized antibody or antigen binding fragment thereof lacks polyreactivity to
(a) double-stranded DNA and insulin;
(b) baculovirus particles;
(c) human cell surface and secreted proteins; or
(d) any combination of (a) to (c).

Embodiment 42. The isolated antibody or antigen binding fragment thereof of any one of embodiments 1-41, wherein the humanized antibody or antigen binding fragment thereof has minimal to undetectable off-target binding.

Embodiment 43. The isolated antibody or antigen binding fragment thereof of any one of embodiments 1-42, comprising a thiolated lysine residue at Lys248 or Lys288 of the constant region.

Embodiment 44. The isolated antibody or antigen binding fragment thereof of any one of embodiments 1-14 that is an F(ab), F(ab′), F(ab′)₂, scFv, diabody, or minibody.

Embodiment 45. An F(ab′) analog comprising the VH and VL of The isolated antibody or antigen binding fragment thereof of any one of embodiments 1-11.

Embodiment 46. An F(ab′) analog comprising a relocated interchain disulfide bond and an antigen binding domain that binds the CT8 epitope of CD8.

Embodiment 47. An F(ab′) analog comprising an antigen binding domain that competes for binding to the epitope bound by the anti-CD8 antibody CT8, TRX2, or YTC182.20.

Embodiment 48. The F(ab′) analog of embodiment 46 or embodiment 47 comprising means for binding the CT8 epitope or means for binding to the same epitope as bound by CT8, TRX2, and/or YTC182.20.

Embodiment 49. The F(ab′) analog of any one of embodiments 46-48 comprising an IgG1 or IgG4 CH1 F174C substitution and a Cκ S162C substitution.

Embodiment 50. The F(ab′) analog of embodiment 49 further comprising a Cκ C214S substitution, or a Cκ C214S substitution and a IgG1 hinge C233S substitution, or a Cκ C214S substitution and a IgG1 hinge truncation at T238.

Embodiment 51. The F(ab′) analog of embodiment 49 further comprising an IgG4 CH1 C127S substitution, or an IgG4 CH1 C127S substitution and a Cκ C214S substitution.

Embodiment 52. The F(ab′) analog of any one of embodiments 46-51 comprising a VH and VL,
wherein the VH comprises a heavy chain CDR1 (VH-CDR1) having the amino acid sequence of SEQ ID NO: 220, a VH-CDR2 having the amino acid sequence of SEQ ID NO: 221, and a VH-CDR3 having the amino acid sequence of SEQ ID NO: 222; and
wherein the VL comprises a CDR1 (VL-CDR1) comprising the amino acid sequence of SEQ ID NO: 223, VL-CDR2 having the amino acid sequence of SEQ ID NO: 224, and a VL-CDR3 having the amino acid sequence of SEQ ID NO: 225.

Embodiment 53. The F(ab′) analog of any one of embodiments 46-51 comprising the VH and VL of YTC182.20.

Embodiment 54. The F(ab′) analog of any one of embodiments 46-51 comprising the VH and VL of CT8.

Embodiment 55. The F(ab′) analog of any one of embodiments 46-51 comprising:
(a) heavy chain variable region (VH) comprising an amino acid sequence that has at least 90% identity with the amino acid sequence of framework regions of SEQ ID NO: 9 or 31 wherein the VH comprises a heavy chain CDR1 (VH-CDR1) comprising the amino acid sequence RYTFTDYX₁LH (SEQ ID NO: 45), a VH-CDR2 comprising the amino acid sequence FIYPYX₁GGTG (SEQ ID NO: 46) or FIYPYX₂GGTG (SEQ ID NO: 47), and a VH-CDR3 having the amino acid sequence DHRYX₁EGVSFDY (SEQ ID NO: 48); and
(b) a light chain variable region (VL) comprising an amino acid sequence that has at least 90% identity with the amino acid sequence of framework regions of SEQ ID NO: 15 or 37, wherein the VL comprises a CDR1 (VL-CDR1) comprising the amino acid sequence RASESVX₃GFGX₁SFMN (SEQ ID NO: 49), VL-CDR2 comprising the amino acid sequence LASX₂LES (SEQ ID NO: 50), and a VL-CDR3 having the amino acid sequence QQX₂X₂EX₃PYT (SEQ ID NO: 51),
wherein each X₁ is independently N, S, Q, or A; each X₂ is independently N, Q, D, S, or A; and each X₃ is independently D, E, S, or A.

Embodiment 56. The F(ab′) analog of embodiment 55, wherein X₁ of VH-CDR2 is S, Q, or A.

Embodiment 57. The F(ab′) analog of embodiment 55 or embodiment 56, wherein X₂ of VL-CDR1 is S or A.

Embodiment 58. The F(ab′) analog of embodiment 55 or embodiment 56, wherein the VH comprises the amino acid sequence of SEQ ID NO: 27, SEQ ID NO: 66, SEQ ID NO: 28, SEQ ID NO: 67, SEQ ID NO: 29, or SEQ ID NO: 68.

Embodiment 59. The F(ab′) analog of embodiment 55 or embodiment 57, wherein the VL comprises the amino acid sequence of SEQ ID NO: 64 or SEQ ID NO: 65.

Embodiment 60. The F(ab′) analog of embodiment 55, comprising:
(a) a human heavy chain variable region (VH) wherein the VH comprises a heavy chain CDR1 (VH-CDR1) comprising the amino acid sequence of SEQ ID NO:2, a VH-CDR2 comprising the amino acid sequence of SEQ ID NO: 3, 58, 59, or 60, a VH-CDR3 comprising the amino acid sequence of SEQ ID NO: 4; and
(b) a light chain variable region (VL) wherein the VL comprises a CDR1 (VL-CDR1) comprising the amino acid sequence of SEQ ID NO: 6, 227, or 228, a VL-CDR2 comprising the amino acid sequence of SEQ ID NO: 7, and a VL-CDR3 comprising the amino acid sequence of SEQ ID NO: 8.

Embodiment 61. The isolated antibody or antigen binding fragment thereof of embodiment 55 and 60, wherein:
(a) the VH comprising an amino acid sequence that has at least 90% identity with the amino acid sequence of SEQ ID NO: 10, SEQ ID NO: 11, SEQ ID NO: 12, SEQ ID NO: 13, or SEQ ID NO: 14, and wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4; and
(b) the VL comprising an amino acid sequence that has at least 90% identity with the amino acid sequence of SEQ ID NO: 16, SEQ ID NO: 17, SEQ ID NO: 18, and wherein the VL-CDR1 comprises the amino acid sequence of SEQ ID NO: 6, the VL-CDR2 comprises the amino acid sequence of SEQ ID NO: 7, and the VL-CDR3 comprises the amino acid sequence of SEQ ID NO: 8.

Embodiment 62. The isolated antibody or antigen binding fragment thereof of embodiment 55 or 60, wherein:
(a) the VH comprising an amino acid sequence that has at least 90% identity with the amino acid sequence of SEQ ID NO: 33, SEQ ID NO: 34, SEQ ID NO: 35, or SEQ ID NO: 36, and wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4; and
(b) the VL comprising an amino acid sequence that has at least 90% identity with the amino acid sequence of SEQ ID NO: 38, SEQ ID NO: 39, or SEQ ID NO: 40, and wherein the VL-CDR1 comprises the amino acid sequence of SEQ ID NO: 6, the VL-CDR2 comprises the amino acid sequence of SEQ ID NO: 7, and the VL-CDR3 comprises the amino acid sequence of SEQ ID NO: 8.

Embodiment 63. The isolated antibody or antigen binding fragment thereof of embodiment 60,
wherein the VH comprising an amino acid sequence that has at least 90% identity with the amino acid sequence of SEQ ID NO: 10, SEQ ID NO: 11, SEQ ID NO: 12, SEQ ID NO: 13, SEQ ID NO: 14, SEQ ID NO: 27, SEQ ID NO: 28, SEQ ID NO: 29, SEQ ID NO: 33, SEQ ID NO 34, SEQ ID NO: 35, SEQ ID NO: 36, SEQ ID NO: 66, SEQ ID NO: 67, or SEQ ID NO: 68, and
wherein VL comprising an amino acid sequence that has at least 90% identity with the amino acid sequence of SEQ ID NO: 16, SEQ ID NO: 17, SEQ ID NO: 18, SEQ ID NO: 38, SEQ ID NO: 39, SEQ ID NO: 40, SEQ ID NO: 64, or SEQ ID NO: 65.

Embodiment 64. The isolated antibody or antigen binding fragment thereof of embodiment 60 or embodiment 63, wherein:
(a) the VH comprises the amino acid sequence of SEQ ID NO: 10, SEQ ID NO: 11, SEQ ID NO: 12, SEQ ID NO: 13, SEQ ID NO: 14, SEQ ID NO: 27, SEQ ID NO: 28, SEQ ID NO: 29, SEQ ID NO: 35, or SEQ ID NO: 36, and the VL comprises the amino acid sequence of SEQ ID NO: 16;
(b) the VH comprises the amino acid sequence of SEQ ID NO: 10, SEQ ID NO: 11, SEQ ID NO: 12, SEQ ID NO: 13, SEQ ID NO: 14, SEQ ID NO: 27, SEQ ID NO: 28, SEQ ID NO: 29, SEQ ID NO: 35, or SEQ ID NO: 36, and the VL comprises the amino acid sequence of SEQ ID NO: 17;
(c) the VH comprises the amino acid sequence of SEQ ID NO: 10, SEQ ID NO: 11, SEQ ID NO: 12, SEQ ID NO: 13, SEQ ID NO: 14, SEQ ID NO: 27, SEQ ID NO: 28, SEQ ID NO: 29, SEQ ID NO: 35, or SEQ ID NO: 36, and the VL comprises the amino acid sequence of SEQ ID NO: 18; or
(d) the VH comprising the amino acid sequence of SEQ ID NO: 10, SEQ ID NO: 11, SEQ ID NO: 12, SEQ ID NO: 13, SEQ ID NO: 14, SEQ ID NO: 27, SEQ ID NO: 28, SEQ ID NO: 29, SEQ ID NO: 35, or SEQ ID NO: 36, and the VL comprises the amino acid sequence of SEQ ID NO: 39.

Embodiment 65. The isolated antibody or antigen binding fragment thereof of embodiment 55 or 60, wherein:
(a) the VH comprising an amino acid sequence that has at least 90% identity with the amino acid sequence of SEQ ID NO: 11, and wherein the VH-CDR1 comprises the amino acid sequence of SEQ ID NO: 2, the VH-CDR2 comprises the amino acid sequence of SEQ ID NO: 3, and the VH-CDR3 comprises the amino acid sequence of SEQ ID NO: 4; and
(b) the VL comprising an amino acid sequence that has at least 90% identity with the amino acid sequence of SEQ ID NO: 17, and wherein the VL-CDR1 comprises the amino acid sequence of SEQ ID NO: 6, the VL-CDR2 comprises the amino acid sequence of SEQ ID NO: 7, and the VL-CDR3 comprises the amino acid sequence of SEQ ID NO: 8.

Embodiment 66. A lipid nanoparticle (LNP), comprising the isolated antibody or antigen binding fragment thereof of any one of embodiments 1-65 conjugated to the LNP.

Embodiment 67. The LNP of embodiment 66, wherein the LNP comprises a lipid formulation comprising:
about 35 to about 65 mol % of an ionizable cationic lipid of structure

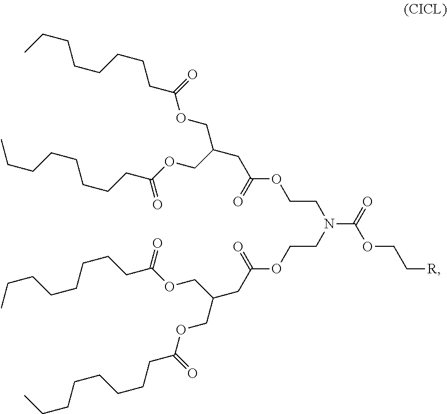

wherein R is

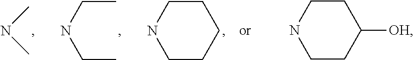

about 0.5 to about 3 mol % of a PEG-lipid, wherein the PEG-lipid comprises a functionalized PEG-lipid and a non-functionalized PEG-lipid,
about 7 to about 13 mol % of a phospholipid, and
about 27 to about 50 mol % of a sterol,
wherein the antibody or antigen-binding fragment thereof is conjugated to the functionalized PEG-lipid.

Embodiment 68. The LNP of embodiment 66 or embodiment 67, wherein the LNP comprising a lipid composition comprising:
a) about 40 mol % to about 62 mol % ionizable cationic lipid, about 7 mol % to about 13 mol % phospholipid, about 30 mol % to about 50 mol % sterol, about 0.5 mol % to about 3 mol % total functionalized and non-functionalized PEG-lipid, and about 0.1 mol % to 0.3 mol % functionalized PEG-lipid;
b) about 50 mol % CLCL, about 10 mol % phospholipid, about 38.5 mol % sterol, about 1.4 mol % non-functionalized PEG-lipid, and about 0.1 mol % functionalized PEG-lipid.
c) about 58 mol % CLCL, about 10 mol % phospholipid, about 30.5 mol % sterol, about 1.4 mol % non-functionalized PEG-lipid, and about 0.1 mol % functionalized PEG-lipid; or
d) about 62 mol % CLCL, about 10 mol % phospholipid, about 26.5 mol % sterol, about 1.4 mol % non-functionalized PEG-lipid, and about 0.1 mol % functionalized PEG-lipid.

Embodiment 69. The LNP of embodiment 67 or embodiment 68, wherein the R of CICL is

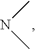

the phospholipid is distearoylphosphatidylcholine (DSPC), the sterol is cholesterol, the non-functionalized PEG-lipid is 1,2-distearoyl-glycero-3-phosphoethanolamine-3-methoxypolyethylene glycol-2000 (DSPE-PEG(2 k)), and the functionalized PEG-lipid is DSPE-PE(2 k)-maleimide (DSPE-PE(2 k)-MAL).

Embodiment 70. The LNP of any of embodiments 67-69, wherein the isolated humanized monoclonal antibody or antigen binding fragment thereof is covalently attached to a functionalized PEG-lipid via a modified lysine residue or a cysteine residue of the antibody or binding fragment thereof.

Embodiment 71. A lipid nanoparticle (LNP), comprising the isolated antibody or the antigen binding fragment thereof of any one of embodiments 1-44 conjugated to the LNP.

Embodiment 72. A lipid nanoparticle (LNP), comprising the F(ab′) analog of any one of embodiments 45-65 conjugated to the LNP.

Embodiment 73. A lipid nanoparticle (LNP) conjugated with an F(ab′) analog that comprises a relocated interchain disulfide bond.

Embodiment 74. A composition comprising the isolated antibody or antigen binding fragment thereof of any one of embodiments 1-44, the F(ab′) analog of embodiments 45-65, or the LNP of any one of embodiments 66-73, and a pharmaceutically acceptable carrier or excipient.

Embodiment 75. A method of delivering a payload into a CD8-positive cell, comprising contacting the LNP of any one of embodiments 66-73 or the composition of embodiment 74 with the CD8-positive cell.

Embodiment 76. The method of embodiment 75, wherein delivering the payload comprises transfecting the CD8-positive cell.

Embodiment 77. The method of embodiment 76, wherein the payload comprises an mRNA, circular RNA, self-amplifying RNA, or guide RNA.

Embodiment 78. The method of embodiment 75, wherein the contacting takes place in vivo, extracorporeally, or ex vivo.

Embodiment 79. The method of embodiment 75, wherein the payload mediates reprogramming of the CD8-positive cell.

Embodiment 80. The method of embodiment 79, wherein the payload comprises a nucleic acid encoding an immune receptor or immune cell engager.

Embodiment 81. The method of embodiment 79, wherein the payload comprises a nucleic acid encoding a gene/genome editing enzyme and/or a guide RNA or other component of a gene/genome editing system.

All publications, patents, and patent applications mentioned in this specification areherein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

1. An isolated antibody or antigen binding fragment thereof comprising a humanized immunoglobulin antigen binding domain that specifically binds to human CD8, comprising:
(a) a heavy chain variable region (VH) comprising an amino acid sequence that has at least 90% identity with the amino acid sequence of framework regions of SEQ ID NO: 9 or 31 wherein the VH comprises a heavy chain CDR1 (VH-CDR1) comprising the amino acid sequence RYTFTDYX₁LH (SEQ ID NO: 45), a VH-CDR2 comprising the amino acid sequence FIYPYX₁GGTG (SEQ ID NO: 46) or FIYPYX₂GGTG (SEQ ID NO: 47), and a VH-CDR3 comprising the amino acid sequence DHRYX₁EGVSFDY (SEQ ID NO: 48); and
(b) a light chain variable region (VL) comprising an amino acid sequence that has at least 90% identity with the amino acid sequence of framework regions of SEQ ID NO: 15 or 37, wherein the VL comprises a light chain CDR1 (VL-CDR1) comprising the amino acid sequence RASESVX₃GFGX₁SFMN (SEQ ID NO: 49), VL-CDR2 comprising the amino acid sequence LASX₂LES (SEQ ID NO: 50), and a VL-CDR3 comprising the amino acid sequence QQX₂X₂EX₃PYT (SEQ ID NO: 51),
wherein each X₁ is independently N, S, Q, or A; each X₂ is independently N, Q, D, S, or A; and each X₃ is independently D, E, S, or A.

2-71. (canceled)

72. A targeted lipid nanoparticle (tLNP), comprising:
(a) a lipid composition comprising a cationic lipid, a neutral lipid, a sterol, and a polymer-conjugated lipid; and
(b) an antibody or antigen-binding fragment thereof conjugated to the tLNP, wherein the antibody or antigen-binding fragment thereof specifically binds to human CD8 and comprises:
a heavy chain CDR1 (VH-CDR1) comprising the amino acid sequence RYTFTDYX₁LH (SEQ ID NO:45), a VH-CDR2 comprising the amino acid sequence FIYPYX₁GGTG (SEQ ID NO:46) or FIYPYX₂GGTG (SEQ ID NO:47), a VH-CDR3 comprising the amino acid sequence DHRYX₁EGVSFDY (SEQ ID NO:48); and
a light chain CDR1 (VL-CDR1) comprising the amino acid sequence RASESVX₃GFGX₁SFMN (SEQ ID NO:49), VL-CDR2 comprising the amino acid sequence LASX₂LES (SEQ ID NO:50), and VL-CDR3 comprising the amino acid sequence QQX₂X₂EX₃PYT (SEQ ID NO:51),
wherein each X₁ is independently N, S, Q, or A; each X₂ is independently N, Q, D, S, or A; and each X₃ is independently D, E, S, or A.

73. The tLNP of claim 72, wherein the antibody or antigen-binding fragment thereof comprises a whole antibody.

74. The tLNP of claim 73, wherein the antibody comprises a human IgG1, human IgG2, or human IgG4 heavy chain constant domain.

75. The tLNP of claim 73, wherein the antibody comprises a heavy chain constant domain having a silenced Fc region.

76. The tLNP of claim 74, wherein the antibody comprises a human IgG1 constant domain comprising the amino acid sequence of SEQ ID NO: 42.

77. The tLNP of claim 74, wherein the human IgG1 constant domain comprises a silenced Fc region comprising the amino acid sequence of SEQ ID NO: 43 or SEQ ID NO: 44.

78. The tLNP of claim 72, wherein the antibody comprises a light chain constant domain comprising the amino acid sequence of SEQ ID NO: 41.

79. The tLNP of claim 77, wherein the antibody comprises a light chain constant domain comprising the amino acid sequence SEQ ID NO: 41.

80. The tLNP of claim 72, wherein the antibody or antigen-binding fragment thereof comprises:
(a) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYNGGTG (SEQ ID NO: 3), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence RASESVDGFGNSFMN (SEQ ID NO: 6), a VL-CDR2 comprising the amino acid sequence LASNLES (SEQ ID NO: 7), and a VL-CDR3 comprising the amino acid sequence QQNNEDPYT (SEQ ID NO: 8);
(b) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYNGGTG (SEQ ID NO: 3), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence of RASESVSGFGNSFMN (SEQ ID NO:227), a VL-CDR2 comprising the amino acid sequence of LASNLES (SEQ ID NO: 7), and a VL-CDR3 comprising the amino acid sequence of QQNNEDPYT (SEQ ID NO: 8);
(c) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYNGGTG (SEQ ID NO: 3), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence RASESVAGFGNSFMN (SEQ ID NO: 228), a VL-CDR2 comprising the amino acid sequence LASNLES (SEQ ID NO: 7), and a VL-CDR3 comprising the amino acid sequence QQNNEDPYT (SEQ ID NO: 8);
(d) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYSGGTG (SEQ ID NO: 58), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence RASESVDGFGNSFMN (SEQ ID NO: 6), a VL-CDR2 comprising the amino acid sequence of LASNLES (SEQ ID NO: 7), and a VL-CDR3 comprising the amino acid sequence QQNNEDPYT (SEQ ID NO: 8);
(e) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYSGGTG (SEQ ID NO: 58), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence RASESVSGFGNSFMN (SEQ ID NO: 227), a L-CDR2 comprising the amino acid sequence LASNLES (SEQ ID NO: 7), and a VL-CDR3 comprising the amino acid sequence of QQNNEDPYT (SEQ ID NO: 8);
(f) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYSGGTG (SEQ ID NO: 58), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence RASESVAGFGNSFMN (SEQ ID NO: 228), a L-CDR2 comprising the amino acid sequence LASNLES (SEQ ID NO: 7), and a L-CDR3 comprising the amino acid sequence QQNNEDPYT (SEQ ID NO: 8);
(g) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYQGGTG (SEQ ID NO: 59), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence RASESVDGFGNSFMN (SEQ ID NO: 6), a VL-CDR2 comprising the amino acid sequence LASNLES (SEQ ID NO: 7), and a VL-CDR3 comprising the amino acid sequence QQNNEDPYT (SEQ ID NO: 8);
(h) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYQGGTG (SEQ ID NO: 59), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence RASESVSGFGNSFMN (SEQ ID NO: 227), a VL-CDR2 comprising the amino acid sequence LASNLES (SEQ ID NO: 7), and a VL-CDR3 comprising the amino acid sequence QQNNEDPYT (SEQ ID NO: 8);
(i) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYQGGTG (SEQ ID NO: 59), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence RASESVAGFGNSFMN (SEQ ID NO: 228), a VL-CDR2 comprising the amino acid sequence LASNLES (SEQ ID NO: 7), and a VL-CDR3 comprising the amino acid sequence QQNNEDPYT (SEQ ID NO: 8);
(j) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYAGGTG (SEQ ID NO: 60), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence RASESVDGFGNSFMN (SEQ ID NO: 6), a VL-CDR2 comprising the amino acid sequence LASNLES (SEQ ID NO: 7), and a VL-CDR3 comprising the amino acid sequence QQNNEDPYT (SEQ ID NO: 8);
(k) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYAGGTG (SEQ ID NO: 60), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence RASESVSGFGNSFMN (SEQ ID NO: 227), a VL-CDR2 comprising the amino acid sequence LASNLES (SEQ ID NO: 7), and a VL-CDR3 comprising the amino acid sequence QQNNEDPYT (SEQ ID NO: 8); or
(l) a heavy chain variable region (VH) wherein the VH comprises a heavy chain CDR1 (VH-CDR1) comprising the amino acid sequence RYTFTDYNLH (SEQ ID NO: 2), a VH-CDR2 comprising the amino acid sequence FIYPYAGGTG (SEQ ID NO: 60), and a VH-CDR3 comprising the amino acid sequence DHRYNEGVSFDY (SEQ ID NO: 4); and a light chain variable region (VL) wherein the VL comprises a light chain CDR1 (VL-CDR1) comprising the amino acid sequence RASESVAGFGNSFMN (SEQ ID NO: 228), a VL-CDR2 comprising the amino acid sequence LASNLES (SEQ ID NO: 7), and a VL-CDR3 comprising the amino acid sequence QQNNEDPYT (SEQ ID NO: 8).

81. The tLNP of claim 72, wherein the antibody or antigen-binding fragment thereof comprises:
(a) a heavy chain variable region (VH) comprising an amino acid sequence that has at least 90%, at least 91%, at least 92%, at least 93%, at least 94%, at least 95%, at least 96%, at least 97%, at least 98%, at least 99%, or 100% identity with the amino acid sequence of framework regions of SEQ ID NO: 9 or SEQ ID NO: 31; and
(b) a light chain variable region (VL) comprising an amino acid sequence that has at least 90%, at least 91%, at least 92%, at least 93%, at least 94%, at least 95%, at least 96%, at least 97%, at least 98%, at least 99%, or 100% identity with the amino acid sequence of framework regions of SEQ ID NO: 15 or SEQ ID NO: 37.

82. The tLNP of claim 72, wherein the antibody or antigen-binding fragment thereof comprises:
(a) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 11; and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 17;
(b) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 12; and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 17;
(c) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 13; and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 17;
(d) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 14; and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 17;
(e) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 11; and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 18;
(f) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 12; and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 18; or
(g) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 14; and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 18.

83. The tLNP of claim 72, wherein the antibody or antigen-binding fragment thereof comprises:
(a) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 11, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 64;
(b) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 11, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 65;
(c) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 27, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 17;
(d) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 28, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 17;
(e) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 29, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 17;
(f) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 66, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 17;
(g) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 67, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 17;
(h) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 68, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 17;
(i) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 13, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 64;
(j) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 13, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 65;
(k) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 66, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 64;
(l) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 67, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 64;
(m) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 68, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO:64;
(n) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 66, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO:65;
(o) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO:67, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO:65; or
(p) a VH comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO: 68, and a VL comprising an amino acid sequence that has at least 90%, at least 91%, at least 92%, at least 93%, at least 94%, at least 95%, at least 96%, at least 97%, at least 98%, at least 99%, or 100% identity with the amino acid sequence of SEQ ID NO:65.

84. The tLNP of claim 72, wherein the antibody or antigen-binding fragment thereof comprises a heavy chain and a light chain, wherein:
(a) the heavy chain comprises an amino acid sequence that has at least 90%, at least 91%, at least 92%, at least 93%, at least 94%, at least 95%, at least 96%, at least 97%, at least 98%, at least 99%, or 100% identity to the heavy chain amino acid sequence of SEQ ID NO: 61;
and the light chain comprises an amino acid sequence that has at least 90%, at least 91%, at least 92%, at least 93%, at least 94%, at least 95%, at least 96%, at least 97%, at least 98%, at least 99%, or 100% identity to the light chain amino acid sequence of SEQ ID NO: 62;
(b) the heavy chain comprises an amino acid sequence that has at least 90%, at least 91%, at least 92%, at least 93%, at least 94%, at least 95%, at least 96%, at least 97%, at least 98%, at least 99%, or 100% identity to the heavy chain amino acid sequence of SEQ ID NO: 61;
and the light chain comprises an amino acid sequence that has at least 90%, at least 91%, at least 92%, at least 93%, at least 94%, at least 95%, at least 96%, at least 97%, at least 98%, at least 99%, or 100% identity to the light chain amino acid sequence of SEQ ID NO: 71;
(c) the heavy chain comprises an amino acid sequence that has at least 90%, at least 91%, at least 92%, at least 93%, at least 94%, at least 95%, at least 96%, at least 97%, at least 98%, at least 99%, or 100% identity to the heavy chain amino acid sequence of SEQ ID NO: 69 and the light chain comprises an amino acid sequence that has at least 90%, at least 91%, at least 92%, at least 93%, at least 94%, at least 95%, at least 96%, at least 97%, at least 98%, at least 99%, or 100% identity to the light chain amino acid sequence of SEQ ID NO: 62;
(d) the heavy chain comprises an amino acid sequence that has at least 90%, at least 91%, at least 92%, at least 93%, at least 94%, at least 95%, at least 96%, at least 97%, at least 98%, at least 99%, or 100% identity to the heavy chain amino acid sequence of SEQ ID NO: 69 and the light chain comprises an amino acid sequence that has at least 90%, at least 91%, at least 92%, at least 93%, at least 94%, at least 95%, at least 96%, at least 97%, at least 98%, at least 99%, or 100% identity to the light chain amino acid sequence of SEQ ID NO: 71;
(e) the heavy chain comprises an amino acid sequence that has at least 90%, at least 91%, at least 92%, at least 93%, at least 94%, at least 95%, at least 96%, at least 97%, at least 98%, at least 99%, or 100% identity to the heavy chain amino acid sequence of SEQ ID NO: 70 and the light chain comprises an amino acid sequence that has at least 90%, at least 91%, at least 92%, at least 93%, at least 94%, at least 95%, at least 96%, at least 97%, at least 98%, at least 99%, or 100% identity to the light chain amino acid sequence of SEQ ID NO: 62;
(f) the heavy chain comprises an amino acid sequence that has at least 90%, at least 91%, at least 92%, at least 93%, at least 94%, at least 95%, at least 96%, at least 97%, at least 98%, at least 99%, or 100% identity to the heavy chain amino acid sequence of SEQ ID NO: 70 and the light chain comprises an amino acid sequence that has at least 90%, at least 91%, at least 92%, at least 93%, at least 94%, at least 95%, at least 96%, at least 97%, at least 98%, at least 99%, or 100% identity to the light chain amino acid sequence of SEQ ID NO: 71; or
(h) the heavy chain comprises an amino acid sequence that has at least 90%, at least 91%, at least 92%, at least 93%, at least 94%, at least 95%, at least 96%, at least 97%, at least 98%, at least 99%, or 100% identity to the heavy chain amino acid sequence of SEQ ID NO: 75 and the light chain comprises an amino acid sequence that has at least 90%, at least 91%, at least 92%, at least 93%, at least 94%, at least 95%, at least 96%, at least 97%, at least 98%, at least 99%, or 100% identity to the light chain amino acid sequence of SEQ ID NO: 62.

85. The tLNP of claim 72, wherein the lipid composition comprises:
(a) about 35 to about 65 mol % of an ionizable cationic lipid of structure 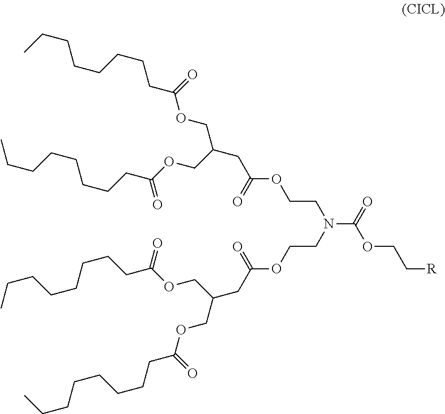
wherein R is 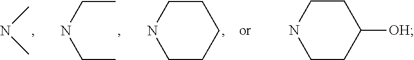
(b) about 7 to about 13 mol % of a phospholipid;
(c) about 27 to about 50 mol % of a sterol; and
(d) about 0.5 to about 3 mol % of a PEG-lipid, wherein the PEG-lipid comprises a functionalized PEG-lipid and a non-functionalized PEG-lipid, wherein the antibody or antigen-binding fragment thereof is conjugated to the functionalized PEG-lipid.

86. The tLNP of claim 85, wherein the lipid composition comprises:
(a) about 40 mol % to about 62 mol % CICL, about 7 mol % to about 13 mol % phospholipid, about 30 mol % to about 50 mol % sterol, about 0.5 mol % to about 3 mol % total functionalized and non-functionalized PEG-lipid, and about 0.1 mol % to 0.3 mol % functionalized PEG-lipid;
(b) about 50 mol % CLCL, about 10 mol % phospholipid, about 38.5 mol % sterol, about 1.4 mol % non-functionalized PEG-lipid, and about 0.1 mol % functionalized PEG-lipid;
(c) about 58 mol % CLCL, about 10 mol % phospholipid, about 30.5 mol % sterol, about 1.4 mol % non-functionalized PEG-lipid, and about 0.1 mol % functionalized PEG-lipid; or
(d) about 62 mol % CLCL, about 10 mol % phospholipid, about 26.5 mol % sterol, about 1.4 mol % non-functionalized PEG-lipid, and about 0.1 mol % functionalized PEG-lipid.

87. The LNP of claim 86, wherein the ionizable cationic lipid is CLCL, the phospholipid is distearoylphosphatidylcholine (DSPC), the sterol is cholesterol, the non-functionalized PEG-lipid is 1,2-distearoyl-glycero-3-methoxypolyethylene glycol-2000 (DSG-PEG(2000)), and the functionalized PEG-lipid is 1,2-distearoyl-glycero-3-phosphoethanolamine-3-methoxypolyethylene glycol-2000 (DSPE-PEG(2000))-maleimide (DSPE-PEG(2000)-MAL).

88. The LNP of claim 85, wherein the antibody or antigen-binding fragment thereof is covalently attached to a functionalized PEG-lipid via a modified lysine residue or a cysteine residue of the antibody or binding fragment thereof.

89. The tLNP of claim 88, wherein the modified lysine residue is a thiolated lysine residue at Lys248 or Lys288 of the constant region.

90. The tLNP of claim 72, wherein:
(a) the tLNP comprises
(i) the lipid composition comprising about 58 mol % CLCL1, about 10 mol % DSPC, about 30.5 mol % cholesterol, about 1.4 mol % non-functionalized PEG-lipid DSG-PEG-2000, and about 0.1 mol % functionalized PEG-lipid DSPE-PEG-2000 MAL;
(ii) the antibody or antigen-binding fragment thereof comprising an antibody, the antibody comprising a heavy chain of SEQ ID NO: 61 and a light chain of SEQ ID NO: 62 conjugated to the functionalized PEG-lipid DSPE-PEG-2000 MAL via modified lysine residue Lys248 of the heavy chain; and
(iii) a payload comprising an mRNA encoding a chimeric antigen receptor (CAR) specific for human CD19, wherein the CAR comprises a CD19 binder having the amino acid sequence of SEQ ID NO: 149; or
(b) the tLNP comprises
(i) the lipid composition comprising about 58 mol % CLCL1, about 10 mol % DSPC, about 30.5 mol % cholesterol, about 1.4 mol % non-functionalized PEG-lipid DSG-PEG-2000, and about 0.1 mol % functionalized PEG-lipid DSPE-PEG-2000 MAL;
(ii) the antibody or antigen-binding fragment thereof comprising an antibody, the antibody comprising a heavy chain of SEQ ID NO: 69 and a light chain of SEQ ID NO: 62 conjugated to the functionalized PEG-lipid DSPE-PEG-2000 MAL via modified lysine residue Lys248 of the heavy chain; and
(iii) a payload comprising an mRNA encoding a chimeric antigen receptor (CAR) specific for human CD19, wherein the CAR comprises a CD19 binder having the amino acid sequence of SEQ ID NO: 149; or
(c) the tLNP comprises
(i) the lipid composition comprising about 58 mol % CLCL1, about 10 mol % DSPC, about 30.5 mol % cholesterol, about 1.4 mol % non-functionalized PEG-lipid DSG-PEG-2000), and about 0.1 mol % functionalized PEG-lipid DSPE-PEG-2000 MAL;
(ii) the antibody or antigen-binding fragment thereof comprising an antibody, the antibody comprising a heavy chain of SEQ ID NO: 70 and a light chain of SEQ ID NO: 62 conjugated to the functionalized PEG-lipid DSPE-PEG-2000 MAL via modified lysine residue Lys248 of the heavy chain; and
(iii) a payload comprising an mRNA encoding a chimeric antigen receptor (CAR) specific for human CD19, wherein the CAR comprises a CD19 binder having the amino acid sequence of SEQ ID NO: 149,
wherein CICL1 has a structure of: 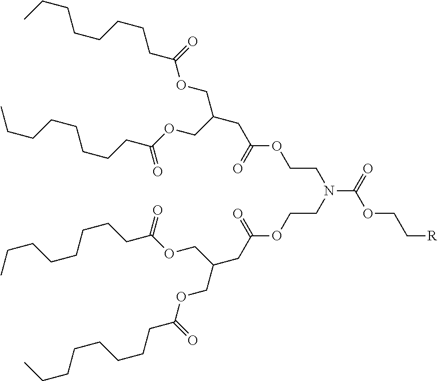
with R being

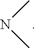

91. A composition, comprising the tLNP of claim 72 and a pharmaceutically acceptable carrier or excipient.

92. The composition of claim 91, wherein the tLNP comprises:
(a) the lipid composition comprising about 58 mol % CLCL1, about 10 mol % DSPC, about 30.5 mol % cholesterol, about 1.4 mol % non-functionalized PEG-lipid DSG-PEG-2000, and about 0.1 mol % functionalized PEG-lipid DSPE-PEG-2000 MAL;
(b) the antibody or antigen-binding fragment thereof comprising an antibody, the antibody comprising a heavy chain of SEQ ID NO: 61 and a light chain of SEQ ID NO: 62 conjugated to the functionalized PEG-lipid DSPE-PEG-2000 MAL via modified lysine residue Lys248 of the heavy chain; and
(c) a payload comprising an mRNA encoding a chimeric antigen receptor (CAR) specific for human CD19, wherein the CAR comprises a CD19 binder having the amino acid sequence of SEQ ID NO: 149,
wherein CICL1 has a structure of: 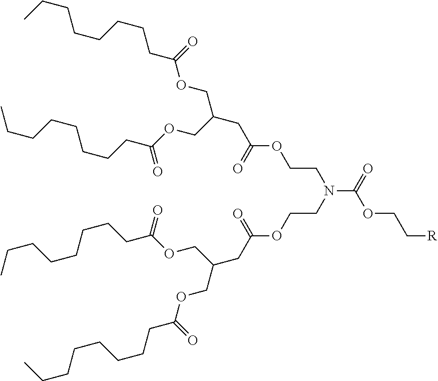
with R being

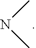

93. The composition of claim 91, wherein the tLNP comprises:
(a) the lipid composition comprising about 58 mol % CLCL1, about 10 mol % DSPC, about 30.5 mol % cholesterol, about 1.4 mol % non-functionalized PEG-lipid DSG-PEG-2000, and about 0.1 mol % functionalized PEG-lipid DSPE-PEG-2000 MAL;
(b) the antibody or antigen-binding fragment thereof comprising an antibody, the antibody comprising a heavy chain of SEQ ID NO: 69 and a light chain of SEQ ID NO: 62 conjugated to the functionalized PEG-lipid DSPE-PEG-2000 MAL via modified lysine residue Lys248 of the heavy chain; and
(c) a payload comprising an mRNA encoding a chimeric antigen receptor (CAR) specific for human CD19, wherein the CAR comprises a CD19 binder having the amino acid sequence of SEQ ID NO: 149.

94. The composition of claim 91, wherein the tLNP comprises:
(a) the lipid composition comprising about 58 mol % CLCL1, about 10 mol % DSPC, about 30.5 mol % cholesterol, about 1.4 mol % non-functionalized PEG-lipid DSG-PEG-2000, and about 0.1 mol % functionalized PEG-lipid DSPE-PEG-2000 MAL;
(b) the antibody or antigen-binding fragment thereof comprising an antibody, the antibody comprising a heavy chain of SEQ ID NO: 70 and a light chain of SEQ ID NO: 62 conjugated to the functionalized PEG-lipid DSPE-PEG-2000 MAL via modified lysine residue Lys248 of the heavy chain; and
(c) a payload comprising an mRNA encoding a chimeric antigen receptor (CAR) specific for human CD19, wherein the CAR comprises a CD19 binder having the amino acid sequence of SEQ ID NO: 149.

95. A method of delivering a payload into a CD8-positive cell, comprising contacting the tLNP of claim 72 with a CD8-positive cell, thereby delivering the payload to the CD8-positive cell.

96. The method of claim 95, wherein the CD8-positive cell is contacted in vivo, extracorporeally, or ex vivo.

97. The method of claim 95, wherein the delivered payload comprises an mRNA, circular RNA, or self-amplifying RNA.

98. The method of claim 97, wherein the payload comprises an mRNA and the mRNA encodes a chimeric antigen receptor (CAR), a T cell receptor (TCR), an immune cell engager, a cytokine, a chemokine, a chemokine receptor, or a dominant negative cytokine receptor.


SEQUENCE LISTING (232 sequences)

SEQ ID NO: 1 - protein, 121 aa, synthetic construct
EVQLQQSGPELVKPGASVKISCKASRYTFTDYNLHWVKLSHEKSLEWIGFIYPYNGGTGYNQKFKNKAKLTVDYSSSTAYMELRSLTSVDAAVYYCARDHRYNEGVSFDYWGQGTTLTVSS

SEQ ID NO: 2 - protein, 10 aa, synthetic construct
RYTFTDYNLH

SEQ ID NO: 3 - protein, 10 aa, synthetic construct
FIYPYNGGTG

SEQ ID NO: 4 - protein, 12 aa, synthetic construct
DHRYNEGVSFDY

SEQ ID NO: 5 - protein, 111 aa, synthetic construct
NIVLTQSPASLAVSLGQRATISCRASESVDGFGNSFMNWYQQKPGQSPKLLIYLASNLESGVPARFSGSGSRTDFTLTIDPVEADDAATYYCQQNNEDPYTFGGGTKLEIK

SEQ ID NO: 6 - protein, 15 aa, synthetic construct
RASESVDGFGNSFMN

SEQ ID NO: 7 - protein, 7 aa, synthetic construct
LASNLES

SEQ ID NO: 8 - protein, 9 aa, synthetic construct
QQNNEDPYT

SEQ ID NO: 9 - protein, 109 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARWGQGTTVTVSS

SEQ ID NO: 10 - protein, 121 aa, synthetic construct
QVQLVQSGAEVKKPGASVKVSCKASRYTFTDYNLHWVRQAPGQGLEWMGFIYPYNGGTGYAQKFQGRVTMTRDTSTSTVYMELSSLRSEDTAVYYCARDHRYNEGVSFDYWGQGTTVTVSS

SEQ ID NO: 11 - protein, 121 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSS

SEQ ID NO: 12 - protein, 121 aa, synthetic construct
QVQLVQSGAEVKKPGASVKISCKASRYTFTDYNLHWVRQAPGQGLEWIGFIYPYNGGTGYAQKFQGRATLTVDTSTSTAYMELSSLRSEDTAVYYCARDHRYNEGVSFDYWGQGTTLTVSS

SEQ ID NO: 13 - protein, 121 aa, synthetic construct
QVQLVQSGAEVKKPGASVKVSCKASRYTFTDYNLHWVRQAPGQGLEWMGFIYPYNGGTGYAQKFQGRVTMTVDYSTSTAYMELSSLRSEDTAVYYCARDHRYNEGVSFDYWGQGTTVTVSS

SEQ ID NO: 14 - protein, 121 aa, synthetic construct
QVQLVQSGAEVKKPGASVKISCKASRYTFTDYNLHWVRQAPGQGLEWIGFIYPYNGGTGYAQKFQGRATLTVDYSTSTAYMELSSLRSEDTAVYYCARDHRYNEGVSFDYWGQGTTLTVSS

SEQ ID NO: 15 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPFGQGTKLEIK

SEQ ID NO: 16 - protein, 111 aa, synthetic construct
DIQMTQSPSSLSASVGDRVTITCRASESVDGFGNSFMNWYQQKPGKAPKLLIYLASNLESGVPSRFSGSGSGTDFTLTISSLQPEDFATYYCQQNNEDPYTFGQGTKLEIK

SEQ ID NO: 17 - protein, 111 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIK

SEQ ID NO: 18 - protein, 111 aa, synthetic construct
DIQLTQSPSSLSASVGDRATITCRASESVDGFGNSFMNWYQQKPGKAPKLLIYLASNLESGVPSRFSGSGSRTDFTLTISSVQPEDFATYYCQQNNEDPYTFGQGTKLEIK

SEQ ID NO: 19 - protein, 657 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 677 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 700 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSLDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 720 aa, synthetic construct
METDTLLLWVLLLWVPGSTGDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSLDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 118 aa, synthetic construct [sequence omitted]

SEQ ID NO: 24 - protein, 107 aa, synthetic construct
DVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIK

SEQ ID NO: 25 - protein, 118 aa, synthetic construct
EVQLQQSGAELVKPGASVKLSCTASGFNIKDTYIHFVRQRPEQGLEWIGRIDPANDNTLYASKFQGKATITADTSSNTAYMHLCSLTSGDTAVYYCGRGYGYYVFDHWGQGTTLTVSS

SEQ ID NO: 26 - protein, 107 aa, synthetic construct
DVQINQSPSFLAASPGETITINCRTSRSISQYLAWYQEKPGKTNKLLIYSGSTLQSGIPSRFSGSGSGTDFTLTISGLEPEDFAMYYCQQHNENPLTFGAGTKLELK

SEQ ID NO: 27 - protein, 121 aa, synthetic construct
QVQLVQSGAEVKKPGASVKVSCKASRYTFTDYNLHWVRQAPGQGLEWMGFIYPYSGGTGYAQKFQGRVTMTVDTSTSTAYMELSSLRSEDTAVYYCARDHRYNEGVSFDYWGQGTTVTVSS

SEQ ID NO: 28 - protein, 121 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSS

SEQ ID NO: 29 - protein, 121 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSS

SEQ ID NO: 30 - protein, 121 aa, synthetic construct
QVQLVQSGAEVKKPGASVKVSCKASRYTFTDYNLHWVRQAPGQGLEWMGFIYPYDGGTGYAQKFQGRVTMTVDTSTSTAYMELSSLRSEDTAVYYCARDHRYNEGVSFDYWGQGTTVTVSS

SEQ ID NO: 31 - protein, 108 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARWGQGTLVTVS

SEQ ID NO: 32 - protein, 121 aa, synthetic construct
QVQLVQSGAEVKKPGASVKVSCKASRYTFTDYNLHWVRQAPGQGLEWMGFIYPYNGGTNYAQKFQGRVTITADTSTSTAYMELSSLRSEDTAVYYCARDHRYNEGVSFDYWGQGTLVTVSS

SEQ ID NO: 33 - protein, 121 aa, synthetic construct
QVQLVQSGAEVKKPGASVKVSCKASRYTFTDYNLHWVRQAPGQGLEWIGFIYPYNGGTGYAQKFQGRVTITADTSTSTAYMELSSLRSEDTAVYYCARDHRYNEGVSFDYWGQGTLVTVSS

SEQ ID NO: 34 - protein, 121 aa, synthetic construct
QVQLVQSGAEVKKPGASVKVSCKASRYTFTDYNLHWVRQAPGQGLEWIGFIYPYNGGTGYAQKFKGRVTITADTSTSTAYMELSSLRSEDTAVYYCARDHRYNEGVSFDYWGQGTLVTVSS

SEQ ID NO: 35 - protein, 121 aa, synthetic construct
QVQLVQSGAEVKKPGASVKVSCKASRYTFTDYNLHWVRQAPGQGLEWMGFIYPYNGGTGYAQKFQGRVTLTVDYSTSTAYMELSSLRSEDTAVYYCARDHRYNEGVSFDYWGQGTLVTVSS

SEQ ID NO: 36 - protein, 121 aa, synthetic construct
QVQLVQSGAEVKKPGASVKVSCKASRYTFTDYNLHWVRQAPGQGLEWIGFIYPYNGGTGYAQKFQGRVTLTVDYSTSTAYMELSSLRSEDTAVYYCARDHRYNEGVSFDYWGQGTLVTVSS

SEQ ID NO: 37 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 38 - protein, 111 aa, synthetic construct
EIVLTQSPGTLSLSPGERATLSCRASESVDGFGNSFMNWYQQKPGQAPRLLIYLASNLESGIPARFSGSGSGTDFTLTISRLEPEDFAVYYCQQNNEDPYTFGQGTKVEIK

SEQ ID NO: 39 - protein, 111 aa, synthetic construct
EIVLTQSPGTLSLSPGERATLSCRASESVDGFGNSFMNWYQQKPGQAPRLLIYLASNLESGVPARFSGSGSGTDFTLTISRLEPEDFAVYYCQQNNEDPYTFGQGTKVEIK

SEQ ID NO: 40 - protein, 111 aa, synthetic construct
EIVLTQSPGTLSLSPGERATLSCRASESVDGFGNSFMNWYQQKPGQAPRLLIYLASNLESGVPARFSGSGSRTDFTLTISRLEPEDFAVYYCQQNNEDPYTFGQGTKVEIK

SEQ ID NO: 41 - protein, 107 aa, synthetic construct [sequence omitted]

SEQ ID NO: 42 - protein, 330 aa, synthetic construct [sequence omitted]

SEQ ID NO: 43 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 330 aa, synthetic construct [sequence omitted]

SEQ ID NO: 45 - protein, 10 aa, synthetic construct
  VARIANT 8: note = X is N, S, Q, or A
RYTFTDYXLH

SEQ ID NO: 46 - protein, 10 aa, synthetic construct
  VARIANT 6: note = X is N, S, Q, or A
FIYPYXGGTG

SEQ ID NO: 47 - protein, 10 aa, synthetic construct
  VARIANT 6: note = X is N, Q, D, S, or A
FIYPYXGGTG

SEQ ID NO: 48 - protein, 12 aa, synthetic construct
  VARIANT 5: note = N is N, Q, S, or A
DHRYXEGVSFDY

SEQ ID NO: 49 - protein, 15 aa, synthetic construct
  VARIANT 7: note = X is D, E, S, or A
  VARIANT 11: note = X is N, S, Q, or A
RASESVXGFGXSFMN

SEQ ID NO: 50 - protein, 7 aa, synthetic construct
  VARIANT 4: note = X is N, Q, D, S, or A
LASXLES

SEQ ID NO: 51 - protein, 9 aa, synthetic construct
  VARIANT 3: note = X is N, Q, D, S, or A
  VARIANT 6: note = X is D, E, S, or A
  VARIANT 4: note = X is N, Q, D, S, or A
QQXXEXPYT

SEQ ID NO: 52 - protein, 121 aa, synthetic construct
  VARIANT 33: note = X is N, S, Q, or A
  VARIANT 55: note = X is N, S, Q, or A
  VARIANT 103: note = X is N, S, Q, or A
QVQLVQSGAEVKKPGASVKVSCKASRYTFTDYXLHWVRQAPGQGLEWMGFIYPYXGGTGYAQKFQGRVTMTRDTSTSTVYMELSSLRSEDTAVYYCARDHRYXEGVSFDYWGQGTTVTVSS

SEQ ID NO: 53 - protein, 121 aa, synthetic construct; identical to SEQ ID NO: 52
  VARIANT 33: note = N is N, S, Q, or A
  VARIANT 55: note = X is N, Q, D, S, or A
  VARIANT 103: note = X is N, S, Q, or A

SEQ ID NO: 54 - protein, 111 aa, synthetic construct
  VARIANT 30: note = X is D, E, S, or A
  VARIANT 34: note = X is N, S, Q, or A
  VARIANT 95: note = X is N, Q, D, S, or A
  VARIANT 98: note = X is D, E, S, or A
  VARIANT 96: note = X is N, Q, D, S, or A
DIQMTQSPSSLSASVGDRVTITCRASESVXGFGXSFMNWYQQKPGKAPKLLIYLASXLESGVPSRFSGSGSGTDFTLTISSLQPEDFATYYCQQXXEXPYTFGQGTKLEIK

SEQ ID NO: 55 - protein, 120 aa, synthetic construct
  VARIANT 33: note = X is N, S, Q, or A
  VARIANT 55: note = X is N, S, Q, or A
  VARIANT 103: note = X is N, S, Q, or A
QVQLVQSGAEVKKPGASVKVSCKASRYTFTDYXLHWVRQAPGQGLEWMGFIYPYXGGTGYAQKFQGRVTMTRDTSTSTVYMELSSLRSEDTAVYYCARDHRYXEGVSFDYWGQGTLVTVS

SEQ ID NO: 56 - protein, 120 aa, synthetic construct; identical to SEQ ID NO: 55
  VARIANT 33: note = X is N, S, Q, or A
  VARIANT 55: note = X is N, Q, D, S, or A
  VARIANT 103: note = X isN, S, Q, or A

SEQ ID NO: 57 - protein, 111 aa, synthetic construct
  VARIANT 30: note = X is D, E, S, or A
  VARIANT 34: note = is N, S, Q, or AX
  VARIANT 57: note = X is N, Q, D, S, or A
  VARIANT 95: note = X is N, Q, D, S, or A
  VARIANT 98: note = X is D, E, S, or A
  VARIANT 96: note = X is N, Q, D, S, or A
EIVLTQSPGTLSLSPGERATLSCRASESVXGFGXSFMNWYQQKPGQAPRLLIYLASXLESGIPDRFSGSGSGTDFTLTISRLEPEDFAVYYCQQXXEXPYTFGQGTKVEIK

SEQ ID NO: 58 - protein, 10 aa, synthetic construct
FIYPYSGGTG

SEQ ID NO: 59 - protein, 10 aa, synthetic construct
FIYPYQGGTG

SEQ ID NO: 60 - protein, 10 aa, synthetic construct
FIYPYAGGTG

SEQ ID NO: 61 - protein, 450 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 218 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 111 aa, synthetic construct
DIQLTQSPSSLSASVGDRATITCRASESVEGFGNSFMNWYQQKPGKAPKLLIYLASNLESGVPSRFSGSGSGTDFTLTISSVQPEDFATYYCQQNNEDPYTFGQGTKLEIK

SEQ ID NO: 64 - protein, 111 aa, synthetic construct
DIQLTQSPSSLSASVGDRATITCRASESVSGFGNSFMNWYQQKPGKAPKLLIYLASNLESGVPSRFSGSGSGTDFTLTISSVQPEDFATYYCQQNNEDPYTFGQGTKLEIK

SEQ ID NO: 65 - protein, 111 aa, synthetic construct
DIQLTQSPSSLSASVGDRATITCRASESVAGFGNSFMNWYQQKPGKAPKLLIYLASNLESGVPSRFSGSGSGTDFTLTISSVQPEDFATYYCQQNNEDPYTFGQGTKLEIK

SEQ ID NO: 66 - protein, 121 aa, synthetic construct
QVQLVQSGAEVKKPGASVKVSCKASRYTFTDYNLHWVRQAPGQGLEWMGFIYPYSGGTGYAQKFQGRVTMTVDYSTSTAYMELSSLRSEDTAVYYCARDHRYNEGVSFDYWGQGTTVTVSS

SEQ ID NO: 67 - protein, 121 aa, synthetic construct
QVQLVQSGAEVKKPGASVKVSCKASRYTFTDYNLHWVRQAPGQGLEWMGFIYPYQGGTGYAQKFQGRVTMTVDYSTSTAYMELSSLRSEDTAVYYCARDHRYNEGVSFDYWGQGTTVTVSS

SEQ ID NO: 68 - protein, 121 aa, synthetic construct
QVQLVQSGAEVKKPGASVKVSCKASRYTFTDYNLHWVRQAPGQGLEWMGFIYPYAGGTGYAQKFQGRVTMTVDYSTSTAYMELSSLRSEDTAVYYCARDHRYNEGVSFDYWGQGTTVTVSS

SEQ ID NO: 69 - protein, 450 aa, synthetic construct
QVQLVQSGAEVKKPGASVKISCKASRYTFTDYNLHWVRQAPGQGLEWIGFIYPYNGGTGYAQKFQGRATLTVDTSTSTAYMELSSLRSEDTAVYYCARDHRYNEGVSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 450 aa, synthetic construct
QVQLVQSGAEVKKPGASVKISCKASRYTFTDYNLHWVRQAPGQGLEWIGFIYPYNGGTGYAQKFQGRATLTVDYSTSTAYMELSSLRSEDTAVYYCARDHRYNEGVSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 218 aa, synthetic construct
DIQLTQSPSSLSASVGDRATITCRASESVDGFGNSFMNWYQQKPGKAPKLLIYLASNLESGVPSRFSGSGSRTDFTLTISSVQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 450 aa, synthetic construct
QVQLVQSGAEVKKPGASVKVSCKASRYTFTDYNLHWVRQAPGQGLEWMGFIYPYSGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 450 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 450 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 450 aa, synthetic construct
QVQLVQSGAEVKKPGASVKVSCKASRYTFTDYNLHWVRQAPGQGLEWMGFIYPYNGGTGYAQKFQGRVTMTVDYSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 119 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLG

SEQ ID NO: 77 - protein, 218 aa, synthetic construct; identical to SEQ ID NO: 62

SEQ ID NO: 78 - protein, 240 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLG

SEQ ID NO: 79 - protein, 116 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLG

SEQ ID NO: 80 - protein, 237 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLG

SEQ ID NO: 81 - protein, 115 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAP

SEQ ID NO: 82 - protein, 236 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAP

SEQ ID NO: 83 - protein, 112 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAP

SEQ ID NO: 84 - protein, 233 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAP

SEQ ID NO: 85 - protein, 111 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 86 - protein, 232 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAA

SEQ ID NO: 87 - protein, 108 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCAA

SEQ ID NO: 88 - protein, 229 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCAA

SEQ ID NO: 89 - protein, 107 aa, synthetic construct
RTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 90 - protein, 111 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 91 - protein, 218 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 92 - protein, 232 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 93 - protein, 108 aa, synthetic construct
ASTKGPSVFPLAPSSRSTSESTAALGCLVKDYFPEPVTVSWNSGALTSGVHTCPAVLQSSGLYSLSSVVTVPSSSLGTKTYTCNVDHKPSNTKVDKRVESKYGPPCAA

SEQ ID NO: 94 - protein, 229 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSRSTSESTAALGCLVKDYFPEPVTVSWNSGALTSGVHTCPAVLQSSGLYSLSSVVTVPSSSLGTKTYTCNVDHKPSNTKVDKRVESKYGPPCAA

SEQ ID NO: 95 - protein, 110 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 96 - protein, 231 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 97 - protein, 108 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 98 - protein, 229 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 99 - protein, 108 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHT

SEQ ID NO: 100 - protein, 107 aa, synthetic construct
RTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGEC

SEQ ID NO: 101 - protein, 218 aa, synthetic construct
DIQLTQSPSSLSASVGDRATITCRASESVDGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGEC

SEQ ID NO: 102 - protein, 229 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHT

SEQ ID NO: 103 - protein, 106 aa, synthetic construct
ASTKGPSVFPLAPSSRSTSESTAALGCLVKDYFPEPVTVSWNSGALTSGVHTCPAVLQSSGLYSLSSVVTVPSSSLGTKTYTCNVDHKPSNTKVDKRVESKYGPPC

SEQ ID NO: 104 - protein, 227 aa, synthetic construct
QVQLVQSGAEVKKPGASVKVSCKASRYTFTDYNLHWVRQAPGQGLEWMGFIYPYNGGTGYAQKFQGRVTMTVDTSTSTAYMELSSLRSEDTAVYYCARDHRYNEGVSFDYWGQGTTVTVSSASTKGPSVFPLAPSSRSTSESTAALGCLVKDYFPEPVTVSWNSGALTSGVHTCPAVLQSSGLYSLSSVVTVPSSSLGTKTYTCNVDHKPSNTKVDKRVESKYGPPC

SEQ ID NO: 105 - protein, 232 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 106 - protein, 232 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCAA

SEQ ID NO: 107 - protein, 218 aa, synthetic construct
DIQLTQSPSSLSASVGDRATITCRASESVSGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGES

SEQ ID NO: 108 - protein, 231 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 109 - protein, 231 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCP

SEQ ID NO: 110 - protein, 229 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 111 - protein, 229 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSCDKTHT

SEQ ID NO: 112 - protein, 218 aa, synthetic construct
DIQLTQSPSSLSASVGDRATITCRASESVSGFGNSFMNWYQQKPGKAPKLLIYLASNLESGVPSRFSGSGSGTDFTLTISSVQPEDFATYYCQQNNEDPYTFGQGTKLEIKRTVAAPSVFIFPPSDEQLKSGTASVVCLLNNFYPREAKVQWKVDNALQSGNSQECVTEQDSKDSTYSLSSTLTLSKADYEKHKVYACEVTHQGLSSPVTKSFNRGEC

SEQ ID NO: 113 - protein, 229 aa, synthetic construct
QVQLVQSGAEVKKPGASVKVSCKASRYTFTDYNLHWVRQAPGQGLEWMGFIYPYQGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHT

SEQ ID NO: 114 - protein, 229 aa, synthetic construct
QVQLVQSGAEVKKPGASVKVSCKASRYTFTDYNLHWVRQAPGQGLEWMGFIYPYAGGTGYAQKFQGRVTMTVDTSTSTAYMELSSLRSEDTAVYYCARDHRYNEGVSFDYWGQGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHT

SEQ ID NO: 115 - protein, 55 aa, synthetic construct
KNRKAKAKPVTRGAGAGGRQRGQNKERPPPVPNPDYEPIRKGQRDLYSGLNQRRI

SEQ ID NO: 116 - protein, 25 aa, synthetic construct
KTNIRSSTRDWKDHKFKWRKDPQDK

SEQ ID NO: 117 - protein, 21 aa, synthetic construct
MALPVTALLLPLALLLHAARP

SEQ ID NO: 118 - protein, 21 aa, synthetic construct
MALPVTALLLPLALLLHAARP

SEQ ID NO: 119 - protein, 22 aa, synthetic construct
MLLLVTSLLLCELPHPAFLLIP

SEQ ID NO: 120 - protein, 45 aa, synthetic construct
TTTPAPRPPTPAPTIASQPLSLRPEACRPAAGGAVHTRGLDFACD

SEQ ID NO: 121 - protein, 39 aa, synthetic construct
IEVMYPPPYLDNEKSNGTIIHVKGKHLCPSPLFPGPSKP

SEQ ID NO: 122 - protein, 12 aa, synthetic construct
ESKYGPPCPPCP

SEQ ID NO: 123 - protein, 5 aa, synthetic construct
GGGGS

SEQ ID NO: 124 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 125 - protein, 24 aa, synthetic construct
IYIWAPLAGTCGVLLLSLVITLYC

SEQ ID NO: 126 - protein, 27 aa, synthetic construct
FWVLVVVGGVLACYSLLVTVAFIIFWV

SEQ ID NO: 127 - protein, 42 aa, synthetic construct
KRGRKKLLYIFKQPFMRPVQTTQEEDGCSCRFPEEEEGGCEL

SEQ ID NO: 128 - protein, 41 aa, synthetic construct
RSKRSRLLHSDYMNMTPRRPGPTRKHYQPYAPPRDFAAYRS

SEQ ID NO: 129 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 132 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 133 - protein, 6 aa, synthetic construct
QDISKY

SEQ ID NO: 134 - skipped

SEQ ID NO: 135 - protein, 9 aa, synthetic construct
QQGNTLPYT

SEQ ID NO: 136 - protein, 18 aa, synthetic construct
GSTSGSGKPGSGEGSTKG

SEQ ID NO: 137 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 138 - protein, 8 aa, synthetic construct
GVSLPDYG

SEQ ID NO: 139 - protein, 7 aa, synthetic construct
IWGSETT

SEQ ID NO: 140 - protein, 14 aa, synthetic construct
AKHYYYGGSYAMDY

SEQ ID NO: 141 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 142 - protein, 15 aa, synthetic construct
GGGGSGGGGSGGGGS

SEQ ID NO: 143 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 144 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - DNA, 1383 bp, synthetic construct
  source 1..1383: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 146 - protein, 461 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - DNA, 1467 bp, synthetic construct
  source 1..1467: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 148 - protein, 489 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 245 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 150 - protein, 55 aa, synthetic construct
FVPVFLPAKPTTTPAPRPPTPAPTIASQPLSLRPEACRPAAGGAVHTRGLDFACD

SEQ ID NO: 151 - protein, 28 aa, synthetic construct
IYIWAPLAGTCGVLLLSLVITLYCNHRN

SEQ ID NO: 152 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 153 - protein, 106 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 154 - protein, 10 aa, synthetic construct
RASSSVNYMD

SEQ ID NO: 155 - protein, 7 aa, synthetic construct
ATSNLAS

SEQ ID NO: 156 - protein, 9 aa, synthetic construct
QQWSFNPPT

SEQ ID NO: 157 - protein, 122 aa, synthetic construct
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 158 - protein, 5 aa, synthetic construct
SYNMH

SEQ ID NO: 159 - protein, 17 aa, synthetic construct
AIYPGNGDTSYNQKFKG

SEQ ID NO: 160 - protein, 246 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 161 - protein, 111 aa, synthetic construct
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 162 - protein, 15 aa, synthetic construct
RASESVSVIGAHLIH

SEQ ID NO: 163 - protein, 7 aa, synthetic construct
LASNLET

SEQ ID NO: 164 - protein, 9 aa, synthetic construct
LQSRIFPRT

SEQ ID NO: 165 - protein, 117 aa, synthetic construct
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 166 - protein, 5 aa, synthetic construct
DYSIN

SEQ ID NO: 167 - protein, 17 aa, synthetic construct
WINTETREPAYAYDFRG

SEQ ID NO: 168 - protein, 8 aa, synthetic construct
DYSYAMDY

SEQ ID NO: 169 - protein, 246 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 170 - protein, 111 aa, synthetic construct
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 171 - protein, 15 aa, synthetic construct
RASESVTILGSHLIY

SEQ ID NO: 172 - protein, 7 aa, synthetic construct
LASNVQT

SEQ ID NO: 173 - protein, 9 aa, synthetic construct
LQSRTIPRT

SEQ ID NO: 174 - protein, 117 aa, synthetic construct
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 175 - protein, 5 aa, synthetic construct
HYSMN

SEQ ID NO: 176 - protein, 17 aa, synthetic construct
RINTESGVPIYADDFKG

SEQ ID NO: 177 - protein, 8 aa, synthetic construct
DYLYSLDF

SEQ ID NO: 178 - protein, 121 aa, synthetic construct
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 179 - protein, 8 aa, synthetic construct
GFTFSSYA

SEQ ID NO: 180 - protein, 8 aa, synthetic construct
ISGSGDYI

SEQ ID NO: 181 - protein, 14 aa, synthetic construct
AKEGTGANSSLADY

SEQ ID NO: 182 - protein, 243 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 183 - protein, 106 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 184 - protein, 6 aa, synthetic construct
QSISSY

SEQ ID NO: 185 - skipped

SEQ ID NO: 186 - protein, 8 aa, synthetic construct
QQKYDLLT

SEQ ID NO: 187 - protein, 119 aa, synthetic construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 188 - protein, 10 aa, synthetic construct
GGSISSSSYY

SEQ ID NO: 189 - protein, 7 aa, synthetic construct
ISYSGST

SEQ ID NO: 190 - protein, 11 aa, synthetic construct
ARDRGDTILDV

SEQ ID NO: 191 - protein, 23 aa, synthetic construct
MALPVTALLLPLALLLHAARPGS

SEQ ID NO: 192 - protein, 243 aa, synthetic construct
QVQLQQPGAELVKPGASVKLSCKASGYTITSYSLHWVKQRPGQGLEWIGEINPANGDHNFSEKFEIKATLTVDSSSNTAFMQLSRLTSEDSAVYYCTRLDDSRFHWYFDVWGAGTTVTVSSGGGGSGGGGSGGGGSQIVLTQSPALMSASPGEKVTMTCTASSSVSYMYWYQQKPRSSPKPWIFLTSNLASGVPARFSGRGSGTSFSLTISSMEAEDAATYYCQQWSGYPPITFGSGTKLEIK

SEQ ID NO: 193 - protein, 47 aa, synthetic construct
SGTTTPAPRPPTPAPTIASQPLSLRPEACRPAAGGAVHTRGLDFACD

SEQ ID NO: 194 - protein, 243 aa, synthetic construct
QIVLTQSPALMSASPGEKVTMTCTASSSVSYMYWYQQKPRSSPKPWIFLTSNLASGVPARFSGRGSGTSFSLTISSMEAEDAATYYCQQWSGYPPITFGSGTKLEIKGGGGSGGGGSGGGGSQVQLQQPGAELVKPGASVKLSCKASGYTITSYSLHWVKQRPGQGLEWIGEINPANGDHNFSEKFEIKATLTVDSSSNTAFMQLSRLTSEDSAVYYCTRLDDSRFHWYFDVWGAGTTVTVSS

SEQ ID NO: 195 - RNA, 1776 nt, synthetic construct
  source 1..1776: mol_type = other RNA
agcataaaagtcacatttgcttctgacatagttgtgttgactcacaaccccagaaacagacatcgaattcgctgccaccatggccctgcccgtgaccgccctcctgctgcccctggccctgctcctgcacgccgcccggcccgagatcgtgctgacccagagccccggcaccctgagcctgagccccggcgagcgggccaccctgagctgccgggccagccagagcgtgagcagcagttacctggcctggtaccagcagaagcccggccaggccccacggctgctgatctacggcgccagcagccgggccaccggcatccccgaccggttcagcggctccggcagcggcaccgacttcaccctgaccatcagccggctggagcccgaggacttcgccgtgtactactgccagcagtacggcagcagccggttcaccttcggccccggcaccaaggtggacatcaagggcagcaccagcggcagcggcaagcccggcagcggcgagggcagcaccaagggccaggtgcagctggtgcagagcggcgccgaggtgaagaagcccggcagcagcgtgaaggtgagctgcaaggacagcggcggcaccttcagcagctacgccatcagctgggtgcggcaggccccaggccagggcctggagtggatgggcggcatcatccccatcttcggcaccaccaactacgcccagcagttccagggccgggtgaccatcaccgccgacgagagcaccagcaccgcctacatggagctgagcagcctgcggagcgaggacaccgccgtctactactgcgcccgggaggccgtggccgccgactggctggacccctggggccagggcaccctggtgaccgtgagcagcatcgaggtgatgtacccaccaccctacctggacaacgagaaaagcaacggcaccatcatccacgtgaagggcaagcacctgtgccccagcccactgttccccggccccagcaagcccttctgggtgctggtcgtggtgggcggcgtgctggcctgctacagcctgctggtgaccgtggccttcatcatcttctgggtgcggagcaagcggagccggctgctgcacagcgactacatgaacatgaccccacggcggcccggccccacccggaagcactaccagccctacgccccaccccgggacttcgccgcctaccggagccgggtgaagttcagccggagcgccgacgccccagcctaccagcagggccagaaccagctctacaacgagctgaacctgggccggcgggaggagtacgacgtgctggacaagcggcggggccgggaccccgagatgggcggcaagccccggcggaagaacccacaggagggcctctacaatgagctgcagaaggacaagatggccgaggcctacagcgagatcggcatgaagggcgagcggcggaggggcaagggccacgacggcctgtaccagggcctgagcaccgccaccaaggacacctacgacgccctgcacatgcaggccctgccaccccggtgataaaggatccgagactactgttatttgaccaatgaattgacttctaataaaatctagtggtgatgcagttcagacttcaaatagtggcaaataaaaagtgctatttgtgatggtttgcttctgtgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 196 - RNA, 1899 nt, synthetic construct
  source 1..1899: mol_type = other RNA
agcataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcgaattcgctgccaccatggccctgcctgtgacggctctcctgctgcctctggcgttgctccttcatgcagcccgcccggaaatagtgcttacccagagtccaggtacactcagcctgagtccgggagagcgggctacgctgtcatgccgggctagccagagcgtgtcatcttcctacttggcttggtaccagcaaaagcctggccaggcaccgagacttttgatctatggggcgtcatcacgcgccactgggattcccgaccgcttcagcggcagcggttctggaactgactttactctgacaatatcccggttggagcctgaagattttgccgtctattattgtcagcaatatggcagtagccgctttacctttggtcctggcaccaaagtcgatattaaaggaagtacgagcggtagcggtaaaccggggagtggggaaggatcaacaaaagggcaagttcaattggtgcagagtggtgccgaggtcaagaagcctggcagtagcgtaaaggtttcatgtaaggacagtggcggcacgttcagctcttatgcgataagctgggttcgccaagcacctggccaggggctcgaatggatgggggggatcatacccatttttggtactactaattatgcgcaacagtttcaaggacgggtcacaattaccgcagacgagtccactagtacagcgtacatggaactctcaagcttgcgctctgaggacaccgccgtttattattgcgcaagagaggcggtcgccgccgactggctcgatccatggggccagggcactcttgtgacagtctcaagtatcgaagtaatgtaccctcccccgtacttggataacgaaaagagcaatgggactatcatccatgtgaagggaaaacacctttgcccgtcaccactttttcccggtccctccaaaccgttttgggttctggtagtggtgggaggtgttcttgcatgttactctctcctggttactgtcgcctttataatattttgggtgagatctaagcgatctcgattgttgcatagtgattacatgaatatgacgccgcgaagaccggggccaacgcgaaagcattaccaaccctatgcgcctccgagagactttgcagcatatcggagccgcgtcaaattctctcggtctgctgatgcgccggcgtaccagcaggggcagaatcaattgtataacgagctgaatctcggccgccgagaggagtacgacgttctggacaagcgacgaggccgcgatccagaaatggggggaaaaccaaggagaaagaatccgcaggagggcctctacaacgagcttcagaaggataaaatggcagaggcttactctgaaattggcatgaaaggggagagacgacgcggcaaaggccacgatgggctctatcaagggctctcaacggcgaccaaggatacatacgatgccctccatatgcaggccttgcccccacggtgataaaggatccagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctgtacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 197 - protein, 5 aa, synthetic construct
LPXTG

SEQ ID NO: 198 - protein, 481 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 199 - protein, 502 aa, synthetic construct
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 200 - protein, 464 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 201 - protein, 485 aa, synthetic construct
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 202 - protein, 13 aa, synthetic construct
SNYYGSSYWFFDV

SEQ ID NO: 203 - protein, 246 aa, synthetic construct
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGGGSGGGSGGGGSSEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 204 - protein, 18 aa, synthetic construct
GSTSGGGSGGGSGGGGSS

SEQ ID NO: 205 - protein, 249 aa, synthetic construct
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSS

SEQ ID NO: 206 - protein, 112 aa, synthetic construct
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIK

SEQ ID NO: 207 - protein, 16 aa, synthetic construct
RSSQSLVSRDGNTYLS

SEQ ID NO: 208 - protein, 7 aa, synthetic construct
KISNRFS

SEQ ID NO: 209 - protein, 9 aa, synthetic construct
MQATQFPLT

SEQ ID NO: 210 - protein, 122 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSS

SEQ ID NO: 211 - protein, 5 aa, synthetic construct
SYWIG

SEQ ID NO: 212 - protein, 17 aa, synthetic construct
IIYPGDSDTRYSPSFQG

SEQ ID NO: 213 - protein, 13 aa, synthetic construct
QGDFWSGYGGMDV

SEQ ID NO: 214 - protein, 468 aa, synthetic construct
DIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 215 - protein, 489 aa, synthetic construct
MALPVTALLLPLALLLHAARPDIVMTQTPHSSPVTLGQPASISCRSSQSLVSRDGNTYLSWLQQRPGQPPRLLIYKISNRFSGVPNRFSGSGAGTDFTLKISRVKAEDVGVYYCMQATQFPLTFGQGTRLEIKGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQGDFWSGYGGMDVWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 216 - protein, 450 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 217 - protein, 213 aa, synthetic construct
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 121 aa, synthetic construct
QVQLVESGGGVVQPGRSLRLSCAASGFTFSDFGMNWVRQAPGKGLEWVALIYYDGSNKFYADSVKGRFTISRDNSKNTLYLQMNSLRAEDTAVYYCAKPHYDGYYHFFDSWGQGTLVTVSS

SEQ ID NO: 219 - protein, 106 aa, synthetic construct
DIQMTQSPSSLSASVGDRVTITCKGSQDINNYLAWYQQKPGKAPKLLIYNTDILHTGVPSRFSGSGSGTDFTFTISSLQPEDIATYYCYQYNNGYTFGQGTKVEIK

SEQ ID NO: 220 - protein, 8 aa, synthetic construct
GFTFSDFG

SEQ ID NO: 221 - protein, 8 aa, synthetic construct
IYYDGSNK

SEQ ID NO: 222 - protein, 14 aa, synthetic construct
AKPHYDGYYHFFDS

SEQ ID NO: 223 - protein, 11 aa, synthetic construct
KGSQDINNYLA

SEQ ID NO: 224 - protein, 7 aa, synthetic construct
NTDILHT

SEQ ID NO: 225 - protein, 8 aa, synthetic construct
YQYNNGYT

SEQ ID NO: 226 - protein, 42 aa, synthetic construct
RGAAASPTFLLTLSDFRRENEGYYFCSALSNSIMYFSHFVPV

SEQ ID NO: 227 - protein, 15 aa, synthetic construct
RASESVSGFGNSFMN

SEQ ID NO: 228 - protein, 15 aa, synthetic construct
RASESVAGFGNSFMN

SEQ ID NO: 229 - protein, 107 aa, synthetic construct
DVQITQSPSSLSASVGDRVTITCRTSRSISQYLAWYQQKPGKVPKLLIYSGSTLQSGVPSRFSGSGSGTDFTLTISSLQPEDVATYYCQQHNENPLTFGGGTKVEIK

SEQ ID NO: 230 - protein, 118 aa, synthetic construct
EVQLVESGGGLVQPGGSLRLSCAASGFNIKDTYIHFVRQAPGKGLEWIGRIDPANDNTLYASKFQGKATISADTSKNTAYLQMNSLRAEDTAVYYCGRGYGYYVFDHWGQGTLVTVSS

SEQ ID NO: 231 - protein, 214 aa, synthetic construct
DVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 232 - protein, 360 aa, synthetic construct
EVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMT